(19) United States
(12) Patent Application Publication
(10) Pub. No.: US 2022/0332799 A1
(43) Pub. Date: Oct. 20, 2022

(54) HERV INHIBITORS FOR USE IN TREATING TAUOPATHIES

(71) Applicant: Deutsches Zentrum Für Neurodegenerative Erkrankungen E.V. (DZNE), Bonn (DE)

(72) Inventors: Ina Maja Vorberg, Bonn (DE); Shu Liu, Bonn (DE); Philip Denner, Bonn (DE); Stefan Lichtenthaler, Graefelfing (DE); Stephan Mueller, Munchen (DE)

(21) Appl. No.: 17/640,119

(22) PCT Filed: Sep. 4, 2020
(86) PCT No.: PCT/EP2020/074809

§ 371 Date: Mar. 3, 2022

(30) Foreign Application Priority Data
Sep. 4, 2019 (EP) 19195304.1

(51) Int. Cl. C07K 16/10; C12N 15/113; G01N 33/569; C12Q 1/6883

(52) U.S. Cl. CPC C07K 16/1036; C12N 15/1132; G01N 33/56983; C12Q 1/6883; G01N 2333/15; G01N 2800/2821; G01N 2800/2835; C12N 2310/14

(57) ABSTRACT

The present invention relates to inhibitors of HERV proteins comprising HERV Env and/or Gag, or fragments thereof, for use in treating a tauopathy, Parkinson's disease, or ALS (Amyothrophic Lateral Sclerosis). The present invention further relates to inhibitors of receptors which bind HERV Env proteins for use in treating a tauopathy, Parkinson's disease, or ALS (Amyothrophic Lateral Sclerosis). The present invention further relates to molecules binding to HERV Env and/or Gag, or fragments thereof, or to a nucleic acid molecule encoding said HERV Env and/or Gag, or fragments thereof, for use in diagnosing a tauopathy, Parkinson's disease, or ALS.

The present invention relates to inhibitors of endogenous retrovirus (ERV) components, including Env and Gag proteins or fragments thereof, for use in treating a tauopathy, Parkinson's disease, or ALS (Amyothrophic Lateral Sclerosis). The present invention further relates to inhibitors of receptors which bind HERV Env proteins for use in treating a tauopathy, Parkinson's disease, or ALS (Amyothrophic Lateral Sclerosis). The present invention further relates to molecules binding to HERV Env and/or Gag proteins, or fragments thereof, or to a nucleic acid molecule encoding said HERV Env and/or Gag proteins, or fragments thereof, for use in diagnosing a tauopathy, Parkinson's disease, or ALS.

The deposition of intracellular fibrillar phosphorylated Tau in the central nervous system is a pathologic hallmark of a heterogeneous group of at least 20 neurodegenerative diseases, collectively termed tauopathies. These include Alzheimer's Disease (AD), Cortical Basal Degeneration (CBD), Progressive Supranuclear Palsy (PSP), Pick's Disease (PiD) and Frontotemporal Dementia with Parkinsonism related to chromosome 17 (FTDP-17) and others (Williams, Intern Med J (2006), 36: 652-660). AD, the most common tauopathy, is characterized by the presence of senile plaques composed of amyloid-8 peptide, as well as by the formation of intraneuronal tangles composed of phosphorylated Tau. With a prevalence of approx. 46.8 million AD patients worldwide in 2015 and an estimated number of 131.5 million cases by 2050 due to demographic changes, AD and other tauopathies pose a tremendous social and economic burden to society. Unfortunately, therapeutic strategies are lacking that could halt the disease. Recent approaches that aimed at reducing amyloid-8 levels in AD have been unsuccessful, and clinical trials are gradually centering on Tau as the major drug target Congdon et al., Nat Rev Neurol (2018), 14: 399-415). Tau is a microtubule-stabilizing protein involved in intracellular trafficking (Vershinin et al., PNAS (2007), 87-92). Aberrant Tau aggregation is believed to start locally in specific brain regions, from where Tau pathology spreads to other regions of the brain (Braak et al., Acta Neuropathol (1991), 82: 239-259; Braak et al., Neurobiol Aging (1997), 18: 351-357). Misfolded Tau has been implicated in neuronal loss and disease severity (Nelson et al., J Neuropathol Exp Neurol (2007), 66: 1136-1146). Tau is subject to several posttranslational modifications such as hyperphosphorylation, acetylation or truncation, which can contribute to aberrant Tau polymerization into small oligomers and filaments. Tau aggregates associated with different tauopathies differ in their anatomical distribution, cell tropism, isoform composition and structure (Kovacs, Neuropathol Appl Neurobiol (2015): 41: 3-23).

Several lines of evidence suggest that transcellular spreading of distinct Tau aggregates may underlie the diversity and progression of tauopathies (Goedert, Science (2015), 349: 1255555). For example, in the case of sporadic AD, Tau aggregate spreading has been proposed to cause disease progression from the locus coeruleus to the transentorhinal cortex and finally to hippocampus and surrounding brain regions (Braak et al., Acta Neuropathol (2011), 121: 589-595). The mechanism of intercellular Tau aggregate transmission is only insufficiently understood. It is possible that so-far unidentified factors contribute to the sequential pathologic Tau spreading pattern and clinical diversity of distinct tauopathies (Braak et al., Neurobiol Aging (1995), 16: 271-278). Increasing evidence argues that extracellular vesicles (EVs) play a prominent role in disseminating aberrant Tau species to neighboring cells. Insoluble Tau was detected in EVs isolated either from cell culture or from patient CSF (Asai et al., Nat Neuroscie (2015), 18: 1584-1593; Saman et al., J Biol Chem (2012), 287: 3842-3849). Although EV-associated Tau represented only a small fraction of released Tau, it showed pathology-dependent phosphorylation at Thr-181 in CSF of AD patients early in disease (Saman, loc. cit.; Wang et al., Mol Neurodegener (1017), 12: 5; Frost et al., J Biol Chem (2009), 284: 12845-12852). Importantly, both pharmacological inhibition of EV release and disruption of exosomal membranes by sonication successfully halted Tau propagation in recipient cells or in a Tau transgenic mouse model, highlighting the important role of EVs in spreading of Tau pathology (Sama, loc. cit.; Wang, loc. cit.). So far it is unclear how exactly Tau is sorted into EVs and how these EVs target recipient cells.

As Tau pathology correlates better with the degree of cognitive decline in AD patients than with amyloid-β pathology, greater clinical efficacy may be achieved with Tau-based-therapeutic approaches (Nelson, loc. cit.). Current therapeutic interventions aim at reducing Tau expression, the formation of pathogenic Tau seeds or clearing pathogenic Tau. Strategies include reducing Tau expression by antisense oligonucelotides (Guo et al., Acta Neuropathol (2017), 133: 665-704), increasing cellular Tau degradation or reducing Tau posttranslational modifications, using phosphatase activators, kinase inhibitors, acetylation and deglycosylation inhibitors (Congdon, loc. cit.). Tau aggregation inhibitors such as a methylene blue derivative or curcumin so far had limited effects in phase II/III clinical trials (Congdon, loc. cit.). Likewise, a phase III clinical trial with microtubule stabilizer Davunetide in PSP patients did not result in cognitive improvements (Boxer et al., Lancet Neurol (2014), 13: 676-685).

An alternative approach to treating tauopathies is to halt disease progression by targeting the spreading of pathologic Tau throughout the brain. Antibodies against different forms of phosphorylated, oligomeric or misfolded Tau have been shown to modulate Tau pathology in mouse models. For example, passive immunotherapies with Tau specific antibodies were shown to slow progression of Tau pathology in transgenic mice (d'Abramo et al., PLoS One (2013), 8: e62402; Castillo-Carranza et al., J Neurosci (2014), 34: 4260-4272; Yanamandra et al., Neuron (2013), 80: 402-414). However, little is known how anti-Tau antibodies can reduce intracellular neurofibrillary tangle burden (Congdon, loc. cit.). It has been proposed that Tau antibodies achieve therapeutic effects by capturing extracellular Tau and thereby preventing the spreading of Tau pathology. Alternatively, antibodies might also enter neurons and target cytosolic Tau. Several antibodies used in these studies are currently undergoing early clinical trials. A difficulty with Tau-directed antibodies is the insufficient knowledge about the specific type of Tau that causes toxicity and/or is capable of transcellular spreading. Moreover, Tau efficiently spreads via EVs and is thus most likely shielded from extracellular antibody recognition. Consequently, antibodies targeting Tau directly might not be able to terminate the progression of Tau pathology. One way to prevent EV mediated spreading of Tau is to impair efficient transmission of Tau aggregates by targeting EV uptake. The advantage of such approach is that it could impair Tau spreading regardless of the Tau species packaged into EVs. However, very little is known how EVs dock onto target cells and release their cargo into the cytosol.

The gold standard for the diagnosis of tauopathies is still autopsy. Three major tauopathies, PSP, CBD and Pick's disease, are classified based on their characterized neuropathological characteristics. For example, PSP exhibits Tau-positive glial inclusions in the form of “tufted astrocytes” in gray matter; CBD is characterized by typical Tau-positive ballooned neurons and astrocytic deposits; Pick's disease contains Tau-positive “Pick bodies” in neurons. The autopsy diagnosis correlates not well with the syndrome diagnosis. Although CBD and PSP are most commonly associated with CBD clinical syndrome and PSP clinical syndrome, respectively, these syndromes are also shared by other tauopathies. For example, only 50% of cases with CBD clinical syndrome are CBD cases, the rest of cases are diagnosed with AD, PSP, Pick's disease and FTLD-TDP at autopsy (Coughlin et al., Curr Neurol Neurosci Rep (2017), 17: 72). Thus, for specific therapy, it is essential to establish antemortem diagnosis for classification of these tauopathies.

Several different methods have been developed for antemortem diagnosis, including neuroimaging, molecular imaging, and fluid biomarkers. Neuroimaging techniques using MRI proved useful in the diagnosis of PSP with 72.7% specificity but not for other tauopathies (Coughlin, loc. cit.). Molecular imaging, Tau positron emission tomography (PET), has been used in some clinical studies to distinguish AD from control patients. However, the second-generation of Tau specific PET ligands showed very weak binding to pure 3R- or 4R-Tau pathology in post mortem tissue in PSP, CBD and Pick's disease. Moreover, the observed potential off-target binding in susceptible brain regions in PSP and CBD, limits its use in diagnosis of tauopathies that represent with mixtures of 3R- and 4R-Tau pathology (Scholl et al., Mol Cell Neurosci (2018), doi: 10.1016/j.mcn.2018.12.001).

Fluid biomarkers for tauopathies include CSF total Tau and phosphorylated Tau. Total Tau in CSF has been postulated to correlate with neurodegenerative changes in AD patients. However, the increase is not specific for AD and also occurs in other neurodegenerative diseases, such as Creutzfeldt-Jakob disease and brain injury. Phosphorylated Tau in CSF is significantly increased in AD but not in other tauopathies, like PSP. Plasma Tau correlates poorly with CSF Tau levels, which limits its use in the diagnosis of AD (Scholl, loc. cit.). Thus, while determination of phosphorylated Tau levels in CSF in combination with Tau PET imaging could have implications for clinical diagnosis of AD, no suitable ante mortem methods are available for identification of other tauopathies.

Accordingly, the technical problem underlying the present invention was to comply with the disadvantages set out above.

The present invention addresses the technical problem by providing compounds for use in treating tauopathy, Parkinson's disease and ALS as set forth herein below and as defined by the claims.

Accordingly, the present invention relates to an inhibitor of HERV proteins comprising HERV Env and Gag, or a fragment thereof (i.e. fragment of HERV Env and/or HERV Gag protein(s)), for use in treating tauopathies (e.g., Alzheimer's Disease (AD), Argyrophilic Grain Disease (AGD), Cortical Basal Degeneration (CBD), Progressive Supranuclear Palsy (PSP), Pick's Disease (PiD), and Frontotemporal Dementia with Parkinsonism related to chromosome 17 (FTDP-17)), Parkinson's disease, or ALS.

As surprisingly found in context with the present invention, human endogenous retrovirus (HERV) Env proteins (e.g., from HERV species HERV-W (Syncytin 1), HERV-K, HERV-H, HERV-T, HERV-FRD, HERV-F(c)1, HERV-F(c)2, HERV-E, HERV-P(b1), HERV-VR(b), and HERV-MER34) are upregulated in tauopathies. This applies mutatis mutandis also for HERV Gag proteins given that env and gag mRNA is transcribed together. As has been shown in context with the present invention, there is a correlation between increased HERV Env expression and accelerated intercellular Tau aggregate spreading. Particularly, in accordance with the present invention, ERV Env proteins serve as ligands that mediate the interaction of cellular membranes, resulting in increased transfer of protein aggregate seeds from one cell to another. As shown herein, interruptions of involved ligand-receptor interactions significantly impair the spreading of diverse proteopathic seeds, including Tau aggregates. Furthermore, retroviruses generally depend on proteolytic maturation of the structural protein Gag. Thus, in accordance with the present invention, Env activity is regulated by the maturation status of Gag (cf. Johnson, Nature Reviews Microbiol (2019), 17: 355-370). Without being bound by theory, the maturation of Gag changes the conformation of Gag in the particle and renders Env fusogenic. In other words, again without being bound by theory, in accordance with the present invention, (maturating) Gag activates Env. Accordingly, in context with the present invention, it has surprisingly been found that inhibition of HERV proteins (e.g., inhibition of the maturation or expression of ERV Env and Gag proteins, and/or binding of said ERV Env protein to a receptor; or inhibiting a HERV Env protein receptor from binding the HERV Env protein) is useful for treating tauopathies (e.g., Alzheimer's Disease (AD), Argyrophilic Grain Disease (AGD), Cortical Basal Degeneration (CBD), Progressive Supranuclear Palsy (PSP), Pick's Disease (PiD), and Frontotemporal Dementia with Parkinsonism related to chromosome 17 (FTDP-17)). Without being bound by theory, by inhibiting HERV proteins (Env or Gag, or fragments thereof) as described and provided herein, spreading of pathogenic Tau seeds is inhibited or decreased. Also, again without being bound by theory, protein aggregates and the subsequent aggregation of proteins of the same kind in recipient cells is thus inhibited or decreased. Accordingly, in accordance with the present invention, inhibition of HERV proteins (Env or Gag, or fragments thereof) as described and provided herein can also be used for treating associated disorders undelaying such mechanisms such as Parkinson's disease and ALS (Amyothrophic Lateral Sclerosis).

As used herein, the term “HERV Env and/or Gag proteins” comprises all types and variants of HERV Env and/or Gag proteins, respectively, including—but not limited to—those Env and Gag proteins of the HERV species HERV-W (Syncytin 1), HER-K, HERV-H, HERV-T, HERV-FRD, HERV-F(c)1, HERV-F(c)2, HERV-E, HERV-P(b1), HERV-R, HERV-R(b), HERV-V and HERV-MER34. In one embodiment of the present invention, specifically those HERV Env and Gag proteins are meant which have been shown to be increased, upregulated or overexpressed in one or more tauopathies, Parkinson's disease, and/or ALS (Amyothrophic Lateral Sclerosis).

As shown in context with the present invention, HERV transcripts are upregulated in different tauopathies. For example, as has been found in context with the present invention, HERV-W is upregulated in AD patients, while HERV-FRD, —H and -R(b) are associated with CBD disease, and HERV-K and -F(c)1 are increased in PSP patients. Accordingly, in a specific embodiment of the present invention, the HERV Env and/or Gag protein is selected from the group consisting of Env and/or Gag of HERV-W, -FRD, —H, -R(b), —K, and -F(c)1, preferably HERV-W and HERV-K. Accordingly, in one embodiment of the present invention, an inhibitor of a specific HERV Env and/or Gag protein (or fragments thereof) may be particularly useful in treating a specific tauopathy which is associated with upregulation/increase/overexpression of the respective HERV Env protein. For example, in accordance with the present invention, an inhibitor of Env and/or Gag (or fragments thereof) from HERV-W may be used for treating AD, an inhibitor of Env and/or Gag (or fragments thereof) from HERV-FRD, -H and/or -R(b) may be used for treating CBD, and an inhibitor of Env and/or Gag (or fragments thereof) from HERV-K and/or -F(c)1 may be used for treating PSP.

The HERV Env and Gag proteins from HERV-W (Syncytin 1), HER-K, HERV-H, HERV-T, HERV-FRD, HERV-F(c)1, HERV-F(c)2, HERV-E, HERV-P(b1), HERV-R, HERV-R(b), HERV-V and HERV-MER34 are well known in the art.

For example, in accordance with the present invention, HERV Env proteins (or fragments thereof) as used and described herein may have amino acid sequences encoded by nucleotide sequences being at least about 85%, at least about 90%, at least about 91%, at least about 92%, at least about 93%, at least about 94%, at least about 95%, at least about 96%, at least about 97%, at least about 98%, at least about 99%, at least about 99.5%, at least about 99.8%, or 100% identical to those nucleotide sequences shown in SEQ ID NOs. 1 to 13 (particularly Env from HERV-H: SEQ ID NO: 1; HERV-K: SEQ ID NO: 2; HERV-T: SEQ ID NO: 3; HERV-W: SEQ ID NO: 4; HERV-FRD: SEQ ID NO 5; HERV-R: SEQ ID NO: 6; HERV-R(b): SEQ ID NO: 7; HERV-F(c)2: SEQ ID NO: 8; HERV-F(c)1: SEQ ID NO: 9; HERV-E: SEQ ID NO: 10; HERV-P(b1): SEQ ID NO: 11; HERV-V: SEQ ID NO: 12; HERV-MER34: SEQ ID NO: 13). That is, for example, in accordance with the present invention, HERV-H Env as used herein may have an amino acid sequence encoded by a nucleotide sequence being at least about 85%, at least about 90%, at least about 91%, at least about 92%, at least about 93%, at least about 94%, at least about 95%, at least about 96%, at least about 97%, at least about 98%, at least about 99%, at least about 99.5%, at least about 99.8%, or 100% identical to the nucleotide sequence shown in SEQ ID NO: 1, and HERV-H Env as used herein may have an amino acid sequence being at least about 85%, at least about 90%, at least about 91%, at least about 92%, at least about 93%, at least about 94%, at least about 95%, at least about 96%, at least about 97%, at least about 98%, at least about 99%, at least about 99.5%, at least about 99.8%, or 100% identical to the amino acid sequence shown in SEQ ID NO: 132 to 137, HERV-K Env as used herein may have an amino acid sequence encoded by a nucleotide sequence being at least about 85%, at least about 90%, at least about 91%, at least about 92%, at least about 93%, at least about 94%, at least about 95%, at least about 96%, at least about 97%, at least about 98%, at least about 99%, at least about 99.5%, at least about 99.8%, or 100% identical to the nucleotide sequence shown in SEQ ID NO: 2, and HERV-K Env as used herein may have an amino acid sequence being at least about 85%, at least about 90%, at least about 91%, at least about 92%, at least about 93%, at least about 94%, at least about 95%, at least about 96%, at least about 97%, at least about 98%, at least about 99%, at least about 99.5%, at least about 99.8%, or 100% identical to the amino acid sequence shown in SEQ ID NO: 101 to 128, HERV-T Env as used herein may have an amino acid sequence encoded by a nucleotide sequence being at least about 85%, at least about 90%, at least about 91%, at least about 92%, at least about 93%, at least about 94%, at least about 95%, at least about 96%, at least about 97%, at least about 98%, at least about 99%, at least about 99.5%, at least about 99.8%, or 100% identical to the nucleotide sequence shown in SEQ ID NO: 3, HERV-W Env as used herein may have an amino acid sequence encoded by a nucleotide sequence being at least about 85%, at least about 90%, at least about 91%, at least about 92%, at least about 93%, at least about 94%, at least about 95%, at least about 96%, at least about 97%, at least about 98%, at least about 99%, at least about 99.5%, at least about 99.8%, or 100% identical to the nucleotide sequence shown in SEQ ID NO: 4, and HERV-W Env as used herein may have an amino acid sequence being at least about 85%, at least about 90%, at least about 91%, at least about 92%, at least about 93%, at least about 94%, at least about 95%, at least about 96%, at least about 97%, at least about 98%, at least about 99%, at least about 99.5%, at least about 99.8%, or 100% identical to the amino acid sequence shown in SEQ ID NO: 129 to 131, HERV-R Env as used herein may have an amino acid sequence being at least about 85%, at least about 90%, at least about 91%, at least about 92%, at least about 93%, at least about 94%, at least about 95%, at least about 96%, at least about 97%, at least about 98%, at least about 99%, at least about 99.5%, at least about 99.8%, or 100% identical to the amino acid sequence shown in SEQ ID NO: 138 to 139, and so forth. In this context, preferred are those nucleotide sequences encoding a HERV Env protein as used and described herein which differ from the respective nucleotide sequences of SEQ ID NOs. 1 to 13, respectively, in a manner that any nucleotide difference results only in a conservative or highly conservative amino acid substitution (compared to the amino acid sequences encoded by the nucleotide sequence of the respective SEQ ID NO.), or is silent (i.e. does not translate into an amino acid substitution compared to the amino acid sequences encoded by the nucleotide sequence of the respective SEQ ID NO.).

As a further example, in accordance with the present invention, HERV Gag proteins (or fragments thereof) as used and described herein may have amino acid sequences encoded by nucleotide sequences being at least about 85%, at least about 90%, at least about 91%, at least about 92%, at least about 93%, at least about 94%, at least about 95%, at least about 96%, at least about 97%, at least about 98%, at least about 99%, at least about 99.5%, at least about 99.8%, or 100% identical to those nucleotide sequences shown in SEQ ID NOs. 14 to 21 (particularly Gag from HERV-H: SEQ ID NO: 14; HERV-K (consensus): SEQ ID NO: 15; HERV-K (orico, codon optimized): SEQ ID NO: 16; HERV-T: SEQ ID NO: 17; HERV-W: SEQ ID NO: 18; HERV-R: SEQ ID NO: 19; HERV-E: SEQ ID NO: 20; HERV-V: SEQ ID NO: 21), or have amino acid sequences being at least about 85%, at least about 90%, at least about 91%, at least about 92%, at least about 93%, at least about 94%, at least about 95%, at least about 96%, at least about 97%, at least about 98%, at least about 99%, at least about 99.5%, at least about 99.8%, or 100% similar or (preferably) identical to those amino acid sequences shown in SEQ ID NOs. 22 (Gag HERV-F(c)2) to 23 (Gag HERV-F(c)2). That is, for example, in accordance with the present invention, HERV-H Gag as used herein may have an amino acid sequence encoded by a nucleotide sequence being at least about 85%, at least about 90%, at least about 91%, at least about 92%, at least about 93%, at least about 94%, at least about 95%, at least about 96%, at least about 97%, at least about 98%, at least about 99%, at least about 99.5%, at least about 99.8%, or 100% identical to the nucleotide sequence shown in SEQ ID NO: 14, HERV-K (consensus) Gag as used herein may have an amino acid sequence encoded by a nucleotide sequence being at least about 85%, at least about 90%, at least about 91%, at least about 92%, at least about 93%, at least about 94%, at least about 95%, at least about 96%, at least about 97%, at least about 98%, at least about 99%, at least about 99.5%, at least about 99.8%, or 100% identical to the nucleotide sequence shown in SEQ ID NO: 15, HERV-T Gag as used herein may have an amino acid sequence encoded by a nucleotide sequence being at least about 85%, at least about 90%, at least about 91%, at least about 92%, at least about 93%, at least about 94%, at least about 95%, at least about 96%, at least about 97%, at least about 98%, at least about 99%, at least about 99.5%, at least about 99.8%, or 100% identical to the nucleotide sequence shown in SEQ ID NO: 17, HERV-W Gag as used herein may have an amino acid sequence encoded by a nucleotide sequence being at least about 85%, at least about 90%, at least about 91%, at least about 92%, at least about 93%, at least about 94%, at least about 95%, at least about 96%, at least about 97%, at least about 98%, at least about 99%, at least about 99.5%, at least about 99.8%, or 100% identical to the nucleotide sequence shown in SEQ ID NO: 18, and so forth. In this context, preferred are those nucleotide sequences encoding a HERV Gag protein as used and described herein which differ from the respective nucleotide sequences of SEQ ID NOs. 14 to 21, respectively, in a manner that any nucleotide difference results only in a conservative or highly conservative amino acid substitution (compared to the amino acid sequences encoded by the nucleotide sequence of the respective SEQ ID NO.), or is silent (i.e. does not translate into an amino acid substitution compared to the amino acid sequences encoded by the nucleotide sequence of the respective SEQ ID NO.). Likewise, preferred are those HERV Gag proteins as used and described herein having amino acid sequences which differ from the respective amino acid sequences of SEQ ID NOs. 22 to 23, respectively, in a manner that only in a conservative or highly conservative amino acid substitutions/insertions/additions/deletions appear (compared to the amino acid sequences of the respective SEQ ID NO.).

Likewise, as used herein and in accordance with the present invention, HERV Env proteins as used and described herein may have amino acid sequences being at least about 85%, at least about 90%, at least about 91%, at least about 92%, at least about 93%, at least about 94%, at least about 95%, at least about 96%, at least about 97%, at least about 98%, at least about 99%, at least about 99.5%, at least about 99.8%, or 100% similar or identical to those amino acid sequences encoded by nucleotide sequences shown in SEQ ID NOs. 1 to 13 (particularly Envs from HERV-H: SEQ ID NO: 1; HERV-K: SEQ ID NO: 2; HERV-T: SEQ ID NO: 3; HERV-W: SEQ ID NO: 4; HERV-FRD: SEQ ID NO 5; HERV-R: SEQ ID NO: 6; HERV-R(b): SEQ ID NO: 7; HERV-F(c)2: SEQ ID NO: 8; HERV-F(c)1: SEQ ID NO: 9; HERV-E: SEQ ID NO: 10; HERV-P(b1): SEQ ID NO: 11; HERV-V: SEQ ID NO: 12; HERV-MER34: SEQ ID NO: 13). That is, for example, in accordance with the present invention, HERV-H Env as used herein may have an amino acid sequence being at least about 85%, at least about 90%, at least about 91%, at least about 92%, at least about 93%, at least about 94%, at least about 95%, at least about 96%, at least about 97%, at least about 98%, at least about 99%, at least about 99.5%, at least about 99.8%, or 100% similar or identical to the amino acid sequence encoded by the nucleotide sequence shown in SEQ ID NO: 1, HERV-K Env as used herein may have an amino acid sequence being at least about 85%, at least about 90%, at least about 91%, at least about 92%, at least about 93%, at least about 94%, at least about 95%, at least about 96%, at least about 97%, at least about 98%, at least about 99%, at least about 99.5%, at least about 99.8%, or 100% similar or identical to the amino acid sequence encoded by the nucleotide sequence shown in SEQ ID NO: 2, HERV-T Env as used herein may have an amino acid sequence being at least about 85%, at least about 90%, at least about 91%, at least about 92%, at least about 93%, at least about 94%, at least about 95%, at least about 96%, at least about 97%, at least about 98%, at least about 99%, at least about 99.5%, at least about 99.8%, or 100% similar or identical to the amino acid sequence encoded by the nucleotide sequence shown in SEQ ID NO: 3, HERV-W Env as used herein may have an amino acid sequence being at least about 85%, at least about 90%, at least about 91%, at least about 92%, at least about 93%, at least about 94%, at least about 95%, at least about 96%, at least about 97%, at least about 98%, at least about 99%, at least about 99.5%, at least about 99.8%, or 100% similar or identical to the amino acid sequence encoded by the nucleotide sequence shown in SEQ ID NO: 4, and so forth.

Likewise, as used herein and in accordance with the present invention, HERV Gag proteins as used and described herein may have amino acid sequences being at least about 85%, at least about 90%, at least about 91%, at least about 92%, at least about 93%, at least about 94%, at least about 95%, at least about 96%, at least about 97%, at least about 98%, at least about 99%, at least about 99.5%, at least about 99.8%, or 100% similar or identical to those amino acid sequences encoded by nucleotide sequences shown in SEQ ID NOs. 14 to 21 (particularly Gag from HERV-H: SEQ ID NO: 14; HERV-K (consensus): SEQ ID NO: 15; HERV-K (orico, codon optimized): SEQ ID NO: 16; HERV-T: SEQ ID NO: 17; HERV-W: SEQ ID NO: 18; HERV-R: SEQ ID NO: 19; HERV-E: SEQ ID NO: 20; HERV-V: SEQ ID NO: 21). That is, for example, in accordance with the present invention, HERV-H Gag as used herein may have an amino acid sequence being at least about 85%, at least about 90%, at least about 91%, at least about 92%, at least about 93%, at least about 94%, at least about 95%, at least about 96%, at least about 97%, at least about 98%, at least about 99%, at least about 99.5%, at least about 99.8%, or 100% similar or identical to the amino acid sequence encoded by the nucleotide sequence shown in SEQ ID NO: 14, HERV-K (consensus) Gag as used herein may have an amino acid sequence being at least about 85%, at least about 90%, at least about 91%, at least about 92%, at least about 93%, at least about 94%, at least about 95%, at least about 96%, at least about 97%, at least about 98%, at least about 99%, at least about 99.5%, at least about 99.8%, or 100% similar or identical to the amino acid sequence encoded by the nucleotide sequence shown in SEQ ID NO: 15, HERV-T Gag as used herein may have an amino acid sequence being at least about 85%, at least about 90%, at least about 91%, at least about 92%, at least about 93%, at least about 94%, at least about 95%, at least about 96%, at least about 97%, at least about 98%, at least about 99%, at least about 99.5%, at least about 99.8%, or 100% similar or identical to the amino acid sequence encoded by the nucleotide sequence shown in SEQ ID NO: 17, HERV-W Gag as used herein may have an amino acid sequence being at least about 85%, at least about 90%, at least about 91%, at least about 92%, at least about 93%, at least about 94%, at least about 95%, at least about 96%, at least about 97%, at least about 98%, at least about 99%, at least about 99.5%, at least about 99.8%, or 100% similar or identical to the amino acid sequence encoded by the nucleotide sequence shown in SEQ ID NO: 18, and so forth.

In accordance with the present invention, as used herein in context with amino acid sequences, the term “similar” means that a given amino acid sequence comprises identical amino acids or only conservative or highly conservative substitutions compared to the amino acid sequence of the respective SEQ ID NO. As used herein, “conservative” substitutions mean substitutions as listed as “Exemplary Substitutions” in Table I herein. “Highly conservative” substitutions as used herein mean substitutions as shown under the heading “Preferred Substitutions” in Table I herein.

TABLE I
Amino Acid Substitutions

| Original | Exemplary Substitutions | Preferred Substitutions |
|---|---|---|
| Ala (A) | val; leu; ile | Val |
| Arg (R) | lys; gln; asn | lys |
| Asn (N) | gln; his; asp, lys; arg | gln |
| Asp (D) | glu; asn | glu |
| Cys (C) | ser; ala | ser |
| Gln (Q) | asn; glu | asn |
| Glu (E) | asp; gln | asp |
| Gly (G) | ala | ala |
| His (H) | asn; gln; lys; arg | arg |
| Ile (I) | leu; val; met; ala; phe; | leu |
| Leu (L) | norleucine; ile; val; met; ala; | ile |
| Lys (K) | arg; gin; asn | arg |
| Met (M) | leu; phe; ile | leu |
| Phe (F) | leu; val; ile; ala; tyr | tyr |
| Pro (P) | ala | ala |
| Ser (S) | thr | thr |
| Thr (T) | ser | ser |
| Trp (W) | tyr; phe | tyr |
| Tyr (Y) | trp; phe; thr; ser | Phe |
| Val (V) | ile; leu; met; phe; ala; | leu |

As used herein, unless specifically defined otherwise, the term “nucleic acid” or “nucleic acid molecule” is used synonymously with “oligonucleotide”, “nucleic acid strand”, or the like, and means a polymer comprising one, two, or more nucleotides. In this context, also the term “target sequence” as used herein comprises nucleic acid molecules.

As used herein, “silent” mutations mean base substitutions within a nucleic acid sequence which do not change the amino acid sequence encoded by the nucleic acid sequence. “Conservative” substitutions mean substitutions as listed as “Exemplary Substitutions” in Table I. “Highly conservative” substitutions as used herein mean substitutions as shown under the heading “Preferred Substitutions” in Table I.

The term “position” when used in accordance with the present invention means the position of an amino acid within an amino acid sequence depicted herein. The term “corresponding” in this context also includes that a position is not only determined by the number of the preceding nucleotides/amino acids.

The level of identity between two or more sequences (e.g., nucleic acid sequences or amino acid sequences) can be easily determined by methods known in the art, e.g., by BLAST analysis. Generally, in context with the present invention, if two sequences (e.g., polynucleotide sequences or amino acid sequences) to be compared by, e.g., sequence comparisons differ in identity, then the term “identity” may refer to the shorter sequence and that part of the longer sequence that matches said shorter sequence. Therefore, when the sequences which are compared do not have the same length, the degree of identity may preferably either refer to the percentage of nucleotide residues in the shorter sequence which are identical to nucleotide residues in the longer sequence or to the percentage of nucleotides in the longer sequence which are identical to nucleotide sequence in the shorter sequence. In this context, the skilled person is readily in the position to determine that part of a longer sequence that matches the shorter sequence. Furthermore, as used herein, identity levels of nucleic acid sequences or amino acid sequences may refer to the entire length of the respective sequence and is preferably assessed pair-wise, wherein each gap is to be counted as one mismatch. These definitions for sequence comparisons (e.g., establishment of “identity” values) are to be applied for all sequences described and disclosed herein.

Moreover, the term “identity” as used herein means that there is a functional and/or structural equivalence between the corresponding sequences. Nucleic acid/amino acid sequences having the given identity levels to the herein-described particular nucleic acid/amino acid sequences may represent derivatives/variants of these sequences which, preferably, have the same biological function. They may be either naturally occurring variations, for instance sequences from other varieties, species, etc., or mutations, and said mutations may have formed naturally or may have been produced by deliberate mutagenesis. Furthermore, the variations may be synthetically produced sequences. The variants may be naturally occurring variants or synthetically produced variants, or variants produced by recombinant DNA techniques. Deviations from the above-described nucleic acid sequences may have been produced, e.g., by deletion, substitution, addition, insertion and/or recombination. The term “addition” refers to adding a nucleic acid residue/amino acid to the beginning or end of the given sequence, whereas “insertion” refers to inserting a nucleic acid residue/amino acid within a given sequence. The term “deletion” refers to deleting or removal of a nucleic acid residue or amino acid residue in a given sequence. The term “substitution” refers to the replacement of a nucleic acid residue/amino acid residue in a given sequence. Again, these definitions as used here apply, mutatis mutandis, for all sequences provided and described herein.

Generally, as used herein, the terms “polynucleotide” and “nucleic acid” or “nucleic acid molecule” are to be construed synonymously. Generally, nucleic acid molecules may comprise inter alia DNA molecules, RNA molecules, oligonucleotide thiophosphates, substituted ribo-oligonucleotides or PNA molecules. Furthermore, the term “nucleic acid molecule” may refer to DNA or RNA or hybrids thereof or any modification thereof that is known in the art (see, e.g., U.S. Pat. Nos. 5,525,711, 4,711,955, 5,792,608 or EP 302175 for examples of modifications). The polynucleotide sequence may be single- or double-stranded, linear or circular, natural or synthetic, and without any size limitation. For instance, the polynucleotide sequence may be genomic DNA, cDNA, mitochondrial DNA, mRNA, antisense RNA, ribozymal RNA or a DNA encoding such RNAs or chimeroplasts (Gamper, Nucleic Acids Research, 2000, 28, 4332-4339). Said polynucleotide sequence may be in the form of a vector, plasmid or of viral DNA or RNA. Also described herein are nucleic acid molecules which are complementary to the nucleic acid molecules described above and nucleic acid molecules which are able to hybridize to nucleic acid molecules described herein. A nucleic acid molecule described herein may also be a fragment of the nucleic acid molecules in context of the present invention. Particularly, such a fragment is a functional fragment. Examples for such functional fragments are nucleic acid molecules which can serve as primers.

The term “amino acid” or “amino acid residue” as used herein typically refers to an amino acid having its art recognized definition such as an amino acid selected from the group consisting of: alanine (Ala or A); arginine (Arg or R); asparagine (Asn or N); aspartic acid (Asp or D); cysteine (Cys or C); glutamine (Gln or Q); glutamic acid (Glu or E); glycine (Gly or G); histidine (His or H); isoleucine (He or I): leucine (Leu or L); lysine (Lys or K); methionine (Met or M); phenylalanine (Phe or F); pro line (Pro or P); serine (Ser or S); threonine (Thr or T); tryptophan (Trp or W); tyrosine (Tyr or Y); and valine (Val or V), although modified, synthetic, or rare amino acids may be used as desired. Generally, amino acids can be grouped as having a nonpolar side chain (e.g., Ala, Cys, He, Leu, Met, Phe, Pro, Val); a negatively charged side chain (e.g., Asp, Glu); a positively charged sidechain (e.g., Arg, His, Lys); or an uncharged polar side chain (e.g., Asn, Cys, Gln, Gly, His, Met, Phe, Ser, Thr, Trp, and Tyr).

As has been shown in context with the present invention, inhibitors that block maturation of HERV Env proteins also impair spreading of pathogenic aggregates. The same applies mutatis mutandis for inhibitors of HERV Gag protein maturation as this inhibits activation of Env as described above. Ways of inhbiting processing/maturation of Env or Gag proteins are generally known in the art; cf. Freed, Nature Review Microbiol (2015), 13: 484-496; Tedbury et al., Curr Topics Microbiol and Immunol (2015), 389: 171-201; Venanzi Rullo et al., Mol Med Reports (2019), 19: 1987-1995; Waheed et al., AIDS Res Human Retroviruses (2012), 28: 54-75. Accordingly, in one embodiment of the present invention, in context with the inhibitor of HERV Env and/or Gag proteins (or fragments thereof) as described and provided herein, HERV Env and/or Gag proteins (or fragments thereof) can be inhibited by inhibiting maturation of the respective HERV Env and/or Gag protein (or fragments thereof). Accordingly, in this embodiment of the present invention, the inhibitor of HERV Env and/or Gag proteins may inhibit maturation of the respective HERV Env and/or Gag protein (or fragments thereof). In a specific embodiment of the present invention, the inhibitor of HERV Env and/or Gag proteins (or fragments thereof) inhibits maturation of the respective HERV proteins, wherein said inhibitor is a HERV protease inhibitor which inhibits the respective protease which processes the respective Env or Gag protein for maturation. Due to the similarity to other retroviral gene products (e.g., those of HIV), protease inhibitors which are applied or applicable for treating HIV may also be used in accordance with the present invention to inhibit HERV Env and/or Gag protein (or fragments thereof) for use in treating tauopathy (e.g., Alzheimer's Disease (AD), Argyrophilic Grain Disease (AGD), Cortical Basal Degeneration (CBD), Progressive Supranuclear Palsy (PSP), Pick's Disease (PiD), and Frontotemporal Dementia with Parkinsonism related to chromosome 17 (FTDP-17)), Parkinson's disease and ALS as described herein. Accordingly, in a specific embodiment of the present invention, the inhibitor of HERV Env and/or Gag proteins (or fragments thereof) may be an HIV-1 retroviral protease inhibitor, e.g., a (HERV) protease inhibitor selected from the group consisting of amprenavir, lopinavir, darunavir, indinavir, atazanavir, fosamprenavir, nelfinavir, ritonavir, saquinavir, and tipranavir, preferably amprenavir and atazanavir. Envisaged herein is a (HERV) protease inhibitor for use in treating Tauopathy, or Parkinson's disease. Said protease inhibitor may be any one of amprenavir, lopinavir, darunavir, indinavir, atazanavir, fosamprenavir, nelfinavir, ritonavir, saquinavir, tipranavir, amprenavir or atazanavir, preferably amprenavir, atazanavir, or lopinavir.

In accordance with the present invention, the inhibitor of HERV Env and/or Gag proteins (or fragments thereof) as described and provided herein may also be an inhibitor of expression of respective HERV Env and/or Gag proteins. For example, it may inhibit transcription or translation of the HERV Env and/or Gag genes or mRNAs, respectively. Accordingly, in one embodiment of the present invention, the inhibitors of HERV Env and/or Gag proteins as described and provided herein are nucleic acid molecules hybridizing or being complementary to at least a portion of the nucleic acid sequence encoding said HERV Env and/or Gag proteins, thereby inhibiting or preventing transcription or translation of the nucleic acid molecules encoding the HERV Env and/or Gag proteins. In context with the present invention, a suitable nucleic acid molecule for inhibiting expression (e.g., transcription or translation) of a HERV Env and/or Gag protein may be a small interference RNA (siRNA), microRNA (miRNA, miR), Tough Decoys (TuD) (e.g., Tough Decoy RNA), Decoys, antisense oligonucleotides (antisense RNA or DNA, chimeric antisense molecules), ribozymes, external guide sequence (EGS), oligonucleotides, small temporal RNA (stRNA), short hairpin RNA (shRNA), small RNA-induced gene activation (RNAa), small activating RNA (saRNA), locked nucleic acids (LNA), antagomirs, aptamers (DNA, RNA, XNA), peptide nucleic acids (PNA), and other oligomeric nucleic acid molecules, which are able to inhibit or suppress the expression (e.g., transcription or translation) of the nucleic acid molecule encoding the respective HERV Env and/or Gag proteins (e.g., by hybridizing to at least a portion of the nucleic acid molecule encoding the respective HERV Env and/or Gag proteins).

As used herein, an inhibitor hybridizing or being complementary to “at least a portion” of the nucleic acid sequence encoding said HERV Env protein means that said inhibitor (preferably itself being a nucleic acid molecule as described above) hybridizes or is complementary to at least about 3, 4, 5, 6, 7, 8, 9, 20, 11, 12, 13, 14 or 15 (preferably consecutive) nucleotides of the nucleotide sequence of the respective HERV Env protein (e.g., gene or transcribed mRNA thereof). For example, such inhibitor hybridizes or is complementary to at least about 3, 4, 5, 6, 7, 8, 9, 20, 11, 12, 13, 14 or 15 (preferably consecutive) nucleotides of a nucleotide sequence shown in any one of SEQ ID NOs. 1 to 13 (particularly HERV-H: SEQ ID NO: 1; HERV-K: SEQ ID NO: 2; HERV-T: SEQ ID NO: 3; HERV-W: SEQ ID NO: 4; HERV-FRD: SEQ ID NO 5; HERV-R: SEQ ID NO: 6; HERV-R(b): SEQ ID NO: 7; HERV-F(c)2: SEQ ID NO: 8; HERV-F(c)1: SEQ ID NO: 9; HERV-E: SEQ ID NO: 10; HERV-P(b1): SEQ ID NO: 11; HERV-V: SEQ ID NO: 12; HERV-MER34: SEQ ID NO: 13). That is, for example, in accordance with this embodiment of the present invention, an inhibitor (preferably itself being a nucleic acid molecule as described above) of HERV-H hybridizes or is complementary to at least about 3, 4, 5, 6, 7, 8, 9, 20, 11, 12, 13, 14 or 15 (preferably consecutive) nucleotides of the nucleotide sequence of SEQ ID NO: 1, an inhibitor (preferably itself being a nucleic acid molecule as described above) of HERV-K hybridizes or is complementary to at least about 3, 4, 5, 6, 7, 8, 9, 20, 11, 12, 13, 14 or 15 (preferably consecutive) nucleotides of the nucleotide sequence of SEQ ID NO: 2, an inhibitor (preferably itself being a nucleic acid molecule as described above) of HERV-T hybridizes or is complementary to at least about 3, 4, 5, 6, 7, 8, 9, 20, 11, 12, 13, 14 or 15 (preferably consecutive) nucleotides of the nucleotide sequence of SEQ ID NO: 3, an inhibitor (preferably itself being a nucleic acid molecule as described above) of HERV-W hybridizes or is complementary to at least about 3, 4, 5, 6, 7, 8, 9, 20, 11, 12, 13, 14 or 15 (preferably consecutive) nucleotides of the nucleotide sequence of SEQ ID NO: 4, and so forth. The same applies mutatis mutandis with respect to the Gag HERV proteins encoded by nucleotide sequences corresponding to SEQ ID NOs. 14 to 21 as set forth herein, as well as to Gag HERV proteins having amino acids corresponding to SEQ ID NOs. 22 and 23. In specific examples of the present invention, an inhibitor of a HERV Env protein may have a nucleotide sequence according to any one of SEQ ID NOs. 42-72 (upper letters; corresponding to the respective HERV species as indicated below), wherein up to 3, 2, 1, or (preferably) 0 nucleotides are added, inserted, substituted or deleted compared to said respective nucleotide sequences of SEQ ID NOs. 42-72. Further examples of inhibitors of a HERV Env protein may have a nucleotide sequence according to any one of the murine RNAs shown in SEQ ID NOs: 25-29, wherein up to 3, 2, 1, or (preferably) 0 nucleotides are added, inserted, substituted or deleted compared to said respective nucleotide sequences of SEQ ID NOs: 25 to 29. Additionally, SEQ ID NO: 98 may be another example of such an inhibitor with a loop sequence capable of silencing syncytin-1.

It is apparent from the abovemetioned, that the HERV Gag protein may be encoded by a nucleotide sequence shown in any one of SEQ ID NOs: 14 to 21, or encoded by a nucleotide sequence that has at least about 85% sequence identity to any one of SEQ ID NOs: 14 to 21. Thus, a nucleic acid molecule hybridizing to at least a portion of the nucleotide sequence of HERV Gag as shown in any one of SEQ ID NOs: 14 to 21, is envisaged herein and may be used for treating Tauopathy, or Parkinson's disease. Further examples of a nucleic acid molecule may comprise any one of the nucleotide sequence shown in any one of SEQ ID NOs: 30 to 35 which may be use for treating Tauopathy, or Parkinson's disease.

The term “hybridization”, “hybridizing” or “hybridizes” as used herein in context of nucleic acid molecules/DNA or RNA sequences may relate to hybridizations under stringent or non-stringent conditions. If not further specified, the conditions are preferably non-stringent. Said hybridization conditions may be established according to conventional protocols described, for example, in Sambrook, Russell “Molecular Cloning, A Laboratory Manual”, Cold Spring Harbor Laboratory, N. Y. (2001); Current Protocols in Molecular Biology, Update May 9, 2012, Print ISSN: 1934-3639, Online ISSN: 1934-3647; Ausubel, “Current Protocols in Molecular Biology”, Green Publishing Associates and Wiley Interscience, N. Y. (1989), or Higgins and Hames (Eds.) “Nucleic acid hybridization, a practical approach” IRL Press Oxford, Washington D.C., (1985). The setting of conditions is well within the skill of the artisan and can be determined according to protocols described in the art. Thus, the detection of only specifically hybridizing sequences will usually require stringent hybridization and washing conditions such as 0.1×SSC, 0.1% SDS at 65° C. Non-stringent hybridization conditions for the detection of homologous or not exactly complementary sequences may be set at 6×SSC, 1% SDS at 65° C. As is well known, the length of the probe and the composition of the nucleic acid to be determined constitute further parameters of the hybridization conditions. Variations in the above conditions may be accomplished through the inclusion and/or substitution of alternate blocking reagents used to suppress background in hybridization experiments. Typical blocking reagents include Denhardt's reagent, BLOTTO, heparin, denatured salmon sperm DNA, and commercially available proprietary formulations. The inclusion of specific blocking reagents may require modification of the hybridization conditions described above, due to problems with compatibility. In accordance to the invention described herein, low stringent hybridization conditions for the detection of homologous or not exactly complementary sequences may, for example, be set at 6×SSC, 1% SDS at 65° C. As is well known, the length of the probe and the composition of the nucleic acid to be determined constitute further parameters of the hybridization conditions.

Hybridizing nucleic acid molecules also comprise fragments of the above described molecules. Such fragments may represent nucleic acid molecules which code for a functional aaRS as described herein or a functional fragment thereof which can serve as a primer. Furthermore, nucleic acid molecules which hybridize with any of the aforementioned nucleic acid molecules also include complementary fragments, derivatives and variants of these molecules. Additionally, a hybridization complex refers to a complex between two nucleic acid sequences by virtue of the formation of hydrogen bonds between complementary G and C bases and between complementary A and T bases; these hydrogen bonds may be further stabilized by base stacking interactions. The two complementary nucleic acid sequences hydrogen bond in an antiparallel configuration. A hybridization complex may be formed in solution (e.g., Cot or Rot analysis) or between one nucleic acid sequence present in solution and another nucleic acid sequence immobilized on a solid support (e.g., membranes, filters, chips, pins or glass slides to which, e.g., cells have been fixed). The terms complementary or complementarity refer to the natural binding of polynucleotides under permissive salt and temperature conditions by base-pairing. For example, the sequence “A-G-T” binds to the complementary sequence “T-C-A”. Complementarity between two single-stranded molecules may be “partial”, in which only some of the nucleic acids bind, or it may be complete when total complementarity exists between single-stranded molecules. The degree of complementarity between nucleic acid strands has significant effects on the efficiency and strength of hybridization between nucleic acid strands. This is of particular importance in amplification reactions, which depend upon binding between nucleic acids strands. The term “hybridizing sequences” preferably refers to sequences which display a sequence identity of at least 45%, more preferably at least 50%, more preferably at least 55%, more preferably at least 60%, more preferably at least 65%, more preferably at least 70%, more preferably at least 75%, more preferably at least 80%, more preferably at least 85%, more preferably at least 90%, more preferably at least 95%, more preferably at least 96%, more preferably at least 97%, more preferably at least 98% more preferably at least 99%, more preferably at least 99.5%, and most preferably 100% identity with a nucleic acid sequence as described herein encoding an aaRS as described and provided herein.

In accordance with the present invention, the inhibitor of HERV Env proteins as described and provided herein may also be an inhibitor of binding of said HERV Env protein to a receptor of said HERV Env protein. As used herein, such receptors of HERV Env proteins generally comprise any receptor for which a HERV Env protein as used and described herein may be a natural ligand, or to which a HERV Env protein as used and described herein binds to with specific affinity. Typically, as generally used herein unless specified otherwise, binding is considered “specific” when the binding affinity is higher than 10⁻⁶ M. Preferably, as generally used herein unless specified otherwise, binding is considered specific when binding affinity is about 10⁻¹¹ to 10⁻⁶ M (K_{D}), preferably of about 10⁻¹¹ to 10⁻⁹ M. If necessary, nonspecific binding can be reduced without substantially affecting specific binding by varying the binding conditions. Whether the recognition molecule specifically reacts as defined herein above can easily be tested, inter alia, by comparing the reaction of said recognition molecule with an epitope with the reaction of said recognition molecule with (an) other protein(s). In accordance with the present invention, non-limiting examples for receptors for HERV Env proteins, e.g., for HERV-W, comprise ASCT1 (gene: SLC1A4) or ASCT2 (gene: SLC1A5). A further possible receptor of HERV-K Env may be a complex of CD98HC and LAT1, as recent studies suggested. This complex may serve as another example of an HERV-K Env receptor.

In context with the present invention, binding of a HERV Env protein as used and described herein to a corresponding HERV Env protein receptor as described herein can be inhibited by any binding agent either (specifically) binding the respective HERV Env protein, or the corresponding HERV Env protein receptor. In accordance with the present invention, non-limiting examples for such binding agents binding to HERV Env protein or to its respective receptor comprise antibodies, protein aptamers, affimers, small compounds (e.g., those blocking the binding center of a HERV Env receptor), etc.

In one embodiment of the present invention, the inhibitor of Env protein is an antibody (specifically) binding to a respective Env protein as used and described herein. Preferably, such antibody thus inhibits binding of the respective Env protein to a receptor of the Env protein. In another embodiment of the present invention, the inhibitor is a binding agent as defined herein (e.g., an antibody) (specifically) binding to a receptor of a respective HERV Env protein as used and described herein. Preferably, such antibody thus inhibits binding of a respective HERV Env protein to the receptor of the HERV Env protein.

Accordingly, the present invention further relates to an inhibitor of a receptor binding a HERV Env protein as used and described herein for use in treating tauopathy (e.g., Alzheimer's Disease (AD), Argyrophilic Grain Disease (AGD), Cortical Basal Degeneration (CBD), Progressive Supranuclear Palsy (PSP), Pick's Disease (PiD), and Frontotemporal Dementia with Parkinsonism related to chromosome 17 (FTDP-17)), Parkinson's disease and ALS. In context with the present invention, in one example, such inhibitor of a HERV Env receptor may be a binding agent (e.g., antibody) as described herein (thereby inhibiting binding of a respective HERV Env protein to the receptor of the HERV Env protein), for example an antibody (specifically) binding to said HERV Env protein receptor. In another example of the present invention, the HERV Env protein receptor inhibitor may inhibit the expression (e.g., transcription or translation) of the HERV Env protein receptor as described herein. For example, it may inhibit transcription or translation of the HERV Env receptor gene or mRNA, respectively. Accordingly, in one embodiment of the present invention, the inhibitor of HERV Env protein receptor as described and provided herein is a nucleic acid molecule hybridizing or being complementary to at least a portion of the nucleic acid sequence encoding said HERV Env protein receptor, thereby inhibiting or preventing transcription or translation of the nucleic acid molecule encoding the HERV Env protein receptor. In context with the present invention, a suitable nucleic acid molecule for inhibiting expression (e.g., transcription or translation) of a HERV Env protein receptor may be a small interference RNA (siRNA), microRNA (miRNA, miR), Tough Decoys (TuD) (e.g., Tough Decoy RNA), Decoys, antisense oligonucleotides (antisense RNA or DNA, chimeric antisense molecules), ribozymes, external guide sequence (EGS), oligonucleotides, small temporal RNA (stRNA), short hairpin RNA (shRNA), small RNA-induced gene activation (RNAa), small activating RNA (saRNA), locked nucleic acids (LNA), antagomirs, aptamers (DNA, RNA, XNA), peptide nucleic acids (PNA), and other oligomeric nucleic acid molecules, which are able to inhibit or suppress the expression (e.g., transcription or translation) of the nucleic acid molecule encoding the respective HERV Env protein receptor (e.g., by hybridizing to at least a portion of the nucleic acid molecule encoding the respective HER Env protein receptor).

As used herein, an inhibitor hybridizing or being complementary to “at least a portion” of the nucleic acid sequence encoding said HERV Env protein receptor means that said inhibitor (preferably itself being a nucleic acid molecule as described above) hybridizes or is complementary to at least about 3, 4, 5, 6, 7, 8, 9, 20, 11, 12, 13, 14 or 15 (preferably consecutive) nucleotides of the nucleotide sequence of the respective HERV Env protein receptor (e.g., gene or transcribed mRNA thereof).

Generally, as used herein, such receptors of HERV Env proteins comprise any receptor for which a HERV Env protein as used and described herein may be a natural ligand, or to which a HERV Env protein as used and described herein binds to with specific affinity. In accordance with the present invention, non-limiting examples for receptors for HERV Env proteins, e.g., for HERV-W, comprise SLC1A4 or SLC1A5. Accordingly, in a specific embodiment of the present invention, the HERV Env protein receptor is selected from the group consisting of SLC1A4 and SLC1A5 (Lavillette et al., J Virol (2002), 76: 6442-6452; and Marin et al., J Virol (2003), 77: 2936-2945). In this context, an inhibitor of SLC1A4 or SLC1A5 may be particularly useful in treating tauopathies.

The term “antibody” as used herein may be a protein comprising one or more polypeptides (comprising one or more binding domains, preferably antigen binding domains) substantially or partially encoded by immunoglobulin genes or fragments of immunoglobulin genes. The term “immunoglobulin” (Ig) is used interchangeably with “antibody” herein. The recognized immunoglobulin genes include the kappa, lambda, alpha, gamma, delta, epsilon and mu constant region genes, as well as myriad immunoglobulin variable region genes.

In particular, an “antibody” when used herein, may comprise tetrameric glycosylated proteins composed of two light (L) chains of approximately 25 kDa each and two heavy (H) chains of approximately 50 kDa each. Two types of light chain, termed lambda and kappa, may be found in antibodies. Depending on the amino acid sequence of the constant domain of heavy chains, immunoglobulins can be assigned to five major classes: A, D, E, G, and M, and several of these may be further divided into subclasses (isotypes), e.g., IgG1, IgG2, IgG3, IgG4, IgA1, and IgA2. An IgM antibody consists of 5 of the basic heterotetramer unit along with an additional polypeptide called a J chain, and contains 10 antigen binding sites, while IgA antibodies comprise from 2-5 of the basic 4-chain units which can polymerize to form polyvalent assemblages in combination with the J chain. In the case of IgGs, the 4-chain unit comprises in most cases about 150,000 daltons.

Each light chain includes an N-terminal variable (V) domain (VL) and a constant (C) domain (CL). Each heavy chain includes an N-terminal V domain (VH), three or four C domains (CHs), and a hinge region. The constant domains are not involved directly in binding an antibody to an antigen.

The pairing of a VH and VL together forms a single antigen-binding site. The CH domain most proximal to VH is designated as CH1. Each L chain is linked to an H chain by one covalent disulfide bond, while the two H chains are linked to each other by one or more disulfide bonds depending on the H chain isotype. The VH and VL domains consist of four regions of relatively conserved sequences called framework regions (FR1, FR2, FR3, and FR4), which form a scaffold for three regions of hypervariable sequences (complementarity determining regions, CDRs). The CDRs contain most of the residues responsible for specific interactions of the antibody with the antigen. CDRs are referred to as CDR 1, CDR2, and CDR3. Accordingly, CDR constituents on the heavy chain are referred to as H1, H2, and H3, while CDR constituents on the light chain are referred to as L1, L2, and L3. The term “variable” refers to the portions of the immunoglobulin domains that exhibit variability in their sequence and that are involved in determining the specificity and binding affinity of a particular antibody (i.e. the “variable domain(s)”). Variability is not evenly distributed throughout the variable domains of antibodies; it is concentrated in sub-domains of each of the heavy and light chain variable regions. These sub-domains are called “hypervariable” regions or “complementarity determining regions” (CDRs). The more conserved (i.e. non-hypervariable) portions of the variable domains are called the “framework” regions (FRM). The variable domains of naturally occurring heavy and light chains each comprise four FRM regions, largely adopting a β-sheet configuration, connected by three hypervariable regions, which form loops connecting, and in some cases forming part of, the β-sheet structure. The hypervariable regions in each chain are held together in close proximity by the FRM and, with the hypervariable regions from the other chain, contribute to the formation of the antigen-binding site. The constant domains are not directly involved in antigen binding, but exhibit various effector functions, such as, for example, antibody-dependent, cell-mediated cytotoxicity and complement activation.

The terms “CDR”, and its plural “CDRs”, refer to a complementarity determining region (CDR) of which three make up the binding character of a light chain variable region (CDRL1, CDRL2 and CDRL3) and three make up the binding character of a heavy chain variable region (CDRH1, CDRH2 and CDRH3). CDRs contribute to the functional activity of an antibody molecule and are separated by amino acid sequences that comprise scaffolding or framework regions. The exact definitional CDR boundaries and lengths are subject to different classification and numbering systems. Despite differing boundaries, each of these systems has some degree of overlap in what constitutes the so called “hypervariable regions” within the variable sequences. CDR definitions according to these systems may therefore differ in length and boundary areas with respect to the adjacent framework region. See for example Kabat, Chothia, and/or MacCallum (Kabat et al., loc. cit.; Chothia et al., J Mol Biol (1987), 196: 901; and MacCallum et al., J Mol Biol (1996), 262: 732). However, the numbering in accordance with the so-called Kabat system is preferred.

The term “hypervariable region” (also known as “complementarity determining regions” or CDRs) as used herein refers to the amino acid residues of an antibody which are (usually three or four short regions of extreme sequence variability) within the V-region domain of an immunoglobulin which form the antigen-binding site and are the main determinants of antigen specificity. There are at least two methods for identifying the CDR residues: (1) An approach based on cross-species sequence variability (i.e. Kabat et al., loc. cit.); and (2) An approach based on crystallographic studies of antigen-antibody complexes (Chothia, C. et al., J Mol Biol (1987), 196: 901-917). However, to the extent that two residue identification techniques define regions of overlapping, but not identical regions, they can be combined to define a hybrid CDR. However, in general, the CDR residues are preferably identified in accordance with the so-called Kabat (numbering) system. The term “framework region” refers to the art-recognized portions of an antibody variable region that exist between the more divergent (i.e. hypervariable) CDRs. Such framework regions are typically referred to as frameworks 1 through 4 (FR1, FR2, FR3, and FR4) and provide a scaffold for the presentation of the six CDRs (three from the heavy chain and three from the light chain) in three dimensional space, to form an antigen-binding surface. The term “canonical structure” refers to the main chain conformation that is adopted by the antigen binding (CDR) loops. From comparative structural studies, it has been found that five of the six antigen binding loops have only a limited repertoire of available conformations. Each canonical structure can be characterized by the torsion angles of the polypeptide backbone. Correspondent loops between antibodies may, therefore, have very similar three dimensional structures, despite high amino acid sequence variability in most parts of the loops (Chothia and Lesk, J Mol Biol (1987), 196: 901; Chothia et al., Nature (1989), 342: 877; Martin and Thornton, J Mol Biol (1996), 263: 800, each of which is incorporated by reference in its entirety). Furthermore, there is a relationship between the adopted loop structure and the amino acid sequences surrounding it. The conformation of a particular canonical class is determined by the length of the loop and the amino acid residues residing at key positions within the loop, as well as within the conserved framework (i.e. outside of the loop). Assignment to a particular canonical class can therefore be made based on the presence of these key amino acid residues. The term “canonical structure” may also include considerations as to the linear sequence of the antibody, for example, as catalogued by Kabat (Kabat et al., loc. cit.). The Kabat numbering scheme (system) is a widely adopted standard for numbering the amino acid residues of an antibody variable domain in a consistent manner and is the preferred scheme applied in the present invention as also mentioned elsewhere herein. Additional structural considerations can also be used to determine the canonical structure of an antibody. For example, those differences not fully reflected by Kabat numbering can be described by the numbering system of Chothia et al and/or revealed by other techniques, for example, crystallography and two or three-dimensional computational modeling. Accordingly, a given antibody sequence may be placed into a canonical class which allows for, among other things, identifying appropriate chassis sequences (e.g., based on a desire to include a variety of canonical structures in a library).

CDR3 is typically the greatest source of molecular diversity within the antibody-binding site. H3, for example, can be as short as two amino acid residues or greater than 26 amino acids. The subunit structures and three-dimensional configurations of different classes of immunoglobulins are well known in the art. For a review of the antibody structure, see Antibodies: A Laboratory Manual, Cold Spring Harbor Laboratory, eds. Harlow et al., 1988. One of skill in the art will recognize that each subunit structure, e.g., a CH, VH, CL, VL, CDR, FR structure, comprises active fragments, e.g., the portion of the VH, VL, or CDR subunit the binds to the antigen, i.e., the antigen-binding fragment, or, e.g., the portion of the CH subunit that binds to and/or activates, e.g., an Fc receptor and/or complement. The CDRs typically refer to the Kabat CDRs, as described in Sequences of Proteins of immunological Interest, US Department of Health and Human Services (1991), eds. Kabat et al. Another standard for characterizing the antigen binding site is to refer to the hypervariable loops as described by Chothia. See, e.g., Chothia et al., J Mol Biol (1992), 227:799-817; and Tomlinson et al., EMBO J (1995), 14: 4628-4638. Still another standard is the AbM definition used by Oxford Molecular's AbM antibody modelling software. See, generally, e.g., Protein Sequence and Structure Analysis of Antibody Variable Domains. In: Antibody Engineering Lab Manual (Ed.: Duebel, S. and Kontermann, R., Springer-Verlag, Heidelberg). The sequence of antibody genes after assembly and somatic mutation is highly varied, and these varied genes are estimated to encode 10¹⁰ different antibody molecules (Immunoglobulin Genes, 2nd ed., eds. Jonio et al., Academic Press, San Diego, Calif., 1995). Accordingly, the immune system provides a repertoire of immunoglobulins. The term “repertoire” refers to at least one nucleotide sequence derived wholly or partially from at least one sequence encoding at least one immunoglobulin. The sequence(s) may be generated by rearrangement in vivo of the V, D, and J segments of heavy chains, and the V and J segments of light chains. Alternatively, the sequence(s) can be generated from a cell in response to which rearrangement occurs, e.g., in vitro stimulation. Alternatively, part or all of the sequence(s) may be obtained by DNA splicing, nucleotide synthesis, mutagenesis, and other methods, see, e.g., U.S. Pat. No. 5,565,332. A repertoire may include only one sequence or may include a plurality of sequences, including ones in a genetically diverse collection.

As used herein, unless specified otherwise, the term “antibody” does not only refer to an immunoglobulin (or intact antibody), but also to a fragment thereof, and encompasses any polypeptide comprising an antigen-binding fragment or an antigen-binding domain. Preferably, the fragment such as Fab, F(ab)₂, Fv, scFv, Fd, dAb, and other antibody fragments that retain antigen-binding function. Typically, such fragments would comprise an antigen-binding domain and have the same properties as the antibodies described herein.

The term “antibody” as used herein includes antibodies that compete for binding to the same epitope as the epitope bound by the antibodies of the present invention, preferably obtainable by the methods for the generation of an antibody as described herein elsewhere. To determine if a test antibody can compete for binding to the same epitope, a cross-blocking assay e.g., a competitive ELISA assay can be performed. In an exemplary competitive ELISA assay, epitope-coated wells of a microtiter plate, or epitope-coated sepharose beads, are pre-incubated with or without candidate competing antibody and then a biotin-labeled antibody of the invention is added. The amount of labeled antibody bound to the epitope in the wells or on the beads is measured using avidin-peroxidase conjugate and appropriate substrate. Alternatively, the antibody can be labeled, e.g., with a radioactive or fluorescent label or some other detectable and measurable label. The amount of labeled antibody that binds to the antigen will have an inverse correlation to the ability of the candidate competing antibody (test antibody) to compete for binding to the same epitope on the antigen, i.e. the greater the affinity of the test antibody for the same epitope, the less labeled antibody will be bound to the antigen-coated wells. A candidate competing antibody is considered an antibody that binds substantially to the same epitope or that competes for binding to the same epitope as an antibody of the invention if the candidate competing antibody can block binding of the antibody by at least 20%, preferably by at least 20-50%, even more preferably, by at least 50% as compared to a control performed in parallel in the absence of the candidate competing antibody (but may be in the presence of a known noncompeting antibody). It will be understood that variations of this assay can be performed to arrive at the same quantitative value.

The term “antibody” also includes but is not limited to polyclonal, monoclonal, monospecific, polyspecific such as bispecific, non-specific, humanized, human, single-chain, chimeric, synthetic, recombinant, hybrid, mutated, grafted, and in vitro generated antibodies. In one embodiment of the present invention, the antibody binding to an HERV Env protein or to its respective receptor is a monoclonal antibody (mAb; MAb). In context with the present invention, commercially available antibodies that may be employed in accordance with the present invention comprise those listed in Table II herein.

TABLE II
Antibodies against HERV

| Antigen | Catalogue number | specy | company |
|---|---|---|---|
| HERV K | HERM-1811-5 | mouse | Austral |
| Env | | | |
| (79.5 kDa) | | | |
| HERV K | HERM-1821-5 | mouse | Austral |
| Env | | | |
| (42.8 kDa) | | | |
| HERV K | HERM-1841-5 | mouse | Austral |
| Gag | | | |
| (82 kDa) | | | |
| HERV K | HERM-1831-5 | mouse | Austral |
| Gag | | | |
| (74 kDa) | | | |
| HERV W | ab129943 | rabbit | Abcam |
| HERV W | ab179693 | rabbit | Abcam |
| HERV W | ab71115 | rabbit | Abcam |
| HERV FRD | ab90733 | rabbit | Abcam |
| HERV FRD | ab230235 | rabbit | Abcam |
| HERV FRD | ab171382 | rabbit | Abcam |
| HERV R | ab116723 | rabbit | Abcam |
| HERV W | abx029235 | rabbit | abbexa |
| Env | | | |
| HERV R | ABIN570897 | goat | antibodies-online |
| Env | | | |

Accordingly, the term “antibody” also relates to a purified serum, i.e. a purified polyclonal serum. Accordingly, said term preferably relates to a serum, more preferably a polyclonal serum and most preferably to a purified (monoclonal or polyclonal) serum. The antibody/serum is obtainable, and preferably obtained, for example, by the method or use described herein. “Polyclonal antibodies” or “polyclonal antisera” refer to immune serum containing a mixture of antibodies specific for one (monovalent or specific antisera) or more (polyvalent antisera) antigens which may be prepared from the blood of animals immunized with the antigen or antigens. A non-limiting example of such an antibody beyond the examples given in table II may be an antibody which is capable of binding to the amino acid sequence shown in SEQ ID NOs: 22-23 or to a portion thereof.

Furthermore, the term “antibody” as employed in the invention also relates to derivatives or variants of the antibodies described herein which display the same specificity as the described antibodies. Examples of “antibody variants” include humanized variants of non-human antibodies, “affinity matured” antibodies (see, e.g., Hawkins et al., J Mol Biol (1992), 254, 889-896; and Lowman et al., Biochemistry (1991), 30: 10832-10837) and antibody mutants with altered effector function (s) (see, e.g., U.S. Pat. No. 5,648,260). The terms “antigen-binding domain”, “antigen-binding fragment” and “antibody binding region” when used herein refer to a part of an antibody molecule that comprises amino acids responsible for the specific binding between antibody and antigen. The part of the antigen that is specifically recognized and bound by the antibody is referred to as the “epitope” as described herein above. As mentioned above, an antigen-binding domain may typically comprise an antibody light chain variable region (VL) and an antibody heavy chain variable region (VH); however, it does not have to comprise both. Fd fragments, for example, have two VH regions and often retain some antigen-binding function of the intact antigen-binding domain. Examples of antigen-binding fragments of an antibody include (1) a Fab fragment, a monovalent fragment having the VL, VH, CL and CH1 domains; (2) a F(ab′)2 fragment, a bivalent fragment having two Fab fragments linked by a disulfide bridge at the hinge region; (3) a Fd fragment having the two VH and CH1 domains; (4) a Fv fragment having the VL and VH domains of a single arm of an antibody, (5) a dAb fragment (Ward et al., (1989) Nature 341:544-546), which has a VH domain; (6) an isolated complementarity determining region (CDR), and (7) a single chain Fv (scFv). Although the two domains of the Fv fragment, VL and VH> are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al., (1988) Science (1988), 242: 423-426; and Huston et al., (1988) PNAS USA (1988), 85: 5879-5883). These antibody fragments are obtained using conventional techniques known to those with skill in the art, and the fragments are evaluated for function in the same manner as are intact antibodies.

The term “monoclonal antibody” as used herein refers to an antibody obtained from a population of substantially homogeneous antibodies, i.e. the individual antibodies comprising the population are identical except for possible naturally occurring mutations and/or post-translation modifications (e.g., isomerizations, amidations) that may be present in minor amounts. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen. In addition to their specificity, the monoclonal antibodies are advantageous in that they are synthesized by the hybridoma culture, uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al., Nature (1975), 256: 495, or may be made by recombinant DNA methods (see, e.g., U.S. Pat. No. 4,816,567). The “monoclonal antibodies” may also be isolated from phage antibody libraries using the techniques described in Clackson et al., Nature (1991), 352: 624-628; and Marks et al., J Mol Biol (1991), 222: 581-597, for example. The monoclonal antibodies herein specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain (s) is (are) identical with or homologous to corresponding sequences in antibodies derived from another species or belonging to another antibody class or subclass, as well as fragments of such antibodies, so long as they exhibit the desired biological activity (U.S. Pat. No. 4,816,567; Morrison et al., PNAS USA (1984), 81: 6851-6855). Chimeric antibodies of interest herein include “primitized” antibodies comprising variable domain antigen-binding sequences derived from a non-human primate (e.g., Old World Monkey, Ape etc.) and human constant region sequences. “Humanized” forms of non-human (e.g., murine) antibodies are chimeric immunoglobulins, immunoglobulin chains or fragments thereof (such as Fv, Fab, Fab′, F (ab′) 2 or other antigen-binding subsequences of antibodies) of mostly human sequences, which contain minimal sequence derived from non-human immunoglobulin. For the most part, humanized antibodies are human immunoglobulins (recipient antibody) in which residues from a hypervariable region (also CDR) of the recipient are replaced by residues from a hypervariable region of a non-human species (donor antibody) such as mouse, rat or rabbit having the desired specificity, affinity, and capacity. In some instances, Fv framework region (FR) residues of the human immunoglobulin are replaced by corresponding non-human residues. Furthermore, “humanized antibodies” as used herein may also comprise residues which are found neither in the recipient antibody nor the donor antibody. These modifications are made to further refine and optimize antibody performance. The humanized antibody optimally also will comprise at least a portion of an immunoglobulin constant region (Fc), typically that of a human immunoglobulin. For further details, see Jones et al., Nature (1986), 321: 522-525; Reichmann et al., Nature (1988), 332: 323-329; and Presta, Curr. Op. Struct Biol (1992), 2: 593-596.

The term “human antibody” includes antibodies having variable and constant regions corresponding substantially to human germline immunoglobulin sequences known in the art, including, for example, those described by Kabat et al. (See Kabat et al., loc. cit.). The human antibodies of the invention may include amino acid residues not encoded by human germline immunoglobulin sequences (e.g., mutations introduced by random or site-specific mutagenesis in vitro or by somatic mutation in vivo), for example in the CDRs, and in particular, CDR3. The human antibody can have at least one, two, three, four, five, or more positions replaced with an amino acid residue that is not encoded by the human germline immunoglobulin sequence. As used herein, “in vitro generated antibody” refers to an antibody where all or part of the variable region (e.g., at least one CDR) is generated in a non-immune cell selection (e.g., an in vitro phage display, protein chip or any other method in which candidate sequences can be tested for their ability to bind to an antigen). This term thus preferably excludes sequences generated by genomic rearrangement in an immune cell. A “bispecific” or “bifunctional antibody” is an artificial hybrid antibody having two different heavy/light chain pairs and two different binding sites. Bispecific antibodies can be produced by a variety of methods including fusion of hybridomas or linking of Fab′ fragments. See, e.g., Songsivilai & Lachmann, Clin Exp Immunol (1990), 79: 315-321; Kostelny et al., J Immunol (1992), 148: 1547-1553. In one embodiment, the bispecific antibody comprises a first binding domain polypeptide, such as a Fab′ fragment, linked via an immunoglobulin constant region to a second binding domain polypeptide.

Numerous methods known to those skilled in the art are available for obtaining antibodies or antigen-binding fragments thereof. For example, antibodies can be produced using recombinant DNA methods (U.S. Pat. No. 4,816,567). Monoclonal antibodies may also be produced by generation of hybridomas (see e.g., Kohler and Milstein, Nature (1975), 256: 495-499) in accordance with known methods. Hybridomas formed in this manner are then screened using standard methods, such as enzyme-linked immunosorbent assay (ELISA) and surface plasmon resonance (BIACORE™) analysis, to identify one or more hybridomas that produce an antibody that specifically binds with a specified antigen. Any form of the specified antigen may be used as the immunogen, e.g., recombinant antigen, naturally occurring forms, any variants or fragments thereof, as well as antigenic peptide thereof. One exemplary method of making antibodies includes screening protein expression libraries, e.g., phage or ribosome display libraries. Phage display is described, for example, in U.S. Pat. No. 5,223,409; Smith, Science (1985), 228: 1315-1317; Clackson et al., Nature (1991), 352: 624-628; Marks et al., J Mol Biol (1991), 222: 581-597WO 92/18619; WO 91/17271; WO 92/20791; WO 92/15679; WO 93/01288; WO 92/01047; WO 92/09690; and WO 90/02809. In another embodiment, a monoclonal antibody may be obtained from the non-human animal, and then modified, e.g., humanized, deimmunized, chimeric, may be produced using recombinant DNA techniques known in the art. A variety of approaches for making chimeric antibodies have been described. See, e.g., Morrison et al., PNAS USA (1985), 81: 6851; Takeda et al., Nature (1985), 314: 452; U.S. Pat. Nos. 4,816,567; 4,816,397; EP 171496; EP 173494, GB 2177096. Humanized antibodies may also be produced, for example, using transgenic mice that express human heavy and light chain genes, but are incapable of expressing the endogenous mouse immunoglobulin heavy and light chain genes. Winter describes an exemplary CDR-grafting method that may be used to prepare the humanized antibodies described herein (U.S. Pat. No. 5,225,539). All of the CDRs of a particular human antibody may be replaced with at least a portion of a non-human CDR, or only some of the CDRs may be replaced with non-human CDRs. It is only necessary to replace the number of CDRs required for binding of the humanized antibody to a predetermined antigen. Humanized antibodies or fragments thereof can be generated by replacing sequences of the Fv variable domain that are not directly involved in antigen binding with equivalent sequences from human Fv variable domains. Exemplary methods for generating humanized antibodies or fragments thereof are provided by Morrison, Science (1985), 229: 1202-1207; Oi et al., BioTechniques (1986), 4: 214; U.S. Pat. Nos. 5,585,089; 5,693,761; 5,693,762; 5,859,205; and 6,407,213. Those methods include isolating, manipulating, and expressing the nucleic acid sequences that encode all or part of immunoglobulin Fv variable domains from at least one of a heavy or light chain. Such nucleic acids may be obtained from a hybridoma producing an antibody against a predetermined target, as described above, as well as from other sources. The recombinant DNA encoding the humanized antibody molecule can then be cloned into an appropriate expression vector. In certain embodiments, humanized antibody may be optimized by the introduction of conservative substitutions, consensus sequence substitutions, germline substitutions and/or backmutations. Such altered immunoglobulin molecules can be made by any of several techniques known in the art, (e.g., Teng et al., PNAS USA (1983), 80: 7308-731; Kozbor et al., Immunology Today (1983), 4: 7279; Olsson et al., Meth Enzymol (1982), 92: 3-16), and may be made according to the teachings of WO 92/06193 or EP 239400).

Examples of anti-HERV Env protein-antibodies may bind to the amino acid sequence of the HERV Env protein. Illustrative and non-limiting examples of such amino acid sequences are shown in SEQ ID NO: 101 to 139. Thus, an anti-HERV Env protein-antibody capable of binding to the amino acid sequence of HERV K shown in SEQ ID NOs: 101 to 128 or to a portion thereof may be used in treating Tauopathy, or Parkinson's disease is envisaged herein. Further, an anti-HERV Env protein-antibody capable of binding to the amino acid sequence of HERV W shown in SEQ ID NOs: 129 to 131 or to a portion thereof may be used treating Tauopathy, or Parkinson's disease is encompassed. Likewise an anti-HERV Env protein-antibody capable of binding to the amino acid sequence of HERV H shown in SEQ ID NOs: 132 to 137 or to a portion thereof may be used in treating Tauopathy, or Parkinson's disease is an illustrative example. Finally, an anti-HERV Env protein-antibody capable of binding to the amino acid sequence of HERV R shown in SEQ ID NOs: 138 to 139 or to a portion thereof may be used in treating Tauopathy, or Parkinson's disease is also envisaged herein.

In a similar manner HERV Gag amino acid sequences may be targeted by antibodies and these antibodies thus may fulfill the task of an inhibitor as mentioned herein. Therefore, an anti-HERV Env protein-antibody capable of binding to the amino acid sequence of HERV Gag shown in SEQ ID NOs: 22 to 23 or to a portion thereof may be used in treating Tauopathy, or Parkinson's disease.

As has further been found in context with the present invention, HERV Env proteins as described and defined herein can be used as biomarkers for diagnosing a tauopathy (e.g., Alzheimer's Disease (AD), Argyrophilic Grain Disease (AGD), Cortical Basal Degeneration (CBD), Progressive Supranuclear Palsy (PSP), Pick's Disease (PiD), and Frontotemporal Dementia with Parkinsonism related to chromosome 17 (FTDP-17)), Parkinson's disease and ALS. Particularly, as found in context with the present invention, HERV Env proteins are upregulated and overexpressed in different tauopathies. For example, as has been found in context with the present invention, HERV-W is upregulated in AD patients, while HERV-FRD, H and R(b) are associated with CBD disease, and HERV-K and F(c)1 are increased in PSP patients. Accordingly, in accordance with the present invention, molecules binding to HERV Env proteins or (a) fragment(s) or to nucleic acid molecules encoding such HERV Env proteins or (a) fragment(s) thereof may be used for diagnosing tauopathy (e.g., Alzheimer's Disease (AD), Argyrophilic Grain Disease (AGD), Cortical Basal Degeneration (CBD), Progressive Supranuclear Palsy (PSP), Pick's Disease (PiD), and Frontotemporal Dementia with Parkinsonism related to chromosome 17 (FTDP-17), Parkinson's disease and ALS. In specific embodiments of the present invention, molecules binding to specific HERV-Env proteins or (a) fragment(s) which are associated with specific tauopathies or with nucleic acid molecules encoding such specific HERV Env proteins or (a) fragment(s) thereof may be used for diagnosing corresponding specific tauopathies. For example, in this context, such specific tauopathy-HERV Env protein association has been shown in context with the present invention for (1) HERV-W and AD, (2) HERV-FRD & HERV-H & HERV-R(b) and CBD, and (3) HERV-K & HERV-F(c)1 and PSP.

Accordingly, the present invention also relates to molecules binding to HERV Env protein or a fragment thereof, or to a nucleic acid molecule encoding said HERV Env protein or a fragment thereof, for use in diagnosing a tauopathy (e.g., Alzheimer's Disease (AD), Argyrophilic Grain Disease (AGD), Cortical Basal Degeneration (CBD), Progressive Supranuclear Palsy (PSP), Pick's Disease (PiD), and Frontotemporal Dementia with Parkinsonism related to chromosome 17 (FTDP-17)), Parkinson's disease, or ALS. In one embodiment of the present invention, such molecule binding to HERV Env protein or a fragment thereof may be any binding agent as described and defined herein in context with inhibitors of HERV Env proteins. Preferably, such binding agent is suitable to be employed in a protein detection system, for example—but not limited to—ELISA, ELIA, Western blot, IHC, and other protein detection systems known in the art. In one embodiment, such HERV Env protein binding agent is an antibody as described and defined herein. In a further embodiment, such binding agents may be labelled (depending on the assay used as readily clear for the skilled person).

In another embodiment of the present invention, such molecule binding to a nucleic acid molecule encoding a HERV Env protein or a fragment thereof may be any nucleic acid molecule as described and defined herein in context with inhibitors of HERV Env protein expression (e.g., transcription or translation) which is suitable for specifically detecting a nucleic acid molecule. Preferably, it is a nucleotide probe, hybridizing (preferably under stringent conditions) or being complementary to at least a portion of a nucleic acid molecule encoding a HERV Env protein or a fragment thereof. For example, such nucleic acid molecules may be selected from the group consisting of decoy nucleic acid molecules, primers, or other probe molecules suitable in corresponding assays for specific DNA or RNA sequence identification. Such assays include inter alia PCR (incl. RT, real-time, quantitative), Southern/Northern blot, microarray, etc. In one embodiment, such probe molecules for specific DNA or RNA sequence identification may be labelled (depending on the assay used as readily clear for the skilled person).

In context with the present invention, the tauopathy to be treated or diagnosed as described herein is to be understood as known in the art and may comprise any disease or disorder associated with generation, aggregation, or overexpression of Tau protein. In accordance with the present invention, examples of tauopathies comprise Alzheimer's Disease (AD), Argyrophilic Grain Disease (AGD), Cortical Basal Degeneration (CBD), Progressive Supranuclear Palsy (PSP), Pick's Disease (PiD), and Frontotemporal Dementia with Parkinsonism related to chromosome 17 (FTDP-17), particularly AD, AGD, CBD and PSP, more particularly AD, CBD and PSP.

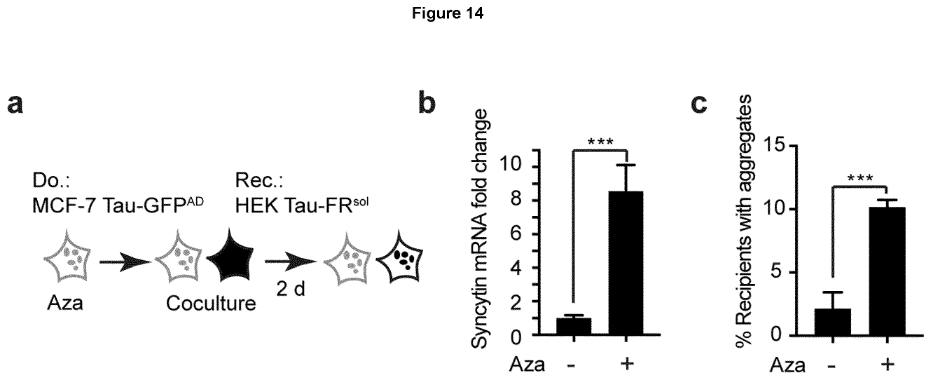

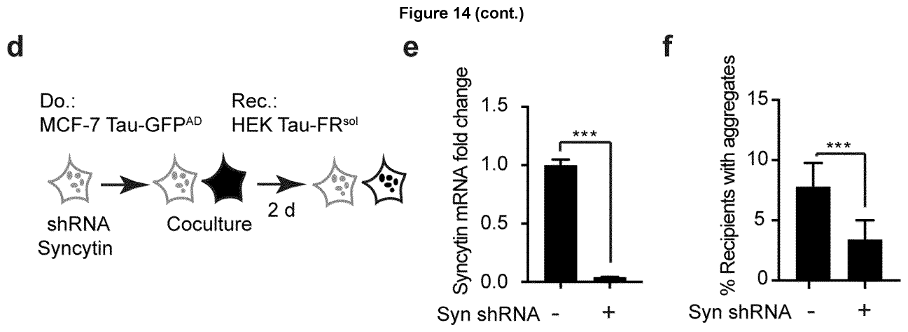

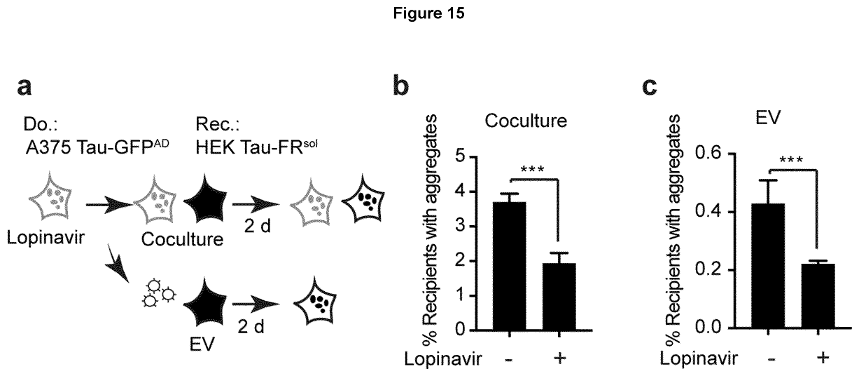

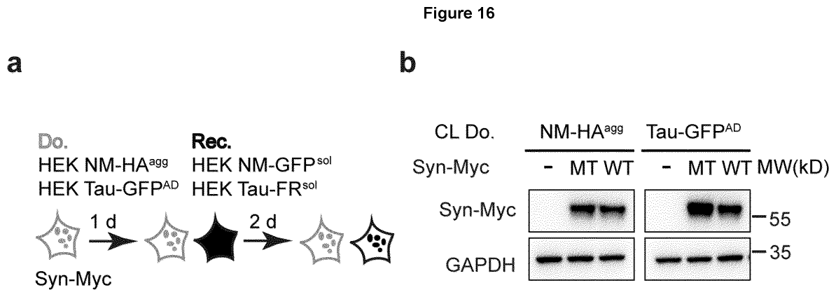

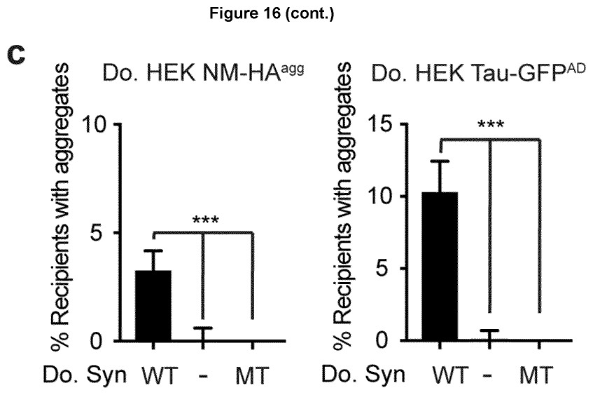

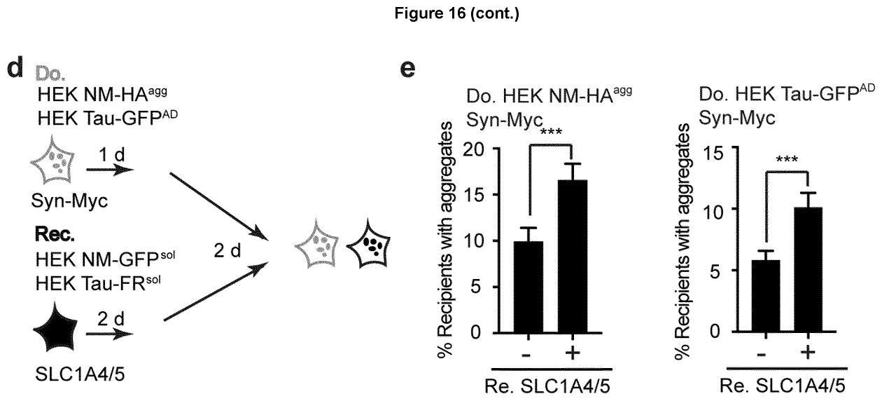

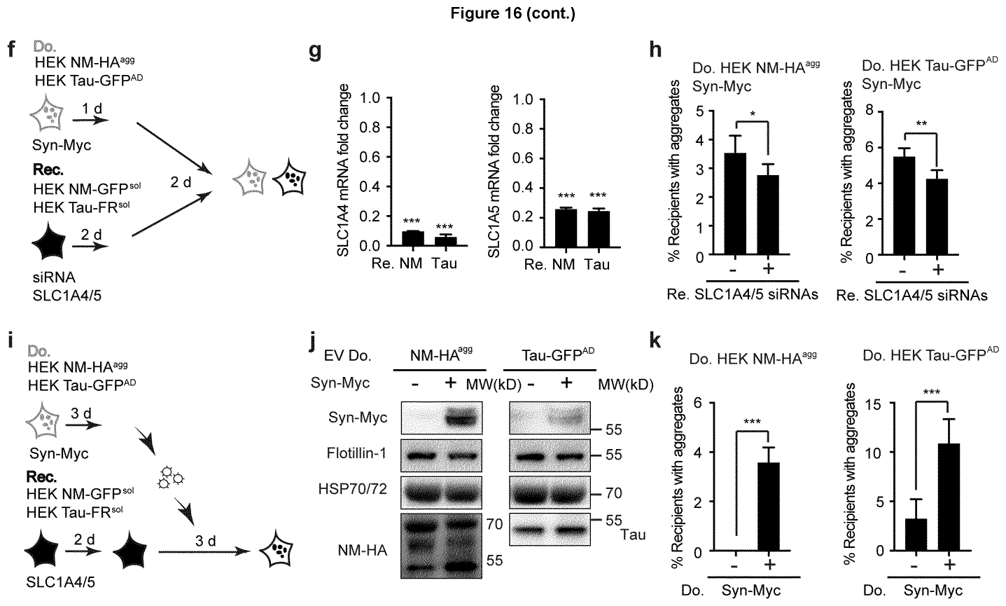

The inventors were able to demonstrate that an shRNA directed against HERV Env (here: HERV-W Syncytin-1) can reduce intercellular Tau aggregate spreading (breast cancer cell line MCF-7, FIG. 14), that the (HIV) protease inhibitor Lopinavir reduces intercellular Tau aggregate spreading (melanoma cells expressing HERV (FIG. 15), and that the receptor interactions between HERV Env (HERV-W Syncytin-1) and its receptors (here: ASCT1 and ASCT2; genes SLC1A4/SLC1A5) enhances intercellular protein aggregate spreading (HEK cells, FIG. 16).

Thus, it is encompassed to use a nucleotide sequence against HERV Env proteins (e.g. HERV-W Syncytin-1 or HERV-W Syncytin-2), or the (HIV) protease inhibitor Lopinavir, to reduce Tau aggregate spreading. A nucleotide sequence directed against the nucleotide sequence of HERV Env shown in any one of SEQ ID NOs: 1 to 13, for use in treating Tauopathy, or Parkinson's disease is encompassed by the present invention. An illustrative example of such a nucleotide sequence may be the sequence shown in SEQ ID NOs: 42 to 72, or SEQ ID NO: 98 or the murine sequences shown in SEQ ID NOs: 25 to 29. Further, a nucleotide sequence directed against HERV-W Syncytin-1 for use in treating Tauopathy, or Parkinson's disease as well as a nucleotide sequence directed against HERV-W Syncytin-2 for use in treating Tauopathy, or Parkinson's disease are embodiments of the present invention.

Likewise, a nucleotide sequence directed against the nucleotide sequence of HERV Gag shown in any one of SEQ ID NOs: 14 to 21 or encoded by a nucleotide sequence that has at least about 85% sequence identity to any one of SEQ ID NOs: 14 to 21, for use in treating Tauopathy, or Parkinson's disease, is envisaged herein. A nucleic acid molecule hybridizing to at least a portion of the nucleotide sequence of HERV Gag shown in any one of SEQ ID NOs: 14 to 21 for use in treating Tauopathy, or Parkinson's disease, is directed to bind anywhere in the respective DNA sequence. Further, a nucleic acid molecule comprising any one of the nucleotide sequence shown in any one of SEQ ID NOs: 30 to 35 for use in treating Tauopathy, or Parkinson's disease, depicts different nucleotide sequence variants, capable of binding selectively to the respective Gag proteins. It is further envisaged that the interaction between HERV Env and its respective receptors ASCT1 (gene SLC1A4), ASCT2 (gene SLC1A4), (and the complex of CD98HC and LAT1 which may functions as HERV Env receptor) are blocked, to reduce aggregate spreading. Thus, an inhibitor of a HERV Env protein capable of blocking the binding of HERV Env to its receptor ASCT1 (gene SLC1A4), ASCT2 (gene SLC1A5) for use in treating Tauopathy, Parkinson's disease, is encompassed herein. Said inhibitor may be a nucleic acid hybridizing to at least a portion of the nucleotide sequence of SLC1A4 (SEQ ID NO: 99) or SLCA4 (SEQ ID NO: 100). Thus, a nucleic acid molecule capable of blocking the HERV Env receptor ASCT1 (gene SLC1A4) or ASCT2 (gene SLC1A5) by hybridizing to at least a portion of the nucleotide sequence shown in SEQ ID NO: 99 or SEQ ID NO: 100 for use in treating Tauopathy, Parkinson's disease.

The present invention may also be characterized by the following items:
Item 1. Inhibitor of a HERV Env protein, proteins comprising HERV Env, or a fragment thereof, for use in treating Tauopathy, Parkinson's disease, or ALS.
Item 2. Inhibitor of item 1, wherein said inhibitor inhibits maturation or expression of said HERV Env protein, and/or binding of said HERV Env protein to a receptor.
Item 3. Inhibitor of item 1 or 2, wherein said inhibitor inhibits maturation of said HERV Env protein, and wherein said inhibitor is a HERV protease inhibitor.
Item 4. Inhibitor of item 1 or 2, wherein said inhibitor inhibits expression of said HERV Env protein, and wherein said inhibitor is a nucleic acid molecule hybridizing to at least a portion of the nucleic acid sequence encoding said HERV Env protein.
Item 5. Inhibitor of item 1 or 2, wherein said inhibitor inhibits binding of said HERV Env protein to a receptor.
Item 6. Inhibitor of item 5, which is an anti-HERV Env protein-antibody.
Item 7. Inhibitor of a receptor binding a HERV Env protein for use in treating Tauopathy, Parkinson's disease, or ALS.
Item 8. Inhibitor of item 7, wherein said inhibitor inhibits maturation or expression of said receptor, and/or binding of HERV Env protein to said receptor.
Item 9. Inhibitor of item 7 or 8, which is a nucleic acid molecule complementary to at least a portion of the nucleic acid sequence encoding said receptor.
Item 10. Inhibitor of item 7 or 8, which is an antibody binding to said receptor or a fragment thereof.
Item 11. Inhibitor of any one of items 7 to 10, wherein said receptor is selected from the group consisting of ASCT1 and ASCT2 (genes SLC1A4/SLC1A5).
Item 12. Molecule binding to HERV Env protein or a fragment thereof, or to a nucleic acid molecule encoding said HERV Env protein or a fragment thereof, for use in diagnosing a Tauopathy, Parkinson's disease, or ALS.
Item 13. Molecule of item 12, which is an anti-HERV Env protein-antibody.
Item 14. Molecule of item 12, which is a nucleic acid molecule binding to the nucleic acid molecule encoding HERV Env protein or a fragment thereof.
Item 15. Inhibitor of any one of items 1 to 11 or molecule of any one of items 12 to 14, wherein said Tauopathy is selected from the group consisting of Alzheimer's Disease (AD), Argyrophilic Grain Disease (AGD), Cortical Basal Degeneration (CBD), Progressive Supranuclear Palsy (PSP), Pick's Disease (PiD), and Frontotemporal Dementia with Parkinsonism related to chromosome 17 (FTDP-17).
Item 16. The inhibitor of any one of the preceding items, wherein the inhibitor is any one of a HERV protease inhibitor, a nucleic acid binding to the nucleic acid molecule encoding a HERV Env protein, or an anti-HERV Env protein-antibody.
Item 17. A HERV protease inhibitor for use in treating Tauopathy, or Parkinson's disease.
Item 18. The HERV protease inhibitor of item 17, wherein the protease inhibitor is any one of amprenavir, lopinavir, darunavir, indinavir, atazanavir, fosamprenavir, nelfinavir, ritonavir, saquinavir, tipranavir, amprenavir or atazanavir.
Item 19. The HERV protease inhibitor of any one of items 17 or 18, wherein the protease inhibitor is preferably amprenavir, atazanavir, or lopinavir.
Item 20. The inhibitor of any one of the preceding items, wherein the HERV Env protein is encoded by the nucleotide sequence shown in any one of SEQ ID NOs: 1 to 13 or encoded by a nucleotide sequence that has at least about 85% sequence identity to any one of SEQ ID NOs: 1 to 13.
Item 21. A nucleic acid molecule hybridizing to at least a portion of the nucleotide sequence encoding the HERV Env protein shown in any one of SEQ ID NOs: 1 to 13 or encoded by a nucleotide sequence that has at least about 85% sequence identity to any one of SEQ ID NOs: 1 to 13 for use in treating Tauopathy, or Parkinson's disease.
Item 22. A nucleic acid molecule comprising any one of the nucleotide sequence shown in any one of SEQ ID NOs: 42 to 72 for use in treating Tauopathy, or Parkinson's disease.
Item 23. An anti-HERV Env protein-antibody capable of binding to the amino acid sequence of HERV K shown in SEQ ID NOs: 101 to 128 or to a portion thereof for use in treating Tauopathy, or Parkinson's disease.
Item 24. An anti-HERV Env protein-antibody capable of binding to the amino acid sequence of HERV W shown in SEQ ID NOs: 129 to 131 or to a portion thereof for use in treating Tauopathy, or Parkinson's disease.
Item 25. An anti-HERV Env protein-antibody capable of binding to the amino acid sequence of HERV H shown in SEQ ID NOs: 132 to 137 or to a portion thereof for use in treating Tauopathy, or Parkinson's disease.
Item 26. An anti-HERV Env protein-antibody capable of binding to the amino acid sequence of HERV R shown in SEQ ID NOs: 138 to 139 or to a portion thereof for use in treating Tauopathy, or Parkinson's disease.
Item 27. The inhibitor of any one of the preceding items, wherein the HERV Gag protein is encoded by the nucleotide sequence shown in any one of SEQ ID NOs: 14 to 21 or encoded by a nucleotide sequence that has at least about 85% sequence identity to any one of SEQ ID NOs: 14 to 21.
Item 28. A nucleic acid molecule hybridizing to at least a portion of the nucleotide sequence of HERV Gag shown in any one of SEQ ID NOs: 14 to 21 or encoded by a nucleotide sequence that has at least about 85% sequence identity to any one of SEQ ID NOs: 14 to 21 for use in treating Tauopathy, or Parkinson's disease.
Item 29. A nucleic acid molecule comprising any one of the nucleotide sequence shown in any one of SEQ ID NOs: 30 to 35 for use in treating Tauopathy, or Parkinson's disease.
Item 30. An anti-HERV Env protein-antibody capable of binding to the amino acid sequence of HERV Gag shown in SEQ ID NOs: 22 to 23 or to a portion thereof for use in treating Tauopathy, or Parkinson's disease.
Item 31. A nucleic acid molecule capable of blocking the HERV Env receptor ASCT1 (gene SLC1A4) or ASCT2 (gene SLC1A5) by hybridizing to at least a portion of the nucleotide sequence shown in SEQ ID NO: 99 or SEQ ID NO: 100 for use in treating Tauopathy, Parkinson's disease.
Item 32. The inhibitor of item 16, wherein the inhibitor comprises the HERV protease inhibitor of any one of item 17 to 19, the nucleic acid of any one of item 22 to 23 or item 28 to 29, or item 31, or the anti-HERV Env protein-antibody of any one of item 23 to 26 or item 30.

The embodiments which characterize the present invention are described herein, shown in the Figures, illustrated in the Examples, and reflected in the claims.

It must be noted that as used herein, the singular forms “a”, “an”, and “the”, include plural references unless the context clearly indicates otherwise. Thus, for example, reference to “a reagent” includes one or more of such different reagents and reference to “the method” includes reference to equivalent steps and methods known to those of ordinary skill in the art that could be modified or substituted for the methods described herein.

Unless otherwise indicated, the term “at least” preceding a series of elements is to be understood to refer to every element in the series. Those skilled in the art will recognize or be able to ascertain using no more than routine experimentation, many equivalents to the specific embodiments of the invention described herein. Such equivalents are intended to be encompassed by the present invention.

The term “and/or” wherever used herein includes the meaning of “and”, “or” and “all or any other combination of the elements connected by said term”.

The term “about” or “approximately” as used herein means within 20%, preferably within 10%, and more preferably within 5% of a given value or range.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein the term “comprising” can be substituted with the term “containing” or “including” or sometimes when used herein with the term “having”.

When used herein “consisting of” excludes any element, step, or ingredient not specified in the claim element. When used herein, “consisting essentially of” does not exclude materials or steps that do not materially affect the basic and novel characteristics of the claim.

In each instance herein any of the terms “comprising”, “consisting essentially of” and “consisting of” may be replaced with either of the other two terms.

It should be understood that this invention is not limited to the particular methodology, protocols, and reagents, etc., described herein and as such can vary. The terminology used herein is for the purpose of describing particular embodiments only and is not intended to limit the scope of the present invention, which is defined solely by the claims.

All publications and patents cited throughout the text of this specification (including all patents, patent applications, scientific publications, manufacturer's specifications, instructions, etc.), whether supra or infra, are hereby incorporated by reference in their entirety. Nothing herein is to be construed as an admission that the invention is not entitled to antedate such disclosure by virtue of prior invention. To the extent the material incorporated by reference contradicts or is inconsistent with this specification, the specification will supersede any such material.

Sequences referred to herein comprise:

SEQ ID NO: 1
H. sapiens
DNA
>HERV-H Env consensus sequence (AJ289711, AJ289710, AJ289709)
ATGACAGAGAAATTCCTTTTCCTTTATCTTTCCCTCCTTCCCATGCCCCTACTCTCACAG
GCACAGTGGAATGAAAATTCCCTTGTCAGTTTTTCCAAAATAATTGCTTCGGGAAACCAT
CTAAGCAACTGTTGGATCTGCCACAACTTCATCACCAGGTCCTCATCTTACCAATATATT
TTGGTAAGAAATTTTTCTTTAAACCTAACATTTGGTTCAGGAATCCCTGAAGGCCAACAT
AAATCTGTTCCGCTCCAGGTTTCGCTTGCTAACTCAGCGCACCAAGTCCCCTGCCTGGA
TCTCACTCCACCTTTCAATCAAAGCTCTAAAACTTCTTTCTATTTCTACAACTGCTCTTCT
CTAAACCAAACCTGTTGTCCATGCCCTGAAGGACACTGTGACAGGAAGAACACCTCTGA
GGAGGGATTCCCCAGTCCCACCATCCATCCCATGAGCTTCTCCCCAGCAGGCTGCCAC
CCTAACTTGACTCACTGGTGTCCAGCTAAACAAATGAACGATTATCGAGACAAGTCACC
CCAAAACCGCTGTGCAGCTTGGGAAGGAAAAGAGCTAATCACATGGAGGGTTCTATATT
CGCTTCCCAAGGCACACACTGTCCCCACATGGCCAAAATCTACTGTTCCCCTGGGAGG
GCCTCTATCCCCTGCATGCAATCAAACTATTCCAGCAGGGTGGAAATCGCAGTTACACA
AGTGGTTCGACAGCCACATCCCCCGGTGGGCCTGTACCCCTCCTGGCTATGTATTTTTA
TGTGGGCCACAAAAAAATAAACTGCCCTTTGATGGAAGTCCTAAGATAACCTATTCAACC
CCCCCTGTGGCAAACCTCTACACTTGCATTAATAACATCCAACATACGGGAGAATGTGC
TGTGGGACTTTTGGGACCACGGGGGATAGGTGTGACCATTTATAACACCACCCAACCCA
GACAGAAAAGAGCTCTGGGTCTAATACTGGCAGGGATGGGTGCGGCCATAGGAATGAT
CGCCCCATGGGGAGGGTTCACTTATCATGATGTCACCCTCAGAAATCTCTCCAGACAAA
TAGACAACATAGCTAAGAGTACCAGAGATAGCATCTCTAAACTCAAGGCCTCCATAGATT
CTCTAGCAAATGTAGTCATGGACAACAGATTGGCCTTAGATTACCTCTTAGCAGAGCAG
GGTGGAGTCTGTGCAGTGATCAATAAATCCTGTTGCGTTTATGTCAATAACAGTGGGGC
GATAGAGGAGGATATAAAAAAGATCTATGATGAGGCTACGTGGCTCCATGACTTTGGAA
AAGGAGGTGCTTCAGCAAGGGCCATCTGGGAGGCTGTGAAGTCTGCCCTCCCCTCCCT
CAACTGGTTTGTCCCTTTACTGGGACCAGCAACAGTTATACTCTTACTTTTCCTCTTTGG
CCCTTGTTTCTTTAATTTACTGATTAAGTGTGTCTCTTCTAGGATAAAGCAATTTCACATG
AAGTCCCCCCAAATGGAAAGATATCAGCTATCTGTCATTGGAGGCCCCAGCACCTATAA
GCACATCTCCCCCTTGGATGCCAGTGGGCAAAGATTCCGGGAAACTATGGAGGAATTTT
CTCTCTGA
SEQ ID NO: 2
H. sapiens
DNA
>HERV K Env consensus sequence from 11 genes (Lee et al., PLoS
Pathogens(2007), 3(1): e10)
ATGAACCCATCGGAGATGCAAAGAAAAGCACCTCCGCGGAGACGGAGACACCGCAATC
GAGCACCGTTGACTCACAAGATGAACAAAATGGTGACGTCAGAAGAACAGATGAAGTTG
CCATCCACCAAGAAGGCAGAGCCGCCGACTTGGGCACAACTAAAGAAGCTGACGCAGT
TAGCTACAAAATATCTAGAGAACACAAAGGTGACACAAACCCCAGAGAGTATGCTGCTT
GCAGCCTTGATGATTGTATCAATGGTGGTAAGTCTCCCTATGCCTGCAGGAGCAGCTGC
AGCTAACTATACCTACTGGGCCTATGTGCCTTTCCCGCCCTTAATTCGGGCAGTCACAT
GGATGGATAATCCTATAGAAGTATATGTTAATGATAGTGTATGGGTACCTGGCCCCATAG
ATGATCGCTGCCCTGCCAAACCTGAGGAAGAAGGGATGATGATAAATATTTCCATTGGG
TATCGTTATCCTCCTATTTGCCTAGGGAGAGCACCAGGATGTTTAATGCCTGCAGTCCA
AAATTGGTTGGTAGAAGTACCTACTGTCAGTCCCATCAGTAGATTCACTTATCACATGGT
AAGCGGGATGTCACTCAGGCCACGGGTAAATTATTTACAAGACTTTTCTTATCAAAGATC
ATTAAAATTTAGACCTAAAGGGAAACCTTGCCCCAAGGAAATTCCCAAAGAATCAAAAAA
TACAGAAGTTTTAGTTTGGGAAGAATGTGTGGCCAATAGTGCGGTGATATTACAAAACAA
TGAATTTGGAACTATTATAGATTGGGCACCTCGAGGTCAATTCTACCACAATTGCTCAGG
ACAAACTCAGTCGTGTCCAAGTGCACAAGTGAGTCCAGCTGTTGATAGCGACTTAACAG
AAAGTTTAGACAAACATAAGCATAAAAAATTGCAGTCTTTCTACCCTTGGGAATGGGGAG
AAAAAGGAATCTCTACCCCAAGACCAAAAATAGTAAGTCCTGTTTCTGGTCCTGAACATC
CAGAATTATGGAGGCTTACTGTGGCCTCACACCACATTAGAATTTGGTCTGGAAATCAAA
CTTTAGAAACAAGAGATCGTAAGCCATTTTATACTGTCGACCTAAATTCCAGTCTAACAG
TTCCTTTACAAAGTTGCGTAAAGCCCCCTTATATGCTAGTTGTAGGAAATATAGTTATTAA
ACCAGACTCCCAGACTATAACCTGTGAAAATTGTAGATTGCTTACTTGCATTGATTCAAC
TTTTAATTGGCAACACCGTATTCTGCTGGTGAGAGCAAGAGAGGGCGTGTGGATCCCTG
TGTCCATGGACCGACCGTGGGAGGCCTCACCATCCGTCCATATTTTGACTGAAGTATTA
AAAGGTGTTTTAAATAGATCCAAAAGATTCATTTTTACTTTAATTGCAGTGATTATGGGAT
TAATTGCAGTCACAGCTACGGCTGCTGTAGCAGGAGTTGCATTGCACTCTTCTGTTCAG
TCAGTAAACTTTGTTAATGATTGGCAAAAAAATTCTACAAGATTGTGGAATTCACAATCTA
GTATTGATCAAAAATTGGCAAATCAAATTAATGATCTTAGACAAACTGTCATTTGGATGG
GAGACAGACTCATGAGCTTAGAACATCGTTTCCAGTTACAATGTGACTGGAATACGTCA
GATTTTTGTATTACACCCCAAATTTATAATGAGTCTGAGCATCACTGGGACATGGTTAGA
CGCCATCTACAGGGAAGAGAAGATAATCTCACTTTAGACATTTCCAAATTAAAAGAACAA
ATTTTCGAAGCATCAAAAGCCCATTTAAATTTGGTGCCAGGAACTGAGGCAATTGCAGG
AGTTGCTGATGGCCTCGCAAATCTTAACCCTGTCACTTGGGTTAAGACCATTGGAAGTA
CTACGATTATAAATCTCATATTAATCCTTGTGTGCCTGTTTTGTCTGTTGTTAGTCTGCAG
GTGTACCCAACAGCTCCGAAGAGACAGCGACCATCGAGAACGGGCCATGATGACGATG
GCGGTTTTGTCGAAAAGAAAAGGGGGAAATGTGGGGAAAAGCAAGAGAGATCAGATTG
TTACTGTGTCTGTGTAG
SEQ ID NO: 3
H. sapiens
DNA
>HERV T Env (AC078899)
ATGGGTCCCGAAGCCTGGGTCAGGCCCCTTAAAACTGCGCCTAAGCCGGGTGAAGCCA
TTAGATTAATTCTTTTTATTTACCTCTCTTGTTTCTTTTTGCCTGTTATGTCCTCTGAGCCT
TCCTACTCCTTTCTCCTCACCTCTTTCACAACAGGACGTGTATTCGCAAACACTACTTGG
AGGGCCGGTACCTCCAAGGAAGTCTCCTTTGCAGTTGATTTATGTGTACTGTTCCCAGA
GCCAGCTCGTACCCATGAAGAGCAACATAATTTGCCGGTCATAGGAGCAGGAAGTGTC
GACCTTGCAGCAGGATTTGGACACTCTGGGAGCCAAACTGGATGTGGAAGCTCCAAAG
GTGCAGAAAAAGGGCTCCAAAATGTTGACTTTTACCTCTGTCCTGGAAATCACCCTGAC
GCTAGCTGTAGAGATACTTACCAGTTTTTCTGCCCTGATTGGACATGTGTAACTTTAGCC
ACCTACTCTGGGGGATCAACTAGATCTTCAACTCTTTCCATAAGTCGTGTTCCTCATCCT
AAATTATGTACTAGAAAAAATTGTAATCCTCTTACTATAACTGTCCATGACCCTAATGCAG
CTCAATGGTATTATGGCATGTCATGGGGATTAAGACTTTATATCCCAGGATTTGATGTTG
GGACTATGTTCACCATCCAAAAGAAAATCTTGGTCTCATGGAGCTCCCCCAAGCCAATC
GGGCCTTTAACTGATCTAGGTGACCCTATATTCCAGAAACACCCTGACAAAGTTGATTTA
ACTGTTCCTCTGCCATTCTTAGTTCCTAGACCCCAGCTACAACAACAACATCTTCAACCC
AGCCTAATGTCTATACTAGGTGGAGTACACCATCTCCTTAACCTCACCCAGCCTAAACTA
GCCCAAGATTGTTGGCTATGTTTAAAAGCAAAACCCCCTTATTATGTAGGATTAGGAGTA
GAAGCCACACTTAAACGTGGCCCTCTATCTTGTCATACACGACCCCGTGCTCTCACAAT
AGGAGATGTGTCTGGAAATGCTTCCTGTCTGATTAGTACCGGGTATAACTTATCTGCTTC
TCCTTTTCAGGCTACTTGTAATCAGTCCCTGCTTACTTCCATAAGCACCTCAGTCTCTTA
CCAAGCACCCAACAATACCTGGTTGGCCTGCACCTCAGGTCTCACTCGCTGCATTAATG
GAACTGAACCAGGACCTCTCCTGTGCGTGTTAGTTCATGTACTTCCCCAGGTATATGTG
TACAGTGGACCAGAAGGACGACAACTCATCGCTCCCCCTGAGTTACATCCCAGGTTGCA
CCAAGCTGTCCCACTTCTGGTTCCCCTATTGGCTGGTCTTAGCATAGCTGGATCAGCAG
CCATTGGTACGGCTGCCCTGGTTCAAGGAGAAACTGGACTAATATCCCTGTCTCAACAG
GTGGATGCTGATTTTAGTAACCTCCAGTCTGCCATAGATATACTACATTCCCAGGTAGAG
TCTCTGGCTGAAGTAGTTCTTCAAAACTGCCGATGCTTAGATCTGCTATTCCTCTCTCAA
GGAGGTTTATGTGCAGCTCTAGGAGAAAGTTGTTGCTTCTATGCCAATCAATCTGGAGT
CATAAAAGGTACAGTAAAAAAAGTTCGAGAAAATCTAGATAGGCACCAACAAGAACGAG
AAAATAACATCCCCTGGTATCAAAGCATGTTTAACTGGAACCCATGGCTAACTACTTTAA
TCACTGGGTTAGCTGGACCTCTCCTCATCCTACTATTAAGTTTAATTTTTGGGCCTTGTA
TATTAAATTCGTTTCTTAATTTTATAAAACAACGCATAGCTTCTGTCAAACTTACGTATCTT
AAGACTCAATATGACACCCTTGTTAATAACTGA
SEQ ID NO: 4
H. sapiens
DNA
>HERV W Env / Syncytin 1 (T0264)
ATGGCCCTCCCTTATCATATTTTTCTCTTTACTGTTCTTTTACCCTCTTTCACTCTCACTG
CACCCCCTCCATGCCGCTGTATGACCAGTAGCTCCCCTTACCAAGAGTTTCTATGGAGA
ATGCAGCGTCCCGGAAATATTGATGCCCCATCGTATAGGAGTCTTTCTAAGGGAACCCC
CACCTTCACTGCCCACACCCATATGCCCCGCAACTGCTATCACTCTGCCACTCTTTGCA
TGCATGCAAATACTCATTATTGGACAGGAAAAATGATTAATCCTAGTTGTCCTGGAGGAC
TTGGAGTCACTGTCTGTTGGACTTACTTCACCCAAACTGGTATGTCTGATGGGGGTGGA
GTTCAAGATCAGGCAAGAGAAAAACATGTAAAAGAAGTAATCTCCCAACTCACCCGGGT
ACATGGCACCTCTAGCCCCTACAAAGGACTAGATCTCTCAAAACTACATGAAACCCTCC
GTACCCATACTCGCCTGGTAAGCCTATTTAATACCACCCTCACTGGGCTCCATGAGGTC
TCGGCCCAAAACCCTACTAACTGTTGGATATGCCTCCCCCTGAACTTCAGGCCATATGT
TTCAATCCCTGTACCTGAACAATGGAACAACTTCAGCACAGAAATAAACACCACTTCCGT
TTTAGTAGGACCTCTTGTTTCCAATCTGGAAATAACCCATACCTCAAACCTCACCTGTGT
AAAATTTAGCAATACTACATACACAACCAACTCCCAATGCATCAGGTGGGTAACTCCTCC
CACACAAATAGTCTGCCTACCCTCAGGAATATTTTTTGTCTGTGGTACCTCAGCCTATCG
TTGTTTGAATGGCTCTTCAGAATCTATGTGCTTCCTCTCATTCTTAGTGCCCCCTATGAC
CATCTACACTGAACAAGATTTATACAGTTATGTCATATCTAAGCCCCGCAACAAAAGAGT
ACCCATTCTTCCTTTTGTTATAGGAGCAGGAGTGCTAGGTGCACTAGGTACTGGCATTG
GCGGTATCACAACCTCTACTCAGTTCTACTACAAACTATCTCAAGAACTAAATGGGGACA
TGGAACGGGTCGCCGACTCCCTGGTCACCTTGCAAGATCAACTTAACTCCCTAGCAGCA
GTAGTCCTTCAAAATCGAAGAGCTTTAGACTTGCTAACCGCTGAAAGAGGGGGAACCTG
TTTATTTTTAGGGGAAGAATGCTGTTATTATGTTAATCAATCCGGAATCGTCACTGAGAA
AGTTAAAGAAATTCGAGATCGAATACAACGTAGAGCAGAGGAGCTTCGAAACACTGGAC
CCTGGGGCCTCCTCAGCCAATGGATGCCCTGGATTCTCCCCTTCTTAGGACCTCTAGCA
GCTATAATATTGCTACTCCTCTTTGGACCCTGTATCTTTAACCTCCTTGTTAACTTTGTCT
CTTCCAGAATCGAAGCTGTAAAACTACAAATGGAGCCCAAGATGCAGTCCAAGACTAAG
ATCTACCGCAGACCCCTGGACCGGCCTGCTAGCCCACGATCTGATGTTAATGACATCAA
AGGCACCCCTCCTGAGGAAATCTCAGCTGCACAACCTCTACTACGCCCCAATTCAGCAG
GAAGCAGTTAG
SEQ ID NO: 5
H. sapiens
DNA
>HERV FRD Env /Syncytin2 (NM_207582.3)
ATGGGCCTGCTCCTGCTGGTTCTCATTCTCACGCCTTCACTAGCAGCCTACCGCCATCC
TGATTTCCCGTTATTGGAAAAAGCTCAGCAACTGCTCCAAAGTACAGGATCCCCTTACTC
CACCAATTGCTGGTTATGTACTAGCTCTTCCACTGAAACACCAGGGACAGCTTATCCAG
CCTCGCCCAGAGAATGGACAAGCATAGAGGCGGAATTACATATTTCCTATCGATGGGAC
CCTAATCTGAAAGGACTGATGAGGCCTGCAAATAGTCTTCTTTCAACAGTAAAGCAAGAT
TTCCCTGATATCCGCCAGAAACCTCCCATTTTCGGACCCATCTTTACTAATATCAACCTA
ATGGGAATAGCCCCTATTTGTGTTATGGCCAAAAGGAAAAATGGAACAAATGTAGGCAC
TCTTCCAAGTACAGTCTGTAATGTTACTTTCACTGTAGATTCTAACCAACAGACTTACCAA
ACATACACCCACAACCAATTCCGCCATCAACCAAGATTCCCCAAACCTCCAAATATTACT
TTTCCTCAGGGAACTTTGCTAGATAAATCCAGCCGGTTTTGCCAGGGACGCCCAAGCTC
ATGCAGTACTCGAAACTTCTGGTTCCGGCCTGCTGATTATAACCAATGTCTGCAAATTTC
CAACCTCAGCTCTACAGCGGAATGGGTTCTATTGGACCAAACTCGAAATTCTCTTTTTTG
GGAAAATAAAACCAAGGGAGCTAACCAGAGCCAAACACCCTGCGTCCAAGTCTTAGCA
GGCATGACTATAGCCACCAGCTACCTGGGCATATCAGCAGTCTCAGAATTTTTTGGAAC
CTCCCTCACCCCCTTATTTCATTTCCATATCTCTACATGCCTTAAAACTCAAGGAGCCTTT
TATATTTGTGGCCAGTCGATTCACCAATGCCTCCCCAGTAACTGGACTGGAACTTGTAC
CATAGGCTATGTAACCCCAGACATCTTCATAGCCCCTGGCAATCTCTCTCTTCCAATACC
AATCTATGGGAATTCCCCGTTGCCCAGGGTGAGGAGGGCAATCCATTTCATTCCCCTTC
TCGCGGGACTCGGCATTCTAGCTGGTACGGGAACCGGAATTGCTGGAATCACAAAAGC
TTCCCTCACCTATAGCCAGCTCTCAAAGGAAATAGCCAACAACATTGACACCATGGCTA
AAGCCTTAACGACCATGCAAGAACAAATCGACTCTTTAGCAGCCGTAGTCCTTCAAAATC
GTCGAGGACTAGACATGTTAACGGCAGCACAGGGAGGAATTTGTTTGGCCTTAGATGAA
AAATGTTGCTTTTGGGTAAATCAATCAGGAAAAGTACAAGACAACATCAGACAACTCCTA
AATCAAGCCTCCAGTTTACGGGAACGAGCCACTCAGGGTTGGTTAAATTGGGAAGGAAC
TTGGAAATGGTTCTCTTGGGTTCTTCCCCTTACAGGCCCACTTGTTAGTCTCCTACTTTT
GCTCCTTTTTGGTCCATGTCTCCTAAATCTAATAACCCAATTTGTCTCCTCTCGCCTTCA
GGCCATAAAGCTCCAGACGAATCTCAGTGCAGGACGCCATCCTCGCAATATTCAAGAGT
CACCCTTCTAA
SEQ ID NO: 6
H. sapiens
DNA
>HERV R Env /ERV3 (AAA88027)
ATGACTAAAACCCTGTTGTATCACACTTATTATGAGTGTGCTGGGACCTGCCTAGGAACT
TGTACTCACAACCAGACAACCTACTCAGTCTGTGACCCAGGAAGGGGCCAGCCTTATGT
GTGTTATGACCCTAAGTCTTCACCTGGGATCTGGTTTGAAATTCATGTCGGGTCAAAGG
AAGGGGATCTTCTAAACCAAACCAAGGTATTTCCCTCTGGCAAGGATGTCGTATCCTTAT
ACTTTGATGTTTGCCAGATAGTATCCATGGGCTCACTCTTTCCCGTAATCTTCAGTTCCA
TGGAGTACTATAGTAGCTGCCATAAAAATAGGTATGCACACCCTGCTTGTTCCACCGATT
CCCCAGTAACAACTTGCTGGGACTGCACAACGTGGTCCACTAACCAACAATCACTAGGG
CCAATTATGCTTACCAAAATACCATTAGAACCAGATTGTAAAACAAGCACTTGCAATTCT
GTAAATCTTACCATCTTAGAGCCAGATCAGCCCATATGGACAACAGGTTTAAAGACACC
GCTAGGGGCACGAGTCAGCGATGAAGAAATTGGCCCAGGAGCCTATGTCTATCTATATA
TCATAAAGAAAACTCGGACCCGCTCAACCCAACAGTTGCGAGTTTTTGAGTCATTCTATG
AGCATGTTAACCAGAAATTGCCTGAGCCCCCTCCCTTGGCCAGTAATTTATTCGCCCAA
CTGGCTGAAAACATAGCCAGCAGCCTGCACGTTGCTTCATGTTATGTCTGTGGGGGAAT
GAACATGGGAGACCAATGGCCATGGGAAGCAAGGGAACTAATGCCCCAAGATAATTTC
ACACTAACCGCCTCTTCCCTCGAACCTGCACCATCAAGTCAGAGCATCTGGTTCTTAAA
AACCTCCATTATTGGAAAATTCTGTATTGCTCGCTGGGGAAAGGCCTTTACAGACCCAG
TAGGAGAGTTAACTTGCCTAGGACAACAATATTACAACGAGACACTAGGAAAGACTTTAT
GGAGGGGCAAAAGCAATAATTCTGAATCACCACACCCAAGCCCATTCTCTCGTTTCCCA
TCTTTAAACCATTCTTGGTACCAACTTGAAGCTCCAAATACCTGGCAGGCACCCTCTGG
CCTCTACTGGATCTGTGGGCCACAAGCATATCGACAACTGCCAGCTAAATGGTCAGGG
GCCTGTGTACTGGGGACAATTAGGCCGTCCTTCTTCCTAATGCCCCTAAAACAGGGAGA
AGCCTTAGGATACCCCATCTATGATGAAACTAAAAGGAAAAGCAAAAGAGGCATAACTAT
AGGAGATTGGAAGGACAGTGAATGGCCTCCTGAAAGAATAATTCAATATTATGGCCCAG
CCACCTGGGCAGAAGATGGAATGTGGGGATACCGCACCCCAGTTTACATGCTTAACCG
CATTATAAGATTGCAGGCAGTACTAGAAATCATTACCAATGAAACTGCAGGGGCCTTGA
ATCTGCTTGCCCAGCAAGCCACAAAAATGAGAAATGTCATTTATCAAAATAGACTGGCCT
TAGACTACCTCCTAGCCCAGGAAGAGGGAGTATGCGGAAAGTTCAGCCTTACTAACTGC
TGCCTGGAACTTGATGACGAAGGAAAGGTTATCAAAGAAATAACTGCTAAAATCCAAAA
GTTAGCTCACATCCCAGTTCAGACTTGGAAAGGATAG
SEQ ID NO: 7
H. sapiens
DNA
>HERV R(b) Env (AC093488)
ATGGATCCACTACACACGATTGAAAAAGTTCCTGCAAGAAGAAACATCCACGACAGAGG
ACACCAAGGCCACCGAATGGGAGATGGAACCCCTGGAAGGCCTAAGATTTCTGTTCAA
CAAATGACAAGATTTTCCCTTATAATATTTTTCCTTTCTGCTCCTTTTGTTGTTAATGCCTC
TACCTCTAACGTTTTCCTACAATGGGCACACAGTTATGCAGATGGCTTACAACAAGGAG
ACCCTTGCTGGGTCTGTGGTTCGTTACCCGTCACTAACACCATGGAGCTACCTTGGTGG
GTCTCCCCGCTACAAGGGAAAGACTGGGTTTTTTTTCAAAGCTTTATAGGGGATCTTAAA
CAATGGACAGGGGCACAGATGACTGGGGTAACTAGAAAAAACATTTCAGAATGGCCTAT
AAATAAAACTTTAAATGAGCCAGGGCATGATAAACCATTCTCAGTAAATGAGACAAGGGA
TAAAGTAATAGCCTTTGCCATCCCCTTGTTGGATACCAAGGTGTTTGTCCAGACTTCCAG
ACCTCAGAACACTCAATATAGAAATGGGTTTCTCCAGATATGGGACGGGTTCATTTGGC
TGACAGCCACTAAGGGACACTTAAGCCAGATAGCTCCCTTATGCTGGGAGCAAAGAAAT
CACTCCCTTGATAACTGGCCAAACACAACTCGTGTTATGGGATGGATTCCACCTGGACA
GTGCCGACATACTATACTGTTACAACAGAGGGACCTATTTGCCACAGACTGGTCTCAGC
AACCTGGCTTGAATTGGTATGCTCCCAACGGAACCCAGTGGCTCTGCAGCCCAAACTTA
TGGCCTTGGCTTCCCTCAGGTTGGTTAGGATGCTGCACTCTAGGTATTCCCTGGGCACA
AGGACGCTGGGTAAAAACCATGGAAGTCTATCCTTATCTTCCACATGTGGTTAACCAAG
GGACTAGGGCCATTGTTCACAGGAATGATCACCTACCCACAATCTTTATGCCCTCAGTA
GGTTTAGGAACTGTAATACAGCACATAGAGGCTCTAGCCAATTTTACCCAACGGGCCCT
AAATGACAGCCTCCAAAGTATTTCTCTCATGAATGCTGAAGTGTATTATATGCACGAGGA
CATCTTACAAAACCGAATGGCCCTAGATATTTTAACTGCGGCTGAAGGAGGAACCTGTG
CCCTCATCAAAACTGAATGTTGTGTGTATATTCCCAATAACTCTAGAAACATTTCCTTGG
CCTTAGAGGATACATGTCGGCAAATCCAAGTCATCTCCAGCTCTGCACTGTCACTCCAT
GACTGGATAGCATCTCAGTTTAGTGGAAGACCTTCCTGGTGGCAGAAAATCCTCATTGT
CCTTGCCACCCTCTGGAGCGTAGGCATAGCACTGTGTTGTGGACTGTATTTTTGTCGCA
TGTTTTCCCAACACATTCCCCAAACTCATTCGATTATATTTCAACAGGAACTTCCCTTGAG
CCCCCCAAGTCAGGAGCATTACCAGAGCCAAAGAGACATCTTCCACTCTAACGCCCCCT
GA
SEQ ID NO: 8
H. sapiens
DNA
>HERV F(c)2 Env (AC016222)
ATGAATTCTCCATGTGACAGGCTCCAACAATTTATTCAGGTTCTTCTCGAGGAAAGCTGG
TCATTCCCTAGTTTTGCTAACACCCTTCACTGGCCTGAAAATCTGTTGTCCTATATAGAC
GAACTGGTATGGCAAGGCTCCCTCCAGAACTTTCACCAACATGAAGTTCGCTTTGACAA
GCCCCCTCTCAGACTCCCTCTCACTGGGTTTTCTTCCCTCACTGAGAATTGGAGTTCCA
GACAGGCAGTGTCCTCTAGACTAGTAGCCACGGCAGCATCCCCGCCAGCAGGGTGTCA
GGCACCCATAGCTTTCCTAGGTCTAAAATTCTCTTCCCTAGGCCCGGCTAGAAAAAACC
CTGCACTTTGCTTCCTCTATGATCAAAGTAACTCCAAATGCAATACCAGCTGGGTCAAAG
AAAATGTAGGCTGTCCGTGGCACTGGTGCAATATCCATGAGGCATTAATTCGTACTGAA
AAAGGATCTGACCCAATGTTCTATGTCAATACCTCCACTGGAGGACGGGACGGCTTTAA
CGGATTTAACCTCCAAATTTCTGACCCTTGGGACCCCCGCTGGGCCTCCGGTGTAGATG
GAGGACTATATGAGCACAAAACTTTTATGTATCCAGTAGCTAAGATCCGCATTGCCAGG
ACCCTTAAAACCACTGTCACAGGGTTATCCGACTTAGCCTCCTCAATCCAGTCAGCCGA
GAAAGAGCTTACCAGCCAGCTTCAACCGGCAGCTGACCAGGCCAAGTCCTCCCGCTTC
TCGTGGTTAACTTTAATCTCAGAAGGTGCACAATTGCTCCAATCCACAGGGGTACAAAA
CCTCTCCCACTGCTTCCTCTGTGCAGCCCTCAGAAGACCTCCATTAGTAGCAGTTCCTC
TCCCTACCCCCTTTAATTATACTATAAATTCATCAACCCCTATACCACCGGTCCCAAAAG
GACAGGTCCCACTATTCTCAGACCCTATAAGACATAAGTTCCCATTCTGTTACTCTACCC
CAAATGCCTCTTGGTGTAACCAGACTAGGATGCTTACCAGCACCCCGGCACCGCCCAG
AGGCTACTTCTGGTGTAACTCCACGCTAACTAAAGTTCTTAACTCAACTGGTAATCACAC
CTTGTGCTTACCCATCTCTCTCATCCCTGGCCTGACCCTATATAGTCAGGATGAACTTAG
CCATCTGCTAGCCTGGACCGAGCCAAGGCCACAAAATAAAAGCAAATGGGCTATTTTCT
TACCCTTAGTACTAGGCATCTCCTTAGCCTCCTCCTTAGTGGCATCAGGGCTAGGAAAA
GGAGCCCTCACCCACTCAATCCAAACATCTCAAGATCTGTCTACTCACCTGCAGTTGGC
CATTGAGGCATCGGCCGAGTCCCTGGACTCCCTACAGCGACAGATCACTACAGTAGCA
CAGGTCGCAGCACAGAACAGGCAGGCCTTAGACTTGCTTATGGCAGAAAAAGGAAGGA
CATGTTTATTCTTACAGGAAGAATGCTGCTATTATCTCAATGAGTCAGGGGTAGTTGAGA
ACAGTTTACAAACTTTAAAAAAAAAAAAAAGCTCCAAGAGGAGTTAA
SEQ ID NO: 9
H. sapiens
DNA
>HERV F(c)1 Env (AL354685)
ATGGCCAGACCTTCCCCACTATGCCTCCTACTCCTCCTGACCCTCCTAACCCCCATAGT
GCCCAGTAACTCCCTCCTAACTGAACCCCCGTTCCGATGGAGGTTCTACCTGCATGAGA
CTTGGACCCAAGGCAACCGGCTCTCCACTGTCACACTGGCAACGGTGGACTGCCAACC
TCACGGTTGTCAGGCCCAAGTAACTTTTAACTTCACTTCCTTTAAAAGTGTTCTGCGGGG
CTGGTCCAATCCCACCATCTGCTTTGTCTATGATCAAACACACAGCAACTGCCGCGACT
ATTGGGTGGACACAAACGGAGGATGCCCCTATGCCTATTGTCGTATGCATGTGACCCAG
CTCCATACCGCCAAGAAACTCCAACACACCTATCGCCTGACATCTGATGGAAGGACAAC
TTACTTCCTGACCATCCCAGACCCATGGGATTCTCGGTGGGTCAGTGGAGTCACTGGTC
GACTGTACCGGTGGCCCACCGACTCCTACCCAGTTGGCAAACTCCGGATATTCCTGACT
TATATACGAGTTATCCCCCAGGTTTTGTCTAATTTAAAGGACCAAGCAGACAACATTAAG
CATCAGGAAGAGGTCATCAATACTTTGGTGCAGTCCCATCCGAAGGCTGACATGGTCAC
CTATGATGACAAGGCTGAGGCAGGACCGTTTTCATGGATAACCCTAGTCCGCCACGGG
GCTCGCCTTGTTAATATGGCAGGCCTAGTTAATCTCTCCCACTGTTTCCTTTGCACCGCC
CTCAGCCAACCACCACTAGTAGCTGTACCCCTACCCCAGGCTTTTAACACCTCTGGTAA
CCACACTGCCCACCCTTCCGGCGTCTTCTCTGAGCAGGTCCCTCTTTTCCGAGACCCCC
TCCAGCCCCAGTTCCCCTTCTGCTACACCACTCCTAACTCATCCTGGTGCAACCAGACC
TATTCTGGCTCCCTATCTAACCTCTCTGCACCGGCAGGTGGCTACTTCTGGTGTAACTT
CACCCTTACAAAACATCTTAATATTTCCTCTAACAATACCCTTTCTAGAAACTTATGCCTC
CCCATCTCTCTGGTGCCTCGACTCACTCTGTACAGCGAGGCTGAACTCTCTTCCCTTGT
CAACCCGCCTATGCGTCAGAAGCGGGCCGTTTTCCCACCGCTGGTAATAGGTGTCTCC
TTGACCTCCTCACTTGTTGCCTCCGGGCTGGGCACAGGTGCTATTGTACATTTCATAAG
CTCTTCCCAAGATCTCTCTATTAAGCTCCAGATGGCCATCGAAGCCTCAGCCGAATCCT
TAGCCTCTCTACAGAGACAGATTACGTCTGTGGCCAAGGTGGCCATGCAGAACCGGAG
AGCCCTAGATCTCCTCACAGCCGACAAAGGCGGAACCTGCATGTTTCTCGGGGAAGAG
TGCTGTTATTACATCAATGAATCAGGCTTAGTAGAAACCAGCCTCCTCACCCTTGATAAA
ATCCGGGACGGTCTCCATCGACCCTCCTCAACTCCCAACTATGGAGGAGGGTGGTGGC
AATCCCCTTTAACCACTTGGATTATCCCTTTCATAAGCCCCATCCTAATCATTTGCCTTTT
ACTTCTCATAGCCCCCTGTGTCCTCAAGTTCATCAAAAACCGCATCAGCGAAGTCTCCC
GGGTGACGGTCAACCAAATGTTACTACACCCTTACTCCCGTCTTCCGACCTCCGAAGAC
CACTATGACGACGCCCTCACTCAGCAGGAAGCAGCCAGATGATTACGTCGCCCCTTTTT
CTTACAGTATGAGGTCGGAATGTTAGGCAGGTCACCCAAGATGGCTGTTCCCCCAGGA
CCCAAGATGGCGGCACGAACCCCTTCTTCCCCGCCCCCCCCACCGCTTGGAGTCTCCC
ACCAGATTTTCCCGCCCGACGGGCACTTTCCGATGACAGCAGCCCCGAGAAGTCGAAA
CCTATCCCAGAAAACCGAAACTTACTAA
SEQ ID NO: 10
H. sapiens
DNA
>HERV E Env (AY208746)
ATGAGGAAGCTCATCGTGGGATTCATTTTTCTTACATTTTGGACTTACACAGTAAGGGCT
TCAACTGACCTTACTCAAACTGGGGACTGTTCCCAGAGTATTCATCAGGTCACCGAGGT
AGGACAGCAAATTAAAACAAACTTTCTGTTCTATAGTTATTATGAATGTATGGGAACATTA
AAAGAAACTTGTTTGTATAATGCCACTCAGTACAAGGTATGTAGCCCAGGAAATGACCG
ACCCGATGTGTGTTATAACCCATCTGAGCCCCCTGCAACCACCGTTTTTGAAATAAGATT
AAGAACTGGCCTTTTCCTAGGTGACACAAGTAAAATAATAACTAGAACAGTAGAAAAAGG
AATCCCCAAACAAATAACTTTAAGATTTGATGCTCGTGCAGCCATTAATAGTAACAAGTT
AGGAACACGATGTGGTTCTCTTAACTGGGAAAGGAGCTACACAGTACAAAATAAATATG
TTTGTCATGAGTCAGGGGTTTGTGAAAATTGTGCCTTTTGGCCATGTGTTATTTGGGCTA
CTTGGAAAAAGAACAAAAAGGACCCTGTTCATCTTCAGAAGGGGGAAGCCAACCCCTCC
TGTGCTGCCGGTCACTGTAACCCACTAGAACTAATAATTACCAATCCCCTAGATCCTCCT
TGGAAAAAGGGAGAACGTGTAACCCTGGGGATCGATGGGACAGGGTTAAACCCCCAAG
TTGCCATTTTAGTTAGAGGGGAAGTCCACAAGCGCTCTCCCAAACCAGTGTTTCAAACC
TTTTATGAGGAGCTGAATCTGCCAGCACCAGAACTTCCGAAAAAGACAAAAAGTTTGTTT
CTCCAATTAGCAGGAAATGTAGCTCATTCACTTAATGTTACTTCCTGTTATGTATGCAGG
GGAACCACTATCGGAGACCGATGGCCTTGGGAAGCCCGAGAGTTGGTGCCTACTGATC
CAGCTCCTGATATAATTCCAGTTCAGAAGGCCCAAGCTAGCAACTTCTGGGTCCTAAAA
ACCTCAATTATTGGACAATACTGTATAGCTAGAGAAGGGAAAGAATTTATCGTCCCTGTA
GGAAAGCTTAATTGTATAGGACAGAAGTTGTACAACAGCACAACAAAGACAATTACTTGG
TGGGGCCTAAACCACACTGAAAAGAACCCATTTAGTAAATTTTCTAAATTAAAAACTGCC
TGGGCTCATCCAGAATCTCATCAGGACTGGACGGCTCCCACCGGACTATACCGGATAT
GTGGGCACACAGCCTACATTCAGTTACCTAATAAATGGGCAGGCAGTTGTGTTATTGGC
ACTATTAAGCTGTCCTTTTTCTTATTACCCATAAAAACGGGTGAGCTCCTAGGTTTCCGT
GTCTATACCTCCCGAGAAAAGAGAGGCATAGTTATAGGAAACTGGAAAGATAATGAGTG
GCCCCCTGAAAGGATCATACAATATTATGGGCCTGCCACATGGGTACAAGACGGCTCAT
GGGGATACCAAACCCCCATCTACATGCTCAATCAGATCATACGGTTGCAGACCGTCTTA
GAAATAATTACTAATGAAACTGGCAGAGCTTTGACTGTTTTAGCTCGGCAGGAAACCCAA
ATGAGGAATGCTATCTATCAGAATAGACTGGCCTTGGACTACTTGCTGGCAGCTGAAGG
AGGAGTTTGTGGAAAATTTAACTTAACCAATTGCTGCCTACAAATAGATGATCAAGGACA
GGTGATTGAAAACATAGTCAGGGACATGACAAAGTTGGCACACACGCCTATACAGGTTT
GGCACAAGTTTGATCCTGAGTCTTTATTTGGAAAATGGTTTCCAGCTATAGGAGGATTTA
AAACCCTCATTGTAGGTGTATTGCTAGTAATAAGAACTTGCTTGCTGCTCCCCTGTGTAT
TACCCTTGCTTTTTCAAATGATAAAAGGTATTGTTGCTACTTTGGTTCATCAGAAAACTTC
AGCACACGTGAATTATATGAATCACTATCGCTCTATCTCGCAAAGAGACTCAAAAAGTGA
GGATGAGAGTGAGAACTCCCACTAA
SEQ ID NO: 11
H. sapiens
DNA
>HERV P(b1) Env /HERV IP (DQ247958)
ATGGACCCATCACACCCGAGTCAAGAAAGCACCGCCCCCTCCAGCGTCATGGGCCATA
GTCCCAGGGGAAAATCCTACCAAACTAAAGCTAAGGAAAGTTTAATTCTCTTTCATCTAT
TCTGTTACTCCTTCTTCTTTCCTTGTGCTCTTGCTAGCCACCTCATTATTAACGTAACTAG
ATCAGACTCACCCCAGACCATTACCTTCGATGCTTGTTTAGTTATACCTTGTGGGGATCT
CCAAAGCCAGAGACAGCTTGCAGCAGCAGAGAAATATCTCTGCCCCTCCGAAGCAGAT
GCTTCTACCTTGTTTAGCTTTCCGTTTTGTCATACTTGGGAATATGTCGTTTGGACCACT
CAACGTCAAGATTGGGTCCCCTCACAGGATTTCCCGCTAGCGGTTCTAAAGCCCTATAT
CCATTTTACTAAAGGAATTGCCCCTCCCAATTGTCGATATAACCAATGTAACCCGGTGCA
AATTTCCATCACCATCCCAACTCTCCAAGATTCCTCCCCCACCCTAAACCGTTTCTATGG
TATGGGAGCAGATGTAAGAGGGAAAGACCCCATAGGATTCTTCGAGTTGCACCTCAGTA
CATCTCCATCCCTCATATCTCCACGTCTATCCTCTTCTACACCTGCTAACCAGACCATTG
TCTCTTCATCTAATGACAAAAGCAAAGTAGCTATTGTAGAGGTTAAAAATTTAAAACAAAC
ACTGACAATTGAAACAGGATACAAAGAAACAAATGCCTGGATGGAATGGATTGAATATTC
CGTTCGCAGTCTAAACAAAAGCGACTGTTACGCTTGTGCGCAAGGTAGGCCAGAGGCC
CAAGTCGTCCCCTTTCCACTTGGATGGTCTTCTGACCAACCGGGCATGGGCTGCATGGT
GGCTCTTTTCCAACACCCTACAGCCTGGGATAGTGAGTTCTGTCGAACTCTCTCTGTGC
TATTCCCTGAAACTCAACACCTTGAGGGTGAGCCCCCGAGGGCCATCCAGCCTCCATCT
CCAGATGCCAAGTTTACTTCCTGTCTCTCACGACAGGGAAAAAATTTGGAGTTTCTTGG
GGACCTAAAGGGATGCAGTGAGCTTAAGTCTTTCCAAGAGCTTACCAATCAGTCTGCAC
TTGTTCATGCCCGAGCTGATGTATGGTGGTATTGTGGTGGTCACCTGCTAGACACTCTG
CCAAGTAACTGGAGTGGTACTTGTGCTCTAATTCAATTGGCCATCCCTTTCACCCTGGC
ATTTCAACAACCAGAAAAAAAGAAACCACAACGCCGTAAAACAAGAGAGGCCCCTCAAG
GATCTTTCGACTCTCATGTCTATGTAGATGAAATTGGAGTCCCACAGGGGGTACCTGAT
AGATTCAAAGCCCGAGACCCAGTAGCTGCAGGATTTGAATCATTATTTCCAATGGTAGC
TATTAATAAAAATGTAGCTTGGATAAATTACATCTATTATAACCAGCAGCGATTTATTAATT
ACACTAGGGATGCTATCCAAGGAATAGCTGAACAATTAGGGCCTACTAGCCAAATGGCT
TGGGAAAACAGAATGGCCTTAGATATGATATTAGCAGAAAAAGGTGGAGTTTGTGTTAT
GATAGGAACCCAATGCTGCACCTACATTCCCAACAATACAGCTCCTGATGGAACAATTA
CAAAAGCTTTACAAGGTCTTACCTCCTTATCAGATGAATTAGCCACAAATTCTGGGATAA
CTGACCCTTTCACAGGATGGTTAGGGCAATGGTTTGGTAAATGGAAAGGACTCATGGCC
TCTATTGTTACCTCTCTCGCAATCGCAATAGCTGTGCTTATTCTTGTTGGATGCTGCATC
ATGCCCTGCATTCGTGGACTAGTCCAAAGACTTATAGAGACAGCTAGTAACAAAACCTT
CCCTAGTTCTTCCCAATCCTATAGTAACAAATTCTTCCCCGTGAACGAACACGAAATCCG
AATCATATTAGATAGGTTTAAAGCAGAACATGTATAA
SEQ ID NO: 12
H. sapiens
DNA
>HERV V Env (NM_001191055)
ATGACAGAGAAATTCCTTTTCCTTTATCTTTCCCTCCTTCCCATGCCCCTACTCTCACAG
GCACAGTGGAATGAAAATTCCCTTGTCAGTTTTTCCAAAATAATTGCTTCGGGAAACCAT
CTAAGCAACTGTTGGATCTGCCACAACTTCATCACCAGGTCCTCATCTTACCAATATATT
TTGGTAAGAAATTTTTCTTTAAACCTAACATTTGGTTCAGGAATCCCTGAAGGCCAACAT
AAATCTGTTCCGCTCCAGGTTTCGCTTGCTAACTCAGCGCACCAAGTCCCCTGCCTGGA
TCTCACTCCACCTTTCAATCAAAGCTCTAAAACTTCTTTCTATTTCTACAACTGCTCTTCT
CTAAACCAAACCTGTTGTCCATGCCCTGAAGGACACTGTGACAGGAAGAACACCTCTGA
GGAGGGATTCCCCAGTCCCACCATCCATCCCATGAGCTTCTCCCCAGCAGGCTGCCAC
CCTAACTTGACTCACTGGTGTCCAGCTAAACAAATGAACGATTATCGAGACAAGTCACC
CCAAAACCGCTGTGCAGCTTGGGAAGGAAAAGAGCTAATCACATGGAGGGTTCTATATT
CGCTTCCCAAGGCACACACTGTCCCCACATGGCCAAAATCTACTGTTCCCCTGGGAGG
GCCTCTATCCCCTGCATGCAATCAAACTATTCCAGCAGGGTGGAAATCGCAGTTACACA
AGTGGTTCGACAGCCACATCCCCCGGTGGGCCTGTACCCCTCCTGGCTATGTATTTTTA
TGTGGGCCACAAAAAAATAAACTGCCCTTTGATGGAAGTCCTAAGATAACCTATTCAACC
CCCCCTGTGGCAAACCTCTACACTTGCATTAATAACATCCAACATACGGGAGAATGTGC
TGTGGGACTTTTGGGACCACGGGGGATAGGTGTGACCATTTATAACACCACCCAACCCA
GACAGAAAAGAGCTCTGGGTCTAATACTGGCAGGGATGGGTGCGGCCATAGGAATGAT
CGCCCCATGGGGAGGGTTCACTTATCATGATGTCACCCTCAGAAATCTCTCCAGACAAA
TAGACAACATAGCTAAGAGTACCAGAGATAGCATCTCTAAACTCAAGGCCTCCATAGATT
CTCTAGCAAATGTAGTCATGGACAACAGATTGGCCTTAGATTACCTCTTAGCAGAGCAG
GGTGGAGTCTGTGCAGTGATCAATAAATCCTGTTGCGTTTATGTCAATAACAGTGGGGC
GATAGAGGAGGATATAAAAAAGATCTATGATGAGGCTACGTGGCTCCATGACTTTGGAA
AAGGAGGTGCTTCAGCAAGGGCCATCTGGGAGGCTGTGAAGTCTGCCCTCCCCTCCCT
CAACTGGTTTGTCCCTTTACTGGGACCAGCAACAGTTATACTCTTACTTTTCCTCTTTGG
CCCTTGTTTCTTTAATTTACTGATTAAGTGTGTCTCTTCTAGGATAAAGCAATTTCACATG
AAGTCCCCCCAAATGGAAAGATATCAGCTATCTGTCATTGGAGGCCCCAGCACCTATAA
GCACATCTCCCCCTTGGATGCCAGTGGGCAAAGATTCCGGGAAACTATGGAGGAATTTT
CTCTCTGA
SEQ ID NO: 13
H. sapiens
DNA
>HERV MER34 Env (NM_001242690.1, NM_024534)
ATGGGCTCCCTTTCAAACTATGCCCTGCTTCAACTAACCCTTACTGCTTTTTTGACAATT
CTAGTACAACCTCAGCACCTGCTTGCTCCAGTTTTCCGGACACTATCTATCTTGACTAAT
CAGTCTAATTGCTGGTTATGTGAACATCTAGATAATGCAGAACAACCCGAACTAGTTTTT
GTTCCTGCCAGTGCAAGCACCTGGTGGACCTATTCTGGACAATGGATGTATGAAAGGGT
GTGGTATCCACAAGCAGAAGTACAGAATCACTCTACTTCCTCCTATCGTAAAGTGACTTG
GCACTGGGAAGCCTCCATGGAAGCTCAAGGTCTATCCTTTGCTCAAGTAAGGTTATTGG
AGGGAAATTTTTCTCTTTGCGTAGAAAATAAAAATGGCAGTGGACCCTTCCTAGGTAATA
TACCTAAACAATACTGTAATCAAATACTATGGTTTGATTCTACAGATGGCACCTTCATGC
CCTCTATAGATGTTACAAATGAATCCAGGAACGATGATGATGATACAAGTGTTTGCCTAG
GCACTAGACAATGTTCCTGGTTTGCAGGTTGCACAAACCGGACCTGGAACAGCTCAGCT
GTTCCCTTGATTGGTCTGCCCAATACCCAAGACTACAAATGGGTAGATCGAAATTCTGG
ATTGACCTGGTCAGGTAATGACACCTGTCTCTATAGCTGCCAAAACCAAACCAAAGGCC
TTCTGTACCAGCTATTTCGCAACCTATTTTGCTCTTATGGCCTGACAGAGGCACATGGGA
AATGGAGATGTGCAGATGCCAGCATAACTAATGACAAAGGTCATGATGGACACCGGACC
CCCACCTGGTGGCTCACAGGTTCCAATCTGACCTTGTCTGTGAACAACTCTGGCCTCTT
TTTTTTGTGCGGCAATGGGGTGTACAAAGGGTTTCCACCTAAATGGTCTGGGCGATGTG
GACTTGGGTATCTTGTACCTTCCCTCACCAGATACCTCACCTTAAATGCTAGCCAAATTA
CAAACCTGAGATCCTTCATTCATAAAGTAACACCGCATAGATGCACCCAAGGAGACACA
GACAATCCACCTCTGTATTGCAACCCCAAGGACAATTCAACAATAAGGGCCCTTTTTCCA
AGTTTGGGAACTTATGATTTAGAAAAGGCAATTCTAAACATTTCCAAAGCAATGGAACAG
GAATTCAGTGCCACTAAGCAGACCTTGGAAGCACACCAATCAAAAGTTAGCAGTTTAGC
CTCTGCATCCCGAAAGGATCATGTCTTGGATATACCGACCACCCAACGACAAACGGCTT
GTGGAACTGTTGGCAAACAGTGTTGCCTCTATATAAATTATTCGGAAGAAATAAAGTCTA
ATATACAGCGTCTCCACGAAGCATCCGAGAACCTGAAGAATGTACCGTTACTTGATTGG
CAAGGCATATTTGCAAAAGTGGGAGACTGGTTCAGATCATGGGGCTATGTGCTTTTAAT
TGTTCTTTTCTGCTTATTCATCTTTGTTTTAATCTATGTTCGTGTCTTTCGCAAATCTCGCA
GATCCCTTAACTCCCAACCTCTGAACCTAGCCTTATCTCCACAGCAATCAGCACAGCTC
CTTGTCAGTGAAACTTCATGTCAAGTTTCAAATAGGGCAATGAAGGGACTAACAACCCAT
CAATATGACACAAGTCTACTTTGA
SEQ ID NO: 14
H. sapiens
DNA
>HERV-H Gag consensus sequence
ATGGGCAACCTTCCACCCTCCATTCCTCCTTCTTCTCCCTTAGCCTGTGTTCTCAAGAAC
TTAAAACCTCTTCAACTCACACCTGACCTAAAACCTAAATGCCTTATTTTCTTCTGCAATA
CCGCTTGGCCCCAATACAAACTTGACAGTGGCTCTAAATGGCCAGAAAATGGCACTTTC
AATTTTTCCATCCTACAAGACCTAAATAATTTTTGTCGAAAAATGGGCAAATGGTCTGAG
GTGCCTGACGTCCAGGCATTTTTCACACATCGTTCCCTCCCTAGTCTCTGTTCCCAATG
CAACTCCTCCCAAATCTTCCTTCTTTCCCTCCCGCCTGTCCCCTCAGTCCCAACCCCAA
GCGTCGCTGAGTCTTTCCAATCTTCCTTTTCTACAGACCCATCTGACCTCTCCCCTCCTC
CCCAGGCTGCTCATCGCCAGGCCGAGCTAGGTCCCAATTCTTCCTCAGCCTCCGCTCC
TCCACCCTATAATCCTTTTATCACCTCCCCTCCTCACACCCGGTCCGGCTTACAGTTTCG
TTCCGTGACTAGCCCTCCCCCACCTGCCCAGCAATTTCCTCTTAAAAAGGTGGCTGGAG
CTAAAGGCATAGTCAAGGTTAATGCTCCTTTTTCTTTATCAGACCTCTCCCAAATCAGTC
AGCGTTTAGGCTCTTTTTCAtCAGACCCCACTAAATATATACAGGAATTCCAACATCTAAC
TCTGTCCTACAATTTAACCTGGAGTGACTTAAATGTCATCCTGACTTCTACCCTCTCCCC
AGATGCACAGGAAAGAGTTTTTTCTCTAGCCCAATCTCATGCTGATAACCGCCGGCTtCA
TGAGCCAGACCTCCAGGAAGGCATTAGAGCAGTTCCCCGAGAGGATCCCCAATGGAAC
TACCAGGCAAATTCCCCAGGTATAGCTAAGCAAGATTACATGGTTTCCTGCGTAGTTGA
AGGGCTTAAAAAAGCAGCTTACAAAGCTATTAATTATGACAAACTtAAAGAAACTACCCAA
GGTAAAGATAAAAACCCAGCCCAGTTCATGGCTCGTTTGGCAGCAACCCTCAGACGCTT
TACAGCCCTAGACCCTAAAAGGTCAAAAGGCCGTCTTATTCTCAATATACATTTTATTAC
CCAATCTGCTCCCGACATTAAAAAACTCCAAAAATTAAATTCTGGCCCTCAAACCCCACA
ACAGGATTTAATTAACCTCGCCTTCAAGGTGTACAATAATAGAAAAAAAGCAGCCAGATG
GCAACGCATTTCTGAGTTGCAATTCCTTGCCTCCACTGTGAGACAAACCCCAGCCACAT
CTCCAGCACACAAGAACTTCCAAATGCCTGAACCGCAGCAGCCAGGCATTCCTCCAGAA
CCTCCTCCCCCAGGAGCTTGCTACAAGTGCCAGAAATCTGGCCACTgggCCAAGGAATG
CCCGCAGCCCAGGATTCCTCCTAAGCCGTGTCCCATCTGTGCAGGACCCCACTGGAAA
TCAGACTgTtCAACTCACCTGGCAGCCACTCCCAGAGCCCCTGGAACTCTGGCCCAAGG
CTCTCTGACTGACTCCTTCCCAGATCTGCTCGGCTTAGCGGCTGAAGACTGA
SEQ ID NO: 15
H. sapiens
DNA
>HERV-K consensus Gag-Pol
ATGGGGCAAACTAAAAGTAAAATTAAAAGTAAATATGCCTCTTATCTCAGCTTTATTAAAA
TTCTTTTAAAAAGAGGGGGAGTTAAAGTATCTACAAAAAATCTAATCAAGCTATTTCAAAT
AATAGAACAATTTTGCCCATGGTTTCCAGAACAAGGAACTTTAGATCTAAAAGATTGGAA
AAGAATTGGTAAGGAACTAAAACAAGCAGGTAGGAAGGGTAATATCATTCCACTTACAG
TATGGAATGATTGGGCCATTATTAAAGCAGCTTTAGAACCATTTCAAACAGAAGAAGATA
GCGTTTCAGTTTCTGATGC
CCCTGGAAGCTGTATAATAGATTGTAATGAAAACACAAGGAAAAAATCCCAGAAAGAAAC
GGAAGGTTTACATTGCGAATATGTAGCAGAGCCGGTAATGGCTCAGTCAACGCAAAATG
TTGACTATAATCAATTACAGGAGGTGATATATCCTGAAACGTTAAAATTAGAAGGAAAAG
GTCCAGAATTAGTGGGGCCATCAGAGTCTAAACCACGAGGCACAAGTCCTCTTCCAGCA
GGTCAGGTGCCCGTAACATTACAACCTCAAAAGCAGGTTAAAGAAAATAAGACCCAACC
GCCAGTAGCCTATCAATACTGGCCTCCGGCTGAACTTCAGTATCGGCCACCCCCAGAAA
GTCAGTATGGATATCCAGGAATGCCCCCAGCACCACAGGGCAGGGCGCCATACCCTCA
GCCGCCCACTAGGAGACTTAATCCTACGGCACCACCTAGTAGACAGGGTAGTGAATTAC
ATGAAATTATTGATAAATCAAGAAAGGAAGGAGATACTGAGGCATGGCAATTCCCAGTAA
CGTTAGAACCGATGCCACCTGGAGAAGGAGCCCAAGAGGGAGAGCCTCCCACAGTTGA
GGCCAGATACAAGTCTTTTTCGATAAAAATGCTAAAAGATATGAAAGAGGGAGTAAAACA
GTATGGACCCAACTCCCCTTATATGAGGACATTATTAGATTCCATTGCTCATGGACATAG
ACTCATTCCTTATGATTGGGAGATTCTGGCAAAATCGTCTCTCTCACCCTCTCAATTTTTA
CAATTTAAGACTTGGTGGATTGATGGGGTACAAGAACAGGTCCGAAGAAATAGGGCTGC
CAATCCTCCAGTTAACATAGATGCAGATCAACTATTAGGAATAGGTCAAAATTGGAGTAC
TATTAGTCAACAAGCATTAATGCAAAATGAGGCCATTGAGCAAGTTAGAGCTATCTGCCT
TAGAGCCTGGGAAAAAATCCAAGACCCAGGAAGTACCTGCCCCTCATTTAATACAGTAA
GACAAGGTTCAAAAGAGCCCTATCCTGATTTTGTGGCAAGGCTCCAAGATGTTGCTCAA
AAGTCAATTGCCGATGAAAAAGCCCGTAAGGTCATAGTGGAGTTGATGGCATATGAAAA
CGCCAATCCTGAGTGTCAATCAGCCATTAAGCCATTAAAAGGAAAGGTTCCTGCAGGAT
CAGATGTAATCTCAGAATATGTAAAAGCCTGTGATGGAATCGGAGGAGCTATGCATAAA
GCTATGCTTATGGCTCAAGCAATAACAGGAGTTGTTTTAGGAGGACAAGTTAGAACATTT
GGAGGAAAATGTTATAATTGTGGTCAAATTGGTCACTTAAAAAAGAATTGCCCAGTCTTA
AACAAACAGAATATAACTATTCAAGCAACTACAACAGGTAGAGAGCCACCTGACTTATGT
CCAAGATGTAAAAAAGGAAAACATTGGGCTAGTCAATGTCGTTCTAAATTTGATAAAAAT
GGGCAACCATTGTCGGGAAACGAGCAAAGGGGCCAGCCTCAGGCCCCACAACAAACT
GGGGCATTCCCAATTCAGCCATTTGTTCCTCAGGGTTTTCAGGGACAACAACCCCCACT
GTCCCAAGTGTTTCAGGGAATAAGCCAGTTACCACAATACAACAATTGTCCCCCGCCAC
AAGCGGCAGTGCAGCAGTAGATTTATGTACTATACAAGCAGTCTCTCTGCTTCCAGGGG
AGCCCCCACAAAAAATCCCCACAGGGGTATATGGCCCCCTGCCTGAGGGGACTGTAGG
ACTAATCTTGGGAAGATCAAGTCTAAATCTAAAAGGAGTTCAAATTCATACTAGTGTGGT
TGATTCAGACTATAAAGGCGAAATTCAATTGGTTATTAGCTCTTCAATTCCTTGGAGTGC
CAGTCCAGGAGACAGGATTGCTCAATTATTACTCCTGCCATATATTAAGGGTGGAAATA
GTGAAATAAAAAGAATAGGAGGGCTTGGAAGCACTGATCCAACAGGAAAGGCTGCATAT
TGGGCAAGTCAGGTCTCAGAGAACAGACCTGTGTGTAAGGCCATTATTCAAGGAAAACA
GTTTGAAGGGTTGGTAGACACTGGAGCAGATGTCTCTATCATTGCTTTAAATCAGTGGC
CAAAAAATTGGCCTAAACAAAAGGCTGTTACAGGACTTGTCGGCATAGGCACAGCCTCA
GAAGTGTATCAAAGTACGGAGATTTTACATTGCTTAGGGCCAGATAATCAAGAAAGTACT
GTTCAGCCAATGATTACTTCAATTCCTCTTAATCTGTGGGGTCGAGATTTATTACAACAAT
GGGGTGCGGAAATCACCATGCCCGCTCCATTATATAGCCCCACGAGTCAAAAAATCATG
ACCAAGATGGGATATATACCAGGAAAGGGACTAGGGAAAAATGAAGATGGCATTAAAGT
TCCAGTTGAGGCTAAAATAAATCAAGAAAGAGAAGGAATAGGGTATCC
TTTTTaggggcggccactgtagagcctcctaaacccataccattaacttggaaaacagaaaaaccggtgtgggtaaatca
gtggccgctaccaaaacaaaaactggaggctttacatttattagcaaatgaacagttagaaaagggtcatattgagcctt
cgttctcaccttggaattctcctgtgtttgtaattcagaagaaatcaggcaaatggcgtatgttaactgacttaagggct
gtaaacgccgtaattcaacccatggggcctctccaacccgggttgccctctccggccatgatcccaaaagattggccttt
aattataattgatctaaaggattgcttttttaccatccctctggcagagcaggattgtgaaaaatttgcctttactatac
cagccataaataataaagaaccagccaccaggtttcagtggaaagtgttacctcagggaatgcttaatagtccaactatt
tgtcagacttttgtaggtcgagctcttcaaccagttagagaaaagttttcagactgttatattattcattatattgatga
tattttatgtgctgcagaaacgaaagataaattaattgactgttatacatttctgcaagcagaggttgccaatgctggac
tggcaatagcatctgataagatccaaacctctactccttttcattatttagggatgcagatagaaaatagaaaaattaag
ccacaaaaaatagaaataagaaaagacacattaaaaacactaaatgattttcaaaaattactaggagatattaattggat
tcggccaactctaggcattcctacttatgccatgtcaaatttgttctctatcttaagaggagactcagacttaaatagta
aaagaatgttaaccccagaggcaacaaaagaaattaaattagtggaagaaaaaattcagtcagcgcaaataaatagaata
gatcccttagccccactccaacttttgatttttgccactgcacattctccaacaggcatcattattcaaaatactgatct
tgtggagtggtcattccttcctcacagtacagttaagacttttacattgtacttggatcaaatagctacattaatcggtc
agacaagattacgaataataaaattatgtggaaatgacccagacaaaatagttgtccctttaaccaaggaacaagttaga
caagcctttatcaattctggtgcatggcagattggtcttgctaattttgtgggaattattgataatcattacccaaaaac
aaagatcttccagttcttaaaattgactacttggattctacctaaaattaccagacgtgaacctttagaaaatgctctaa
cagtatttactgatggttccagcaatggaaaagcagcttacacagggccgaaagaacgagtaatcaaaactccatatcaa
tcggctcaaagagcagagttggttgcagtcattacagtgttacaagattttgaccaacctatcaatattatatcagattc
tgcatatgtagtacaggctacaagggatgttgagacagctctaattaaatatagcatggatgatcagttaaaccagctat
tcaatttattacaacaaactgtaagaaaaagaaatttcccattttatattactcatattcgagcacacactaatttacca
gggcctttgactaaagcaaatgaacaagctgacttactggtatcatctgcactcataaaagcacaagaacttcatgcttt
gactcatgtaaatgcagcaggattaaaaaacaaatttgatgtcacatggaaacaggcaaaagatattgtacaacattgca
cccagtgtcaagtcttacacctgcccactcaagaggcaggagttaatcccagaggtctgtgtcctaatgcattatggcaa
atggatgtcacgcatgtaccttcatttggaagattatcatatgttcatgtaacagttgatacttattcacatttcatatg
ggcaacttgccaaacaggagaaagtacttcccatgttaaaaaacatttattgtcttgttttgctgtaatgggagttccag
aaaaaatcaaaactgacaatggaccaggatattgtagtaaagctttccaaaaattcttaagtcagtggaaaatttcacat
acaacaggaattccttataattcccaaggacaggccatagttgaaagaactaatagaacactcaaaactcaattagttaa
acaaaaagaagggggagacagtaaggagtgtaccactcctcagatgcaacttaatctagcactctatactttaaattttt
taaacatttatagaaatcagactactacttctgcagaacaacatcttactggtaaaaagaacagcccacatgaaggaaaa
ctaatttggtggaaagataataaaaataagacatgggaaatagggaaggtgataacgtgggggagaggttttgcttgtgt
ttcaccaggagaaaatcagcttcctgtttggatacccactagacatttgaagttctacaATGAACCCATCGGAGATGC
AAAGAAAAGCACCTCCGCGGAGACGGAGACACCGCAATCGAGCACCGTTGACTCACAA
GATGAACAAAATGGTGACGTCAGAAGAACAGATGAAGTTGCCATCCACCAAGAAGGCA
GAGCCGCCGACTTGGGCACAACTAAAGAAGCTGACGCAGTTAGCTACAAAATATCTAGA
GAACACAAAGGTGACACAAACCCCAGAGAGTATGCTGCTTGCAGCCTTGATGATTGTAT
CAATGGTGGTAAGTCTCCCTATGCCTGCAGGAGCAGCTGCAGCTAA
SEQ ID NO: 16
H. sapiens
DNA
>HERV-K orico Gag-Pol (codon optimized)
gtcgacgagagatcccgagtacgtctacagtcagccttacggtaagcttgtgcgctcggaagaagctagggtgataatgggcca
gaccaagagcaagatcaagagcaagtacgccagctacctgagcttcatcaagatcctgctgaagaggggcggagtgaaggt
gtccaccaagaacctgatcaagctgttccagatcatcgagcagttctgcccctggttccccgagcagggcaccctggacctgaag
gactggaagcggatcggcaaagagctgaagcaggccggcaggaagggcaacatcatccccctgaccgtgtggaacgactg
ggccatcatcaaggccgccctggaacccttccagaccgaagaggacagcgtgagcgtgagcgacgcccctggcagctgcatc
atcgactgcaacgagaaaacgcgtaagaagagccagaaagagaccgagagcctgcactgcgagtacgtggccgagcccgt
gatggcccagagcacccagaacgtggactacaaccagctgcaggaagtgatctaccccgagaccctgaagctggaaggcaa
gggccccgaactggtgggccccagcgagagcaagcccaggggcaccagccctctgcctgccggccaggtgcccgtgaccct
gcagccccagaaacaggtgaaagagaacaagacccagccccccgtggcctaccagtactggccccctgccgagctgcagta
ccggcctccccccgagagccagtacggctaccccggcatgccccctgcccctcagggcagagccccctacccccagcctccc
accaggcggctgaaccccaccgcccctcccagcaggcagggcagcgagctgcacgagatcatcgacaagagcaggaaag
agggcgacaccgaggcctggcagttccctgtgaccctggaacccatgcctcccggcgagggcgcccaggaaggcgagcccc
ccaccgtggaggcccggtacaagagcttcagcatcaagatgctgaaggacatgaaagaaggcgtgaagcagtacggcccca
acagcccctacatgcggaccctgctggacagcatcgcccacggccaccggctgatcccttacgactgggagatcctggccaag
agcagcctgagccccagccagttcctgcagttcaagacctggtggatcgacggcgtgcaggaacaggtgcggcggaacagg
gccgccaacccccccgtgaacatcgacgccgaccagctgctgggcatcggccagaactggtccaccatcagccagcaggcc
ctgatgcagaacgaggccatcgagcaggtgcgggccatctgcctgcgggcctgggagaagatccaggaccccggcagcacc
tgccccagcttcaacaccgtgagacagggcagcaaagagccctaccccgacttcgtggcccggctgcaggacgtggcccaga
agagcatcgccgacgagaaggcccggaaggtgatcgtggagctgatggcctacgagaacgccaaccccgagtgccagagc
gccatcaagcccctgaagggcaaggtgccagccggcagcgacgtgatcagcgagtatgtgaaggcctgcgacggcatcggc
ggagccatgcacaaggccatgctgatggcccaggccatcaccggcgtggtgctgggcggacaggtgcggaccttcggcggca
agtgctacaactgcggccagatcggccacctgaagaaaaactgccccgtgctgaacaagcagaacatcaccatccaggcca
ccaccaccggcagagagccccccgacctgtgcccccggtgcaagaagggcaagcactgggccagccagtgcagaagcaa
gttcgataaaaatgggcaaccattgtcgggaaacgagcaaaggggccagcctcaggccccacaacaaactggggcattccc
aattcagccatttgttcctcagggttttcagggacaacaacccccactgtcccaagtgtttcagggaataagccagttaccacaata
caacaattgtcmccgccacaagcggcagtgcagcagtagatctgtgcacaatccaggccgtgagcctgctgcccggcgagcc
tccccagaagatccccaccggcgtgtacggccctctgcccgagggcaccgtgggcctgatcctgggccggtccagcctgaacct
gaagggcgtgcagatccacactagtgtggtggacagcgactacaagggcgagatccagctggtgatcagcagcagcatcccc
tggtccgccagccctggcgaccggatcgcccagctgctgctgctgccctacatcaagggcggcaacagcgagatcaagcggat
cggcggcctgggcagcaccgaccccacaggcaaggccgcctactgggcctcccaggtgtccgagaaccggcccgtgtgcaa
ggccatcatccagggcaagcagttcgagggcctggtggacacaggcgccgacgtgagcatcatcgccctgaaccagtggccc
aagaactggcccaagcagaaagccgtgaccggcctggtgggcatcgggaccgccagcgaggtgtaccagagcacagagat
cctgcactgtctgggccccgacaaccaggaaagcaccgtgcagcccatgatcaccagcatccccctgaatctgtggggcaggg
acctgctgcagcagtggggagccgagatcaccatgcctgcccccctgtacagccccacaagccagaaaatcatgaccaagat
gggctacatccccggcaagggcctgggcaagaacgaggacggcatcaaagtgcccgtggaggctaaaataaatcaaaaaa
gagaaggaatagggtatcctttttaggggcggccactgtagagcctcctaaacccataccattaacttggaaaacagaaaaacc
agtgtgggtaaatcagtggccgctacctaagcagaaactggaagccctgcacctgctggccaacgaacagctggaaaagggc
cacatcgagcccagcttcagcccctggaacagccccgtgttcgtgatccagaagaaaagcggcaagtggcggatgctgaccg
acctgcgggccgtgaacgccgtgatccagcccatgggccccctgcagcctggcctgcccagccccgccatgatccccaagga
ctggcctctgatcatcatcgatctgaaggactgcttcttcaccatccccctggccgagcaggactgcgagaagttcgcctttaccatc
cccgccatcaacaacaaagagcccgccacccggttccagtggaaggtgctgccccagggcatgctgaacagccccaccatct
gccagaccttcgtgggcagagctctgcagccagtgagagagaagtttagcgactgctacatcatccactacatcgacgacatcct
gtgtgccgccgagaccaaggacaagctgatcgattgctacaccttcctgcaggccgaggtggccaatgccggcctggccatcg
ccagcgacaagatccagaccagcacccccttccactacctgggcatgcagatcgagaaccggaagatcaagcctcagaaga
tcgagatcaggaaggacaccctgaaaaccctgaacgacttccagaagctgctgggggacatcaactggatccggcccaccct
gggcatccccacctacgccatgagcaacctgttcagcatcctgcggggcgacagcgacctgaacagcaagagaatgctgacc
cccgaggccaccaaagaaatcaaactggtggaggaaaagatccagagcgcccagatcaacaggatcgaccccctggcccc
tctgcagctgctgatcttcgccaccgcccacagccctaccggcatcatcatccagaacaccgatctggtggagtggagcttcctgc
cccacagcaccgtgaaaaccttcaccctgtacctggaccagatcgccaccctgatcggccagacccggctgcggatcatcaag
ctgtgcggcaacgaccccgacaagatcgtggtgcccctgaccaaagaacaggtgcgccaggccttcatcaacagcggcgcct
ggcagatcgggctggccaatttcgtgggcatcatcgataaccactaccccaagaccaagatcttccagttcctgaagctgaccac
ctggatcctgcccaagatcacacggcgggagcctctggaaaacgccctgaccgtgttcaccgacggcagcagcaacggcaa
agccgcctataccggccccaaagaacgggtgatcaagaccccctaccagtccgcccagcgggccgaactggtggccgtgatc
accgtgctgcaggacttcgaccagcccatcaacatcatcagcgacagcgcctacgtggtgcaggccacccgggacgtggaga
ccgccctgatcaagtacagcatggacgaccagctgaaccagctgttcaatctgctgcagcagaccgtgcggaagcggaacttc
cccttctacatcacccacatccgggcccacaccaacctgccaggccctctgaccaaggccaatgagcaggccgacctgctggt
gtccagcgccctgattaaggcccaggaactgcacgccctgacacacgtgaatgccgccggactgaagaacaagttcgacgtg
acctggaagcaggccaaggacatcgtgcagcactgcacccagtgccaggtgctgcacctgcccacccaggaagccggcgtg
aaccccaggggcctgtgccccaacgccctgtggcagatggacgtgacccacgtgcccagcttcggcagactgagctacgtgca
cgtgaccgtggacacctacagccacttcatctgggccacctgccagaccggcgagagcaccagccacgtgaagaaacacctg
ctgtcctgcttcgccgtgatgggcgtgcccgagaagatcaagaccgacaacggccctggctactgcagcaaggccttccagaa
gttcctgagccagtggaagatctcccacaccacaggaattccttacaacagccagggccaggccatcgtggagcggaccaac
cggacactgaaaacacagctggtgaagcagaaagaggggggcgactccaaagagtgcaccaccccccagatgcagctga
acctggccctgtacaccctgaactttctgaacatctaccggaaccagaccaccacctccgccgagcagcacctgaccggcaag
aagaacagcccccacgagggcaagctgatttggtggaaggacaacaagaataagacctgggagatcggcaaggtgatcac
ctgggggagaggttttgcttgtgtttcaccaggagaaaatcagcttcctgtttggatacccactagacatttgaagttctacaatgaac
ccatcggagatgcaaagaaaagcacctccgcggagacggagacaccgcaatcgagcaccgttgactcacaagatgaacaa
aatggtgacgtcagaagaacagatgaagttgccatccaccaagaaggcagagccgccgacttgggcacaactaaagaagct
gacgcagttagctacaaaatatctagagaacacaaaggtgacacaaaccccagagagtatgctgcttgcagccttgatgattgt
atcaatggtggtaagtctccctatgcctgcaggagcagctgcagctaa
SEQ ID NO: 17
H. sapiens
DNA
>HERV-T whole genomic sequence Gag-Pol-Env
TGCCTAACCTTGTTTTTACTCTAACTCATTACTTTGAATTTTGTCCTGCTTGTCTCTTTAAT
CACCTATCCTTGCTTCTCATGTAAATAAGACTCTCTGTAGCTGGGAAGGCCAGACAAACT
CCAACTGACCCCTTAATTTACAAGACACTAAGGGCTCCTTTCCCAACCCCCTTTTGTAAG
GAGTTGGCCTGGGTAAACAGATCCTCAGCATTTCAAAGGAGTCCAATTAACTGATAAGG
TACTAACACCAACAATGTATGAAGTTCCCAGGATTTTTCTCAAAGAGATAACAACATAAA
ACCTTGAGTTCATGTCTTCTATAGACTCTATATCTAATTATAATGAAAGATTTAGAACCTT
GCACCTGGTACCTTTGCTCTTCTTGTAACCATTTGTCTTTTAAGTGGTTTATCTCTCTGTA
ACCATTTTGCTTCTTTTGATTCTTGCATGTTTTTACTTCTGTAGAATTATTGCATTTGAGTC
CCCCTCCCCTTCCTAAACCTAGGTATAAAAGTTAATCAAGCCCCTTCCTCGGGGCCGAG
AGAATTTTCAGCATTAGCCATCTCTTTGGCCGCCAGCTTAATAAAGGACTCTTAATTCGT
CTCAAAGTGTGGCATATTTTTTAACTCGCTTGGATACAACATAATGGAGGTCCCAGTGAG
AAATTAACTCCACTGGGTGAGAGCCGGTCTCACTCTGGGCTCCCCCGGAAGGACGGCT
GGCTTGGATGGGGGGCGCCACCTGAGGAAATAATTTTTAAGGTCCCAGAAGAGTAACT
GCCTTCCGGAGGAGAGCGGATCGACCACCGTGTCATTGCCCTAAAATTCAACATCTGA
GTCCTCAGCTTCTGACCCCGGGGTCAGGTAAGTCAGATTTGACTTCGTTTCTGGTAAGA
GGGGAGTGGCCCTGATGAGGGCGTCCCTCTTTTGACTCAGCCTGTTACTCTAGGACAC
TAGTGGGTTGAGCCTTGGTTTTCTGGTAGGTGCTTTTGTATCTTGGTTTGGGTAGGAAG
TGGTCCTGACAAGGACCCTCCACTGACTTAGCTCAGGGCCCAGGAGGCGGGAGCTAAG
CCTTGGTTTCCGGAAGACCGGACTCTCGATCTCTCTCTCTCTTTCTCTCTTGTCTATCTT
ACATCCTTCTGTTGTTCAGGTTTCTTGGACAATCTCTGGGAAAGAAAAGGAAGAAAAAAA
GAGAAAAAAATTGTTATAAACTCTGTGTAAATGGTGTGTGACTGTGGGAGGACAAGGGG
TTGCATTTGTCTTCCAGTTTGTAGCTCTATGGTGAAAGCTACAGAGTTCAAGTGGGCCCT
CACCTGCGGTTCCGTGGCGACCTCCTAAGGCTTAAGGCAGCACCGGGCATAGCTCGAT
CTGAGCCAGGGGTTTATACCAGCCTGTCGATGCTAAGAGGAGCCCAAGTCCCCTCTGG
GGGATCAGCCAGGCGGGCATCTGAATGACAGCATCACGGGAACTCCTTCCCTTGTCTG
TCTAATAAAGAAGGTAAAAAAGGGAAAACTGTCATAATTGTTTACATGCCCTAGGGTCAA
TTGTTTGTTTTATGTTTATTGTTTTTTTCAGTGTCTATTGTCTTGTTTAATAGTTGTCAAGG
TGTTATATGTCAAGACATCGATATTGCCCAAGACGTCTAGGTAAAAACTTCTTCAAGGAC
CTTAGTGCTGATTTTTTGTCACAAGAGGTTAAATTTCTCATCAATCATTTAGGCTGGCCA
CCACAGTCCTGTCTTTTCTGCCAGAAACAAGTAAGGTGTTGTTACGGAAAAGAGTGTGA
AAAACATTCACCTGATCGGAATTTCTGGCACCATGAAGGTTGCAGGTATTTAGATTGTCA
TACCCCACGTCCAAGTGATTAGACCTCCTCTAAACTAAACCAGTGGTGGATTCAAAACA
GCCACCCTGCAGACCTCCTTGCTCACCTCTTTTGTCATTCTGTAACTTTTCCTGTGCCCT
TAAATAAAACACTGTGTAAAGAAACCTACGACCGTAATGCTTTACTTCGTTTAGACTCCT
ACTCTGTTCCCCTGTGGCTACGCTCCCACCTTAAAAATGATCCGAGTGGTCCTTTTCCC
CCTCATCCCTGCCCCCTACCCCACACATCTCGTTTTCCAGTGTGACAGCAAGTTCAGCA
TCTCCAAGACTTGGCTCTGCTCTCACTCCTTAAACCCTTAAAAGAAAAAGCTGAATTTGA
ACTATTTGCCTTTGAATCGTGGAGACACCAAAAATATCTAGGATACAGGTCTAGAGGAA
GAAGAAGAGGGAGAACGCCTAGATCCAACCGACCCAGGAGACCTCAGGCTGACCCCTA
ATCCCCCTCCCTCAATCTAAAAGCTACAGAAATGTAGCAAGTAGTATTAGCTGTTGTAGT
TTTTCTGTTCTTCCTGGTCATGTTAATTCTGTTTTTCCGACACTCCAGCCCCCCAGGGAA
AGAATTTCTCTGCCCGTGTTGGATCAACACTTCCTCCCAGCCCTATAAAGGTTGGAGCC
CTCTCCAAGGTATGCTGCAGAATTTTTCTCTCGGTTTCTCAGAGGATTATGGAGTCCGC
CTTAAAAAAGGCAAGCTCCAGACACTCTGCGAAGTAGAATGGCCAAAGTTTGGAGTCAG
GTGGCCCCCTGAAGGGTCATTGAACCTTACAATTGTTCAAGCTGTGTGGCGGGTTGTTA
CTGAAACTCCCAGCCACCCTGATCAGTTTCCCTACATTGATCAATGGCTAAGTTTGGTCA
GGAGCCCTCCTCCATGGCTCCGTTCATGCGCCATTCATAATTCTACCTCCAAGGTCCTC
CTGAGCCAGACCGCGTTTTCGCCTCGACCCTCAGCCGGTTCAGCTCCCCCTGTACTGC
CTCCCTCTGAAGAAGAGGAGAGTCTCCCTCACCCAGTCCCACCGCCTTACAACCAGCC
TGCTCCCTTAAAGTCATCCCATGTCTCCTCGACGACGTCCCCTGTAGGCTCGCCACCCA
TTGCCTCTCAATCGTGACCGTGGCAGGAAGAAGTAGCCCCTCTACTACCACTGAGAGA
GGCACAAGTCCCTCCAGGTGATGAGCGCTCAGCCCCCTTCTTAGTTTATGTCCCTTTTT
CTACTTCTGACTTGTATAATTAGAAAACCCATAATCCTCCCTTCTCTGAAAAGCCCCAGG
CTTTGACCTCACTGATGGAGTCCGTACTCCGGACTCACCGGCCCACCTAGGATGACTG
CCAACAGCTCCTTTTAACCCTTTTCACCTCTGAAGAGAGGGAACGTATCCGAAGAGAGG
CCAGAAAGTACTTCCTTACATCAGCCGGTAGACCAGAGGAGGAAGCTAGAGACCTCCTT
GAGGAGGTCTTTCCCTCTACCCGGCCTAATTGGGACCCAAATTCCTCAAGTGGAAAGAG
AGCTTTAGACGATTTTCACCGGTATCTCCTCGCGGGTATTAAAGGAGCCGCTCAGAAAC
CCATAAACTTGTCTAAGACAACTGAAGTTGTCCAGGGGCCTGATGAGTCACCAGGAGCG
TTTTTAGAACGCCTCCAGGAGGCTTATCGGATTTACACCCCTTTTGACCCGGCAGCTCC
CGAAAATAGCCGTGCTCTTAATTTGGCATTTGTGGCTCAGGCAGCCCCGGATATTAAAA
GAAAACTCCAAAAACTGGAAGGATTTGCTGGAATGAATATCAGTCAGCTTTTAGAAATAG
CCCAAAAAGTTTTTGACAATCGAGAGTTTGAAAAACAAAAACAAGCAACACAGGCAGCT
GAAAAGGCCGCTGATAAAGCATTCAAAAGACAAACAAAAATCTTGGTAACAGCTATCCAA
GAGGGCAGAAAGGAAAGGCCCCCATTCCAGAAAAATGGCCAAGGAACCTCGGGTTCCC
ACCAGAAAAGTAAAAGAGGTGAACAGGCCCCTCTAGGAAAAGACCAATGTGCTTATTGC
AAGCAGACTGGGCACTGGAAAAAGGAGTGCCCACTACTGCCAAAAGAAAAATCAGAAAA
GAAAAAGGTCCTCACCCTCCCCGCAACAGAGGAGTCTGATGATTGATGGGGCCAGGGC
TCCCTCTCTCTTGGCCCCCAGGAGCCCATGGTGACTGCTACAGTGGGGGGCCAGCCTG
TACGCTTCCTAATAGACACCGGGGCAGAACACTCAGTACTGCAGACACCCTTGGGCAG
TGTCTCTAATAAAAAAGTGGCTGTACAAGTAGGGGCAAACTGGAGCCAGAAAAGAGTAA
CACACTCATTTCTTCATAAGTTACAGGCCTCTATCTCCTTCTCAGCCCAGCAGGCTCACC
TCACACTAGGAAATACAACGCCCCCCACTGCCCAACTCCTGCTAACTACCCCTCTGTCA
GAAGAATACCTTCTGGTTTCACCATCACAACCACCGGAGAATAATACTAATACTCTCCTG
TTGGACCTACAGACACTCTTTCCCCGAGTTTGGGCCGAGTCAAACCCCCCAGGACTGG
CAAAGCACCATCTGCCAGTAGTTGTAGAACTCCTGGCCACTGCCCTGCCAGTCCAGGTA
AAACAATATCCTATGAGTCAGCAGGCTAGAGAGGGGATCAATCCCCATATTCAGTGACT
GTTACAAGCTGGCATACTCACACCATGTCAGTCCGCCTGGAATACTCCATTTTTGCCGG
TCCAGAAACCTGGAACAAATGATTACCAGCCAGTACAGGACTTGAGGGAAGTTAACAAG
TGGACAGTTACTGTCCATCCAACTGTCCCTAACCCTTATACTTTACTCAGCCTGCTCCCA
CCAGAACATACAGTATACACTGTCCTTGACTTAAAGGATGCTTTCTTTGCTATTCCTCTG
GCCCCCAAAAGCCAACCTATCTTTGCTTTTGAATGGACAGATCCTGGCTCAGGAGACAC
CACCCAACTGACTTGGACTCAGTTACCTCACCCTTTTTGGGGAAGCCCTCCAACAAGAT
CTTATACCATTCCGAGCCCTAACTGTACTCTTCTCCAGTATGTAGATGACCTTTTATTAG
CTACTGAAACTACTGACAGCTGCCTGCAACATACTAGGGACCTACTTTACCTCCTTCAG
GAACTCGGGTATCGGGTCTCAGCCAAGAAGGCCCAGCTTTGTCTTCCCAGAGTGTCCT
ACCTGGGGTACGAGATAAACAAAGGAAAAAGGGCACTCACCAGTGCCCGGAAGGAAGC
CATCCTGCGAATCCCCACTCCCACCACCAAGAGACAGGTACATGAATTCCTGGGGGCC
ATGGGATACTGTCATCTATGGATATTGGGGTTCGCAGAGATTGCAAAGCCCCTGTATAC
TGCTACAGGAGGGAATGGCCCACTAATTTGGACTGACACAGAAGAACAGGCTTTTCAAA
ATCTGAAAAAGGCATTAACTGAAGCCCCTGCTTTAGCCCTCCCAAATATCTCAAAGCCAT
TTCACCTGTTTGTCCATGAAAGCCAGGGAGTTGCTAAAGGGGTGCTTACTCAGACTTTA
AGACCCTGGAGACGCCCAGTGGCCTATTTATCTAAGAGGCTGGATCCTGTGGCCTCTG
GATGGCCAAGTTGTCTGCGAGCCATAGCGGCTACAGCAAGCCTAGTCCAAGAAGCTGA
TAAGTTAACTCTAGGCCAAAATTTAACCCTTACAGCTCCTCATGCCGTAGAGACTTTACT
ACGAAGTGCTTCTGGCAAATGGATGTCAAATGCTCGCATCTTGCAGTATCAGAGTTTACT
GTTAGATCAGCCTCGTTTGACTTTCTCTCCCACAAGGTGTTTAAATCCAGCTACACTACT
TCCTGACCCAGACTCCACTATTCCTGTCCATGACTGTCAAGAACTGTTAGAAACTATCGA
AACTGGCCGACCTGATCTTCAAGATGTGCCCCTAGAAAAGGCGGATGCCGCCGTGTTC
ACAGACAGTAGCAGCTTCCTCAAGCAGGAAGTATGAAAAGCCAGTGCAGCTGTTACCAC
CTCAGCACAAAAGGCTGAATTGATCGCCCTCACTCAGGCTCTCCGATGGGGTAAGGATA
AACGTATTAACATTTACACTGACAGCAGGTACGCCTTTGCTACTGTGCATGTACATGGAG
CCATCTACCAAGAGCGAGGGCTACTCACCTCAGCAGGAAAGACTATCAAAAACAAAGAA
GAAATTTTAGCCCTGCTTGAAGCTGTTTGGCTCCCTCAGCAGGTGGCTGTAATCCACTG
CAAAGGACATCAAAAAGAAAACACGGCCGTTGCCCGTGGTAACCAAAAAGCTGATTCAG
CAGCTCAGGAAGTGGCACGGTCTTCAGTTACGCCCATAAACTTGCTGCCCACAGTCTCC
TTTCCACAGCCAGATCTGCCTGACAATCCCGTATACTCAACAGAAGAAAAAAAACTGGC
TTCAGATCTCAGAGCCAATAAAAATCAGGAAGGTTGGTGGATTCTTCCTGACTCTAGAAT
CTTCATACCCCGAGCTCTTGGAGAAACTTTAATCAGTCACCTACATTCTACCACCCATTT
AAGAAGAGCAAAGCTACCTCAGCTCCTCCGGAGCCATTTTAAGATCCCCCATCTTCAAA
GCCTAACAGATCAAGCAGCTCTCCAGTGCACAACCTGCGCCCAGGTAAATGCCAAACAA
GGTCCTAAACCCAGCCCAGGCCACCGTCTCTGAGAAAACTCGCCAGGAGAAAAGTGGG
AAATTGACTTTACAGAAGTAAAACCACACCGGGCTGAGTACAAATACCTTCTAGTACTAG
TAGACACCTTCTCCGGATGGACTGAGGCATTTGCTACCAAAAACGAAACTGCCAACACA
GTAGTTAAGTTTTTACTCAATGAAATCATCCCTCAACATGGGCTGCCTGCTGCCATAGGG
TCTGATAATGGACCAGCCTTCACCTCGTCCATAGCTCAGTCAGTCAGTAAGGCATTAAA
CATTCAATGGAAGCTCCATTGTGCCTATCGACCCCAGAGCTCTGGGCAGGTAGAACGC
ATGAACCACACCCTAAAAAACACTCTTACAAAATTAATCTTAGAAACCGGTGAAAATTGG
GTAAGTCTCCTTCCTTTAGCCCTACTTAGAGTAAGGTGCACCCCTTACCAGGCTAGGTT
CTCACCTTTTGAAATCATGTATAGGAGGGCGCCGCCTATCTTGCCTAAGCTAAGAGATG
CCCATTTAGCAGAAATATCACAAGCTAATTTATTACAGTACCTACAGTCTCTCCAACAGG
TACAAGATATCATCCTGCCACTTGTTCGAGGAGCCCATCCCAATCCAATTCCTGACCAG
ACGGGGTCCTGCCATTCGTTCCAGCCAGGAGACCTAGTGTTTGTTAAAAAGTTCCAGAA
AGAAGGACTCACTCCTGCTTAGAAAAGACCTCACACCGTCATCCTCACGACGCCAACGG
CTCTGAAAGTGGATGGCATTCCTGCTTGGATTCATCACTCCCGCATCAAAAAGGCCAAC
AGAGCCCAGCTAGAAACATGGGTCCCCAAGCCTAGGTCAGGCCCCTTAAAACTGCACC
TAAGTCAGGTGAAGCCATTAGATTAATTCTTTTTATTTACCTCTCTTGTTTGTTTTTGCCT
GTTATGTCCTCTGTGCCTTCCTACTCCTTTCTCCTCACCTCTTTCACAACAGGACGTGTA
TTTGCAAACACCACTTAGAAGGCCAGTACCTCCAAGGAAGTCTCCTTTGCAGTTGATTTA
TGTGTACTGTTCCCAAAGCCAGCCCATACCCACGAAAAGCAACACAATCTGCCAGTCCC
AGGAGCAGGAAGTGTCGACCTTGCAGCAGGATTTAGACACTCCAGGAGCCAAACTAGA
TGTGGAAGCTCCAAAGGTGCAGAAAAAGGACTCCAAAATGTTGACTTTTACCTCTGTCC
TGGAAATCACCCTGACGCTAGCTGTCGAGATACTTATCAGTTTTTCTGCCCTGATTGGAC
ATGTGTAACTTTAGCCACCTACTCTGGAAGATCAACCAGATCTTCAACTCTTTCCATAAG
TCGTGCTTCTCATCCTAAATTATGTACTAGAAAAAATTGTAATCCTCTTACTATAACTGTC
CATGACCCTAATTCAGCTCAATGGTATTATGGCATGTCATGGGGATTAAGACTTTATATC
CCAGGATTTGATGTTGGAACTATGTTCACCATCCAAAAGAAAATCCTGGTCTCATGGAG
CCCACCCAAGCCAATCAGGCCTTTAACTGATCTAGGTGACCCTATGTTCCAGAAACACC
CTGACAAAGTTGATTTAACTGTTCCTCCACCATTCCTAGTTCCTAAACCCCAGCTGCAAA
GACAACACCTCCAACCCAGCCTGATGTCCATACTAGGTGGGGTACATCACCTCCTTAAT
CTCACCCAGCCTAAACTAGCCCAAGATTGTTGGCTATGTCTAAAAGCAAAACCCCCATAT
TATGTAGGATTAGGAGTAGAAGCCACACTTAAAAGTGGCCCTTTATCCTGTCATGCACG
ACCCCATGCCCTCACATTAGGAGATGTGTCTGGAAATGCTTCTTGTCTAATTAGTACTGG
ATATAACTTATCTGCTTCTCCCTTTCAGGCTACCTGTAATCAGTCTCTGCTTACTTCCTTA
AGCACCTCAGTCTCCTACCAGGCACCTAACAATACCTGGTTGGCCTGCACTTCAGGTCT
CACTCGCTGCATCAATGGGACTGAACCAGGACCTCTCCTGTGTGTGTTGGTTCATGTAC
TCCCCCAGGTCTACGTGTACAGTGGGCCAGAAGGACAACTTCTCATTGCTCCCCCTGAA
TTACATCCCAGGCTACACAGAGCTGCCCCACTACTGGTTCCTCTTTTAGCCGGTCTTAG
CATAGCTGGATCAGCAGCCATTGGCACGGCTGCCCTGGTTCAGGGAGAAACTGGACTA
ATGTCCCTGTCTCAACAAGTAGATGCTGATTTAAGCAATCTCCAGTCAGCCATAAATATA
CTACATACCCAGGTAGAGTCTCTAGCTGAAGTAGTTCTTCAAAACCGCCGAGGCTTAGA
TCTGCTATTTCTCTCCCAAGGAGGGTTATGCGCAGCTCTAGGAGAAAGTTGTTGCTTCT
ATGCCAATCAGTCTGGAGTCATAAAAGATACACTCCAAAAAGTTCAAGAAAATCTAGATA
GGCGCCAACAAGAACAAGAAAATAACATCCCCTGGTATCAAAGCATGTTCAACTGGAAC
CCATGGCTAACTACTCTAATCACTGGGTTAGCCGGACCCCTCCCCATCCTACTATTAAG
TCTAATTTTTGGGCCTTGTATATTAAATTGGTTTCTTAATTTTGTAAAACAACGCATAGCTT
CTGTCAAACTTATGTATCTTAAAACTCAATATAACCCCCTTGTTATAACTGAGGAATCAAC
GATTTGATTCCCCTAAAACACAAGTGGGGAAATGAAATGCCTAACCTTGTTTTTACTCTA
ACTCATTACTTTGAATTTTGTCCTGCTTGTCTCTTTAATCACCTATCCTTGCTTCTCATGT
AAATAAGACTCTCTGTAGCTGAGAAGGTCGGACCAACTCCAATTGACCCCTTAATTTATA
AGACACTAAGGGCTCCTTACCCAACCCCCTTTTGTAAGGAGTTGGCCTGGGTAAACAGA
TCCTCAGCATTTCAAAAGAGCCCAAGTAACTGACAAGGTACTAACACCAACAATGTATGA
AGTTCCCAGGATTTTTCTCAAAGAGATGACAACATAAAACCTGGAGTTCATGTCTGGCAT
AGACCCTATATCTAATTATAATGAAAGATTTAGAACCTTGCACCTACTACCATTGCTCTTC
TTGTAACCATTTGTCTTTTAAGTTGTTTATTTCTCTGTAACCAGTTTGCTTCTTTTGATTCT
TGCATGTTTTTACTTCTGTAGAATTATTGCATTTGAGTCCCCCTCCTCTTCCTAAACCTAG
GTATAAAAGTTAATCAAGCCCCTTCCTCGGGGCCGAGAGAATTTTGAGCATTAGCCATC
TCTTTGGCGCCCAGCTTAATAAAGGACTCTTAATTCATCTCAAAGTGTGGTGTATTTTTTA
ACTTGCTTGGGTACAACA
SEQ ID NO: 18
H. sapiens
DNA
>HERV-W whole genomic DNA Gag-Pol-Env
ACTGAGAGACAGGACTAGCTGGATTTCCTAGGCCGACTAAGAATCCCTAAGCCTAGCTG
GGAAGGTGACCACGTCCACCTTTAAACACGGGGCTTGCAACTTAGCTCACACCTGACCA
ATCAGAGAGCTCACTAAAATGCTAATTAGGCAAAGACAGGAGGTAAAGAAATAGCCAAT
CATCTATTGCCTGAGAGCACAGCAGGAGGGACAACAATCGGGATATAAACCCAGGCATT
CGAGCTGGCAACAGCAGCCCCCCTTTGGGTCCCTTCCCTTTGTATGGGAGCTGTTTTCA
TGCTATTTCACTCTATTAAATCTTGCAACTGCACTCTTCTGGTCCATGTTTCTTACGGCTC
GAGCTGAGCTTTTGCTCACCGTCCACCACTGCTGTTTGCCACCACCGCAGACCTGCCG
CTGACTCCCATCCCTCTGGATCCTGCAGGGTGTCCGCTGTGCTCCTGATCCAGCGAGG
CGCCCATTGCCGCTCCCAATTGGGCTAAAGGCTTGCCATTGTTCCTGCACGGCTAAGTG
CCTGGGTTTGTTCTAATTGAGCTGAACACTAGTCACTGGGTTCCATGGTTCTCTTCTGTG
ACCCACGGCTTCTAATAGAACTATAACACTTACCACATGGCCCAAGATTCCATTCCTTGG
AATCCGTGAGGCCAAGAACTCCAGGTCAGAGAATACGAGGCTTGCCACCATCTTGGAA
GCGGCCTGCTACCATCTTGGAAGTGGTTCACCACCATCTTGGGAGCTCTGTGAGCAAG
GACCCCCCGGTAACATTTTGGCAACCACGAACGGACATCCAAAGCGGTGAGTAATATTG
GACCACTTTCACTTGCTATTCTGTCCTATCCTTCCTTAGAATTGGAGGAAAATACCGGGC
ACCTGTCGGCCAGTTAAAAACGATTAGCGTGGCCACCGGACTTAAGACTCAGGTGTGA
GGCTATCTGGGGAAGGGCTTTCTAACAACCCCCAACCCTTCTGGGTTGGGAACGTTGG
TCTGCCTCGAAGCCAGCTTCCACTTTCAATTTTCTTGGGGAAGCCGAGGGCCGACTAGA
GGCAGAAAGCTGTCGTCCCGAACTCCCGGCAGTAGCCGGTTGAGATCATGGCGCAGC
CAGAAGTCTCTACTCAACAGTCGCCCATGCATGCACCCCTACCTTTCCTTCTGACCCAT
ACCTCCTGGGTCCCAACCACAACTTTCTTCAAAGTGTAGCCCCAAAATTCTCCTTACCTC
TGAATATACTTCCTCTGATCCCTGCCTCCTAGGTACTAATGGTTCAGACTTCCATTTCCT
CTAGCAAGTTGTATCTCCAAAGGGATCTAAGGAAGCTCTACGCTGCGTCCTTAGGCACC
TAGGCTATGAACCCAGGGAGTCTTATCCCTGGTGTCCCTCCCAATTTAGGCATACAGCT
CTCGACATGGGCAGTTATGTAGGACCCACTCCCCACCACCCTTGCCAGGGCCCCAAGT
TTGTAAATGGCTAAGAGAAAAGAGAGACAGAGGAGAGAGAGAGAAATGGAGGAGAAAG
AGAGAGAGACAGAGAGGAGAGAGAGACAGAGAGAGAGACAGAGAGAGAGAGAGAGAC
AAAGAGGAGAGAGAGAGAGTCAAAGAGAGAAAGAAAGAGAAAGAAATAGTAAAAAACA
GTGTGCCCTATTCCTTTAAAAGCCAGGGTAAATTTAAAACCTATAATTGATAATTGAAGG
TCTTCTCCATGACCCTATAACACTCCAATACCACCTTGTGGTCAGTGTAAACAAGAGCAT
AGCCCGAAAACACTGAGACCACTGACAACCCGTAGCCTCCCTATCAAAAATCCTTAACC
CAGTAACCCACAGATGGACCAAATGCATTCAATCGGTAGCGGCAACTGCTTTGCTAACA
GAAAAAAGTAGAAAAGTAACTTTTAGAGGAAACCTCATTGTGAGCACACCTCACCAGTTC
AGAATTATTCTAAGTAAAAAAAGCAAAAAGGTAGCTTACTAACTCAAAAATCTTAAAGTAT
GGGGCTATTCTGTTAGAAAAAGGTAATGTAACACCAACCACTGATAATTCCCTTAACCCA
GCAGATTTCCTAACAGGGGATTTAAATCTTAATTACCATACAAAGGTCCGACCAGACCTA
GGAGGAACTCCCTTCAGGACAGGACGATAGATGGTTCCTCCCAGGTGATTGAGGAAAA
AAACCACAATGGGTATTCAGTAATTGATACGGAGACTCTTGTGGAAGCAGAGTTAGAAA
AATTGCCTAATAACTGGTCTCCTCAAACGTGCGAGCTGTTTGCACTCAGCCAAGCCTTA
AAGTACTTACAGAATCAAAAGACTATCTCAATCCTGACTCAAAAGGTTACCTACACCCTC
TCTGAAATGAATTTGCATAAGAACTGTTGTTTATGGGAATGCATCTTGATGGGGCAGCTG
GGTTGTTATAAAATACTCAGGAACCCAGCCCAGCTCTAGGACTCACCCCTGAGCACAAA
GGCAATGTTGGGCACGCTGGTAAAGGACCACTAGAATCCAGCAGCCCAGACCCCTTTC
TTTGTGGTCAAGAAAGGCGGGAAAACAGGTGCAGGACTGCTACATCGGTAAGCATAACT
AATCCGATAAACAGAGGTCCATGGGTGGTTACGCACCCTGGAAAGGAACTCACCCCTG
AGCACAAAGGCAATGTTGGGCACGCTGGTAAAGGACCACTAGAATCCAGCAGCCCGGA
CCCCTTTCTTTGTGGTCAAGAAAGGCAGGAAAACAGGTGCAGGACTGCAACATCAGTGA
GCATAACTAATCCGATAAGCAGAGGTCCATGGGTGGTGACGCACCCTGGAAAGGAATA
AGCATTAGGACCATAGAGGACACTCCAGGACTAAAGCTCATCGGAAAATGACTAGGGGT
GCTGGCATCCCTATGTTCTTTTTTCAGATGGGAAACGTTCCCCCCAAGGCAAAAACGCC
CCTAAGATGTATTCTGGAGAATTGGGACCAATTTGACCCCTCAGACGCTAAGAAAGAAA
TGACTTATATTCTTCTGCAGTACCGCCTGGCGGGAAGTATAAATTATAACACCATCTTAC
AGCTAGACCTCTTTTGTAGAAAAGAAGGCAAATGGAGTGAAGTGCCATATGTACAAACTT
TCTTTTCATTAAGAGACAACTCGCAATTATGTAAAAAGTGTGATTTATGCCCTACAGGAA
GCCCTCAGAGTCTACCTCCCTACCCCAGCATCCCCCCGACTCCTTCCCCAACTAATAAG
GACCCCCCTTCAACCCAAATGGTCCAAAAGGAGATAGACAAAGGGGTAAACAATGAACC
AAAGAGTGCCAATATTCCCCGATTATGCCCCCTCCAAGCAGTGGGAGGAGGAGAATTC
GGCCCAGCCAGAGTGCATGTACCTTTTTCTCTCTCAGACTTAAAGCAAATTAAAATAGAC
CTAGGTAAATTCTCAGATAACCCTGATGGCTATATTGATGTTTTACAAGGGTTAGGACAA
TCCTTTGATCTGACATGGAGAGATATAATGTTACTGCTAAATCAGACACTAACCCCAAAT
GAGAGAAGTGCCGCCATAACTGCAGCCCGAGAGTTTGGCGATCTCTGGTATCTCAGTC
AGGTCAATGATAGGATGACAACAGAGGAAAGAGAACGATTCCCCACAGGCCAGCAGGC
AGTTCCCAGTGTAGACCCTCACTGGGACACAGAATCAGAACATGGAGATTGGTGCCGC
AGACATTTGCTAACTTGCGTGCTAGAAGGACTAAGGAAAACTAGGAAGAAGCCTATGAA
TTATTCAATGATGTCCACTATAACACAGGGAAAGGAAGAAAATCCTACTGCCTTTCTGGA
GAGACTAAGGGAGGCATTGAGGAAGCATACCTCTCTGTCACCTGACTCTATTGAAGGCC
AACTAATCTTAAAGGATAAGTTTATCACTCAGTCAGCTGCAGACATTAGAAAAAAACTTC
AAAAGTCTGCCTTAGGCCCGGAGCAAAACTTAGAAACCCTATTGAACTTGGCAACCTCG
GTTTTTTATAATAGAGATCAGGAGGAGCAGGCGGAACGGGACAAACGGGATTAAAAAAA
AGGCCACCGCTTTAGTCATGGCCCTCAGGCAAGCGGACTTTGGAGGCTCTGGAAAAGG
GAAAAGCTGGGCAAATCGAATGCCTAATAGGGCTTGCTTCCAGTGCGGTCTACAAGGA
CACTTTAAAAAAGATTGTCCAAGTAGAAATAAGCCGCCCCCTCGTCCATGCCCCTTATGT
CAAGGGAATCACTGGAAGGCCCACTGCCCCAGGGGATGAAGGTCCTCTGAGTCAGAAG
CCACTAACCAGATGATCCAGCAGCAGGACTGAGGGTGCCCGGGGCAAGCGCCAGCCC
ATGCCATCACCCTCACAGAGCCCCGGGTATGCTTGACCATTGAGGGCCAGGAGGTTAA
CTGTCTCCTGGACACTGGCGCGGCCTTCTCAGTCTTACTCTCCTGTCCCGGACAACTGT
CCTCCAGATCTGTCACTATCCGAGGGGTCCTAGGACAGCCAGTCACTAGATACTTCTCC
CAGCCACTAAGTTGTGACTGGGGAACTTTACTCTTTTCACATGCTTTTCTAATTATGCCT
GAAAGCCCCACTCCCTTGTTAGGGAGAGACATTCTAGCAAAAGCAGGGGCCATTATACA
CCTGAACATAGGAGAAGGAACACCCGTTTGTTGTCCCCTGCTTGAGGAAGGAATTAATC
CTGAAGTCTGGGCAACAGAAGGACAATATGGACGAGCAAAGAATGCCCATCCTGTTCAA
GTTAAACTAAAGGATTCCGCCTCCTTTCCCTACCAAAGGCAGTACCCCCTTAGACCCGA
GGCCCAACAAGGACTCCAAAAGATTGTTAAGGACCTAAAAGCCCAAGGCCTAGTAAAAC
CATGCAATAGCCCCTGCAATACTCCAATTTTAGGAGTACAGAAACCCAACGGACAGTGG
AGGTTAGTGCAAGATCTCAGGATTATCAATGAGGCCGTTGTCCCTCTATACCCAGCTGT
ACCTAACCCTTATACTCTGCTTTCCCAAATACCAGAGGAAGCAGAGTGGTTTACAGTCCT
GGACCTTAAGGATGCCTTTTTCTGCATCCCTGTACATCCTGACTCTCAATTCTTGTTTGC
CTTTGAAGATCCTTCGAACCCAACGTCTCAACTCACCTGGACTGTTTTACCCCAAGGGTT
CAGGGATAGCCCCCATCTATTTGGCCAGGCATTAGCCCAAGACTTGAGCCAGTTCTCAT
ACCTGGACACTCTTGTCCTTCGGTACGTGGATGATTTACTTTTAGCCGCCCGTTCAGAA
ACCTTGTGCCATCAAGCCACCCAAGCGCTCTTAAATTTCCTCGCTACCTGTGGCTACAA
GGTTTCCAAACCAAAGGCTCAGCTCTGCTCACAGCAGGTTAAATACTTAGGGCTAAAAT
TATCCAAAGGCACCAGGGCCCTCAGTGAGGAATGTATCCAGCCTATACTGGCTTATCCT
CATCCCAAAACCCTAAAGCAACTAAGAGGGTTCCTTGGCATAACAGGCTTCTGCCGAAT
ATGGATTCCCAGGTACGGCGAAATAGCCAGGCCATTATATACACTAATTAAGGAAACTC
AGAAAGCCAATACCCATTTAGTAGATGGACACCTGAAGCAGGCCCTAACCCAAGCCCCA
GTGTTAAGCTTGCCAACGGGGCAAGACTTTTCTTTATATGTCACAGAAAAAACAGGAATA
GCTCTAGGAGTCCTTACACAGGTCCGAGGGACGAGCTTGCAACCCGTGGCATACCTGA
GTAAGGAAATTGATGTAGTGGCAAAGGGTTGGCCTCATTGTTTACGGGTAGTGGCGGC
AGTAGCAGTCTTAGTATCTGAAGCAGTTAAAATAATACAGGGAAGAGATCTTACTGTGTG
GACATCTCATGATGTGAATGGCATACTCACTGCTAAAGGAGACTTGTGGCTGTCAGACA
ACCATTTACTTAAATATCAGGCTCTATTACTTGAAGGGCCAGTGCTGCGACTGCGCACTT
GTGCAACTCTTAACCCAGCCACATTTCTTCCAGACAATGAAGAAAAGATAGAACATAACT
GTCAACAAGTAATTGCTCAAACCTACGCCACTCGAGGGGACCTTTTAGAGGTTCCCTTG
ACTGATCCCGACCTCAACTTGTATACTGATGGAAGTTCCTTTGTAGAAAAAGGACTTCGA
AAAGCGGGGTATGCAGTGGTCAGTGATAATGGAATACTTGAAAGTAATCCCCTCACTCC
AGGAACTAGTGCTCAGCTGGCAGAACTAATAGCCCTCACTCGGGCACTAGAATTAGGA
GAAGGAAAAAGGGTAAATATATATACAGACTCTAAGTATGCTTACCTAGTCCTCCATGCC
CATGCAGCAATATGGAGAGAAAGGGAATTCCTAACTTCCGAGGGAACACCTATCAAACA
TCAGGAAGCCATTAGGAGATTATTATTGGCTGTACAGAAACCTAAAGAGGTGGCAGTCT
TACACTGCCGGGGTCATCAGAAAGGAAAGGAAAGGGAAATAGAAGGGAACCGCCAAGC
GGATATTGAAGCCAAAAGAGCCGCAAGGCAGGACCCTCCATTAGAAATGCTTATAGAAG
GACCCCTAGTATGGGGTAATCCCCTCCGGGAAACCAAGCCCCAGTACTCAGCAGAAGA
AATAGAATGGGGAACCTCACGAGGACATAGTTTCCTCCCCTCAGGATGGCTAGCCACC
GAAGAAGGAAAAATACTTTTGCCTGCAGCTAACCAATGGAAATTACTTAAAACCCTTCAC
CAAACCTTTCACTTAGGCATTGATAGCACCCATCAGATGGCCAAATCATTATTTACTGGA
CCAGGCCTTTTCAAAACTATCAAGCAGATAGTCAGGGCCTGTGAAGTGTGCCAAAGAAA
TAATCCCCTGCCTTATCGCCAAGCTCCTTCAGGAGAACAAAGAACAGGCCATTACCCAG
GAGAAGACTGGCAACTAGATTTTACCCACATGCCCAAATCTCAGGGATTTCAGTATCTAC
TAGTCTGGGTAGATACTTTCACTGGTTGGGCAGAGGCCTTCCCTTGTAGGACAGAAAAG
GCCCAAGAGGTAATAAAGGCACTAATTCATGAAATAATTCCCAGATTCGGACTTCCCCG
AGGCTTACAGAGTGACAATGGCCCTGCTTTCAAGGCTGCAGTAACCCAGGGAGTATCC
CAGGCGTTAGGCATACAATATCACTTACACTGCGCCTGGAGGCCACAATCCTCAGGAAA
AGTCGAGAAAATGAATGAAACACTCAAACGACATCTAAAAAAGCTAACCCAAGAAACCC
ACCTCGCATGGCCTGCTCTGTTGCCTATAGCCTTACTAAGAATCCAAAACTCTCCCCAAA
AAGCAGGACTTAGCCCATACGAAATGCTGTATGGACAGCCCTTCCTAACCAATGACCTT
GTGCTTGACCGAGAGATGGCCAACTTAGTTGCAGACATCACCTCCTTAGCCAAATATCA
ACAAGTTCTTAAAACATTACAAGGAACCTGTCCCTGAGAGGAGGGAAAGGAATTATTCC
ACCCTGGTGACATGGTATTAGTCAAGTCCCTTCCCTCTAATTCCCCATCCCTAGATACAT
CCTGGGAAGGACCCTACCCAGTCATTTTATCTACCCCAACCGCGGTTAAAGTGGCTGGA
GTGGAGTCTTGGATACATCACACTCGAGTCAAACCCTGGATACTGCCAAAGGAACCCGA
AAATCCAGGAGACAACGCTAGCTATTCCTGTGAACCTCTAGAGGATCTGCGCCTGCTCT
TCAAGCGACAACCGTGAGGAAAGTAACTAAAATCGTAAATCCCCATGGCCCTCCCTTAT
CATATTTTTCTCTTTACTGTTCTCTTACCCCCTTTCACTCTCACTGCACCCCCTCCATGCC
GCTGTACTACCAGTAGCTCCCCTTACCAAGAGCTTCTATGGAGAATGCAGCTTCCCAGA
AATATTGATGCCCCATCGTATAGGAGTTTTTCTAAAGGAAACCCCACTTTCACCGCCCAC
ACCCATATGCCCCACAACTGCTATAATTTGCATGCATGCAAATAGTTGTCCTGGAGGACT
TGGAGCCACTGTCTGTTGGACTTACTTCACCCATACCGGTATGTCTGATGGGGGTGGAG
TTCAAGATCAGGCAAGAGAAAAACACGTAAAGGAAGTAATCTCCCAACTGACCCGGGTA
CATAGCACCCCTAGCCCCTACAAAGGACTAGATTCTCTCAAAACTACATGAAACCCTCC
GTACCCATACTCGCCTGGTAAGCCTATTTAATACCACCCTCACTGGGCTCCATGAGGTC
TCGGCCCAAAACCCTACTAACTGTTGGATGTGCCTCCCCCTGCACTTCAGGCCATACAT
TTCAATCCCTGTACCTGAACAATGGAACAACTTCAGCACAGAAATAAACACCACTTCCGT
TTTAGTAGGACCTCTTGTTTCCAATCTGGAAATAACCCATACCTCAAAACCTCACCTGTG
TAAAATTTAGCAATACTATAGACACAACCAACTCCCAATGCATCAGGTGGGTAACTCCTC
CCACACGAATAGTCTGCCTACCCTCAGGAATATTTTTTGTCTGTGGTACCTCAGCCTATC
GTTGTTTGAATGGCTCTTCAGAATCTATGTGCTTCCTCTCATTCTTAGTGCCCCCTATGA
CCATCTACACTGAACAAGATTTATACAATCATGTTGTACCTAAGCCCCGCAACAAAAGAG
TACCCATTCTTCCTTTTGTTATCGGAGCAGGAGTGCTAGGTGGACTAGGTACTGGCATT
GGCGGTATCACAACCTCTACTCAGTTCTACTACAAACTATCTCAAGAACTAAATGGTGAC
ATGGAACGGGTCGCCGACTCCCTGGTCACCTTGCAAGATCAACTTAACTCCCTAGCAGC
AGTAGTCCTTCAAAATCGAAGAGCTTTAGACTTGCTAACCGCCGAAAGAGGGGGAACCT
GTTTATTTTTAGGGGAAGAATGCTGTTATTATGTTAATCAATCCGGAATCGTCACCGAGA
AAGTTAAAGAAATTCAAGATCGAATACAACGTAGAGCAGAGGAGCTTCAAAACACTGGA
CCCTGGGGCCTCCTCAGCCAATGGATGCCCTGGATTCTCCCCTTCTTAGGACCTCTAGC
AGCTATAATATTGTTACTCCTCTTTGGACCCTGTATCTTTAACCTCCTTGTTAAGTTTGTC
TCTTCCAGAATCGAAGCTGTCAAATGGAGCCCCAGATGCAGTCCATGACTAAGATCTAC
CGCGGACCCCTGGACCGGCCTGCTAGCCCATGCTCCGATGTTAATGACATCGAAGGCA
CCCCTCCCGAGGAAATCTCAACTGCACAACCCCTACTATGCCCCAATTCAGCAGGAAGC
AGTTAGAGCGGTCATCGGCCAACCTCCCCAACAGCACTTAGGTTTTCCTGTTGAGAGGG
GGGACTGAGAGACAGGACTAGCTGGATTTCCTAGGCTGACTAAGAATCCCTAAGCCTA
GCTGGGAAGGTGACCACATCCACCTTTAAACACGGGGCTTGCAACTTAGCTCACACCTG
ACCAATCAGAGAGCTCACTAAAATGCTAATTAGGCAAAGACAGGAGGTAAAGAAATAGC
CAATCATCTATTGCCTGAGAGCACAGCAGGAGGGACAATGATCGGGATATAAACCCAAG
TCTTCGAGCCGGCAACGGCAACCCCCTTTGGGTCCCCTCCCTTTGTATGGGAGCTCTG
TTTTCATGCTATTTCACTCTATTAAATCTTGCAACTGCACTCTTCTGGTCCATGTTTCTTA
CGGCTTGAGCTGAGCTTTCGCTCGCCATCCACCACTGCTGTTTGCCGCCACCGCAGAC
CCGCCGCTGACTCCCATCCCTCTGGATCATGCAGGGTGTCCGCTGTGCTCCTGATCCA
GCGAGGCACCCATTGCCGCTCCCAATCGGGCTAAAGGCTTGCCATTGTTCCTGCATGG
CTAAGTGCCTGGGTTCATCCTAATTGAGCTGAACACTAGTCACTGGGTTCCATGGTTCT
CTTCTGTGACCCACAGCTTCTAATAGAGCTATAACACTCACCGCATGGCCCAAGGTTCC
ATTCCTTGAATCCATAAGGCCAAGAACCCCAGGTCAGAGAACACGAGGCTTGCCACCAT
CTTGGGAGC
SEQ ID NO: 19
H. sapiens
DNA
>HERV-R whole genomic sequence Gag-Pol-Env
GGCAGGAAATATAAAAGGAAAAACAAGTAAAGGGAAAACAAGTCCTTCCCTGATCAGTC
TGACTCACTCCAAAGTCCTGCTGGAGCTATGATAACATTATCTGCAAGGCCAGGCAGGG
ACCCCAAAAGAATGGGCTCCAGGAGCAGAGATGAGAAAAACAAGTTCTCCTTATCAGTT
TCCGCCTTGAAATTCTTTCCCCATACCATTATTCTTTGTTCTGCTCTCACAACTATTTTTG
TAACTATTTCTGCAAGTTTGCAAAGATTTCATAAGTTCCTGTTTTTCTTTCTGTAGCACGG
CAAGGTCACAAGACATGCTTAAGTAAGATAGGGTCATGTTGCAAATCCTGTTGTAAAACC
TGTCACGGTATGATTAACTGTCTTTGTTCTGCTTCTGTAAGACTGCTTTCCTGTCTCACA
GGTTTCATGCCAAAAACCTGACCCGCCCCTGTTGGTTGCATGTATAAAAGTCAAGCCCT
GTCATTGTTCAGGGCTCAGCCTTTGGATGTTCATCGGCTGGGCTGGTGGTCACCTAAAT
AAAATCCTCCTGTTCCACCAAGTGGTCTCTCCAGCCTCCTGATTCCCACAACATTTTGGC
GAGCCAGCCAGGAGGGGAGACTACAGGTTTACTGTCTCCTTTGGCTGTGGGACTGGAG
CCCCGGGCTGGGGGAGACCCGTGACCCAAGGCACCATTGGGAGACCTTCAACCCGGA
GGGGAGATTGGCTCTCCCATGACCCGGCGCCCCTCCCTGACAGTGCATTGGAACCTAA
GGGGCTACAGGACGATTCCAGGACTTCACGCTACAGGACTGCAGTAAGGTTTGGGCCC
AAGGCAGGACCCGTCCCATAAGGATGGAAGGGGAGCCTGATCACCTCCTGGGGTGTAC
CTAGTAATCCGACTCAGGACATGAGAGGGGCTCGCAAAGTTGGAAGAAACCTACACCC
CAACTGACCCAGGACGTGAGAGTGGCTCACTAAGTTGGTTAAGAAAGGAAACTGGAAG
TGGGGAGGTGTGTGAATAAATGTGAAAGAGATGGTTCCAAAAGGAACCAACATGCTGAG
TGACGTGTGTGGAGCCACAGGTCTCTTAGCATAGACTGTACCATCTGAGTGAAGTGTGG
AACCGACCGAGACTAGTGGCAAACATCCTTTGGGGCTACGGCATATGGCTTAGGGAGG
TGCCCCACAACTTATAGATTGTGGTGGTCCGGGTTCAGGACTCATATGAACCCTCCATT
AAATCTAAGAAGTGTCTGAAATACTCCTGCAAGGGACACGGTCTAATCAGTCTGAAATG
AAAGGAAGAGTGAGTGGGTTCTGCCATAACTGGGAGGAAATGGGAGGGAAGTCATCAA
AACCCACCCCATTAGAATGTATGTTAAAGAACTTTAAGAAAGGTTATACAGGGGATTATG
GGATCAAGTTGACCCCCCAGAGGTTGAGAACTCTCTGTGAAATAGAATGGCCCTCTTTT
AATGTTGGATGGCCGGCCGAAGGAACTATAGATAGGGAAACAATTGGCCATGTATTTAA
GGTGGTGACTGGGGTCAGAGGACAGCCAGGGCATCCAGACCAATTTCCTTATATTGAC
TCATGGCTAAATATAGTTCAAACTCAACCTGCATGGCTGCAGCCCTGCCTGGCAGCTTA
TTGCAAAACGCTCGTAGCTCGAGCCGAGCCTAAAGTGAAAGAAAAATCAGCTTCACTGT
CAGCTACAGAGACAAAGGGAAAGCCACAAGAAAAACCAGTTTTGCAGGAACCACCAGA
GGAGATAGAAATCCCTCCTCCCTATACCCCAATCTACCCCCCTTTACCAAGGCCGGCCC
CTGAGCAGCCAGACTCAGATGGTGACACACCCCAGGCTACACCCCAAAGGGAGAAATC
TGAGCCCCCACCCCAGGAGGTCAAGGAGGAAAGTCAGGATGATCAAGCAGGCCGCCTT
CGACCTGGCCACACCAGAGTGTGGCAGATGCCCCTCCAGGAAACACGGGGACCCATCT
ATTATGATGAACAGGGCCAGGTTCAAGGGGGGGCAACCGACTTTTATTTACCAGCCTTT
TTCAACCACTGATCTCCTACTGGAAACACCATACTCCCTACTCCCTCCTACATGGAGAAG
CCCCAGGCCCTCATAGATCTGATGCAGTCCATCTTCCAGACACACAATCCAACCTGGCC
AGATTGCAAACAGCTCCTCCTGACACTGTTTAACACCGAGGAGTGCCGAAGGGTGACC
CAGGCAGCCCTCCACTGGCTAGAAGCCAATGCACCAGAAGGCACACTTAATGTCCAGG
CATATGCTCAGGGCCAGTTCCCAGAAGCAGACCCACACTGGGACCCAAATGATGCAAC
CCAATTACAGCACCTGCAGAGGTACCGAGAGGCACTCCTGCAAGGGCTAAAGGAAGGT
GGGAAAAAGGCAGTCAACATGGGGAAAATCTTGGAAGTGCTGCAGGGAACTGATGAAA
GCCCTAGCCAGTTTTATGAGAGACTCTGTGAGGCATTCCAGCTTTACACTCCGTTTGAC
CCTGAGGCTACTGAAAATCAGTGCATGGTGAACACAGCATTTGTAGGACAAGCCCAGG
GGGACATCAGGCGAAAGCTGCAAAAGCTAGAGGGTTTCGCAGGCATGAATGCCACCCA
GCTTTTAGAAGTGGCCACCAAGGTGTATGTTAACCGTGACCAGGAGGCAAAGAGAGGC
TGATCAGAGACTGGAAAAAGGCCGATCTGCTAGTGGCAGCCCTCACAGAAAGGGAAAC
TAGCATCGCGAGTGGGTGTGGACATAGCCATGGACATGGAGGGGTTCAGGCCAGGCA
GAGACCTGAAAGTCAACCAAGACTATGTAGGAATCAATGTGCACAATGCAAAAAGAAGG
GACACTGGAAAAATGAATGTCCAGAGGGCAATGAAGAAAATGACAGAGACTATAAAACT
GGAAAACTGCCAGCCAAGGGCTATTGTGTCCCAAGGGAGCCAGATACCAACCTGATCA
GGCTGGCAGGGACTGAAGAATATGAAGACTAGGCCAGACTGGGCTCCTTCTCCTTAGG
CCCCCAGGAGCCCATGGTCACATTAGAAGTAGGGGGCCAACTAATGGACTTTATGGTA
GACACTGGGGCTGAACACTCGGTAGTGACCCAGCCCATAGGGCCACTATCCAAAAACC
ATACAACTATTGTTGGGGCTACAGGGGTCCCAGAGAAGAGGCCATTTTGTCAGCCAAG
GAGGTGTGTCATAGGAGGACGAGAAGTCCAACATGAATTCCTGTACCTCCCAAATTGTC
CAGTTCCCTTGCTGGGAAGAGACCTACTCCAAAAACTGCAAGCACAGATTGCTTTTGGG
CCACAAGGAGATATGACTTTAAACCTAACTCACCCAAAGGCCATGGTGTTAACCCTTAC
CATCCCACAGGCTGAGGAATGGAGACTATACATGAAGTACAGGCACCAATGCAGCCTT
GTACGCAGGAGGAAGAGAAATTATTATTTAGGCCAATTGATAAAATTCCTGGAGTATGG
GCTGAAGACAACCCACCTGGGCTGGCTGTAAATCAGGCACTGGTAGTAGAATTAAAACC
AGGAGCAACTCCAGTTCAGGTTCGTCAGTACCCACTTTCCCAAGAGGATATATGGGGCA
TTTATAAACATTTAAAGTGGCTCTGTGAACATGGAATCATAGTCCAATGCCAGTCACCCT
GGAACACTCCACTTTTGCTGGTACAAAAACCATTGCCAGGACCAGGATCTGATGAGTAT
AGACCGGTGCAGGACTTGCATGCTGTAAACCAAGCCACGGTGACCATCCATCCAGTAG
TACCAAACCTGTATACTTTAATGGGACTTATTCCAGCAAGTGCTACCTGGTTTACAGTCC
TGGACTTAAAGGATGCTTTCTTCTGTCTCTACCTGGCACCAATTAGTCAGCCCATCTTTG
CATTTCAATGGGACAATTCAGTCACAGGCACAGGGGAACAGCTCACCTGGACTAGACTC
CCACAAGGATTCAAAAACTCTCCCACAATCTTTGGAGAAGCACTGGCCTCAGACCTCAA
GGCCTACACCCCACCAAATGACAACTGCGCCTTGCTGCAATACATAGACGACCTTCTTT
TGGCAGCCCCAACCCAAGAGGACTGTTACCAAGGAACCCAAGACCTCCTCCATCTCTTA
TGGAAAGCAGGTTATAAAGTATCCAAAAAGAAAGCCCAAATTTGCCATGAAAAGGTTAAA
TATTTAGGCTTCATAGTAAGCCAAGGGGAACGCTGGCTTGGCAATGAATGAAAGCAGGC
TGTTTGTGCACTTCCAACTCCAACCACCCGGCGCCAAATAAGAGAATTCTTAGGGGCAG
CAGGGTTCTGCCATATCTGGATCCCAAATTTCTCACTTATGGCCAAGCCCTTATATGAAG
CCACAAAGGAGGGGGAAAAGGAACCCCTCCTCTGGGAGGCTGACCAGGAGAAGGCAT
TTAAACAAATCAAAGAAGCCTTAACTCAGGCCCCAGCCTTAGGACTGCCAGATATAACTA
AGCCTTTCTTTCTATATGTCCATGAATGAAAGGGAATGGCTATAGGGGTCCTGACTCAA
GTCATAGGATCATGGCATCGCCCGGTGGCATACTTATCCAAACACTTGACTCTGTGGTG
CGGATGGCCTCCTTGCCTTAAAGCACTAGCTGCCACCACCCTATTGGCACAAGAAGCTG
ACAAACTGACTCTAGGGCAACAACTGACCATCCAGGTACCACACTCGGTTATAACTTTAA
TGGATCAGAGAGGGCACCATTGGTTATCAAATCCAAGAATGACTCAGTACCAAGGGCTC
CTATGTGAAAATCCCCACATAACTTTAGAAACAGTAACACCCTTAACCCAGCTACCCTGC
TCCCAGTCAAACCGGGAATCACCCTCCATGACTGTGGAAACAGTAGACGAGGTATTCTC
AAGTCGGGGAGACCTTACAGACCAACCCCTCAAGGACCCAGATGTTGAATACTTCACAG
ATGGAAGCAGTTTTGTACTGGAAGGGGTCCGCTGGGCTGGGTATGCAGTGGTAACATT
AGACTCAGTGGTGGAGGCTCAGCCTCTGCCTACTGGAACATCAGCCCAAAAGGCAGAG
CTAATAGCCCTAACAAGAGCTCTCTTGCTGGCAAAAGACAAAAAGATCAATGTTTATACT
GATTCCAAATATGCTTTTGCCACATTGCATGTTCATGGAGCTATATATAAAGAAAGAGGA
CTCTTAACTGCTGGAGGCAAAGAAATAAAGTACAAAGAAGAAATTCTACAGCTCTTAGAT
GCTGTATGGGCCCCAAAGAAAGTAGCTGTTATGCACTGCAGGGGGCACCAAAAGGCAG
GAACACTAGAGGCCAAAGGAAACAGAAAGGCAGACAGGGAGGCAAAACGGGCAGCAA
TGACTACACCGCATTTTAAAAAGGAAGCCCTAGCTATGCCTCTCCTCCCAGAGCCTCCC
CTCCCAGAGGTCCCAAGTTACTCTCCAAATGAGAAGGCCTGGTTTGCCCAAGAAACTGG
AAAATATATTGAAGGAGGATGGTGGAAATTCTCTGATGGGAGGCTAGCCATTCCTGAAA
TGGTGGCCCCCAAATTTGTGAAGCAATTCCATCAAGGAACTCATATGGGAAAAACGGCA
CTAGAAACGCTACTAGGACACCATTTCTATGTGCCACGGCTCACTGCCATCACCCGAGC
CGTTTGTGAACAATGTCTAACTTGTGCCCAGAACAACCCATGACAAGGGCCCACTCGGC
CCCCAGGAATTCAGGAAATAGGAGCCACACCCTGTGAAAACCTGCTTATGGACTTCACT
GAGCTGCCCCGAGCAGGGGGCTATCGGTACATGTTAGTACTTGTCTGCACCTTTTCAG
GATGGGTCGAGGCTTTCCCCACCAGAACAGAGAAAGCATGAGAAGTGACTAAAGTACT
GTTAAGAGACATTATCCCCAGATTTGGACTGCCCCTGACTCTAGGGTCAGACAATGGAC
CGGCATTTGTAGCTGAAATAGTTCAAGAACTAACACGGCTGTTAAAAATAAAATGGAAAT
TACACACAGCCTACCGGCCACAGAGCTCAGGAAAAGTGGAGCACATGAACTGGACACT
CAAACAGCTACTGAAGAAATATTGCCAAGAAACTCATCTGAGATGGGATCAGGTCCTGC
CCATGGTCCTCCTCCGAGTCAGGTGCACCCCCACCAAACAAACTGGGTATTCGCCCTAT
GAGATCTTGTTCGGCCAGCCACCCCCAATCATAGGTCAAATTAAGGGTGATCTCCGTGA
ACTAGGGGAATTAACTTTAAGAAGGCAAATGCAGGCTTTAGGGATAGCCATGCAAAGTG
TCCATGGCTGGGTACAGGAAAGAATGCCCATAAGCCTGACAGACCCAGCACACCCCTT
TAAACCTGGGGACTCTGTTTGGGTTAAGAAATGGAATCCAACCACTCTGGGACCCATAT
GGGATGGGCCCCATACTGTAATCTTGTCCACTCCCACTGCTGTTAAAGTTGCAGGAATT
GTGCCTTGGATCCACCACAGTAGTCGGCTGAAACCAGCAGCCCAGGACAAGTGGACCA
GCCAACAGGACCCAGACCATCCAACCCAGCTGATCCTACGATGGGACCAAGTTGCCAG
TGAAGATGACAACAGCCCTGCTCTGGTCACTCCGGAAGCTGACCAGTCTATACACGGCT
GAAGCTTGAGGAGACAACAGCCCTGCTCTAGTCACCCCAGAAGCTGACTAGTCTATGCA
CGGCCGAAGCTTGAGTCATCATCAGGGAAGTAAATGTGGTTAGAAATCTTAAGCCTAGT
AATTTTCCTTGTAATATTAATTACTTTACTATTAACCTGTCACTTTGCTCAACCTCCTCCCC
CCAGGAAAAAGCCTTTTCTGTCCATGCTGGGTATGAACATGCTACTCATTACTTTGTTCT
TGCTACTCCCCTTATCCATGTTAAAAGGAGAACCCTGGGAGGGATGCCCCCACTGCACA
CACACTACGTGGTCGGGGAGCACCATAACTAGAACCCTGTTGTACCACACTTATTATGA
GTGTACAGGGACCCACCTAGGAACTTGTACTCACAACCAGACAACCTACTCAGTCTGTG
ACCCAGGAAATGGCCAGCCTTATATATGTTATGACCCTAAGTCCTCACCTGGGACCTGG
TTTGAAATTCATGTCGGGTCAAAGGAAGGGGATCTTCTAAACCAAACCAAGGTATTTCCC
TCTGGCAAGGATGTCGTATCCTTATACTTTGATGTTTGCCAGATAACATCCATGGGCTCA
CTCTTTCCCGTAATCTTCAGTTCCATGGAGTACTATAGTAGCTGCCATAAAAATAGGTAT
GCACACCCTGCTTGTTCCACCGATTCCCCAGTAACAACTTGCTGGGACTGCACAACATG
GTCCACTAACCAACAATCACTAGGGCCAATTATGCTTACCAAAATACCATTAAAACCAGA
TTGTAAAACAAGCACTTGCAATCCTGTAAATCTTACCATCTTAGAGCCAGATCTGCCCAT
ATGGACTACAGGTTTAAAAGCACCGCTAGGGGCACGAGTCAGCGGTGAAGAAATTGGC
CCAGGAGCCTATGTCTATCTATATATTATAAAGAAAACTCGGACCCGTTCAACCCAACAA
TTCCGAGTTTTTGAGTCATTCTATGAGCATATCAACCAGAAGTTGCCTGAACCCCCTCCC
TTGGCCAGAAACCTATTCGCCCAACTGGCTGAAAACATAGCCAGCAGCCTACACATCTC
CTCATGCTATGTCTGTGGGGGAACCAACATGGGAGACCAATGGCCATGGGAAGCAAGG
GAGCTAATGCCCCAAGATAACTTCACTCTAACTGTCTCTTCCCCCGAACCTGCGTTCAC
AAGCCAGAGCATCTGGTTCTTAAAAACCTCTATTATTGGAAGATTCTGCATTGCTCGCTG
GGGAAAAGCCTTTACAGACCCAGTAGGAGAATTAACTTGCCTAGGACAACAATATTACA
ATGAAACACTAGGAAAAACTTTATGGTGGGGCAAAAAGAATAATTCCAAATCACCCCACC
CAAGCCCATTCTCCCGTTTCCCTTCTTTAAACCACTCTTGGTACCAACTTGAAGCTCCAA
ATACCTGGCAGGCACCCTCTGGCCTCTACTGGATCTGTGGGCCACAGGCATATTGGCA
ACTGCCAGCTAAATGGTCAGGGGCCTGTGTACTGGGGACAATTAGGCCGTCCTTCTTC
CTAATCCCTCTAAAGCAAGGAGAAGCCTTAGGATACCCCATCTATGATGAAACTAAAAG
GAGAAACAAAAGGGGCATAACCATAGGAGATTGGAAGGACAATGAATGGCCCCCTGAA
AGAATAATCCAATATTATGGCCCAGCCACCTGGGCAGAAGATGGAATGTGGGGATACC
GCACCCCTATTTACATGCTCAACCGCATCATAAGGTTGCAGGCAGTACTTGAAATCATTA
CTAATGAAACTGCAAGGGCCTTAAATCTGCTGGCCCAGCAAGCCACAAAAATGAGAAAT
GCCATTTATCAAAATAGACTGGCCTTAGACTACCTCCTAGCCCAGGAAGGAGGAGTATG
TGGAAAGTTCAATCTAACTAATTGCTGCCTAGAAATTGATGACAATGGAAAGGTCATCAA
AGAAATAACTGCAAAAATCCAAAAATTAGCCCATGTTCCAGTCCAGACTTGGAAAGGATG
GTCTCCAGATTCCCTCTTCGGAGGCTGGTTTTCATCCCTTGGAGGATTTAAAACCTTAGT
AGGAATAGTTCTGGCCATACTAGGAGGTTGCCTAATACTCCCTTGTCTCTTACCCCTCCT
TGTCAAAAACATCCAAACAGCCATAGAGGCTCTTGTAAACAGACAGACTACCACACAAC
TAATGGCCCTAACTAAGTATTAACCCCTGCCAAGAAAAGAGCTACTTCCTCTTGAAGTAA
ATGAAGATAGTGATGCTTTCTATTAAACTTTACTTATAAAAAGCATCAAAGGGGGGAATG
AAGCAGGAAATATAAAAGGAAAAACAAGTAAAGGGAAAACAAGTCCTTTCCTGACCAGT
CTGACTCACTCCAAAGTCCTGCTGGAGCTATGATAATTATCTGCAAGGCCAGGCAGGGG
CTCCGAAGGAGTGGGCTCCAGGAGCAGGGATGAGAAAAACAAGTTCTCCTTATCAGTTT
CCCTGTTTGAAATTCTCTCCCCATAACATTATTCTTTGTTCTGCTCTCACAACTATTTTTG
TAACTATTTCTGCAAGTCTGTAAAGATTTTGTAAGTTCTTGTTTTTCTTTCTGTAGCATGG
CAAGGTCACAAGACATGTTTAAGTAAGGTAGGCTCATGTTGCAAATCCTGTTGTAAAACC
TGTCACGGTATGATTAACTGCCTTTGTTCTGCTTCTGTAAGACTGCTTTCTCACCTCGCA
GGTTTTGCGCCAAAAACCCGACTTGCCCCTGCCTGATGCATGTATAAAAGTCAAGCCCT
GTCTTTGTTCAGGGCTCAGCCTTTGGATGTTAATCCGCTGGGCCAGTGGCCACCTAAAT
AAAACCTTCCTGTTGCACCCAGTGATCTCTCCGGCCTCCTGATTCCCACAACA
SEQ ID NO: 20
H. sapiens
DNA
>HERV-E whole genomic sequence Gag-Pol-Env
TATGGTAGGAGACCACCACTTCTCCTGTTGTCCTTCCCAGTTTCTCCCCAACCTCCCCTT
TTCCCTAGTTTATAAGACAGGAGAAAAGGGAGAAAGCAAAAAGTTGGAAAGAAACAGAA
GTAAGATAAATAGCTAGACGACCTTGGCGCCACCACCTGGCCCTGGTGGTTAAAATGAT
AATAATATTAACCCCTGACCAAAACTACTGGTGTTATCTGTAAATTCCAGACATTATATGA
GAAAGCACTGTAAAACTTTTTGTACTGTTAGCTGATGTAGGTAGCCCCCAGTCACGTTCC
TCACACTTACTTGATCTGTGATGACTCTTTCACGTAGACCCCTTAGAGTTGTAAGCCCTT
CAAAGGGCTAGGAATTTCTTTTTCGGGGAGCTCGGCTCTTAAGATGCGACTCTGCCGAT
GCTCCTGGCCGAATAAAAACCTCTTCCTTCTTTAATCCAGTGTCTGAGGAGTTTTGTCTG
CGACTCATCCTGCTACATTTCTTGGTTCCCTGCCTGGGAAGCGAGGTAATTGATGGACG
GAGGGTCGAGGCAGCCCCTTAGGTGGCTTAGGCCTGCCCTGTGGAGCATCCCTGTGG
GGGACTCTGGCCAGCTTGAGTGACGCAGATCCTGAGACCACTCCCGGGTAGGCAATTG
CCCCGGTGGAACGCCTCATCAGAGCAGTGCGTGGCAGGCCCCTGTGGAGGATCAACG
CAGTGGCTGAACACCGGGAAGGAATGGGCACTTGGAGTCCGGACATTTGAAACTTGGT
AAGACTGGTCTTTGGAACTTGCCCACTCCATCTGAGTGGAAGCGTGGCCTGATCACCCA
TGGCGTGCCTGTACCGGCACTTTGGTTTTTGTTTTTGACTTGACTTGAATTGCTTGATAT
TTTGGTTTTGGTTTTGACCTGGCTTGGACTTCTGGATACTCGGATTTTGGTTTTGATTCT
GGTTTGGTGTAAACTGAAAAAGTGTGTGTGTGCCCTTTTTACCTGTTCTTTGTTCTGTGG
TGTGTGTGTGGTGTAAGCTTGGTGTTTTGTCTTGAGGAAACACGGGTCAGACACAAAGT
AAGCCTACTCCGCTAGGAACTATGTTGAAAAATTTCAAGAAAGGATTTAATGGAGACTAT
GGAGTTACTATGACACCAGGAAAACTTAGAACTTTGTGTGAAATAGATTGGCCAGCATTA
GAAGTGGGTTGGCCATCAGAAGGAAGCCTGGACAGGTCCCTTGTTTCTAAGGTATGGC
ACAAGGTAACTGGTAAGTCAGGACACCCAGACCAGTTTCCATACATAGACACTTGGTTA
CAGCTGGTTTTAGACCCCCCACAGTGGTTAAGAGGACAGGCAGCAGCAGTACTAGTAG
CAAAGGGACAGATAGCCAAGGAAGGATCCCGCTCCACCCGCTGAGGGAAATCAACTCC
TGAAGTTCTGTTCGACCCAACATCAGAAGATCCATTGCAGGAGATGGCACCAGTGATCC
CAGTGGTGCCCTCCCCTTACCAGGGAGAGAGGCTCCCCACTTTTGAGTCCACAGTGCT
TGCGCCTCCACAAGACAAACATATCCCTAGGCCACCCAGAGTAGACAAGAGAGGAGGT
GAAGCCTCGGGAGAAACCCCTCCCTTGGCAGCTCGTTTAAGACCCAAAACTGGGATAC
AAATGCCCCTGAGAGAGCAGCGGTATACTGGGATAGATGAGGATGGACACATGGTGGA
GAGGCGTGTTTTTGTGTACCAGCCCTTCACCTCTGCCGACCTCCTCAACTGGAAAAATA
ATACCCCATCCTATACTGAAAAGCCTCAAGCTCTAATTGATTTGCTCCAAACTATTATCCA
GACCCATAACCCCACTTGGGCTGATTGCCACCAGTTGCTCATGTACCTCTTTAACACAG
ATGAAAGGCGGAGAGTGCTCCAAGCAGCAACTAAGTGGCTAGAGGAACATGCACCAGC
TGATTACCAAAACCCCCAAGAGTATGTAAGGACCCAGTTACCAGGAACCGACCCCCAGT
GGGACCCAAATGAAAGAGAGGATATGCAAAGGCTAAACCGATACAGGGAAGCTCTCTT
GGAAGGATTAAAGAGGGGAGCCCAGAAGGCCACAAACGTTAACAAGGTCTCTGAGGTC
ATTCAAGGAAAAGAAGAAAGTCCAGCACAATTCTATGAGAGACTGTGTGAGGCCTATCG
TATGTATACTCCCTTTGATCCCGATAGCCCTGAAAATCAGCGCATGATTAACATGGCTTT
AGTTAGTCAAAGCGCAGAAGACATTAGAAGAAAACTGCAGAAACAGGCTGGGTTTGCAG
GGATGAACACATCACAGTTATTAGAAATAGCTAACCAGGTGTTTGTAAACAGGGATGCA
GTAAGCCGTAAGGAAAACCACAGAGAGAATGAACGTCAGGCCCAGCGAAACGCCGACC
TGTTAGCTGCAGCAATCAGAGGGGTCCCCCCAAAGAGGCAAGGGAAGGGGGGCCCCG
GGAAAGAAACTCAGCCTGGCTGTCAGAGCTTGCAGCGTAATCAGTGTGCTTATTGTAAA
GAAATAGGACATTGGAAGAACAAATGCCCTCAGCTAAAAGGAAAACAAGGTGACTCGGA
GCAGGAGGCCCCAGACAAGGAGGAAGGGGCCCTGCTCAACCTGGCAGAAGGGTTATT
GGACTGAGGGGGACTGGGCTCAAGGGCCCCCAAAGAGCCCATGGTCAGGATGACAGT
TGGGGGTAAAGACATTGATTTTCTTGTAGATACTGGTGCTGAACATTCAGTAGTAACCAC
CCCGGTCGCCCCCTTATCCAAAAAGACTATTGATATAATCGGAGCCACAGGAGTTTCAG
CAAAGCAAGCTTTCTGCTTGCCCCGGACTTGTACTGTAGGAGGACATAAAGTGATTCAT
CAGTTTTTGTACATGCCTGACTGTCCCTTGCCCTTGTTGGGAAGGGACTTGCTTAGCAA
GCTGAGAGCCACTATCTCTTTTACAAAGCATGGCTCTTTACAGCTAAAGTTACCCGGAAC
AGGAGTCATTATGACCCTTACGGTCCCCCGAGAGGAAGAATGGAGACTTTTCTTAACTG
AGCCAGGCCAAGAGATAAGACCAGCTCTGGCTAAGCGGTGGCCAAGAGTGTGGGCAG
AAGACAACCCTCCAGGGTTGGCAGTCAACCAAGCCCCCGTACTCATAGAAGTTAAGCCT
GGGGCCCAGCCAGTTAGGCAAAAACAGTATCCGGTCCCCAGAGAAGCTCTTGAAGGTA
TCCAGGTCCATCTCAAGCGCCTAAGAACCTTTGGAATTATAGTTCCTTGTCAGTCTCCAT
GGAACACTCCCCTCCTGCCTGTTCCCAAGCCAGGGACCAAGGACTACAGGCCGGTACA
GGATTTGCGCTTGGTCAATCAAGCTACAGTGACTTTACATCCAACAGTACCTAACCCGTA
CACATTGTTGGGGTTGCTGCCAGCTGAGGACAGCTGGTTCACCTGCCTGGACCTGAAA
GATGCTTTCTTTAGCATCAGATTAGCCCCTGAGAGCCAGAAGCTGTTTGCCTTTCAGTG
GGAAGATCCGGAGTCAGGTGTCACTACTCAGTACACTTGGACCCGGCTTCCCCAAGGG
TTCAAGAACTCCCCCACCATCTTCGGGGAGGCATTGGCTCGAGACCTCCAGAAGTTTCC
CACCAGAGACCTAGGCTGCGTGTTGCTCCAGTACGTTGATGACCTTCTGCTGGGACAC
CCCACGGCAGTCGGGTGCGCCAAAGGAACAGATGCCCTACTCCGGCACCTGGAGGAC
TGTGGGTATAAGGTGTCCAAGAAGAAAGCTCAGATCTGCAGACAGCAGGTACGTTACCT
GGGATTTACTATCCGACAGGGGGAGCGCAGCCTGGGATCAGAAAGAAAGCAGGTCATT
TGCAATCTACCGGAGCCTAAGACCAGAAGGCAGGTGAGAGAATTCTTAGGAGCTGTGG
GGTTTTGCAGACTGTGGATCCCAAACTTTGCAGTATTAGCCAAGCCTTTGTATGAGGTC
ACAAAGGGGGGGGAACGGGAACCTTTTGAATGGGGATCCCAACAACAGCAAGCCTTTC
ATGAGTTAAAGGAAAAACTTATGTCAGCCCCAGCCCTGGGGCTACCAGATCTGACAAAG
CCTTTTACATTGTATGTGTCAGAGAGAGAAAAGATGGCAGTTGGAGTTTTAACCCAAACT
GTGGGGCCCTGGCCGAGGCCAGTGGCCTACCTCTCTAAACAACTAGACGGGGTTTCTA
AAGGATGGCCCCCATGTTTGAGGGCCTTGGCAGCAACTGCCCTGCTAGTACAAGAAGC
AGATAAGCTGACTCTTGGGCAAAACCTGAACATAAAGGCCCCCCATGCTGTGGTGACTT
TAATGAATACCAAAGGACATCATTGGCTAACGAATGCTAGACTAACTAAGTACCAAAGCT
TGCTCTGTGAAAATCCCCGTATAACCATTGAAGTTTGTAACACCCTGAACCCCGCCACC
TTGCTCCCAGTATCAGAGAGCCCTGTCGAGCATAACTGTGTAGAAGTGTTGGACTCAGT
TTATTCTAGCAGACCTGACCTCCGGGACCAGCCTTGGGCATCAGTAGACTGGGAGCTAT
ACGTGGATGGGAGCAGCTTCATCAACCCACAAGGAGAGAGATGTGCAGGATATGCGGT
GGTAACTCTGGACACTGTTATTGAAGCCAAATCGTTGCCCCAGGGCACTTCAGCCCAGA
AAGCTGAACTCATTGCTTTAATTCGGGCCTTAGAACTCAGTGAAGGTAAGACTGTAAACA
TTTACACTGACTCTCGATATGCCTTTTTAACCCTTCAAGTGCATGGAGCATTATATAAAGA
AAAGGGCCTATTGAACTCTGGGGGAAAGGACATAAAATATCAACAAGAAATCTTGCAATT
ATTAGAAGCAGTATGGAAACCCCACAAGGTGGCAGTTATGCATTGCAGAGGACACCAG
CGAGCTTCCACCTTGGTGGGTTTAGGGAATTCCCGCGCTGACTCAGAGGCTCGAAAAG
CAGCATCTACCCCCTTCCGGGCATCAGTCACAGCCCCCCTGCTCCCTCAAGCACCTGA
TCTTGTACCTACTTATTCTAAAGAAGAAAAGGACTTTCTCCAGGCAGAGGGAGGACAAG
TGATAGAGGAAGGATGGATTCGGTTACCAGATGGGAGAGTAGCTGTGCCACAGCTGCT
AGGAGCCGCAGTTGTACTGGCTGTGCATGAAACCACCCATCTAGGCCAGGAGTCACTT
GAAAAGTTGTTAGGCTGGTATTTCTACATCTCACATTTGTCAGCCCTTGCCAAAACAGTG
ACGCAGCGGTGTGTTACCTGCCGACAGCATAATGCGAGGCAAGGTCCAGCTGTTCCAC
CCGGCATACAAGCTTATGGAGCAGCCCCCTTTGAAGATCTCCAGGTGGACTTCACAGA
GATGCCAAAATGTGGAGGTAACAAGTATTTACTAGTTCTTGTGTGTACCTACTCTGGGTG
GGTGGAGGCTTATCCAACACGAACTGAGAAAGCTCGTGAAGTAACCCGTGTGCTTCTTC
GAGATCTTATTCCTAGGTTTGGACTGCCCTTACGGATCGGCTCAGATAATGGGCCGGCA
TTTGTGGCTGACTTGGTACAGAAGACAGCAAAGGTATTGGGGATCACATGGAAACTACA
TGCCGCCTACCGACCTCAGAGTTCCGGAAAGGTGGAGCGAATGAATCGGACTATCAAA
AATAGTTTAGGGAAAGTATGTCAGGAAACAGGATTAAAATGGATACAGGCTCTCCCTAT
GGTATTGTTTAAAATTAGATGTACCCCTTCTAAAAGAACAGGATATTCCCCTTATGAAATA
TTATATCATAGGCCCCCTCCTATATTGCGGGGACTTCCAGGCACTCCCCGAGAGTTAGG
TGAAATTGAGTTACAGCGACAGCTACAGGCTTTAGGAAAAATTACACAAACAATCTCAGC
CTGGGTAAATGAGAGATGCCCTGTTAGCTTATTCTCCCCAGTTCACCCTTTCTCCCCAG
GTGATCGAGTGTGGATCAAGGACTGGAACGTAGCCTCTTTGTGGCCACGGTGGAAAGG
ACCCCAGACTGTCATCCTGACCACTCCCACCGCTGTGAAGGTAGAAGGAATCCCAGCC
TGGATCCACCACAGCCATGTAAAACCTGCAGCGCCTGAAACCTGGGAGGCAAGACCAA
GCCCAGACAACCCCTGCAAAGTGACCCTGAAGAAGACGACAAGCCCTGCTCCAGTCAC
ACCCGGAAGCTGACTGGTCCACGCACGGCCGAAGCATGAGGAAGCTCATCGTGGGAC
TCATTTTTCTTAAATTTTGGACTTATACAGTAAGGGCTTCAACTGACCTTACTCAAACTGG
GGACTGTTCCCAGTGTATTCATCAGGTCACTGAGGTAGGACAACAAGTTAAAACAATCTT
TCTGTTCTATAGTTATTATGAATGTATAGGAACTTTAAAAGGAACTTGTTTGTATAATGCC
ACTCAGTACAAGGTATGTAGCCCAGGAAATGACCGACCTGATGTGTGTTATAACCCATC
TGAGCCCCCTGCAACCACCGTTTTTGAAATAAGATTAAGAACTGGCCTTTTCCTAGGTGA
TACAAGTAAAATAATAACTAGAACAGAAGAAAAAGGAATCCCCAAACAAATAACTTTAAG
ATTTGATGCTTGTGCAGCCATTAATAGTAAAAAGCTAGGAATAGGATGTGGTTCTCTTAA
CTGGGAAAGGAGCTACAGAGTAGAAAATAAATATGTTTGTCATGAGTCAGGGGTTTGTG
AAAATTGTGCCTATTGGCCATGTGTTATTTGGGCTACTTGGAAAAAGAACAAAAAGGACC
CGGTTTATCTTCAGAAGGGGGAAGCCAACCCCTCCTGTGCTGCCGGTCACTGTAACCC
ACTAGAACTAATAATTACCAATCCCCTAGATCCCCATTGGAAAAAGGGAGAACGTGTAAC
CCTGGGGATCAATGGGACAGGGTTAAACCCCCAAGTTGCCATTTTAATTAGAGGGGAG
GTCCACAAGTGCTCTCCCAAACCAGTATTTCAAACCTTTTATGAGGAGCTGAATCTGCCA
GCACCAGAACTTCCGAAAAAGACAAAAAATTTGTTTCTCCAATTAGCAGAAAATGTAGCT
CATTCCCTTAATGTTACTTCTTGTTATGTATGCGGGGGAACCACTATCGGAGACCGATG
GCCTTGGGAAGCCCGAGAGTTGGTGCCTACTGATCCAGCTCCTGATATAATTCCAGTTC
AGAAGGCCCAAGCTAGCAACTTCTGGGTCCTAAAAACCTCAATTATTGGACAATACTGC
ATAGCTAGAGAAGGGAAAGACTTTATCATCCCTGTAGGAAAGCTTAATTGTATAGGACA
GAAGTTGTATAACAGCACAACAAAGACAATTACTTGGTGGGGCCTAAACCACACTGAAA
AGAATCCATTTAGTAAATTTTCTAAATTAAAAACTGCTTGGGCTCATCCAGAATCTCATCA
GGACTGGACAGCTCCCGCTGGACTATACTGGATATGTGGGCACAGAGCCTACATTCGG
TTACCTAATAAATGGGCAGGCAGTTGTGTTATTGGCACTATTAAGCCATCCTTTTTCTTAT
TACCCATAAAAACAGGTGAGCTCCTAGGTTTCCCTGTCTATGCCTCCCGAGAAAAGAGA
AGCATAGCTATAGGAAACTGGAAAGATAATGAGTGGCCCCCTGAAAGAATCATACAGTA
TTATGGGCCTGCCACATGGGCACAAGACGGCTCATGGGGATACCGAACCCCCATCTAC
ATGCTCAACCGGATCATACGGTTACAGGCTGTCTTAGAAATAATTACTAATGAAACTGGC
AGAGCTTTGACTGTTTTAGCTTGGCAAGAAACCCAAATGAGGAATGCTATCTATCAGAAT
AGACTGGCCTTAGACTACTTGCTAGCAGCTGAAGGAGGAGTTTGTGGAAAATTTAACTT
AACCAATTGCTGTCTACAAATAGATGATCAAGGACAAGTGGTTGAAAACATAGTCAGAGA
CATGACAAAGCTGGCACATGTGCCTGTACAGGTTTGGCACGAGTTTGATCCTGAGTCTT
TATTTGGAAAATGGTTTCCAGCTATAGGAGGATTTAAAACCCTCATTGTAGGTGTATTAA
TAGTAATAGGAACCTGCTTGCTGCTCCCCTGTTTGCTACCCTTGCTTTTTCAAATGATAA
AAAGCTTTGTTGCTACTTTAGTTCATCAAAATACTTCAGCACAAGTGTATTATATGAATCA
CTATCGCTCTATCTCACAAAAAGACTCAGAAAGTGAGGATGAGAGTGAGAACTCCCACT
AATAAAAAGTGAAAATTCTCAAAGGGGGGGAATATGGTACGAGACCACCACTTCTCCTG
TTGTCCTTCCCAGTTTCTCCCCAACCTCCCCTTTTCCCTAGTTTATAAGACAGGAGAAAA
GGGAGAAAGCAAAAAGTTGGAAAGAAACAGAAGAAAGATAAATAGCTAGACGACCTTGG
CGCCACCACCTGGCCCTGGTGGTTAAAATGATAATAATATTAACCCCTGACCAAAACTA
CTGGTGTTATCTGTAAATTCCAGACATTGTGTGAGAAAGCACTGTAAAACTTTTTGTAGT
GTTAGCTGATGTAGGTAGCCCACAGTCACATTCCTCACACTTACTTGATCTATTATGACT
CTTTCACGTAGACCCCTTAGAGTTGTAAGCCCTTCAAAGGGCTAGGAATTTCTTTTTCGG
GGAGCTCGGCTCTTAAGATGCGAGTCTGCTGATGCTCCCGGCCGAATAAAAAACCTCTT
CCTTCTTTAATCCGGTGTCTCAGGAGTTTTGTCTGCGACTCGTCCTGCTACA
SEQ ID NO: 21
H. sapiens
DNA
>HERV-V whole genomic sequence Gag-Env
ACAGACTCCTTCTTTTCAGGAGCCCACTCGGTTCTGTCGTTCAGGGTGTCGCTTCTTTCT
GAGCTCCTGTCTCTTTAAATGAAGGGCATTTACCCTTTAACTCTTTCTGCGCATCTCTTG
ACTGAATCCAAGAAGACCAAGAACCAGAGGACTTCCACACCCCTCTGGGTAACATCTGG
TGTTTTCACCTGGTTACCTGGTTACCTCGCCTGGTTATCCAGCTATCAACATTTCTGGCA
TCTCCAGTTGGATACATCAGTCTCAAGTGAAACTGTGGGAAGGTCCCGAAGAACCACAG
AAGAACAACAGAATTCAGCACCTGAGCATTCTTGTGAGCCACTGGAGAACTTAAAATTC
CACTTCAAGTGAAAAGATAAGTAAAGCCTTCTCTCTGTTCACCCAAAACTAAAGTCAATC
TCAGTACGGGGAATCTTGGTTGCGGTGGCATTGGTTCTTCTCCTTATTTTGACCCAACT
GGCATGCCACCTGAAGTCCCGATAACAGCCTGATTTCTCACTAAACACTCCATCGAACC
ACTTCATTATTTGTCTCTCCTATTTTCAGCACTTTCCTTTTGCTTTCACAAAGCTTAAGGG
AGAATCAGCTATGACAGAGAAATTCCTTTTCCTTTATCTTTCCCTCCTTCCCATGCCCCT
ACTCTCACAGGCACAGTGGAATGAAAATTCCCTTGTCAGTTTTTCCAAAATAATTGCTTC
GGGAAACCATCTAAGCAACTGTTGGATCTGCCACAACTTCATCACCAGGTCCTCATCTT
ACCAATATATTTTGGTAAGAAATTTTTCTTTAAACCTAACATTTGGTTCAGGAATCCCTGA
AGGCCAACATAAATCTGTTCCGCTCCAGGTTTCGCTTGCTAACTCAGCGCACCAAGTCC
CCTGCCTGGATCTCACTCCACCTTTCAATCAAAGCTCTAAAACTTCTTTCTATTTCTACAA
CTGCTCTTCTCTAAACCAAACCTGTTGTCCATGCCCTGAAGGACACTGTGACAGGAAGA
ACACCTCTGAGGAGGGATTCCCCAGTCCCACCATCCATCCCATGAGCTTCTCCCCAGCA
GGCTGCCACCCTAACTTGACTCACTGGTGTCCAGCTAAACAAATGAACGATTATCGAGA
CAAGTCACCCCAAAACCGCTGTGCAGCTTGGGAAGGAAAAGAGCTAATCACATGGAGG
GTTCTATATTCGCTTCCCAAGGCACACACTGTCCCCACATGGCCAAAATCTACTGTTCCC
CTGGGAGGGCCTCTATCCCCTGCATGCAATCAAACTATTCCAGCAGGGTGGAAATCGC
AGTTACACAAGTGGTTCGACAGCCACATCCCCCGGTGGGCCTGTACCCCTCCTGGCTA
TGTATTTTTATGTGGGCCACAAAAAAATAAACTGCCCTTTGATGGAAGTCCTAAGATAAC
CTATTCAACCCCCCCTGTGGCAAACCTCTACACTTGCATTAATAACATCCAACATACGGG
AGAATGTGCTGTGGGACTTTTGGGACCACGGGGGATAGGTGTGACCATTTATAACACCA
CCCAACCCAGACAGAAAAGAGCTCTGGGTCTAATACTGGCAGGGATGGGTGCGGCCAT
AGGAATGATCGCCCCATGGGGAGGGTTCACTTATCATGATGTCACCCTCAGAAATCTCT
CCAGACAAATAGACAACATAGCTAAGAGTACCAGAGATAGCATCTCTAAACTCAAGGCC
TCCATAGATTCTCTAGCAAATGTAGTCATGGACAACAGATTGGCCTTAGATTACCTCTTA
GCAGAGCAGGGTGGAGTCTGTGCAGTGATCAATAAATCCTGTTGCGTTTATGTCAATAA
CAGTGGGGCGATAGAGGAGGATATAAAAAAGATCTATGATGAGGCTACGTGGCTCCAT
GACTTTGGAAAAGGAGGTGCTTCAGCAAGGGCCATCTGGGAGGCTGTGAAGTCTGCCC
TCCCCTCCCTCAACTGGTTTGTCCCTTTACTGGGACCAGCAACAGTTATACTCTTACTTT
TCCTCTTTGGCCCTTGTTTCTTTAATTTACTGATTAAGTGTGTCTCTTCTAGGATAAAGCA
ATTTCACATGAAGTCCCCCCAAATGGAAAGATATCAGCTATCTGTCATTGGAGGCCCCA
GCACCTATAAGCACATCTCCCCCTTGGATGCCAGTGGGCAAAGATTCCGGGAAACTATG
GAGGAATTTTCTCTCTGAGACAGAGCAAGAGAGGGAGACCCTGATGACTTCTTCGCCCC
ATGTCAGCAGGAAGTAGTTACAGAAGACCCACGACGTCCTTACAACCAGAGCTTTTCAG
GGTCTCCATCTCTTGAGGAGGGAAATATTAGGGTAGGCAGGTAGGCAGGCATGAGCAG
GCAAGAGAGCCCTTGGGAAAGGAATCTTTAGAAACGCAGCCCACTGATAGCTTCCTTGG
TGATGCTGCCCACAGACAGTCAGCACTTCTCTAATAACCCATCCTAGAACA
SEQ ID NO: 22
H. sapiens
protein
>HERV-F(c)2 consensus protein sequence Gag
MGSTQSKIVQNTPLGCLLRNLPTLQLDQDLKRKRLIFFCTVAWPQYTLDNQSRWPPEGTLN
FNILNDLTNFCQKQGKWSEIKYVQGFWDLHSPPDLCAPCSLVQVLLAKTSPKTSPDPDKDD
LSPLSDPIDNLSSPPLQTAAHALPPPYASPPPSAPPSGHAPPTSSRVPPSPITPPPPPPPSSP
PPPPLPSPAPPKSPVAAHTQARPALLAPLREVAGAEGIVRVHVPFSLVDLSKIERHLGSFSTN
PTLFTKEFHYLCQVYDLTWHDIHIILTSTLSPEERERILIAARQHANQLHLTDPNVPVGTQAVP
STDPEWDYQVGQAGRRRRDIMVQCLLAGMQVASNKSVNFDKLKEIVQYPDENPAVFLNRL
TDALVHYTRLDPASPAGATILATYFISQSATDIRKKLKKAEEGLQTPIQDLVKLAFKVFNSREE
AAEVQQQARLKQKVQLQTQALAAAVQPAFPKSPGKKGRGTISRAPSGACFKCGNSGHWA
SRCPSQQQPSCLPYNCFKCGNPGHWAKQCPNPKPPMRPCPNCRQMGHWRSDCPGLRA
AAVSPHGDPSPDGEGTFQLLQLDDD
SEQ ID NO: 23
H. sapiens
protein
>HERV-F(c)1 consensus protein sequence Gag
MGGAQSKIDPKTPLGCLLANFEALGLSMDLKRKRLIFFCLVAWPQYKLDNQSRWPPEGTFD
FQILQDLDNLCRRQGKWSEVPYVQAFWDLRSRPDLCAKCSLGQVLLAKASPSNKEPDSSP
LSEPPEALALPPLPAALPPPYPGSSGPTPTAPPLPPTPPSSPANPPASALPPPSPVSAHTRS
KTDLLCLLREVAGAEGVVRVHVPFSLTDLSKIEKRPGSFSANPTLYIKQFRYLCQAYDLTWR
DLHIILTSTLSPEERERVQAVARQHADQIHLTDPAMPVGTLAVPAAEPDWDYQAGQTGRRR
RDQMVQCLLASMQAASNKTVNFDKLREIIQGSDENPAVFLNCLTEALIQYTRLDPTSPAGAT
VLATHVISQSAGDIRKKLKKVEEGPQTPIQDLVKMAFRVYNSREETAEAQRQARLKQKVQFQ
TQALVAAPRLAGSGSQPKGGSGHRAPPGACFKCGNEGHWAWQCPYPKEPTRPCPNCHQ
MGHWKSECPSVGASTVPLRCENSETTGGAFQLLSMDDD
SEQ ID NO: 24
Mus musculus
siRNA ID: s523645
Sense: Sequence (5′→3′)
CGCUUGGUCCAGUUUGUAAtt
SEQ ID NO: 25
Mus musculus
siRNA ID: s523645 MuERV Env P10404
Antisense: Sequence (5′→3′)
UUACAAACUGGACCAAGCGgt
SEQ ID NO: 26
Mus musculus
siRNA ID: s523644 MuERV Env P10404
Sense: Sequence (5′→3′)
CGGGCAUACUGUACCAACAtt
SEQ ID NO: 27
Mus musculus
siRNA ID: s523644 MuERV Env P10404
Antisense: Sequence (5′→3′)
UGUUGGUACAGUAUGCCCGgg
SEQ ID NO: 28
Mus musculus
siRNA ID: s523643 MuERV Env P10404
Sense: Sequence (5′→3′)
CCAACUUAAUGACAGGACAtt
SEQ ID NO: 29
Mus musculus
siRNA ID: s523643 MuERV Env P10404
Antisense: Sequence (5′→3′)
UGUCCUGUCAUUAAGUUGGta
SEQ ID NO: 30
Mus musculus
siRNA ID: s529374 MuERV Gag
Sense: Sequence (5′→3′)
GGAACCACCUAGUUCUCUAtt
SEQ ID NO: 31
Mus musculus
siRNA ID: s529374 MuERV Gag
Antisense: Sequence (5′→3′)
UAGAGAACUAGGUGGUUCCta
SEQ ID NO: 32
Mus musculus
siRNA ID: s529085 MuERV Gag
Sense: Sequence (5′→3′)
GGUCGAAAGUUAGAGCGGUtt
SEQ ID NO: 33
Mus musculus
siRNA ID: s529085 MuERV Gag
Antisense: Sequence (5′→3′)
ACCGCUCUAACUUUCGACCaa
SEQ ID NO: 34
Mus musculus
siRNA ID: s535486 MuERV Gag
Sense: Sequence (5′→3′)
AGUCCGUAGAUGUCAAGAAtt
SEQ ID NO: 35
Mus musculus
siRNA ID: s535486 MuERV Gag
Antisense: Sequence (5′→3′)
UUCUUGACAUCUACGGACUga
SEQ ID NO: 36
Mus musculus
siRNA ID: 5406 XPR1
Sense: Sequence (5′→3′)
GGAUAUGCUGUAUUCAGCUtt
SEQ ID NO: 37
Mus musculus
siRNA ID: 5406 XPR1
Antisense: Sequence (5′→3′)
AGCUGAAUACAGCAUAUCCtt
SEQ ID NO: 38
Mus musculus
siRNA ID: 5493 XPR1
Sense: Sequence (5′→3′)
GGUAUUGAUAGAAGACACAtt
SEQ ID NO: 39
Mus musculus
siRNA ID: 5493 XPR1
Antisense: Sequence (5′→3′)
UGUGUCUUCUAUCAAUACCtt
SEQ ID NO: 40
Mus musculus
siRNA ID: 60216 Slc7a1 (mCAT-1)
Sense: Sequence (5′→3′)
GGACUGUUAACUCUUGGCGtt
SEQ ID NO: 41
Mus musculus
siRNA ID: 60216 Slc7a1 (mCAT-1)
Antisense: Sequence (5′→3′)
CGCCAAGAGUUAACAGUCCtg
HERV H (SEQ ID NOs. 42-44)
SEQ ID NO: 42
Homo sapiens
siRNA
Sense: Sequence (5′→3′)
GCAAACAACUGCUGGCUUUuu
SEQ ID NO: 43
Homo sapiens
siRNA
Sense: Sequence (5′→3′)
GCAAAGACCCACCGGAAUuu
SEQ ID NO: 44
Homo sapiens
siRNA
Sense: Sequence (5′→3′)
CGGCAAAGACCCACCGGAAuu
HERV K
SEQ ID NO: 45
Homo sapiens
shRNA
Sense: Sequence (5′→3′)
CCTGAACATCCAGAATTAT
HERV T(SEQ ID NOs. 46-48)
SEQ ID NO: 46
Homo sapiens
siRNA
Sense: Sequence (5′→3′)
AGGAGAAACUGGACUAAUAuu
SEQ ID NO: 47
Homo sapiens
siRNA
Sense: Sequence (5′→3′)
GGGUGAAGCCAUUAGAUUAuu
SEQ ID NO: 48
Homo sapiens
siRNA
Sense: Sequence (5′→3′)
ACGCAUAGCUUCUGUCAAAuu
HERV W (SEQ ID NOs. 49-50)
SEQ ID NO: 49
Homo sapiens
shRNA
Sense: Sequence (5′→3′)
GCAGCGTCCCGGAAATATTGA
SEQ ID NO: 50
Homo sapiens
shRNA
Sense: Sequence (5′→3′)
GGAAATCTCAGCTGCACAACC
HERV FRD/Syncytin2 (SEQ ID NOs. 51-53)
SEQ ID NO: 51
Homo sapiens
siRNA
Sense: Sequence (5′→3′)
CCAUAAAGCUCCAGACGAAuu
SEQ ID NO: 52
Homo sapiens
siRNA
Sense: Sequence (5′→3′)
GGGUAAAUCAAUCAGGAAAuu
SEQ ID NO: 53
Homo sapiens
siRNA
Sense: Sequence (5′→3′)
GGAGCUAACCAGAGCCAAAuu
HERV R/ERV3
SEQ ID NO: 54
Homo sapiens
siRNA
Sense: Sequence (5′→3′)
AGGCAUAACUAUAGGAGAU
HERV R(b) (SEQ ID NOs. 55-57)
SEQ ID NO: 55
Homo sapiens
siRNA
Sense: Sequence (5′→3′)
GCCAAACACAACTCGTGTT
SEQ ID NO: 56
Homo sapiens
siRNA
Sense: Sequence (5′→3′)
GCCGACATACTATACTGTT
SEQ ID NO: 57
Homo sapiens
siRNA
Sense: Sequence (5′→3′)
GCTTCCCTCAGGTTGGTTA
HERV F(c)2 (SEQ ID NOs. 58-60)
SEQ ID NO: 58
Homo sapiens
siRNA
Sense: Sequence (5′→3′)
GCAATACCAGCTGGGTCAA
SEQ ID NO: 59
Homo sapiens
siRNA
Sense: Sequence (5′→3′)
GCACTGGTGCAATATCCAT
SEQ ID NO: 60
Homo sapiens
siRNA
Sense: Sequence (5′→3′)
GGTGTAGATGGAGGACTAT
HERV E (SEQ ID NOs. 61-63)
SEQ ID NO: 61
Homo sapiens
siRNA
Sense: Sequence (5′→3′)
CCGACCCGATGTGTGTTAT
SEQ ID NO: 62
Homo sapiens
siRNA
Sense: Sequence (5′→3′)
GCTCGTGCAGCCATTAATA
SEQ ID NO: 63
Homo sapiens
siRNA
Sense: Sequence (5′→3′)
GCAGGAAATGTAGCTCATT
HERV P(b1)/HERV IP (SEQ ID NOs. 64-66)
SEQ ID NO: 64
Homo sapiens
siRNA
Sense: Sequence (5′→3′)
GCTAGCCACCTCATTATTA
SEQ ID NO: 65
Homo sapiens
siRNA
Sense: Sequence (5′→3′)
GCTTGCAGCAGCAGAGAAA
SEQ ID NO: 66
Homo sapiens
siRNA
Sense: Sequence (5′→3′)
CCAGATGCCAAGTTTACTT
HERV V (SEQ ID NOs. 67-69)
SEQ ID NO: 67
Homo sapiens
siRNA
Sense: Sequence (5′→3′)
TCAACTGGTTTGTCCCTTT
SEQ ID NO: 68
Homo sapiens
siRNA
Sense: Sequence (5′→3′)
GGCCCTTGTTTCTTTAATT
SEQ ID NO: 69
Homo sapiens
siRNA
Sense: Sequence (5′→3′)
GGGCAAAGATTCCGGGAAA
HERV MER34 (SEQ ID NOs. 70-72)
SEQ ID NO: 70
Homo sapiens
siRNA
Sense: Sequence (5′→3′)
GCACCTTCATGCCCTCTAT
SEQ ID NO: 71
Homo sapiens
siRNA
Sense: Sequence (5′→3′)
GCAGATGCCAGCATAACTA
SEQ ID NO: 72
Homo sapiens
siRNA
Sense: Sequence (5′→3′)
CCAGATACCTCACCTTAAA
Primers for qRT-PCR (SEQ ID NOs. 73-92)
Numbers in parentheses indicate start binding location to the respective gene
SEQ ID NO: 73
DNA
artificial
qRT PCR product size 149 bp
hActB fwd
ACTCTTCCAGCCTTCCTTCC (780)
SEQ ID NO: 74
DNA
artificial
qRT PCR product size 149 bp
hActB rev
CAATGCCAGGGTACATGGTG (909)
SEQ ID NO: 75
DNA
artificial
HERV H1-3
qRT PCR product size 113 bp
HERV H1-3 fwd
tccccagacatttcacatcag (358)
SEQ ID NO: 76
DNA
artificial
HERV H1-3
qRT PCR product size 113 bp
HERV H1-3 Rev
gagcggccacaggaatagta (471)
SEQ ID NO: 77
DNA
artificial
HERV K 1-6
qRT PCR product size 142 bp
HERV K 1-6 fwd2
TGGCCAATAGTGCGGTGATA (740)
SEQ ID NO: 78
DNA
artificial
HERV K 1-6
qRT PCR product size 142 bp
HERV K 1-6 Rev2
TCGCTATCAACAGCTGGACT (862)
SEQ ID NO: 79
DNA
artificial
HERV T
qRT PCR product size 94 bp
HERV TFwd
ccaggatttgatgttggg (640)
SEQ ID NO: 80
DNA
artificial
HERV T
qRT PCR product size 94 bp
HERV TRev
ggtgtttctggaatatagggtcac (734)
SEQ ID NO: 81
DNA
artificial
HERV W
qRT PCR product size 187 bp
HERV W Fwd
cccatcgtataggagtctt (146)
SEQ ID NO: 82
DNA
artificial
HERV W
qRT PCR product size 187 bp
HERV W Rev
ccccatcagacataccagtt (333)
SEQ ID NO: 83
DNA
artificial
HERV FRD
qRT PCR product size 94 bp
HERV FRD Fwd
gcctgcaaatagtcttcttt (261)
SEQ ID NO: 84
DNA
artificial
HERV FRD
qRT PCR product size 94 bp
HERV FRD Rev
ataggggctattcccattag (355)
SEQ ID NO: 85
DNA
artificial
HERV R
qRT PCR product size 121 bp
HERV R Fwd
ccatgggaagcaagggaact (793)
SEQ ID NO: 86
DNA
artificial
HERV R
qRT PCR product size 121 bp
HERV R Rev
ctttccccagcgagcaatac (914)
SEQ ID NO: 87
DNA
artificial
HERV R(b)
qRT PCR product size 145 bp
HERV R(b) Fwd
ggacagtgccgacatactat (709)
SEQ ID NO: 88
DNA
artificial
HERV R(b)
qRT PCR product size 145 bp
HERV R(b) Rev
tagagtgcagcatcctaacc (854)
SEQ ID NO: 89
DNA
artificial
HERV F(c)2
qRT PCR product size 174 bp
HERV F(c)2 Fwd
atggaggactatatgagcacaa (590)
SEQ ID NO: 90
DNA
artificial
HERV F(c)2
qRT PCR product size 174 bp
HERV F(c)2 Rev
attaaagttaaccacgagaagc (764)
SEQ ID NO: 91
DNA
artificial
HERV F(c)1
qRT PCR product size 84 bp
HERV F(c)1 Fwd
gctacaccactcctaactcatcct (908)
SEQ ID NO: 92
DNA
artificial
HERV F(c)1
qRT PCR product size 84 bp
HERV F(c)1 Rev
ttgtaagggtgaagttacaccaga (992)
SEQ ID NO: 93
DNA
Mus musculus mutated
MuERV Env polyprotein P10404, variable regions VRA, VRC and VRB in the surface domain
SU that determine receptor usage by different X/P-MuLV subtypes MCF247. Env protein of a
typical polytropic MuLV, MCF 247, variable regions VRA, VRC and VRB in the surface
domain SU that determine receptor usage by different X/P-MuLV subtypes
GDDWDETGLG CRTPGGRKRA RTFDFHTVPT GCRGNTPQNQ GPCYDSSAVS
SDIKGATPGGR (61 aac)
SEQ ID NO: 94
DNA
Mus musculus mutated
P10404 (MuERV Env)
Xaa = R, MuERV Env polyprotein P10404, variable regions VRA, VRC and VRB in the surface
domain SU that determine receptor usage by different X/P-MuLV subtypes, Xaa can be any
naturally occurring amino acid
GDDWDETGLG CRTPGGRKRA RTFDFHTVPT GCRXNTPQNQ GPCYDSSAVS
SDIKGATPGGR (61 aac)
SEQ ID NO: 95
DNA
artificial
primer: Syncytin-1 fwd
ccgctcgaga gcggtcgtcg gccaac (26 bp)
SEQ ID NO: 96
DNA
artificial
primer: Syncytin-1 rev
gaagatctcc ttcccagcta ggcttaggg (29 bp)
SEQ ID NO: 97
DNA
artificial
pSIH-shRNA-Syn
ggccctccct tatcatatt (19 bp)
SEQ ID NO: 98
DNA
artificial
loop sequence to silence Syncytin-1
cttcctgtca ga (12 bp)
SEQ ID NO: 99
DNA
Homo sapiens
SLC1A4
ATGGAGAAGAGCAACGAGACCAACGGCTACCTTGACAGCGCTCAGGCGGGGCCTGCG
GCCGGGCCCGGAGCTCCGGGGAC
CGCGGCGGGACGCGCACGGCGTTGCGCGGGCTTCCTGCGGCGCCAAGCGCTGGTGC
TGCTCACCGTGTCCGGGGTGCTGG
CGGGCGCGGGCCTGGGCGCGGCGTTGCGCGGGCTCAGCCTGAGCCGCACGCAGGTC
ACCTACCTGGCCTTCCCCGGCGAG
ATGCTGCTCCGCATGCTGCGCATGATCATCCTGCCGCTGGTGGTCTGCAGCCTGGTGT
CGGGCGCCGCCTCGCTCGATGC
CAGCTGCCTCGGGCGTCTGGGCGGCATCGCTGTCGCCTACTTTGGCCTCACCACACTG
AGTGCCTCGGCGCTCGCCGTGG
CCTTGGCGTTCATCATCAAGCCAGGATCCGGTGCGCAGACCCTTCAGTCCAGCGACCT
GGGGCTGGAGGACTCGGGGCCT
CCTCCTGTCCCCAAAGAGACGGTGGACTCTTTCCTCGACCTGGCCAGAAACCTGTTTCC
CTCCAATCTTGTGGTTGCAGC
TTTCCGTACGTATGCAACCGATTATAAAGTCGTGACCCAGAACAGCAGCTCTGGAAATG
TAACCCATGAAAAGATCCCCA
TAGGCACTGAGATAGAAGGGATGAACATTTTAGGATTGGTCCTGTTTGCTCTGGTGTTA
GGAGTGGCCTTAAAGAAACTA
GGCTCCGAAGGAGAAGACCTCATCCGTTTCTTCAATTCCCTCAACGAGGCGACGATGGT
GCTGGTGTCCTGGATTATGTG
GTACGTACCTGTGGGCATCATGTTCCTTGTTGGAAGCAAGATCGTGGAAATGAAAGACA
TCATCGTGCTGGTGACCAGCC
TGGGGAAATACATCTTCGCATCTATATTGGGCCATGTTATTCATGGAGGAATTGTTCTGC
CACTTATTTATTTTGTTTTC
ACACGAAAAAACCCATTCAGATTCCTCCTGGGCCTCCTCGCCCCATTTGCGACAGCATT
TGCTACCTGCTCCAGCTCAGC
GACCCTTCCCTCTATGATGAAGTGCATTGAAGAGAACAATGGTGTGGACAAGAGGATCA
GCAGGTTTATTCTCCCCATCG
GGGCCACCGTGAACATGGACGGAGCAGCCATCTTCCAGTGTGTGGCCGCGGTGTTCAT
TGCGCAACTCAACAACGTAGAG
CTCAACGCAGGACAGATTTTCACCATTCTAGTGACTGCCACAGCGTCCAGTGTTGGAGC
AGCAGGCGTGCCAGCTGGAGG
GGTCCTCACCATTGCCATTATCCTGGAGGCCATTGGGCTGCCTACTCATGACCTGCCTC
TGATCCTGGCTGTGGACTGGA
TTGTGGACCGGACCACCACGGTGGTGAATGTGGAAGGGGATGCCCTGGGTGCAGGCA
TTCTCCACCACCTGAATCAGAAG
GCAACAAAGAAAGGCGAGCAGGAACTTGCTGAGGTGAAAGTGGAAGCCATCCCCAACT
GCAAGTCTGAGGAGGAGACATC
GCCCCTGGTGACACACCAGAACCCCGCTGGCCCCGTGGCCAGTGCCCCAGAACTGGA
ATCCAAGGAGTCGGTTCTGTGA (1599 bp)
SEQ ID NO: 100
DNA
Homo sapiens
SLC1A5
ATGGTGGCCGATCCTCCTCGAGACTCCAAGGGGCTCGCAGCGGCGGAGCCCACCGCC
AACGGGGGCCTGGCGCTGGCCTC
CATCGAGGACCAAGGCGCGGCAGCAGGCGGCTACTGCGGTTCCCGGGACCAGGTGCG
CCGCTGCCTTCGAGCCAACCTGC
TTGTGCTGCTGACAGTGGTGGCCGTGGTGGCCGGCGTGGCGCTGGGACTGGGGGTGT
CGGGGGCCGGGGGTGCGCTGGCG
TTGGGCCCGGAGCGCTTGAGCGCCTTCGTCTTCCCGGGCGAGCTGCTGCTGCGTCTG
CTGCGGATGATCATCTTGCCGCT
GGTGGTGTGCAGCTTGATCGGCGGCGCCGCCAGCCTGGACCCCGGCGCGCTCGGCC
GTCTGGGCGCCTGGGCGCTGCTCT
TTTTCCTGGTCACCACGCTGCTGGCGTCGGCGCTCGGAGTGGGCTTGGCGCTGGCTCT
GCAGCCGGGCGCCGCCTCCGCC
GCCATCAACGCCTCCGTGGGAGCCGCGGGCAGTGCCGAAAATGCCCCCAGCAAGGAG
GTGCTCGATTCGTTCCTGGATCT
TGCGAGAAATATCTTCCCTTCCAACCTGGTGTCAGCAGCCTTTCGCTCATACTCTACCAC
CTATGAAGAGAGGAATATCA
CCGGAACCAGGGTGAAGGTGCCCGTGGGGCAGGAGGTGGAGGGGATGAACATCCTGG
GCTTGGTAGTGTTTGCCATCGTC
TTTGGTGTGGCGCTGCGGAAGCTGGGGCCTGAAGGGGAGCTGCTTATCCGCTTCTTCA
ACTCCTTCAATGAGGCCACCAT
GGTTCTGGTCTCCTGGATCATGTGGTACGCCCCTGTGGGCATCATGTTCCTGGTGGCT
GGCAAGATCGTGGAGATGGAGG
ATGTGGGTTTACTCTTTGCCCGCCTTGGCAAGTACATTCTGTGCTGCCTGCTGGGTCAC
GCCATCCATGGGCTCCTGGTA
CTGCCCCTCATCTACTTCCTCTTCACCCGCAAAAACCCCTACCGCTTCCTGTGGGGCAT
CGTGACGCCGCTGGCCACTGC
CTTTGGGACCTCTTCCAGTTCCGCCACGCTGCCGCTGATGATGAAGTGCGTGGAGGAG
AATAATGGCGTGGCCAAGCACA
TCAGCCGTTTCATCCTGCCCATCGGCGCCACCGTCAACATGGACGGTGCCGCGCTCTT
CCAGTGCGTGGCCGCAGTGTTC
ATTGCACAGCTCAGCCAGCAGTCCTTGGACTTCGTAAAGATCATCACCATCCTGGTCAC
GGCCACAGCGTCCAGCGTGGG
GGCAGCGGGCATCCCTGCTGGAGGTGTCCTCACTCTGGCCATCATCCTCGAAGCAGTC
AACCTCCCGGTCGACCATATCT
CCTTGATCCTGGCTGTGGACTGGCTAGTCGACCGGTCCTGTACCGTCCTCAATGTAGAA
GGTGACGCTCTGGGGGCAGGA
CTCCTCCAAAATTACGTGGACCGTACGGAGTCGAGAAGCACAGAGCCTGAGTTGATACA
AGTGAAGAGTGAGCTGCCCCT
GGATCCGCTGCCACTCCCCACTGAGGAAGGAAACCCCCTCCTCAAACACTATCGGGGG
CCCGCAGGGGATGCCACGGTCG
CCTCTGAGAAGGAATCAGTCATGTAG (1626 bp)
SEQ ID NO: 101
DNA
Homo sapiens
HERV-K env
>orico HERV K ENV codon optimized
MNPSEMQRKAPPRRRRHRNRAPLTHKMNKMVTSEEQMKLPSTKKAEPPTWAQLKKLTQL
ATKYLENTKVTQTPESMLLAA
LMIVSMVVSLPMPAGAAAANYTYWAYVPFPPLIRAVTWMDNPIEVYVNDSVWVPGPTDDRC
PAKPEEEGMMINISIGYRY
PPICLGRAPGCLMPAVQNWLVEVPTVSPISRFTYHMVSGMSLRPRVNYLQDFSYQRSLKFR
PKGKPCPKEIPKESKNTEV
LVWEECVANSAVILQNNEFGTIIDWAPRGQFYHNCSGQTQSCPSAQVSPAVDSDLTESLDK
HKHKKLQSFYPWEWGEKGI
STPRPKIISPVSGPEHPELWRLTVASHHIRIWSGNQTLETRDRKPFYTIDLNSSLTVPLQSCV
KPPYMLVVGNIVIKPDS
QTITCENCRLLTCIDSTFNWQHRILLVRAREGVWIPVSMDRPWEASPSVHILTEVLKGVLNR
SKRFIFTLIAVIMGLIAV
TATAAVAGVALHSSVQSVNFVNDWQKNSTRLWNSQSSIDQKLANQINDLRQTVIWMGDRL
MSLEHRFQLQCDWNTSDFCI
TPQIYNESEHHWDMVRRHLQGREDNLTLDISKLKEQIFEASKAHLNLVPGTEAIAGVADGLA
NLNPVTWVKTIGSTTIIN
LILILVCLFCLLLVCRCTQQLRRDSDHRERAMMTMVVLSKRKGGNVGKSKRDQIVTVSV
SEQ ID NO: 102
DNA
Homo sapiens
HERV-K env
K10
>AAD51800.1
MNPSEMQRKAPPRRRRHRNRAPLTHKMNKMVTSEEQMKLPSTKKAEPPTWAQLKKLTQL
ATKYLENTKVTQTPESMLLAA
LMIVSMVVSLPMPAGAAAANYTNWAYVPFPPLIRAVTWMDNPIEVYVNDSVWVPGPIDDRC
PAKPEEEGMMINISIGYRY
PICLGRAPGCLMPAVQNWLVEVPIVSPICRFTYHMVSGMSLRPRVNYLQDFSYQRSLKFRP
KGKPCPKEIPKESKNTEVL
VWEECVANSAVILQNNEFGTIIDWTPQGQFYHNCSGQTQSCPSAQVSPAVDSDLTESLDKH
KHKKLQSFYPWEWGEKGIS
TPRPKIISPVSGPEHPELWRLTVASHHIRIWSGNQTLETRDRKPFYTVDLNSSLTLPLQSCVK
PPYMLVVGNIVIKPDSQ
TITCENCRLLTCIDSTFNWQHRILLVRAREGVWIPVSMDRPWEASPSIHILTEVLKGVLNRSK
RFIFTLIAVIMGLIAVT
ATAAVAGVALHSSVQSVNFVNDGQKNSTRLWNSQSSIDQKLANQINDLRQTVIWMGDRLM
SLEHRFQLQCDWNTSDFCIT
PQIYNESEHHWDMVRRHLQGREDNLTLDISKLKEQIFEASKAHLNLVPGTEAIAGVADGLAN
LNPVTWVKTIGSTTIINL
ILILVCLFCLLLVCRCTQQLRRDSDHRERAMMTMAVLSKRKGGNVGKSKRDQIVTVSV
SEQ ID NO: 103
DNA
Homo sapiens
HERV-K env-2
>AAZ91341.1
MVTPVTWMDNPIEVYVNDSVRVPGPTDDRCPIKPEEEGIMINISTGYRYPICLGRAPGCLIHA
VQNWLVEVPTVSPNGRF
TYHMVSGMSLRPRVNYLQDFSYQRSLKFRPKGKPCPKEIPKESKNTEVLVWEECVANSAVI
LQNNEFGTIIDWAPRGQFD
HNCSGQTQLCPSAQVSPAVDSDLTESLDKHKHKKLQSLYPWEWGEKGISTPRPKIISPVSG
PEHPELWRLIVASHHIRIW
SGNQTSETRDRKPFYTIDLNSSLTVPLQSCVKPPYMLVVGNIVIKPDSQTITCENCRLFTCIDS
TFNWQQRILLVRAREG
VWIPVSMDRPWEASPSIHILTEVLKGILNRSKRFIFTLIAVIMGLIAVTATAAVAGVALHSSVQS
VNFVNDWQKNSARLW
NSQSSIDQKLANQINHLRQTHLDRRQTHELRTSFPVTV
SEQ ID NO: 104
DNA
Homo sapiens
HERV-K env
>AAY87455.1
ynepirdakkrastemvtpvtwmdnpievyvndsewvpgptddrcpakpeeegtmlnisigyryppiclgtapgclmpav
qnwlvevpivspisrftyhmvsgmslrprvnylqdfpyqrslkfrpkgkpcpkeipkesknteilvweecvansavilqn
nefgtiidwaprgqfyhncsgqtqscpsaqvspavdsdltesldkhkhkklqsfypwewgekgistprpkiispvsgpeh
pelwrltvashhinwsgnqtletrdrkpfytvdlnssltlplqscvkppymlvvgnivikpdsqtitcencrlltcids
tfnwqhrillvraregvwilvsmdrpweaspsvhiltevlkgvlnrskrfiftliavimgliavtatgavagvalhssvq
svnfvndwqknstrlwnsqssidqklanqindlrqtviwmgdrlmslehrfqlqcdwntsdfcitpqiynesehhwdmvr
hhlqgrednltldisklkeqifeaskahlnlvpgteaiagvadglanlnpvtwvktigsttiinlililvclfclllvcr
ctqqlrrdsdhrerammtmtvlskrkggnvgkskrdqivtvsv
SEQ ID NO: 105
DNA
Homo sapiens
HERV-K env
K108
>Q69384
MNPSEMQRKAPPRRRRHRNRAPLTHKMNKMVTSEEQMKLPSTKKAEPPTWAQLKKLTQL
ATKYLENTKVTQTPESMLLAA
LMIVSMVVSLPMPAGAAAANYTYWAYVPFPPLIRAVTWMDNPTEVYVNDSVWVPGPIDDRC
PAKPEEEGMMINISIGYHY
PPICLGRAPGCLMPAVQNWLVEVPTVSPICRFTYHMVSGMSLRPRVNYLQDFSYQRSLKFR
PKGKPCPKEIPKESKNTEV
LVWEECVANSAVILQNNEFGTIIDWAPRGQFYHNCSGQTQSCPSAQVSPAVDSDLTESLDK
HKHKKLQSFYPWEWGEKGI
STPRPKIVSPVSGPEHPELWRLTVASHHIRIWSGNQTLETRDRKPFYTIDLNSSLTVPLQSCV
KPPYMLVVGNIVIKPDS
QTITCENCRLLTCIDSTFNWQHRILLVRAREGVWIPVSMDRPWEASPSVHILTEVLKGVLNR
SKRFIFTLIAVIMGLIAV
TATAAVAGVALHSSVQSVNFVNDWQKNSTRLWNSQSSIDQKLANQINDLRQTVIWMGDRL
MSLEHRFQLQCDWNTSDFCI
TPQIYNESEHHWDMVRRHLQGREDNLTLDISKLKEQIFEASKAHLNLVPGTEAIAGVADGLA
NLNPVTWVKTIGSTTIIN
LILILVCLFCLLLVCRCTQQLRRDSDHRERAMMTMAVLSKRKGGNVGKSKRDQIVTVSV
SEQ ID NO: 106
DNA
Homo sapiens
HERV-K env
k113
>AAD21098.1
MNPSEMQRKAPPRRRRHRNRAPLTHKMNKMVTSEEQMKLPSTKKAEPPTWAQLKKLTQL
ATKYLENTKVTQTPESMLLAA
LMIVSMVVSLPMPAGAAAANYTYWAYVPFPPLIRAVTWMDNPTEVYVNDSVWVPGPIDDRC
PAKPEEEGMMINISIGYHY
PPICLGRAPGCLMPAVQNWLVEVPTVSPICRFTYHMVSGMSLRPRVNYLQDFSYQRSLKFR
PKGKPCPKEIPKESKNTEV
LVWEECVANSAVILQNNEFGTIIDWAPRGQFYHNCSGQTQSCPSAQVSPAVDSDLTESLDK
HKHKKLQSFYPWEWGEKGI
STPRPKIVSPVSGPEHPELWRLTVASHHIRIWSGNQTLETRDRKPFYTIDLNSSLTVPLQSCV
KPPYMLVVGNIVIKPDS
QTITCENCRLLTCIDSTFNWQHRILLVRAREGVWIPVSMDRPWEASPSVHILTEVLKGVLNR
SKRFIFTLIAVIMGLIAV
TATAAVAGVALHSSVQSVNFVNDWQKNSTRLWNSQSSIDQKLANQINDLRQTVIWMGDRL
MSLEHRFQLQCDWNTSDFCI
TPQIYNESEHHWDMVRRHLQGREDNLTLDISKLKEQIFEASKAHLNLVPGTEAIAGVADGLA
NLNPVTWVKTIGSTTIIN
LILILVCLFCLLLVCRCTQQLRRDSDHRERAMMTMAVLSKRKGGNVGKSKRDQIVTVSV
SEQ ID NO: 107
DNA
Homo sapiens
HERV-K env
ERVK6
>AAF88168.1
MNPSEMQRKAPPRRRRHRNRAPLTHKMNKMVTSEEQMKLPSTKKAEPPTWAQLKKLTQL
ATKYLENTKVTQTPESMLLAA
LMIVSMVVSLPMPAGAAAANYTYWAYVPFPPLIRAVTWMDNPTEVYVNDSVWVPGPIDDRC
PAKPEEEGMMINISIGYHY
PPICLGRAPGCLMPAVQNWLVEVPTVSPICRFTYHMVSGMSLRPRVNYLQDFSYQRSLKFR
PKGKPCPKEIPKESKNTEV
LVWEECVANSAVILQNNEFGTIIDWAPRGQFYHNCSGQTQSCPSAQVSPAVDSDLTESLDK
HKHKKLQSFYPWEWGEKGI
STPRPKIVSPVSGPEHPELWRLTVASHHIRIWSGNQTLETRDRKPFYTIDLNSSLTVPLQSCV
KPPYMLVVGNIVIKPDS
QTITCENCRLLTCIDSTFNWQHRILLVRAREGVWIPVSMDRPWEASPSVHILTEVLKGVLNR
SKRFIFTLIAVIMGLIAV
TATAAVAGVALHSSVQSVNFVNDWQKNSTRLWNSQSSIDQKLANQINDLRQTVIWMGDRL
MSLEHRFQLQCDWNTSDFCI
TPQIYNESEHHWDMVRRHLQGREDNLTLDISKLKEQIFEASKAHLNLVPGTEAIAGVADGLA
NLNPVTWVKTIGSTTIIN
LILILVCLFCLLLVCRCTQQLRRDSDHRERAMMTMAVLSKRKGGNVGKSKRDQIVTVSV
SEQ ID NO: 108
DNA
Homo sapiens
HERV-K env
k113
>AAD51798.1
MNPSEMQRKAPPRRRRHRNRAPLTHKMNKMVTSEEQMKLPSTKKAEPPTWAQLKKLTQL
ATKYLENTKVTQTPESMLLAA
LMIVSMVVSLPMPAGAAAANYTYWAYVPFPPLIRAVTWMDNPTEVYVNDSVWVPGPIDDRC
PAKPEEEGMMINISIGYHY
PPICLGRAPGCLMPAVQNWLVEVPTVSPICRFTYHMVSGMSLRPRVNYLQDFSYQRSLKFR
PKGKPCPKEIPKESKNTEV
LVWEECVANSAVILQNNEFGTIIDWAPRGQFYHNCSGQTQSCPSAQVSPAVDSDLTESLDK
HKHKKLQSFYPWEWGEKGI
STPRPKIVSPVSGPEHPELWRLTVASHHIRIWSGNQTLETRDRKPFYTIDLNSSLTVPLQSCV
KPPYMLVVGNIVIKPDS
QTITCENCRLLTCIDSTFNWQHRILLVRAREGVWIPVSMDRPWEASPSVHILTEVLKGVLNR
SKRFIFTLIAVIMGLIAV
TATAAVAGVALHSSVQSVNFVNDWQKNSTRLWNSQSSIDQKLANQINDLRQTVIWMGDRL
MSLEHRFQLQCDWNTSDFCI
TPQIYNESEHHWDMVRRHLQGREDNLTLDISKLKEQIFEASKAHLNLVPGTEAIAGVADGLA
NLNPVTWVKTIGSTTIIN
LILILVCLFCLLLVCRCTQQLRRDSDHRERAMMTMAVLSKRKGGNVGKSKRDQIVTVSV
SEQ ID NO: 109
DNA
Homo sapiens
HERV-K env
>BAB11758.1
MDNPIEVYVNDSVWVPGPTDDRCPAKPEEERMMINISIGYRYPPICLGRAPGCLMPAVQNW
LVEVPTVSPISRFTYHMVS
GMSLRPQVNYLQDFSYQRSLKFRPKRKPCLKEIPKESKNTEVLV
SEQ ID NO: 110
DNA
Homo sapiens
HERV-K env
>AAA88033.1
mgplqpglpspamipkdwpliiidlkdcfftiplaeqdcekfaftipainnkepatrfqwkvlpqgmlnspticqtfvgr
alqpvrekfsdcyiihyiddilcaaetkdklidcytflqaevanaglaiasdkiqtstpfhylgmqienrkikpqkieir
kdtlktlndfqkllgdinwirptlgiptyamsnlfsilrgdsdlnsqriltpeatkeiklveekiqsaqinridplaplq
llifatahsptgiiiqntdlvewsflphstvktftlyldqiatligqtrlritklcgndpdkivvpltkeqvrqafinsg
awqiglanfvglidnhypktkifqflklttwilpkitrreplenaltvftdgssngkaaytgpkerviktpyqsaqrdel
vavitvlqdfdqpiniisdsayvvqatrdvetalikysmddqlnqlfnllqqtvrkrnfpfyityirahtnlpgpltkan
eqadllvssalikaqelhalthvnaaglknkfdvtwkqakdivqhctqcqvlhlptqeagvnprglcpnalwqmdvthvp
sfgrlsyvhvtvdtyshfiwatcqtgestshvkkhllscfavmgvpekiktdngpgycskafqkflsqwkishttgipyn
sqgqaivertnrtlktqlvkqkeggdskecttpqmqlnlalytlnflniyrnqtttsaeqhltgkknsphegkliwwkdn
knktweigkvitwgrgfacvspgenqlpvwlptrhlkfynepigdakkrastemvtpvtwmdnpievyvndsiwypgpid
drcpakpeeegmminisigyryppiclgrapgclmpavqnwlvevptvspisrftyhmvsgmslrprvnylqdfsyqrsl
kfrpkgkpcpkeipkeskntevlvweecvansavilnnefgtiidwaprgqfyhncsgqtqscpsaqvspavdsdltesl
dkhkhkklqsfypwewgekgistprpkivspvsgpehpelwrltvashhiriwsgnqtletrdckpfytvdlnssltvpl
qscvkppymlvvgnivikpdsqtitcencrlltcidstfnwqhrillvraregvwipvsmdrpweaspsvhiltevlkgv
lnrskrfiftliavimgliavtataavagvalhssvqsvnfvndwqknstrlwnsqssidqklanqindlrqtviwmgdr
lmslehrfqlqcdwntsdfcitpqiynesehhwdmvrrhlqgrednltldisklkeqifeaskahlnlvpgteaiagvad
glanlnpvtwvktigstsiinlililvclfclllvcrctqqlrrdsdhrerammtmavlskrkggnvgkskrdqivtvsv
SEQ ID NO: 111
DNA
Homo sapiens
HERV-K env
>BAB11760.1
MVTPVTWMDNPIEVYVNDSVWVPGPTDDRCPAKPEEEGMMINISIVYRYPPICLGRAPGCL
MPAVQNWLVEVPTVSPNSR
FTYHMVSGMSLRPRVNYLQDFSYQRSLKFRPKGKPCPKEIPKESKNTEVLVWEECVANSAV
ILQNNEFGTIIDWAPRGQF
YHNCSGQTQSCPSAQVSPAVDSDLTESLDKHKHKKLQSFYPWEWGEKGISTPRPEIISPVS
GPEHPELWRLWPDTTLEFG
LEIKL
SEQ ID NO: 112
DNA
Homo sapiens
HERV-K env
>BAB11759.1
MGQTKSKTKSKYASYLSFIKILLKRGGVRVSTKNLIKLFQIIEQFCPWFPEQGTLDLKDWKRIG
EELKQAGRKGNIIPLT
VWNDWAIIKAALEPFQTKEDSVSVSDAPGSCVIDCNEKTGRKSQKETESLHCEYVTEPVMA
QSTQNVDYNQLQGVIYPET
LKLEGKGPELVGPSESKPRGPSPLPAGQVPVTLQPQTQVKENKTQPPVAYQYWPPAELQY
LPPPESQYGYPGMPPALQGR
APYPQPPTVRLNPTASRSGQGGTLHAVIDEARKQGDLEAWRFLVILQLVQAGEETQVGAPA
RAETRCEPFTMKMLKDIKE
GVKQYGSNSPYIRTLLDSIAHGNRLTPYDWESLAKSSLSSSQYLQFKTWWIDGVQEQVRKN
QATKPTVNIDADQLLGTGP
NWSTINQQSVMQNEAIEQVRAICLRAWGKIQDPGTAFPINSIRQGSKEPYPDFVARLQDAAQ
KSITDDNARKVIVELMAY
ENANPECQSAIKPLKGKVPAGVDVITEYVKACDGIGGAMHKAMLMAQAMRGLTLGGQVRTF
GKKCYNCGQIGHLKRSCPV
LNKQNIINQAITAKNKKPSGLCPKCGKGKHWANQCHSKFDKDGQPLSGNRKRGQPQAPQQ
TGAFPVQLFVPQGFQGQQPL
QKIPPLQGVSQLQQSNSCPAPQQAAPQ
SEQ ID NO: 113
DNA
Homo sapiens
HERV-K env
>HERV-K113
mnpsemqrkapprrrrhrnraplthkmnkmvtseeqmklpstkkaepptwaqlkkltqlatkylentkvtqtpesmllaa
lmivsmvvslpmpagaaaanytywayvpfppliravtwmdnpieiyvndsvwvpgptddccpakpeeegmminisigyry
ppiclgrapgclmpavqnwlvevptvspisrftyhmvsgmslrprvnylqdfsyqrslkfrpkgkpcpkeipkeskntev
lvweecvansavilqnnefgtlidwaprgqfyhncsgqtqscpsaqvspavdsdltesldkhkhkklqsfypwewgekgi
starpkiispvsgpehpelwrltvashhiriwsgnqtletrdrkpfytidlnssltvplqscvkppymlvvgnivikpds
qtitcencrlltcidstfnwqhrillvraregvwipvsmdrpweaspsvhiltevlkgvlnrskrfiftliavimgliav
tataavagvalhssvqsvnfvndwqnnstrlwnsqssidqklanqindlrqtviwmgdrlmslehrfqlqcdwntsdfci
tpqiynesehhwdmvrchlqgrednltldisklkeqifeaskahlnlvpgteaiagvadglanlntvtwvktigsttiin
lililvclfclllvyrctqqlrrdsdhrerammtmvvlskrkggnvgkskrdqivtvsv
SEQ ID NO: 114
DNA
Homo sapiens
HERV-K env
>DQ112146.1
MVTPVTWMDNPIEIYVNDSVWVPGPIDDRCPAKPEEEGMMINISIGYRYPPICLGRAPGCLM
PAVQNWLVEVPTVSPISRFTYHMVSGMSLRPRVNYLQDFSYQRSLKFRPKGKPCPKEIPKE
SKNTEVLVWEECVANSAVILQNNEFGTIIDWAPRGQFYHNCSGQTQSCPSAQVSPAVDSDL
TESLDKHKHKKLQSFYPWEWGEKGISTPRPKIVSPVSGPEHPELWRLTVASHHIRIWSGNQ
TLETRDCKPFYTIDLNSSLTVPLQSCVKPPYMLVVGNIVIKPDSQTITCENCRLLTCIDSTFNW
QHRILLVRAREGVWIPVSMDRPWEASPSVHILTEVLKGVLNRSKRFIFTLIAVIMGLIAVTATA
AVAGVALHSSVQSVNFVNDWQKNSTRLWNSQSSIDQKLANQINDLRQTVIWMGDRLMSLE
HRFQLQCDWNTSDFCITPQIYNESEHHWDMVRRHLQGREDNLTLDISKLKEQIFEASKAHL
NLVPGTEAIAGVADGLANLNPVTWVKTIGSTTIINLILILVCLFCLLLVCRCTQQLRRDSDHRE
RAMMTMAVLSKRKGGNVGKSKR
SEQ ID NO: 115
DNA
Homo sapiens
HERV-K env
>CAA63481.1
MNPSEMQRKAPPRRRRHRNRAPLTHKMNKMVTSEEQMKLPSTKK
AEPPTWAQLKKLTQLATKYLENTKVTQTPESMLLAALMIVSMVVSLPMPAGAAAANYT
YWAYVPFPPLIRAVTWMDNPIEVYVNDSVWVPGPTDDHCPAKPEEEGMMINISIGYRY
PPICLGRAPGCLMPAVQNWLVEVPTVSPISRFTYHMVSGMSLRPRVNYLQDFSYQRSF
KFRPKGKPCPKEIPKESKNTEVLVWEECVANSAVILQNNEFGTIIDWAPRGQFYHNCS
GQTQSCPSAQVSPAVDSDLTESLDKHKHKKLQSFYPWEWGEKGISTPRPKIISPVSGP
EHPELWRLTVASHHIRIWSGNQTLETRDRKPFYTVDLNSSVTVPLQSCIKPPYMLVVG
NIVIKPDSQTITCENCRLLTCIDSTFNWQHRILLVRAREGVWIPVSMDRPWETSPSIH
TLTEVLKGVLNRSKRFIFTLIAVIMGLIAVTATAAVAGVALHSSVQSVNFVNDWQKNS
TRLWNS
SEQ ID NO: 116
DNA
Homo sapiens
HERV-K env
>Q902F9
MNPSEMQRKAPPRRRRHRNRAPLTHKMNKMVTSEEQMKLPSTKKAEPPTWAQLKKLTQL
ATKYLENTKVTQTPESMLLAA
LMIVSMVVSLPMPAGAAAANYTYWAYVPFPPLIRAVTWMDNPIEIYVNDSVWVPGPTDDCC
PAKPEEEGMMINISIGYRY
PPICLGRAPGCLMPAVQNWLVEVPTVSPISRFTYHMVSGMSLRPRVNYLQDFSYQRSLKFR
PKGKPCPKEIPKESKNTEV
LVWEECVANSAVILQNNEFGTLIDWAPRGQFYHNCSGQTQSCPSAQVSPAVDSDLTESLDK
HKHKKLQSFYPWEWGEKGI
STARPKIISPVSGPEHPELWRLTVASHHIRIWSGNQTLETRDRKPFYTIDLNSSLTVPLQSCV
KPPYMLVVGNIVIKPDS
QTITCENCRLLTCIDSTFNWQHRILLVRAREGVWIPVSMDRPWEASPSVHILTEVLKGVLNR
SKRFIFTLIAVIMGLIAV
TATAAVAGVALHSSVQSVNFVNDWQNNSTRLWNSQSSIDQKLANQINDLRQTVIWMGDRL
MSLEHRFQLQCDWNTSDFCI
TPQIYNESEHHWDMVRCHLQGREDNLTLDISKLKEQIFEASKAHLNLVPGTEAIAGVADGLA
NLNTVTWVKTIGSTTIIN
LILILVCLFCLLLVYRCTQQLRRDSDHRERAMMTMVVLSKRKGGNVGKSKRDQIVTVSV
SEQ ID NO: 117
DNA
Homo sapiens
HERV-K env
>Q902F8
MNPSEMQRKAPPRRRRHRNRAPLTHKMNKMVTSEEQMKLPSTKKAEPPTWAQLKKLTQL
ATKYLENTKVTQTPESMLLAA
LMIVSMVVSLPMPAGAAVANYTNWAYVPFPPLIRAVTWMDNPIEVYVNDSVWVPGPIDDRC
PAKPEEEGMMINISIGYRY
PPICLGRAPGCLMPAVQNWLVEVPTVSPISRFTYHMVSGMSLRPRVNYLQDFSYQRSLKFR
PKGKPCPKEIPKESKNTEV
LVWEECVANSAVILQNNEFGTIIDWAPRGQFYHNCSGQTQSCPSAQVSPAVDSDLTESLDK
HKHKKLQSFYPWEWGEKRI
STPRPKIVSPVSGPEHPELWRLTVASHHIRIWSGNQTLETRDRKPFYTVDLNSSLTLPLQSC
VKPPYMLVVGNIVIKPDS
QTITCENCRLLTCIDSTFNWQHRILLVRAREGVWIPVSMDRPWEASPSVHILTEVLKGVLNR
SKRFIFTLIAVIMGLIAV
TATAAVAGVALHSSVQSVNFVNDGQKNSTRLWNSQSSIDQKLANQINDLRQTVIWMGDRL
MSLEHRFQLQCDWNTSDFCI
TPQIYNDSEHHWDMVRRHLQGREDNLTLDISKLKEQIFEASKAHLNLVPGTEAIAGVADGLA
NLNPVTWVKTIGSTTIIN
LILILVCLFCLLLVCRCTQQLRRDSDHRERAMMTMAVLSKRKGGNVGKSKRDQIVTVSV
SEQ ID NO: 118
DNA
Homo sapiens
HERV-K env
>Q9QC07
NKSRKRRNRVSFLGVTTVEPPKPIPLTWKTEKLVWVNQWPLPKQKLEALHLLANEQLEKGHI
EPSFSPWNSPVFVIQKKS
SKWRMLTDLRAVNAVIQPMGPLQPGLPSPAMIPKDWPLIIIDLKDCFFTIPLAEQDCEKFAFTI
PAINNKEPATRFQWKV
LPQGMLNSPTICQTFVGRALQPVRDKFSDCYIIHYFDDILCAAETKDKLIDCYTFLQAEVANA
GLAIASDKIQTSTPFHY
LGMQIENRKIKPQKIEIRKDTLKTLNDFQKLLGDINWIRPTLGIPTYAMSNLFSILRGDSDLNSK
RMLTPEATKEIKLVE
EKIQSAQINRIDPLAPLQLLIFATAHSPTGIIIQNTDLVEWSFLPHSTVKTFTLYLDQIATLIGPTR
LRIIKLCGNDPDK
IVVPLTKEQVRQAFINSGAWQIGLANFVGIIDNHYPKTKIFQFLKLTTWILPKITRREPLENALT
VFTDGSSNGKVAYTG
PKERVIKTPYQSAQRAELVAVITVLQDFDQPINIISDSAYVVQATRDVETALIKYSMDDQLNQL
FNLLQQTVRKRNFPFY
ITHIRAHTNLPGPLTKANEQADLLVSSAFIKAQELHALTHVNAAGLKNKFDVTWKQAKDIVQH
CTQCQVLDLPTQEAGVN
PEVCVLMHYGKWMSHMYLHLGRLSYVHVTVDTYSHFMCATCQTGESTSHVKKHLLSCFAV
MGVPEKIKTDNGPGYCSKAF
QKFLSQWKISHTTGIPYNSQGQAIVERTNRTLKTQLVKQKEGGDSKECTTPQMQLNLALYTL
NFLNIYRNQTTTSAEHLT
GKKNSPHEGKLI
SEQ ID NO: 119
DNA
Homo sapiens
HERV-K env
>Q9NX77
MWTVPSFTNDSYQVYNVFSTNSFQLLTVKRTPHEAWRVPLTTKTNKTKGLPDCPKKPTNG
PFIVTSILWDNCNAPKAVVL
QTLAMGIVIDWAPKGHYWQDCSSKNTLCSEFIYSLDYIEHGWQSYTMRQRVSPYPFKWMD
TGIAPPRPKIIHPFFTPEHP
ELWKLAAALSGIKIWNTTYQLLRTKTKTPTFNITLISEWVIPIRSCVKPPYMLLVGNIIMMPDAQ
TIECHNCKLFTCIDA
TFNPTTSILLVRAREGVWIPVSLHRPWESSPSIHIVNEVLKDILKRTKRFIFTLIAVLAGLLAVTA
TAATAGVAIRSSVQ
TAHYVEACQKNSSRLWNSQAQIDQKLANQINDLRQSVTWLGDRVMNLQHRMQLQCDWNT
SDYCITPYAYNQDQHSWENVS
RHLKAWDDNLTLDISQLKEQIFEASQAHLSTVPGSHIFECITKQLPDFNPFKWLKPVRGSLLL
LALLILVCLCCLLLVCR
CL
SEQ ID NO: 120
DNA
Homo sapiens
HERV-K env
>Q9HDB8
MVTPVTWMDNPIEVYVNDSVWVPGPTDDRCPAKPEEEGMMINISIVYRYPPICLGRAPGCL
MPAVQNWLVEVPTVSPNSR
FTYHMVSGMSLRPRVNYLQDFSYQRSLKFRPKGKPCPKEIPKESKNTEVLVWEECVANSAV
ILQNNEFGTIIDWAPRGQF
YHNCSGQTQSCPSAQVSPAVDSDLTESLDKHKHKKLQSFYPWEWGEKGISTPRPEIISPVS
GPEHPELWRLWPDTTLEFG
LEIKL
SEQ ID NO: 121
DNA
Homo sapiens
HERV-K env
>P63123
WASQVSENRPVCKAVIQGKQLEGLVDTGADVSIIALNQWPKNWPKQKTVTGLVGIVTASEV
YQSTEILHCLGPHNQESTV
QPMITSIPLNLWGRDLLQQWGAEITMTATLYSPMSQKIMTKMGYIPGKGLGKNEDGIKVPIEA
KINHGREGTGYPF
SEQ ID NO: 122
DNA
Homo sapiens
HERV-K env
>P61570
MNPSEMQRKAPPRRRRHRNRAPLTHKMNKMVTSEEQMKLPSTKKAEPPTWAQLKKLTQL
ATKYLENTKVTQTPESMLLAA
LMIVSMVVSLPMPAGAAAANYTYWAYVPFPPLIRAVTWMDNPIEVYVNDSVWVPGPIDDRC
PAKPEEEGMMINISIGYRY
PPICLGTAPGCLMPAVQNWLVEVPIVSPISRFTYHMVSGMSLRPRVNYLQDFSYQRSLKFR
PKGKPCPKEIPKESKNTEV
LVWEECVANSAVILQNNEFGTIIDWAPRGQFYHNCSGQTQSCPSAQVSPAVDSDLTESLDK
HKHKKLQSFYPWEWGEKGI
STPRPKIVSPVSGPEHPELWRLTVASHHIRIWSGNQTLETRDRKPFYTVDLNSSLTVPLQSC
VKPPYMLVVGNIVIKPDS
QTITCENCRLLTCIDSTFNWQHRILLVRAREGVWIPVSMDRPWEASPSIHILTEVLKGVLNRS
KRFIFTLIAVIMGLIAV
TATGAVAGVALHSSVQSVNFVNDWQKNSTRLWNSQSSIDQKLANQINDLRQTVIWMGDRL
MSLEHRFQLQCDWNTSDFCI
TPQIYNESEHHWDMVRRHLQGREDNLTLDISKLKEQIFKASKAHLNLVPGTEAIAGVADGLA
NLNPVTWVKTIGSTTIIN
LILILVCLFCLLLVCRCTQQL
SEQ ID NO: 123
DNA
Homo sapiens
HERV-K env
>P61568
MPGAIDDHCPAQPGEEGTAFNVTMGYKYPPLCLGHATRCIHLETQVWAAYLLERLATGKW
GHLVSGLSLCPLRQMKRGVI
GDTPYFQYKPVGKLCPKNFEGPSKTLIWGDCVNSHAVVLKNDSYALVIDWAPKGYLKNTCS
SGGGEFLEATYFISYWEDE
DHHPTLHRWFGSFFTLKWEDKDITLHPQGLV
SEQ ID NO: 124
DNA
Homo sapiens
HERV-K env
>P61567
MVTPVTWMDNPIEIYVNDSVWVPGPIDDRCPAKPEEEGMMINISIGYRYPPICLGRAPGCLM
PAVQNWLVEVPTVSPISR
FTYHMVSGMSLRPRVNYLQDFSYQRSLKFRPKGKPCPKEIPKESKNTEVLVWEECVANSAV
ILQNNEFGTIIDWAPRGQF
YHNCSGQTQSCPSAQVSPAVDSDLTESLDKHKHKKLQSFYPWEWGEKRISTPRPKIVSPVS
GPEHPELWRLTVASHHIRI
WSGNQTLETRDCKPFYTIDLNSSLTVPLQSCVKPPYMLVVGNIVIKPDSQTITCENCRLLSCI
DSTFNWQHRILLVRARE
GVWIPVSMDRPWEASPSVHILTEVLKGVLNRSKRFIFTLIAVIMGLIAVTATAAVAGVALHSSV
QSVNFVNDWQKNSTRL
WNSQSSIDQKLANQINDLRQTVIWMGDRLMSLEHRFQLQCDWNTSDFCITPQIYNESEHHW
DMVRRHLQGREDNLTLDIS
KLKEQIFEASKAHLNLVPGTEAIAGVADGLANLNPVTWVKTIGSTTIINLILILVCLFCLLLVCRC
TQQLRRDSDHRERA
MMTMAVLSKRKGGNVGKSKRDQIVTVSV
SEQ ID NO: 125
DNA
Homo sapiens
HERV-K env
>P61566
MVTPVTWMDNPIEVYVNDSEWVPGPTDDRCPAKPEEEGMMINISIGYRYPPICLGTAPGCL
MPAVQNWLVEVPIVSPISR
FTYHMVSGMSLRPRVNYLQDFPYQRSLKFRPKGKPCPKEIPKESKNTEVLVWEECVANSAV
ILQNNEFGTIIDWAPRGQF
YHNCSGQTQSCPSAQVSPAVDSDLTESLDKHKHKKLQSFYPWEWGEKGISTPRPKIISPVS
GPEHPELWRLTVASHHIRI
WSGNQTLETRDRKPFYTVDLNSSLTLPLQSCVKPPYMLVVGNIVIKPDSQTITCENCRLLTCI
DSTFNWQHRILLVRARE
GVWILVSMDRPWEASPSVHILTEVLKGVLNRSKRFIFTLIAVIMGLIAVTATGAVAGVALHSSV
QSVNFVNDWQKNSTRL
WNSQSSIDQKLANQINDLRQTVIWMGDRLMSLEHRFQLQCDWNTSDFCITPQIYNESEHHW
DMVRHHLQGREDNLTLDIS
KLKEQIFEASKAHLNLVPGTEAIAGVADGLANLNPVTWVKTIGSTTIINLILILVCLFCLLLVCRC
TQQLRRDSDHRERA
MMTMAVLSKRKGGNVGKSKRDQIVTVSV
SEQ ID NO: 126
DNA
Homo sapiens
HERV-K env
>P61565
MHPSEMQRKAPPRRRRHRNRAPLTHKMNKMVTSEQMKLPSTKKAEPPTWAQLKKLTQLA
TKYLENTKVTQTPESMLLAAL
MIVSMVVSLPMPAGAAAANYTNWAYVPFPPLIRAVTWMDNPIEVYVNDSVWVHGPIDDRCP
AKPEEEGMMINISIGYHYP
PICLGRAPGCLMPAVQNWLVEVPTVSPISRFTYNMVSGMSLRPRVNYLQDFSYQRSLKFRP
KGKPCPKEIPKESKNTEVL
VWEECVANSVVILQNNEFGTIIDWAPRGQFYHNCSGQTQSCPSAQVSPAVDSDLTESLDKH
KHKKLQSFYPWEWGEKGIS
TPRPKIISPVSGPEHPELWRLTVASHHIRIWSGNQTLETRDRKPFYTVDLNSSLTVPLQSCVK
PPYMLVVGNIVIKPDSQ
TITCENCRLLTCIDSTFNWQHRILLVRAREGVWIPVSMDRPWEASPSIHILTEVLKGVLNRSK
RFIFTLIAVIMGLIAVT
AMAAVAGVALHSFVQSVNFVNDWQKNSTRLWNSQSSIDQKLANQINDLRQTVIWMGDRLM
SLEHRFQLQCDWNTSDFCIT
PQIYNESEHHWDMVRRHLQGREDNLTLDISKLKEQIFEASKAHLNLVPGTEAIAGVADGLAN
LNPVTWVKTIGSTTIINL
ILILVCLFCLLLVCRCTQQLRRDSDHRERAMMTMVVLSKRKGGNVGKSKRDQIVTVSV
SEQ ID NO: 127
DNA
Homo sapiens
HERV-K env
>O71037
MNPSEMQRKAPPRRRRHRNRAPLTHKMNKMVTSEEQMKLPSTKKAEPPTWAQLKKLTQL
ATKYLENTKVTQTPESMLLAA
LMIVSMVVSLPMPAGAAAANYTYWAYVPFPPLIRAVTWMDNPIEVYVNDSVWVPGPTDDHC
PAKPEEEGMMINISIGYRY
PPICLGRAPGCLMPAVQNWLVEVPTVSPISRFTYHMVSGMSLRPRVNYLQDFSYQRSFKFR
PKGKPCPKEIPKESKNTEV
LVWEECVANSAVILQNNEFGTIIDWAPRGQFYHNCSGQTQSCPSAQVSPAVDSDLTESLDK
HKHKKLQSFYPWEWGEKGI
STPRPKIISPVSGPEHPELWRLTVASHHIRIWSGNQTLETRDRKPFYTVDLNSSVTVPLQSCI
KPPYMLVVGNIVIKPDS
QTITCENCRLLTCIDSTFNWQHRILLVRAREGVWIPVSMDRPWETSPSIHTLTEVLKGVLNRS
KRFIFTLIAVIMGLIAV
TATAAVAGVALHSSVQSVNFVNDWQKNSTRLWNSQSSIDQKLANQINDLRQTVIWMGDRL
MSLEHRFQLQCDWNTSDFSI
TPQIYNESEHHWDMVRRHLQGREDNLTLDISKLKEQIFEASKAHLNLVPGTEAIAGVADGLA
NLNPVTVWKTIGSTTIIN
LILILVCLFCLLLVCRCTQQLRRDSDHRERAMMTMAVLSKRKGGNVGKSKRDQIVTVSV
SEQ ID NO: 128
DNA
Homo sapiens
HERV-K env
>O42043
MVTPVTWMDNPIEVYVNDSVWVPGPTDDRCPAKPEEEGMMINISIGYHYPPICLGRAPGCL
MPAVQNWLVEVPTVSPNSR
FTYHMVSGMSLRPRVNCLQDFSYQRSLKFRPKGKTCPKEIPKGSKNTEVLVWEECVANSV
VILQNNEFGTIIDWAPRGQF
YHNCSGQTQSCPSAQVSPAVDSDLTESLDKHKHKKLQSFYLWEWEEKGISTPRPKIISPVS
GPEHPELWRLTVASHHIRI
WSGNQTLETRYRKPFYTIDLNSILTVPLQSCVKPPYMLVVGNIVIKPASQTITCENCRLFTCID
STFNWQHRILLVRARE
GMWIPVSTDRPWEASPSIHILTEILKGVLNRSKRFIFTLIAVIMGLIAVTATAAVAGVALHSSVQ
SVNFVNYWQKNSTRL
WNSQSSIDQKLASQINDLRQTVIWMGDRLMTLEHHFQLQCDWNTSDFCITPQIYNESEHHW
DMVRRHLQGREDNLTLDIS
KLKEQIFEASKAHLNLVPGTEAIAGVADGLANLNPVTWIKTIRSTMIINLILIVVCLFCLLLVCRC
TQQLRRDSDIENGP
SEQ ID NO: 129
DNA
Homo sapiens
HERV-W Env and Multiple sceloris related virus HERV-W Env
>Q9UQF0
MALPYHIFLFTVLLPSFTLTAPPPCRCMTSSSPYQEFLWRMQRPGNIDAPSYRSLSKGTPTF
TAHTHMPRNCYHSATLCM
HANTHYWTGKMINPSCPGGLGVTVCWTYFTQTGMSDGGGVQDQAREKHVKEVISQLTRV
HGTSSPYKGLDLSKLHETLRT
HTRLVSLFNTTLTGLHEVSAQNPTNCWICLPLNFRPYVSIPVPEQWNNFSTEINTTSVLVGPL
VSNLEITHTSNLTCVKF
SNTTYTTNSQCIRWVTPPTQIVCLPSGIFFVCGTSAYRCLNGSSESMCFLSFLVPPMTIYTEQ
DLYSYVISKPRNKRVPI
LPFVIGAGVLGALGTGIGGITTSTQFYYKLSQELNGDMERVADSLVTLQDQLNSLAAVVLQN
RRALDLLTAERGGTCLFL
GEECCYYVNQSGIVTEKVKEIRDRIQRRAEELRNTGPWGLLSQWMPWILPFLGPLAAIILLLL
FGPCIFNLLVNFVSSRI
EAVKLQMEPKMQSKTKIYRRPLDRPASPRSDVNDIKGTPPEEISAAQPLLRPNSAGSS
SEQ ID NO: 130
DNA
Homo sapiens
HERV-W Env
>AAK18189.1
MALPYHTFLFTVLLPPFALTAPPPCCCTTSSSPYQEFLWRTRLPGNIDAPSYRSLSKGNSTF
TAHTHMPRNCYNSATLCM
HANTHYWTGKMINPSCPGGLGATVCWTYFTHTSMSDGGGIQGQAREKQVKEAISQLTRGH
STPSPYKGLVLSKLHETLRT
HTRLVSLFNTTLTRLHEVSAQNPTNCWMCLPLHFRPYISIPVPEQWNNFSTEINTTSVLVGPL
VSNLEITHTSNLTCVKF
SNTIDTTSSQCIRWVTPPTRIVCLPSGIFFVCGTSAYHCLNGSSESMCFLSFLVPPMTIYTEQ
DLYNHVVPKPHNKRVPI
LPFVIRAGVLGRLGTGIGSITTSTQFYYKLSQEINGDMEQVTDSLVTLQDQLNSLAAVVLQNR
RALDLLTAKRGGTCLFL
GEERCYYVNQSRIVTEKVKEIRDRIQCRAEELQNTERWGLLSQWMPWTLPFLGPLAAIIFLLL
FGPCIFNFLVKFVSSRI
EAVKLQIVLQMEPQMQSMTKIYRGPLDRPARLCSDVNDIEVTPPEEISTAQPLLHSNSVGSS
SEQ ID NO: 131
DNA
Homo sapiens
HERV-W Env
>AAD14545.1
MALPYHIFLFTVVSPSFTLTAPPPCRCMTSSSPYQEFLWRMQRPGNIDAPSYRSLCKGTPTF
TAHTHMPRNCYHSATLCM
HANTHYWTGKMINPSCPGGLGVTVCWTYFTQTGMSDGGGVQDQAREKHVKEVISQLTRV
HGTSSPYKGLDLSKLHETLRT
HTRLVSLFNTTLTGLHEVSAQNPTNCWICLPLNFRPYVSIPVPEQWNNFSTEINTTSVLVGPL
VSNLEITHTSNLTCVKF
SNTTYTTNSQCIRWVTPPTQIVCLPSGIFFVCGTSAYRCLNGSSESMCFLSFLVPPMAIYTEQ
DLYSYVISKPRNKRVPI
LPFVIGAGVLGALGTGIGGITTSTQFYYKLSQELNGDMERVADSLVTLQDQLNSLAAVVLQN
RRALDLLTAERGGTCLFL
GEECCYYVNQ
SEQ ID NO: 132
DNA
Homo sapiens
HERV-H Env
>Q9N2J8
MILAGRAPSNTSTLMKFYSLLLYSLLFSFPFLYHPLPLPSYLHHTINLTHSLPAASNPSLANNC
WLCISLSSSAYIAVPT
LQTDRATSPVSLHLRTSFNSPHLYPPEELIYFLDRSSKTSPDISHQPAAALLHIYLKNLSPYIN
STPPIFGPLTTQTTIP
VAAPLCISRQRPTGIPLGNISPSRCSFTLHLQSPTTHVTETIGVFQLHIIDKPSINTDKLKNVSS
NYCLGRHLPYISLHP
WLPSPCSSDSPPRPSSCLLTPSPQNNSERLLVDTQRFLIHHENRTSSSMQLAHQSPLQPLT
AAALAGSLGVWVQDTPFST
PSHPFSLHLQFCLTQGLFFLCGSSTYMCLPANWTGTCTLVFLTPKIQFANGTKELPVPLMTL
TPQKRVIPLIPLMVGLGL
SASTIALSTGIAGISTSVTTFRSPSNDFSASITDISQTLSVLQAQVDSLAAVVLQNRRGLGLSIL
LNEECCFYLNQSGLV
YENIKKLKDRAQKLANQASNYAESPWALSNWMSWVLPILSPLIPIFLLLLFGPCIFHLVSQFIQ
NRIQAITNHSI
SEQ ID NO: 133
DNA
Homo sapiens
HERV-H Env
>Q9N2K0
MIFAGKAPSNTSTLMKFYSLLLYSLLFSFPFLCHPLPLPSYLHHTINLTHSLLAASNPSLVNNC
WLCISLSSSAYTAVPA
VQTDWATSPISLHLRTSFNSPHLYPPEELIYFLDRSSKTSPDISHQQAAALLRTYLKNLSPYIN
STPPIFGPLTTQTTIP
VAAPLCISWQRPTGIPLGNLSPSRCSFTLHLRSPTTNINETIGAFQLHITDKPSINTDKLKNISS
NYCLGRHLPCISLHP
WLSSPCSSDSPPRPSSCLLIPSPENNSERLLVDTRRFLIHHENRTFPSTQLPHQSPLQPLTA
AALAGSLGVWVQDTPFST
PSHLFTLHLQFCLAQGLFFLCGSSTYMCLPANWTGTCTLVFLTPKIQFANGTEELPVPLMTP
TQQKRVIPLIPLMVGLGL
SASTVALGTGIAGISTSVMTFRSLSNDFSASITDISQTLSVLQAQVDSLAAVVLQNRRGLDLLT
AEKGGLCIFLNEECCF
YLNQSGLVYDNIKKLKDRAQKLANQASNYAEPPWALSNWMSWVLPIVSPLIPIFLLLLFGPCI
FRLVSQFIQNRIQAITN
HSIRQMFLLTSPQYHPLPQDLPSA
SEQ ID NO: 134
DNA
Homo sapiens
HERV-H Env
>CAB94193.2
mifagrassntstlmkfyslllysllfsfpilchplplpsylhhtinlthsllavsnpslakncwlcislpssaypavpa
lqtdwgtspvsphlrtsfnsphlyppekliyfldrssktspdishqqaaallctylknlspyinstpptfgplttqttip
vaaplcisrqrptgiplgnlspsrcsftlhlrsptthitetngafqlhitdkpsintdklknvssnyclgrhlscislhp
wlfspcssdspprpsscllipspknnsesllvdaqrfliyhenrtspstqlphqsplqpltaaplggslrvwvqdtpfst
pshlftlhlqfclvqslfflcgsstymclpanwtgtctlvfltskiqfangteelpvplmtptrqkrvipliplmvglgl
sastvalgtgiagistsvttfrilsndfsasitdisqtlsglqaqvdssaavvlqnrqgldlltaekgglciflneesyf
ylnqsglvydnikklkdkaqnlanqasnyaeppwplsnwmswvlpilsplipiflllffrpcifhlvsqfiqnhiqaitd
his
SEQ ID NO: 135
DNA
Homo sapiens
HERV-H Env
>AAL11492.1
MIFAGKAPSNTSTLMKFYSLLLYSLLFSFPFLCHPLPLPSYLHHTVNLTHSLLAASNPSLANN
CWLCISLSSSAYTAVPV
LQTDWATSPVSLHLRTSFNSPHLYPPEELIYFLDRSSKTSPDISHQQAAALLRTYLKNLSPYI
NSTPPIFGPLTTQTTIP
VAAPLCISRQRPTGIPLGNLSPSRCSFTLHLRSPTTHITETIGAFQLHITDKPSINTDKLKNISS
NYCLGRHVPCISLHP
WLSSPCSSDSPPRPSSCLLIPSPENNSERLLVDTRRFLIHHENRTFPSTQLPHQSPLQPLTA
AALAGSLGVWVQDTPFST
PSHLFTLHLQFCLAQALFFLCGSSTYMCLPANWTGTCTLVFLTRKIQFANGTEELPVPLMTP
TQQKRVIPLIPLMVGLGL
SA
SEQ ID NO: 136
DNA
Homo sapiens
HERV-H Env
>AAL11491.1
MIFAGKAPSNTSTLMKFYSLLLYSLLFSFPFLCHPLPLPSYLHHTINLTHSLLAASNPSLVNNC
WLCISLSSSAYTAVPT
LQTDWATSPISLHLRTSCNSPHLYPPEELIYFLDRSSKTSPDISHQQAAALLRTYLKNLSPYIN
STPPIFGPLTTQTTIP
VAAPLCISWQRPTGIPLGNLSPSRCSFTLHLRSPTTNINETIGAFQLHITDKPSINTDKLKNISS
NYCLGRHLPCISLHP
WLSSPCSSDSPPRPSSCLLIPSPENNSERLLVDTRRFLIHHENRTFPSTQLPHQSPLQPLTA
AALAGSLGVWVQDAPFST
PSHLFTLHLQFCLAQGLFFLCGSSTYMCLPANWTGTCTLVFLTPKIQFANGTEELPVPLMTP
TQQKRVIPLIPLMVGLGL
SA
SEQ ID NO: 137
DNA
Homo sapiens
HERV-H Env
>AAD34324.1
MIFAGKAPSNTSTLMKFYSLLLYSLLFSFPFLCHPLPLPSYLHHTINLTHSLLAASNPSLVNNC
WLCISLSSSAYTAVPA
LQTDWATSPISLHLRTSFNSPHLYPPEELIYFLDRSSKTSPDISHQQAAALLRTYLKNLSPYIN
STPPILGPLTTQTTIP
VAAPLCISWQRPTGIPLGNLSPSRCSFTLHLRSPTTNINETIGAFQLHITDKPSINTDKLKNISS
NYCLGRHLPCISLHP
WLSSPCSSDSPPRPSSCLLIPSPENNSERLLVDTRRFLIHHENRTFPSTQLPHQSPLQPLTA
AALAGSLGVWVQDTPFST
PSHLFTLHLQFCLAQGLFFLCGSSTYMCLPANWTGTCTLVFLTPKIQFANGTEELPVPLMTP
TQQKRVIPLIPLMVGLGL
SASTVALGTGIAGISTSVMTFRSLSNDFSASITDISQTLSVLQAQVDSLAAVVLQNRRGLDLLT
AEKGGLCIFLNEECCF
YLNQSGLVYDNIKKLKDRAQKLANQASNYAEPPWALSNWMSWVLPIVSPLIPIFLLLLFGPCI
FRLVSQFIQNRIQAITN
HSIRQMFLLTSPQYHPLPQDLPSA
SEQ ID NO: 138
DNA
Homo sapiens
HERV-R Env
>Q14264
MLGMNMLLITLFLLLPLSMLKGEPWEGCLHCTHTTWSGNIMTKTLLYHTYYECAGTCLGTCT
HNQTTYSVCDPGRGQPYV
CYDPKSSPGTWFEIHVGSKEGDLLNQTKVFPSGKDVVSLYFDVCQIVSMGSLFPVIFSSMEY
YSSCHKNRYAHPACSTDS
PVTTCWDCTTWSTNQQSLGPIMLTKIPLEPDCKTSTCNSVNLTILEPDQPIWTTGLKAPLGA
RVSGEEIGPGAYVYLYII
KKTRTRSTQQFRVFESFYEHVNQKLPEPPPLASNLFAQLAENIASSLHVASCYVCGGMNMG
DQWPWEARELMPQDNFTLT
ASSLEPAPSSQSIWFLKTSIIGKFCIARWGKAFTDPVGELTCLGQQYYNETLGKTLWRGKSN
NSESPHPSPFSRFPSLNH
SWYQLEAPNTWQAPSGLYWICGPQAYRQLPAKWSGACVLGTIRPSFFLMPLKQGEALGYP
IYDETKRKSKRGITIGDWKD
NEWPPERIIQYYGPATWAEDGMWGYRTPVYMLNRIIRLQAVLEIITNETAGALNLLAQQATK
MRNVIYQNRLALDYLLAQ
EEGVCGKFNLTNCCLELDDEGKVIKEITAKIQKLAHIPVQTWKG
SEQ ID NO: 139
DNA
Homo sapiens
HERV-R Env
>BAG58193.1
mlgmnmllitlflllpIsmlkgepwegclhcthttwsgnimtktllyhtyyecagtclgtcthnqttysvcdpgrgqpyv
cydpksspgiwfeihvgskegdllnqtkvfpsgkdvvslyfdvcqivsmgslfpvifssmeyysschknryahpacstds
pvttcwdcttwstnqqslgpimltkiplepdyktstsnsvnltilepdqpiwttglkaplgarvsgeeigpgayvylyii
kktrtrstqqfrvfesfyehvnqklpeppplasnlfaqlaeniasslhvascyvcggmnmgdqwpwearelmpqdnftlt
asslepapssqsiwflktsiigkfciarwgkaftdpvgeltclgqqyynetlgktlwrgksnnsesphpspfsrfpslnh
swyqleapntwqapsglywicgpqayrqlpakwsgacvlgtirpsfflmplkqgealgypiydetkrkskrgitigdwkd
sewpperiiqyygpatwaedgmwgyrtpvymlnriirlqavleiitnetagalnllaqqatkmrnviyqnrlaldyllaq
eegvcgkfsltncclelddegkvikeitakiqklahipvqtwkg

### FIGURES

The Figures show:

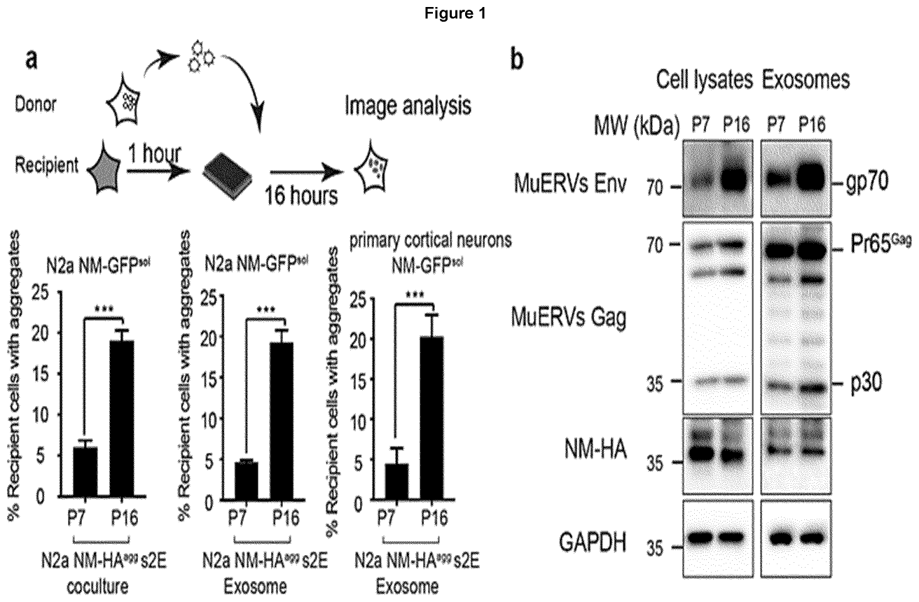

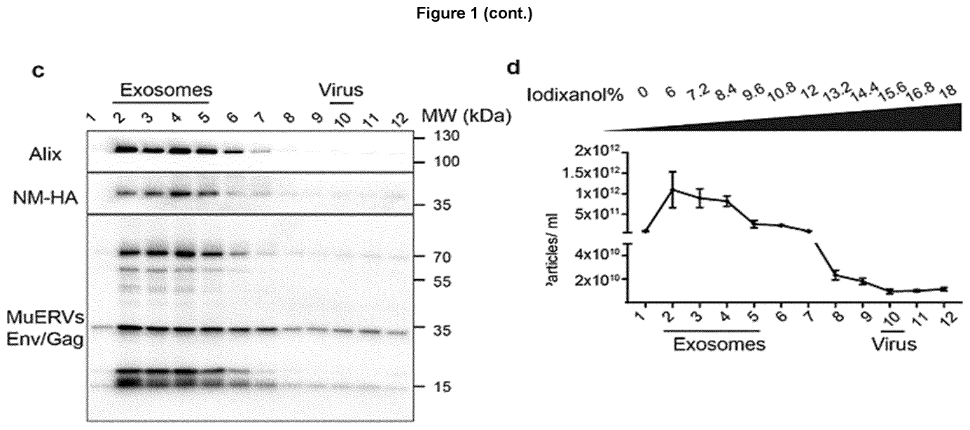

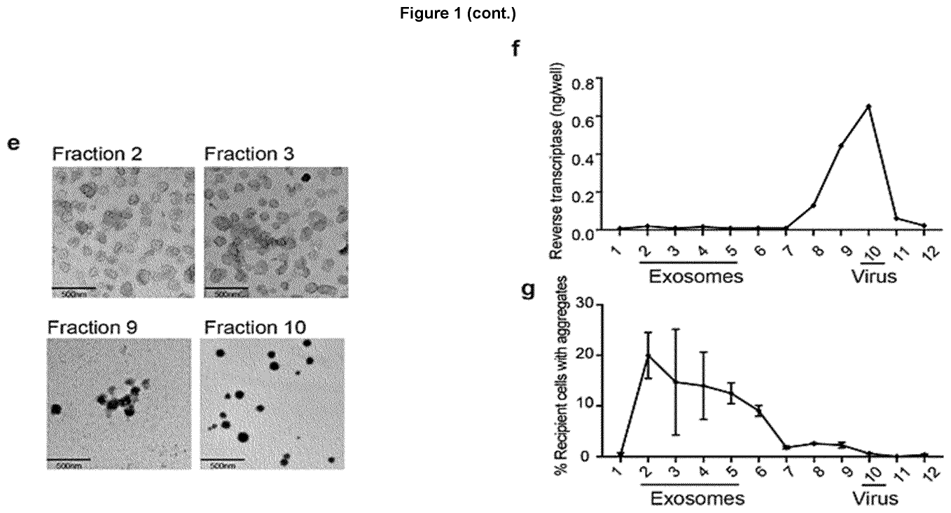

FIG. 1: Upregulation of MuERV (murine ERV) in donor cells increases intercellular aggregate induction.
a. Intercellular protein aggregate induction by direct cell-cell contact or via EVs. Recipient N2a cells expressing soluble NM-GFP (N2a NM-GFP^{sol}) are cocultured with donor cell clone N2a s2E, stably producing NM-HA aggregates (N2a s2E NM-HA^{agg}). Alternatively, EVs isolated from conditioned medium of donor cells are added to recipient cells 1 h post-plating. Quantitative image analysis is performed with images scanned by CellVoyager6000, an automated confocal microscope, after 16 h.
b. Continuous culture of donor cell clone N2a NM-HA^{agg} s2E increases the aggregate induction rate in cocultured recipient N2a NM-GFP^{sol} cells (left panel). Increased percentage of recipient N2a NM-GFP cells with NM-GFP aggregates upon exposure to EVs derived from high passage donor cells (middle panel). Similar results were obtained when EVs from low or high passage donor clones were used to induce NM-GFP aggregation in primary cortical neurons expressing soluble NM-GFP (right panel). P7: Passage 7; P16: Passage 16. Results shown are means±SD (n=6; ***, p<0.001; unpaired student t test).
c. Western blot analysis of cell lysates and EVs from donor clone s2E cells reveals increased MuERV Env and Gag expression upon continuous cell culture. Note that Env comprises the SU subunit gp70 and the TM subunit Pr15/p15E (shown is gp70). Gag subunits include Pr65^{Gag} and p30. Env was detected using mAb83A25 (Env) (Evans et al., J Virol (1990), 64: 6176-6183), Gag was detected using ab100970 (McNally et al., J Chromatogr A (2014), 1340:24-32) antibodies.
d. The 100,000×g pellet from conditioned medium of donor clone s2E NM-HA^{agg} (P21) was subjected to a shallow OptiPrep density gradient to separate EVs from virus particles. Twelve density gradient fractions were collected and analyzed for Alix, endogenous Env/Gag and NM-HA. Endogenous Env and Gag were detected using a goat polyclonal anti-xenotropic MuLV virus antibody ABIN457298 (antibodies-online).
e. Density gradient fractions of (d) were analyzed for particle numbers using ZetaView. Fractions containing EVs or viral particles are indicated.
f. Transmission electron microscopy reveals the typical cup-shaped EVs in fractions 2 and 3 and virus particles with an electron dense core in fractions 9 and 10. Scale bar: 500 nm.
g. Optiprep fractions were tested for reverse transcriptase activity using a colorimetric reverse transcriptase assay.

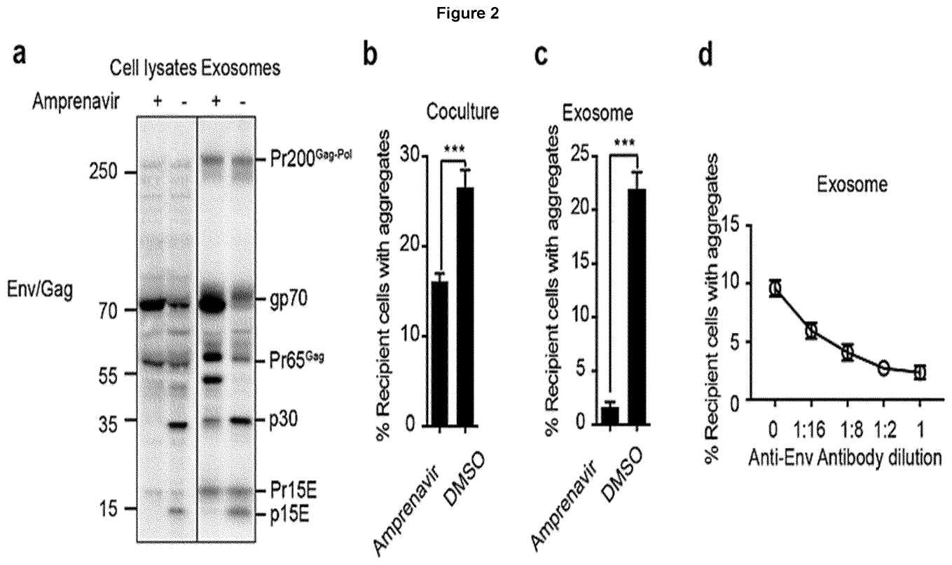

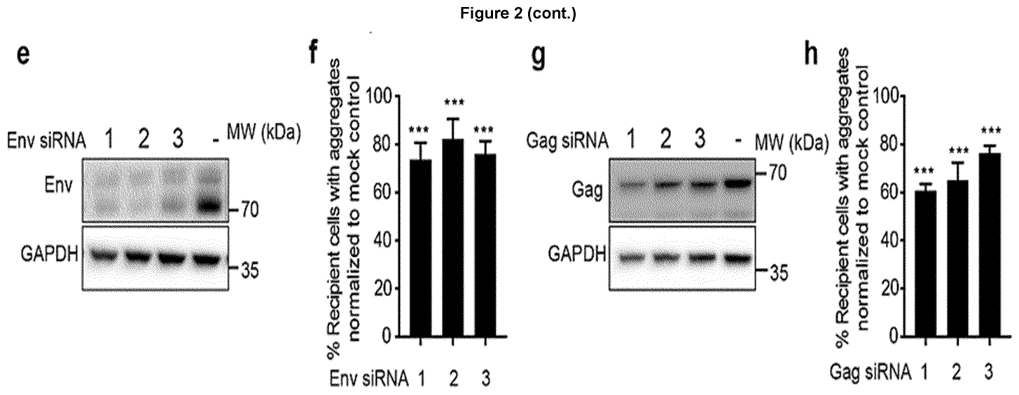

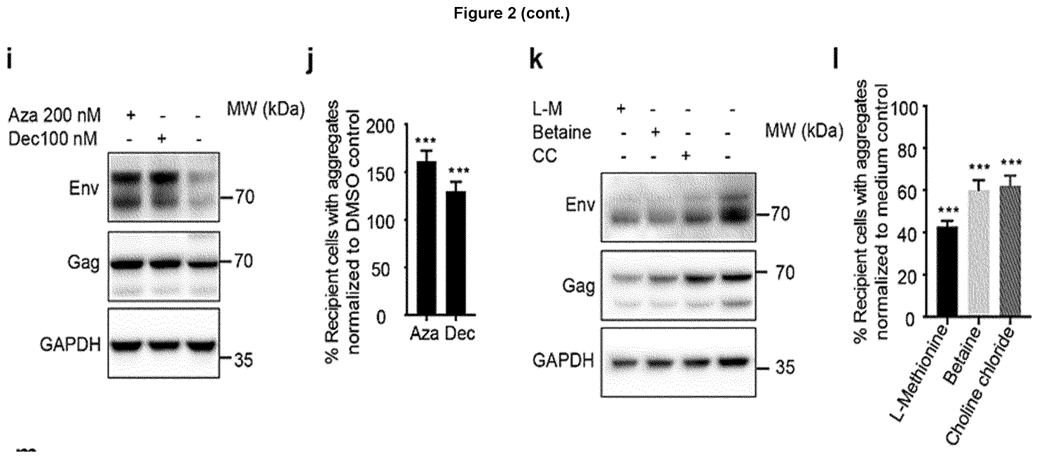

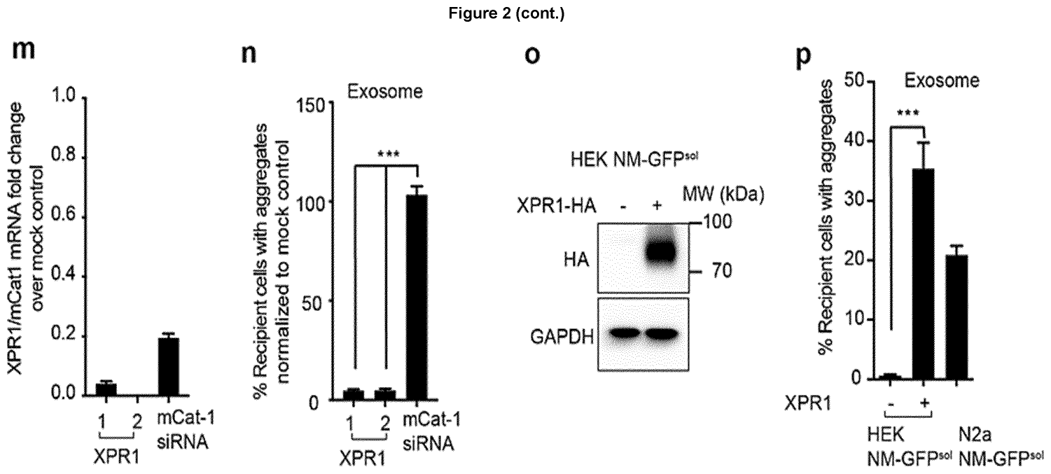

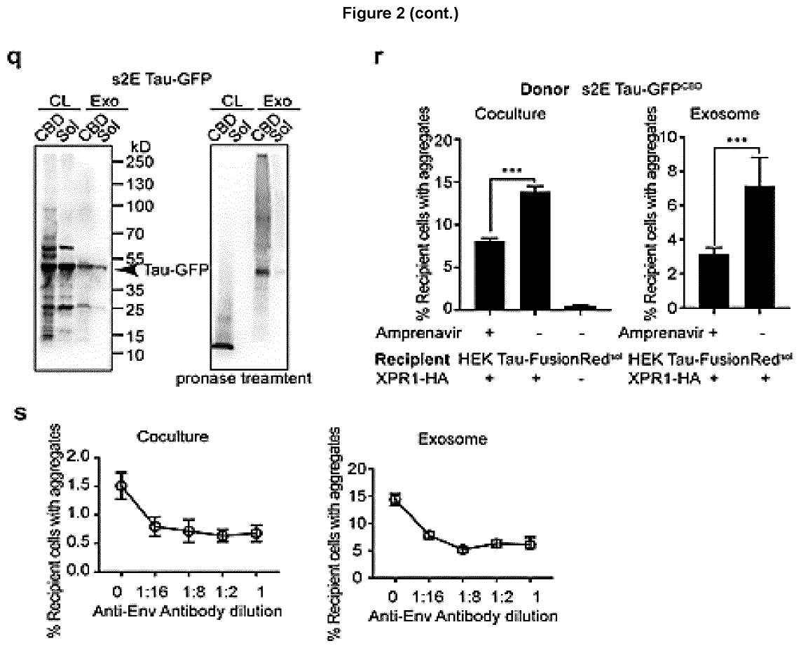

FIG. 2: Intercellular aggregate induction by EVs is greatly enhanced by MuERV Env receptor-ligand interactions.
a. Amprenavir inhibits maturation of endogenous MuERV proteins, including Env. Cell lysates and EVs derived from Amprenavir-treated donor cell clone N2a NM-HA^{agg} s2E were analyzed for Env maturation by Western blot. Amprenavir-treated cell lysate and EVs lack the proteolytical mature Env transmembrane (TM) fragment p15E required for receptor binding. Cleaved products and degradation intermediates were detected by anti-MuLV virus antibody ABIN457298. Presumed bands representing Env precursor protein (gp85 and gp70), Pr15E or cleavage product (p15E) are indicated.
b., c. Aggregate induction in recipient N2a NM-GFP^{sol} cells cocultured with Amprenavir-pretreated N2a NM-HA^{agg} s2E donor cells or (c) in recipient cells exposed to EVs derived from Amprenavir-treated donors.
d. EVs from donor clone N2a NM-HA^{agg} s2E were incubated with increasing concentrations of anti-Env antibody mAb83A25 for 1 h and added to recipient N2a NMGFP^{sol} cells for 16 h.
e, g. Donor clone N2a NM-HA^{agg} s2E was transfected with three individual siRNAs (SEQ ID NO 14-19) against endogenous Env (e) or (SEQ ID NO 20-25) against Gag (g). As control, donor cells were transfected with non-silencing siRNA (−). Western blots were developed using anti-Env antibody mAb83A25 or anti-Gag antibody ab100970, respectively.
f, h. Knock-down of endogenous Env (f) or Gag (h) in donor N2a NM-HA^{agg} s2E cells reduces aggregate induction in recipient N2a NM-GFP^{sol} cells in coculture. Shown is the percentage of recipient cells with NM-GFP^{agg} compared to the percentage of aggregate-bearing recipient cells in coculture upon donor transfection with non-silencing siRNA (set to 100%) (n=6, ***, p<0.001; one way ANOVA).
i. Donor N2a NM-HA^{agg} s2E cells of low passage number (P1) were treated with DNA methyltransferase inhibitors 5-azacytidine (Aza), 5-aza-2′-deoxycytidine (Dec) or DMSO for 2 d. Western blot analysis was performed 5 d post treatment using mAb83A25 (Env) and ab100970 (Gag) antibodies. GAPDH served as a loading control.
j. Five days post treatment, N2a NM-HA^{agg} s2E donor cells of low passage number (treatment initiated P1) were cocultured with recipient N2a NM-GFP^{sol} cells. Shown is the percentage of NM-GFP^{agg} positive recipient cells cocultured with drug-treated donors compared to the percentage of NM-GFP^{agg} positive recipient cells cocultured with solvent-treated donors (set to 100%) (n=6, ***, p<0.001; one-way ANOVA).
k. Donor N2a NM-HA^{agg} s2E cells cultured for prolonged times (treatment initiated P21) cells were treated with methyl group donors L-methionine (L-M), Betaine (B), Choline chloride (CC) or medium control for 6 days. MuERV Env and Gag protein levels were analyzed by Western blot.
l. Subsequently, cells were cocultured with recipient cells for 16 h. The percentage of NM-GFP aggregate containing recipient cells was compared to the percentage of NM-GFP aggregate bearing recipients cocultured with solvent-treated donors (set to 100%) (n=6, ***, p<0.001; one-way ANOVA).
m. Recipient N2a NM-GFP^{sol} cells were transfected with two siRNAs against XPR1 (SEQ ID NO 26-29), siRNA against mCat-1 (SEQ ID NO 30 and 31) or with non-silencing siRNA control. mRNA knock-down was assessed 48 h post siRNA transfection by quantitative PCR. Shown is the fold change in mRNA expression in recipient N2a NM-GFP^{sol} cells normalized to cells transfected with non-silencing siRNA.
n. XPR1 expression is required for EV-mediated NM-GFP aggregate induction in recipient cells. Recipient cells with downregulated XPR1 or mCat-1 expression were exposed to EVs purified from conditioned medium of donor clone s2E (P21) for 16 h. Shown is the percentage of NM-GFP^{agg} bearing recipient cells transfected with XPR1 or mCat-1 siRNA compared to the percentage of NM-GFP^{agg} bearing recipient cells that had been transfected with non-silencing control siRNA (set to 100%). (n=6, ***, p<0.001; one-way ANOVA).
o. Recipient HEK NM-GFP^{sol} cells stably expressing HA-tagged murine XPR1. Expression of murine XPR1-HA was confirmed by Western blot analysis using anti-HA antibodies. GAPDH served as a loading control.
p. Overexpression of murine XPR1-HA in recipient HEK NM-GFP^{sol} cells increases NM-GFP aggregate induction by donor-derived EVs. NM-GFP aggregate induction was measured 16 h post EV addition (n=6, ***, p<0.001; one-way ANOVA).
q. Induction of Tau-GFP aggregates in N2a s2E cells. The N2a s2E NM-HA^{agg} cell cone (P21) expressing high levels MuERV Env and Gag was transduced with lentivirus coding for Tau-GFP. Subsequently, cells were exposed to VSV-G pseudotyped EVs from HEK Tau-GFP^{CBD} cells to induce Tau aggregates. A cell clone was isolated based on persistent Tau-GFP aggregation (Tau-GFP^{CBD}). Cell lysate (CL) and EVs (Exo) isolated from N2a s2E Tau-GFP^{CBD} cells were assessed for the presence of pronase-resistant Tau. The blot was probed with Tau antibody ab64193 (Abcam).
r. Tau aggregate induction in recipient HEK Tau-FusionRed^{sol} cells expressing murine XPR1-HA cocultured with Amprenavir-pretreated s2E Tau-GFP^{CBD} donor cells (left panel). Tau aggregate induction in HEK Tau-FusionRed^{sol} cells expressing XPR1-HA exposed to EVs derived from Amprenavir treated donors. Note that EV-exposed recipients were also cultured in Amprenavir-containing medium to inhibit viral protein maturation in EVs (right panel). (n=6, ***, p<0.001; unpaired student t test).
s. Donor clone s2E Tau-GFP^{CBD} (left panel) or EVs (right panel) from the donor clone were incubated with increasing concentrations of anti-MuERV Env antibody for 1 h and added to recipient HEK Tau-FusionRed^{sol} cells expressing murine XPR1-HA for 3 d (n=6, p<0.001; unpaired student t test).

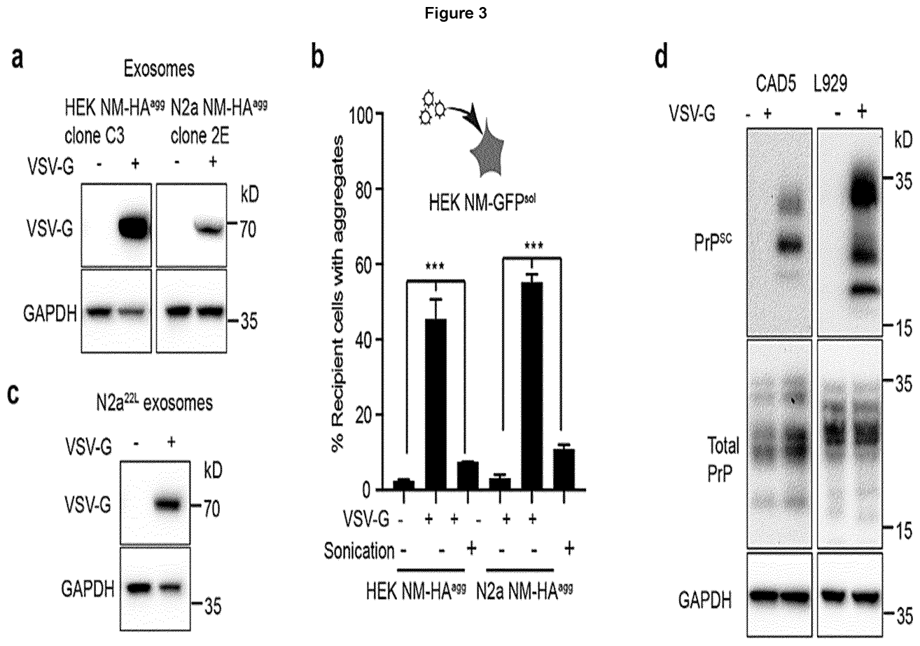

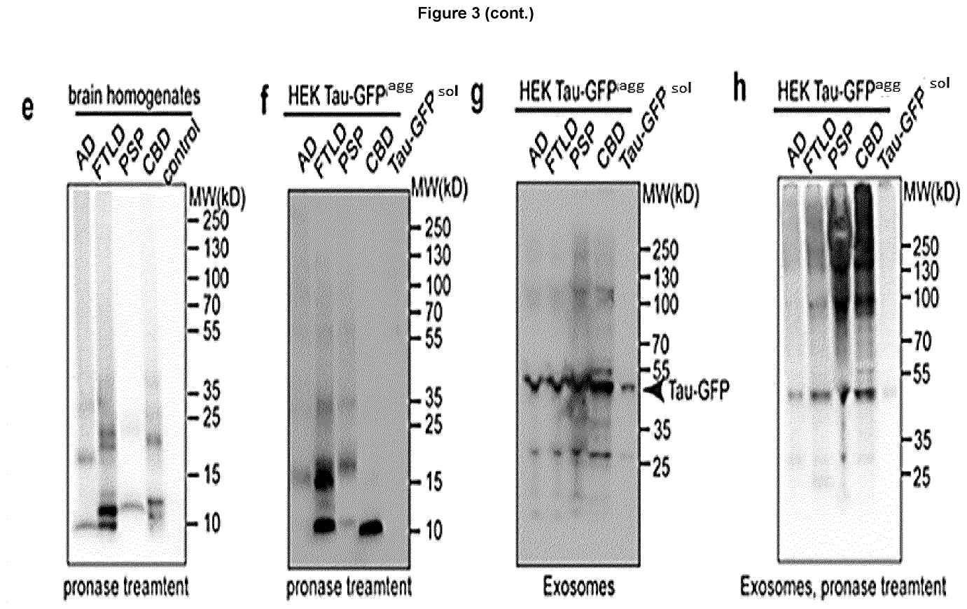

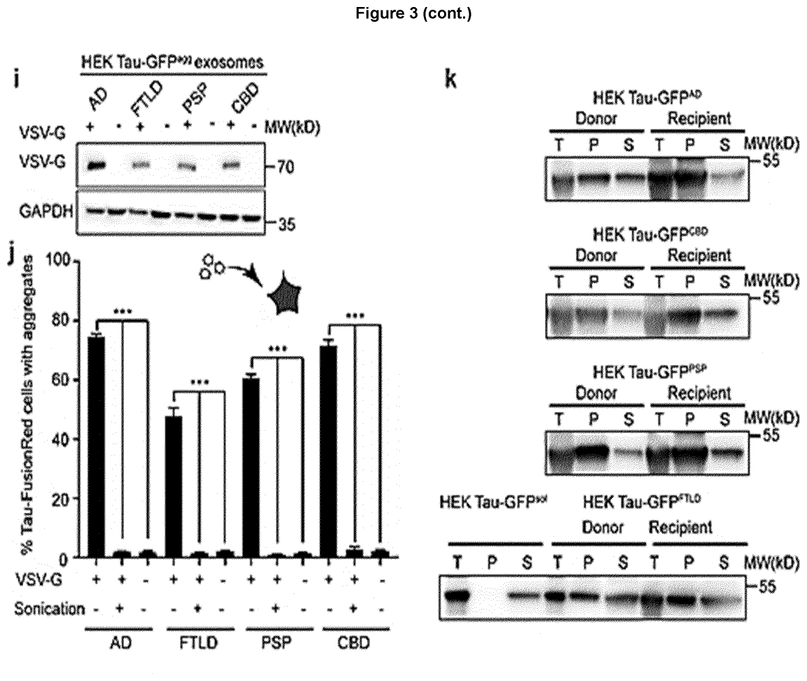

FIG. 3: Viral fusogens increase EV-mediated dissemination of different proteinaceous seeds.
a. Ectopic expression of VSV-G in donor cells. Donor HEK NM-HA^{agg} clone C3 and N2a NM-HA^{agg} clone 2E (precursor clone of s2E, poor inducer clone) were transiently transfected with a plasmid coding for VSV-G. VSV-G coated EVs were isolated 3 d post transfection and analysed by Western blot. GAPDH served as loading control.
b. Expression of VSV-G by donor HEK NM-HA^{agg} clone C3 or N2a NM-HA^{agg} clone 2E increases EV-mediated NM-GFP aggregation in recipient HEK NM-GFP^{sol} cells. Sonication (100% for 6 min) was used to destroy EVs (n=6, ***, p<0.001; one-way ANOVA).
c. Transient transfection of prion-infected N2a cells (N2a^{22L}) with a plasmid coding for VSV-G results in VSV-G pseudotyped EVs. EVs were harvested 3 d post transfection and analysed by Western blot. GAPDH served as a loading control.
d. VSV-G pseudotyped EVs derived from prion-infected N2a^{22L} cells increase infection of prion-permissive CAD5 and L929 cells. Recipient L929 and CAD5 cells were incubated with purified EVs from either mock-transfected or VSV-G plasmid transfected N2a^{22L} cells. Cells were passaged 8 times before accumulation of proteinase K resistant PrP (PrP^{Sc}, upper blot) and total PrP was monitored using anti-PrP mAb 4H11.
e. Different tauopathy patient brain lysates show distinct pronase-resistant patterns. Brain lysates from tauopathy patients prepared in lysis buffer (PBS with 1% Triton-X and protease, phosphatase inhibitors) were subjected to 100 μg/ml pronase. Pronase-resistant Tau was detected by Western blot using anti-Tau antibody ab64193. Differential sensitivity to pronase suggests conformational differences of Tau aggregates depositing in different tauopathies.
f. HEK Tau-GFP cell clones exposed to different tauopathy patient brain homogenates produce Tau-GFP aggregates with distinct pronase-resistant patterns. To induce Tau-GFP aggregation by patient-derived brain homogenates, a HEK cell clone stably expressing soluble Tau-GFP was exposed to brain homogenates from different tauopathy patients for 4 d. Cell populations were subsequently cloned to isolate cell lines with persistent Tau-GFP aggregates. Clones are named based on the tauopathy case from which the brain homogenate was derived. Lysates of individual cell clones were subjected to 100 μg/ml pronase treatment for 1 h at 37° C. Pronase-resistant Tau fragments were detected by Western blot using anti-Tau antibody ab64193.
g. EVs isolated from HEK Tau-GFP^{agg} clones contain Tau species detected with antibody against Tau (ab64193) (Sanders et al., Neuron (2014), 82:1271-1288).
h. EVs isolated from HEK Tau-GFP^{agg} clones contain pronase-resistant Tau species, as revealed by Western blot analysis using anti-Tau antibodies (ab64193).
i. Presence of VSV-G in exosomal fractions of transfected Tau-GFP^{agg} clones. Different HEK Tau-GFP^{agg} cell clones were transiently transfected with a plasmid coding for VSV-G or were mock transfected. Presence of VSV-G was assessed by Western blot analysis. GAPDH serves as a loading control.
j. 3 d later, EVs were harvested and recipient HEK Tau-FusionRed^{sol} cells were subsequently cultured with EVs or donor cells. Percentage of recipient cells with induced Tau-FusionRed aggregates following EV addition (n=6, ***, p<0.001; one-way ANOVA).
k. Sedimentation assay of Tau in lysates of different HEK Tau-GFP^{agg} donor cells and recipient HEK Tau-GFP^{sol} cells previously exposed to donor-derived VSV-G pseudotyped EVs. EV-exposed cells were cultured for 2 passages and analyzed. T: total cell lysate; S: supernatant; P: pellet. Tau-GFP was detected using anti-Tau antibody ab64193.

FIG. 4: Human endogenous retrovirus proteins potentiate intercellular Tau aggregate induction.
a. Workflow of Amprenavir treatment. Human T47D cells stably expressing NM-GFP or Tau-GFP were exposed to either NM fibrils or VSV-G pseudotyped EVs from corresponding cells (HEK Tau-GFP^{FTLD} or Tau-GFP^{AD}) to produce cell lines with NM-GFP^{agg} or Tau-GFP^{agg}. Two days later, these donor cells were incubated with 10 μM Amprenavir or solvent control DMSO for 1 d. Subsequently, donor cells with NM-GFP^{agg}, Tau-GFP^{FTLD} or Tau-GFP^{AD} aggregates were cocultured with recipient HEK NM-mCherry^{sol} or HEK Tau-FusionRed^{sol} cells, respectively, for 1 d (NM) or 3 d (Tau) in the presence of 10 μM Amprenavir or DMSO.
b. Amprenavir impairs NM-mCherry aggregate induction in recipient HEK NM-mCherry^{sol} cells upon coculture with T47D NM-GFP^{agg}. Percentage of recipient HEK NM-Cherry cells that harbor induced NM-mCherry aggregates.
c., d. Amprenavir impairs Tau-FusionRed aggregate induction in recipient HEK Tau-FusionRed^{sol} cells upon coculture with T47D Tau-GFP^{AD} or Tau-GFP^{FTLD}. Percentage of HEK Tau-FusionRed cells that harbor Tau-FusionRed^{agg} after coculture with T47D Tau-GFP^{agg} donor cell lines (n=6, ***, p<0.001; unpaired student t test).

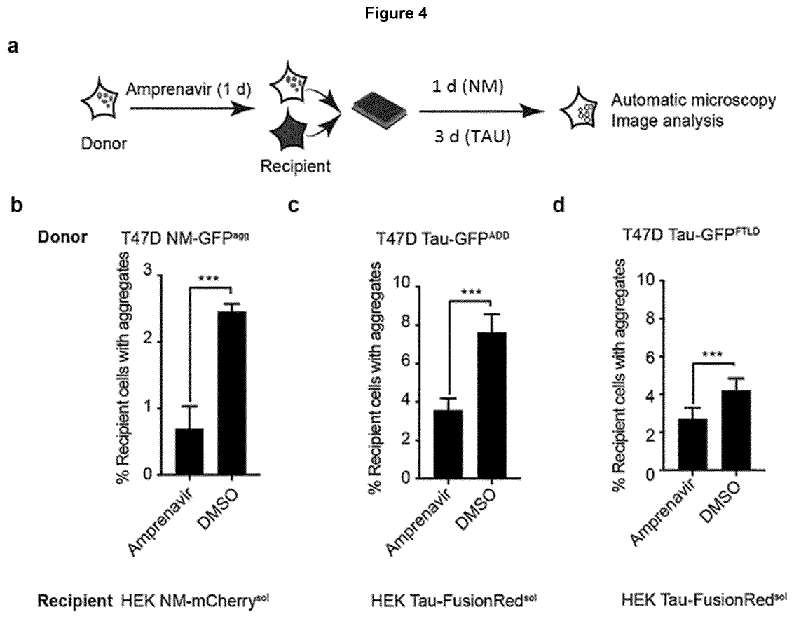

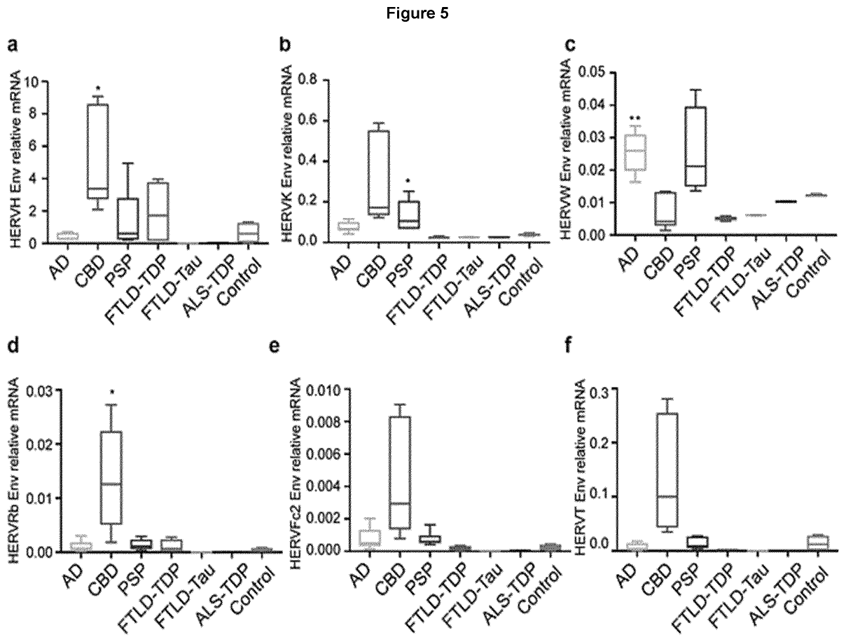

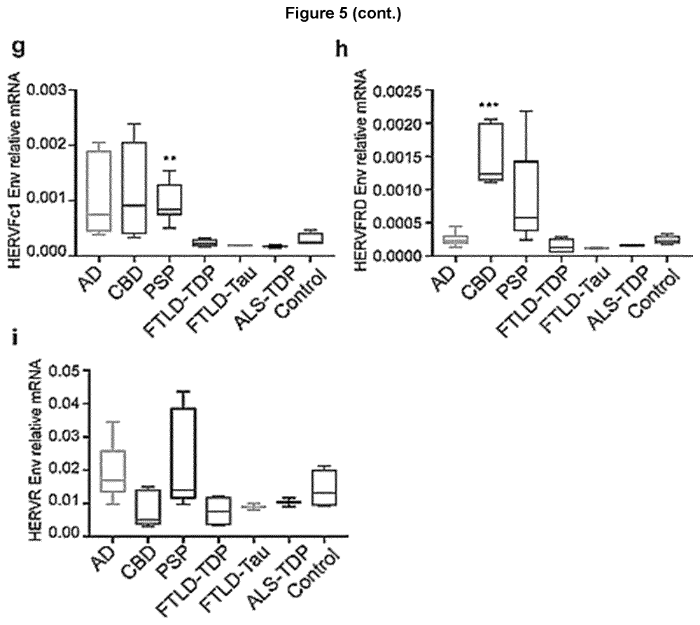

FIG. 5: Distinct HERV Env transcripts are upregulated in postmortem brains of tauopathy patients.
a-i. 9 selected HERV family members were assessed for their env transcript levels in postmortem brains of tauopathy patients. Brain samples from frontal cortex region of three AD, two CBD, two PSP and one FTLD patients, together with brain samples from pons of one CBD and one PSP patient were analyzed. HERV transcript levels in postmortem brain samples from AD, CBD, PSP and FTLD-Tau and ALS-TDP patients were compared to HERV expression levels in healthy controls. (n=3, ***, p<0.001; **, p<0.01; *, p<0.1; one way ANOVA)

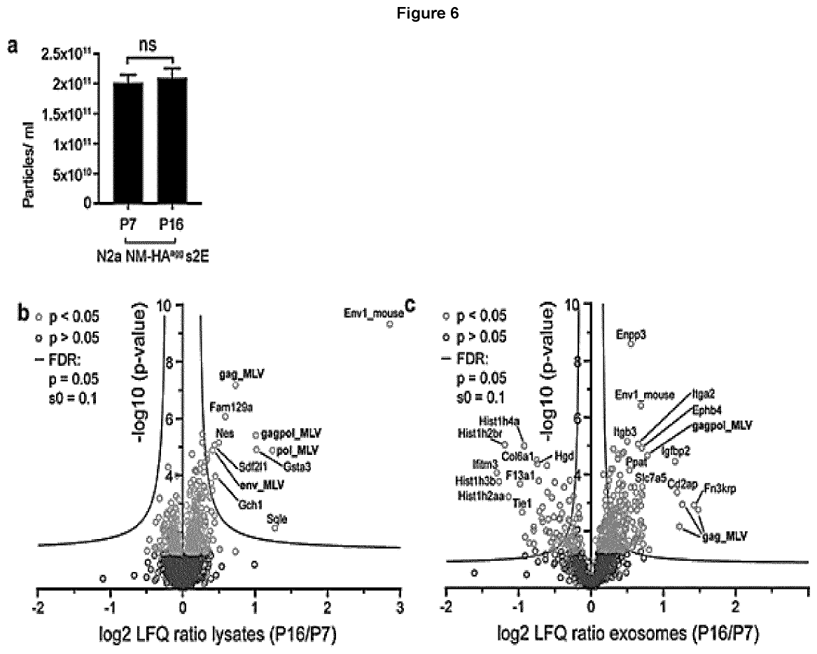

FIG. 6: Continuous culture of donor clone N2a NM-HA^{agg} s2E upregulates MuERV expression.
a. Continuous culture of N2a NM-HA^{agg} clone s2E does not increase EVs release. Numbers of microvesicles isolated from conditioned medium of cells passage 7 (P7) and 16 (P16) post thawing analyzed by ZetaView Nanoparticle Tracking (n=3; unpaired student t-test).
b. Volcano plot of total cell proteome. Cell lysates of donor N2a NM-HA^{agg} clone s2E at lower and higher passage number (P7 and P16) were subjected to quantitative mass spectrometry analysis. Proteins were ranked according to their p values and their relative abundance ratio (log 2 fold change) for cells of P16 compared to cells of P7.
c. Volcano plot of proteome of EVs derived from donor N2a NM-HA^{agg} clone s2E P16 versus P7.
d. Continuous culture of N2a NM-HA^{agg} clone s2E increases endogenous env and gag mRNA as revealed by qRT-PCR. Shown is the fold change in expression in donor cells P16 versus P7 (n=6, ****, p<0.0001; *, p<0.1, unpaired student t test).
e. To assess the release of virus particles, microvesicles were precipitated from conditioned medium of different N2a NM-HA^{agg} clones using polyethylenglycol (PEG). Reverse transcriptase (RT) activities were determined using a colorimetric RT assay (Roche). Mn²⁺-dependent RT activity of particles released from donor clone N2a NM-HA^{agg} s2E increased upon continuous passage. N2a NM-HA^{agg} clones 1C and 3B released only trace amounts of RT activity even upon prolonged culture.
f. Virus particles released from donor clone s2E at P17 are infectious to a murine melanocytic cell line (melan-a) susceptible to MuLV infection. Melan-a and human Hela cells were exposed for 6 d to conditioned medium of the donor clone (P17). Western blot analysis using antibody ABIN457298 against xenotropic MuLV viruses detects expression of Env and Gag only in melan-a cells. Viral proteins are indicated.

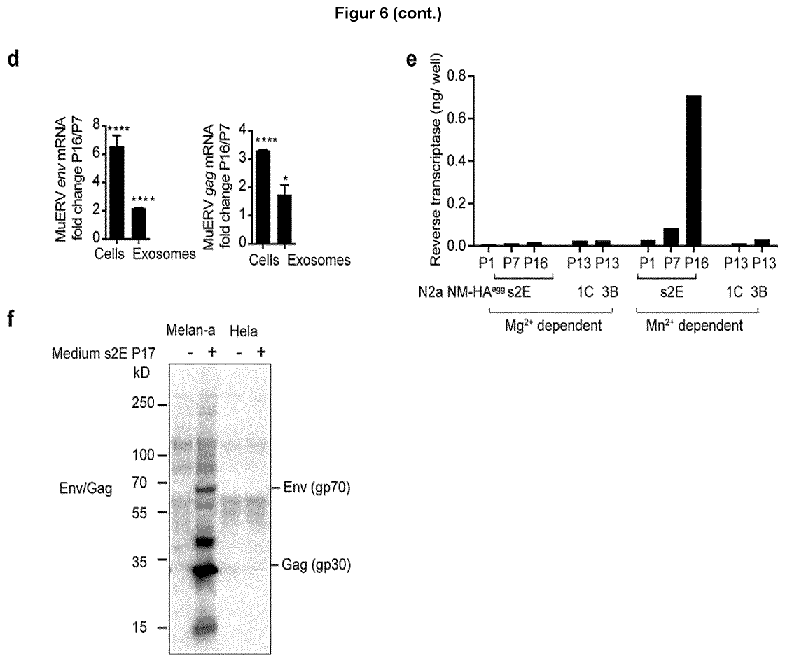

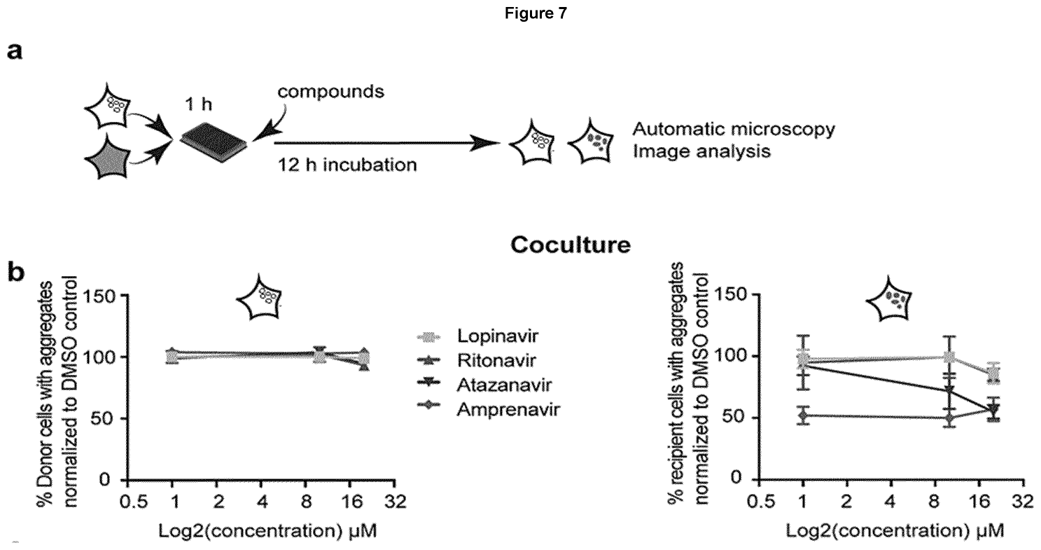

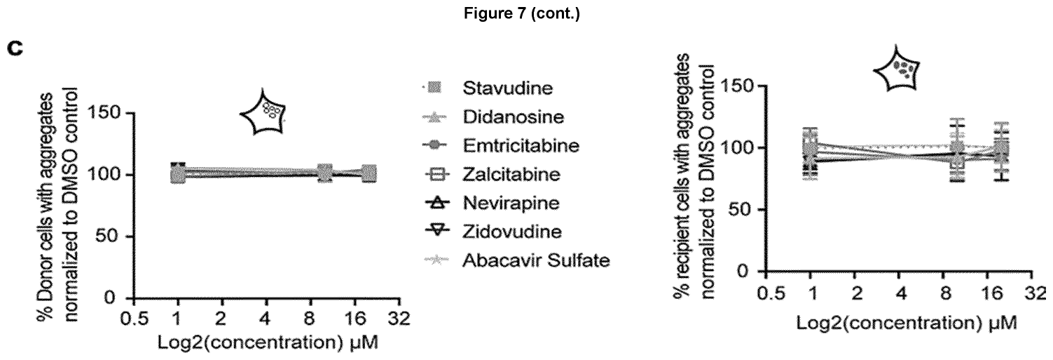

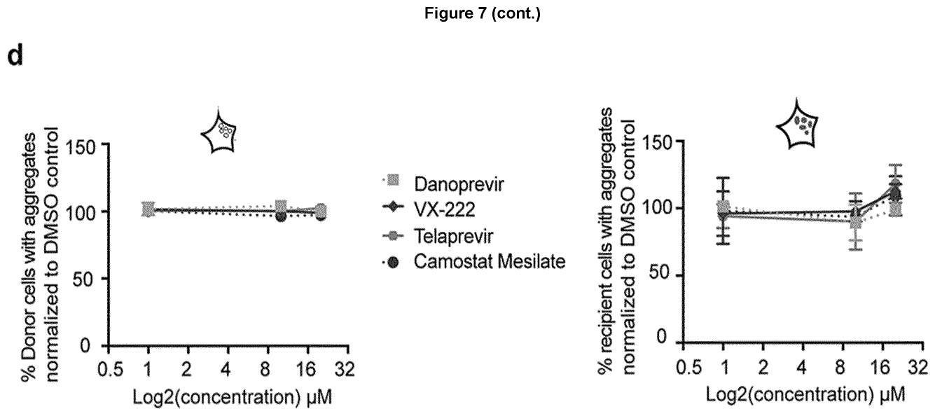

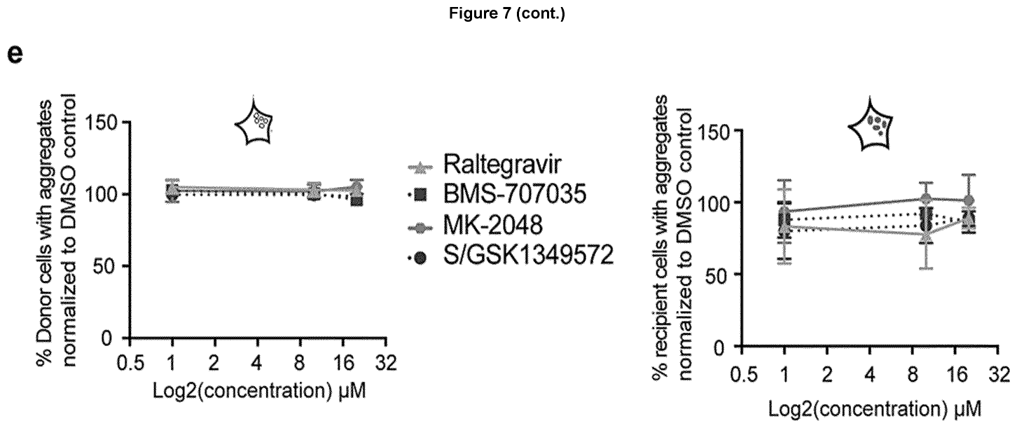

FIG. 7: Protease inhibitors effective against MuLV inhibit intercellular induction of NM-GFP aggregates.
a. Workflow of compound test in cocultures. Donor clone N2a NM-HA^{agg} s2E and recipient N2a NM-GFP^{sol} cells were co-seeded and different viral inhibitors were added at three concentrations 1 h later. Twelve hours post drug treatment, donor and recipient cells were analyzed for the percentage of donor cells with NM-HA^{agg} or recipient cells with induced NM-GFP^{agg}.
b. Effect of HIV protease inhibitors on NM aggregation in donor and recipient cells. Left panel: No effect of HIV protease inhibitors on the number of donor cells containing NM-HA^{agg}. Right panel: HIV protease inhibitors, particularly Amprenavir and Atazenavir, reduce aggregate induction in recipient NM-GFP^{sol} cells.
c-e. Effect of reverse transcriptase inhibitors (c), HCV protease inhibitors (d) and HIV integrase inhibitors (e) on the percentage of donor and recipient cells harboring NM-HA^{agg} or NM-GFP^{agg}, respectively. Cells with NM aggregates (either donor or recipient) that were solvent-treated (DMSO) were set as 100%. The percentage of drug-treated cells with aggregates was normalized to the percentage of aggregate-bearing cells treated with DMSO alone.

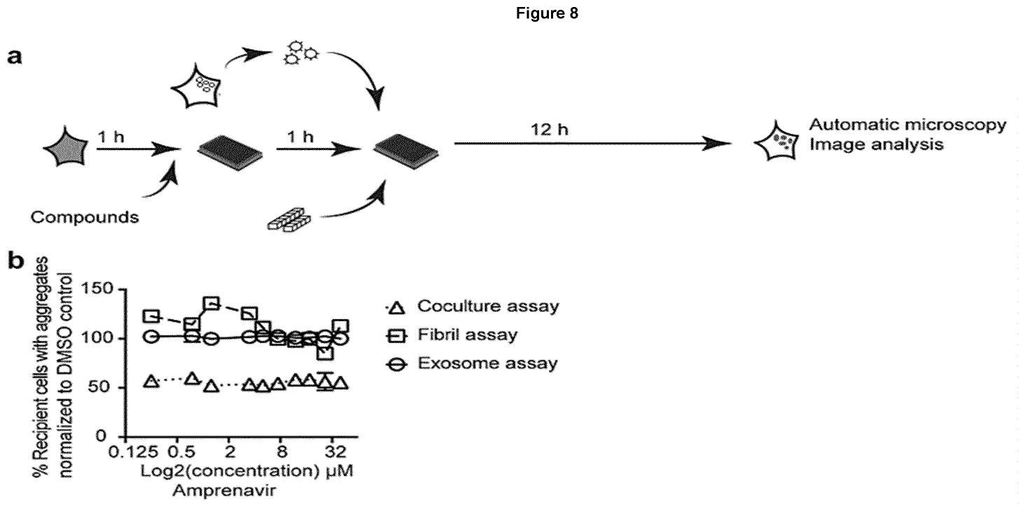

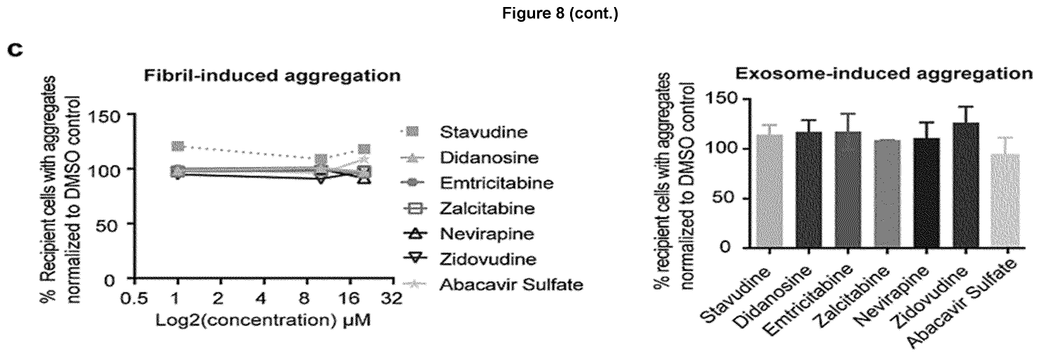

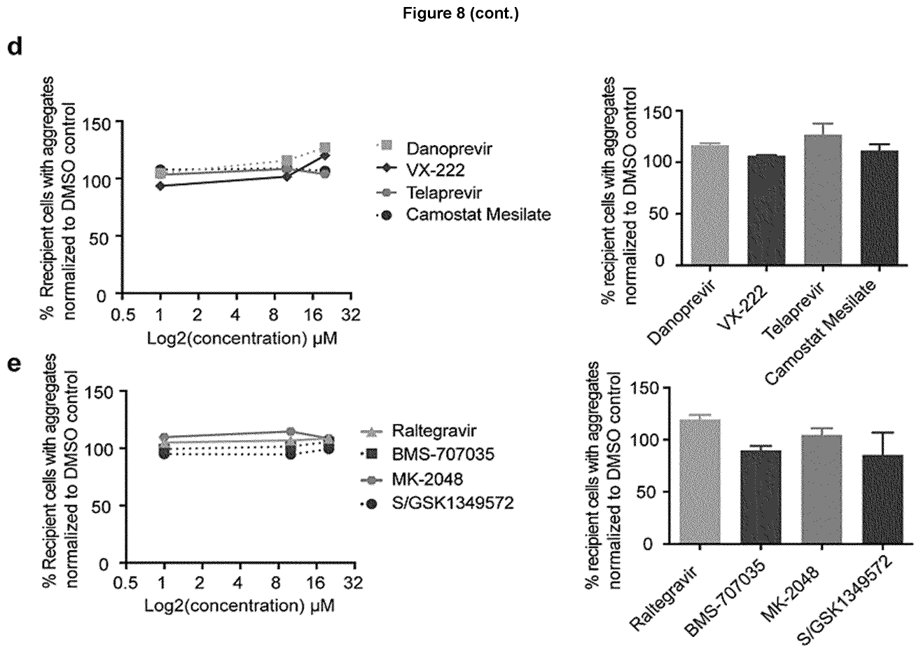

FIG. 8: Amprenavir impairs NM-GFP aggregate induction in cocultured recipient cells.
a. Workflow of compound test in cocultures, in recipient cells exposed to recombinant NM fibrils or in recipient cells exposed to donor-derived EVs (P17). For cocultures, donor clone N2a NM-HA^{agg} s2E (P17) and recipient N2a NM-GFP^{sol} cells were co-seeded and exposed to compounds 1 h later. Alternatively, recipient N2a NM-GFP^{sol} cells were pretreated with different concentrations of HIV inhibitors for 1 h, and cells were subsequently exposed to either recombinant fibrils (5 μM monomer equivalent) or donor-derived EVs for 12 h. Note that donor cells from which EVs were isolated remained untreated.
b. Amprenavir effects on NM-GFP aggregate induction in recipient cells in the three assays (a). The inhibitory effect of Amprenavir on NM-GFP aggregate induction in recipients was observed only when donor cells were cotreated.
c-e. Effect of reverse transcriptase inhibitors (c), HCV protease inhibitors (d) and HIV integrase inhibitors (e) on the induction of NM-GFP aggregates in recipient cells upon exposure to NM fibrils (left graphs) or EVs from NM-HA^{agg} s2E cells (P21) (right graphs). Cells exposed to fibrils were incubated with three concentrations of drugs (1, 10, 20 μM). Cells exposed to EVs were incubated with compounds (10 μM) in triplicate and data were analyzed using one-way ANOVA. Recipient cells with NM-GFP aggregates exposed to DMSO were set to 100%. Drug-treated cells were normalized to cells treated with DMSO alone.

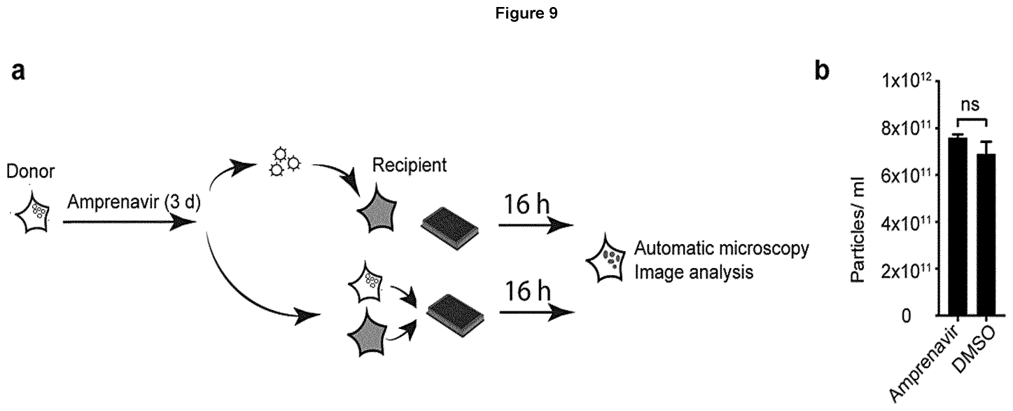

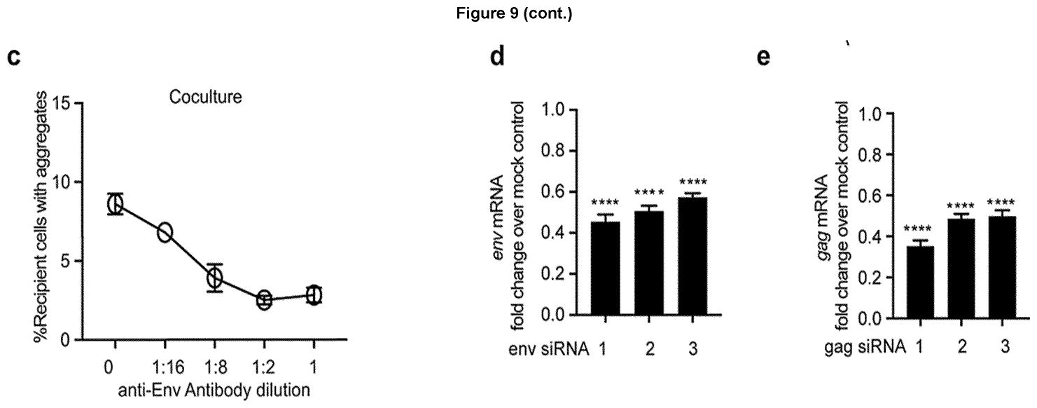

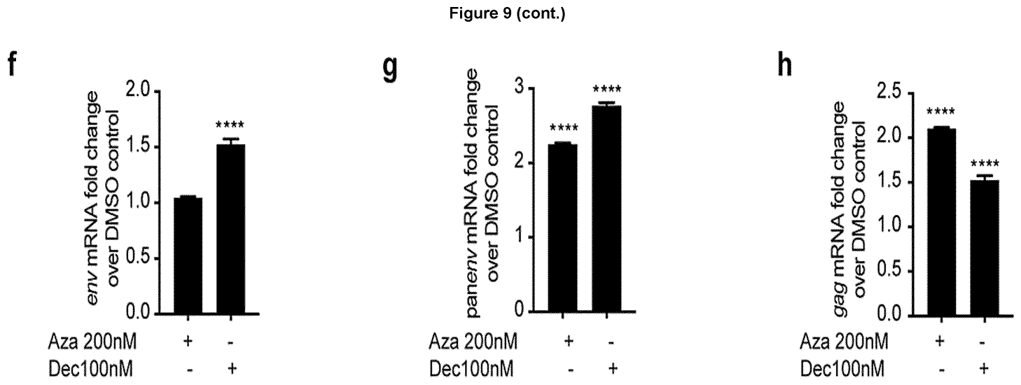

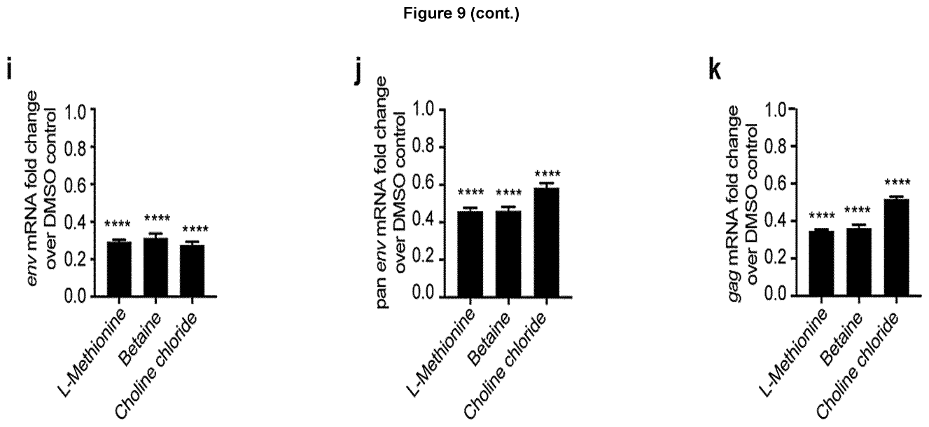

FIG. 9: Targeting MuERV protein maturation reduces NM-GFP aggregate induction in recipient cells.
a. Experimental setup to test if treatment of donor cells with Amprenavir affects NM-GFP^{agg} induction in recipient cells. Donor clone N2a NM-HA^{agg} s2E (P21) was treated with 10 μM Amprenavir or DMSO for 3 d. Pretreated donor cells were subsequently cocultured with recipient N2a NM-GFP^{sol} cells in the absence of the drug. In a second experimental set-up, EVs isolated from Amprenavir-treated donors were added to recipient cells in the absence of Amprenavir. Aggregate induction in recipient cells was assessed 16 h later.
b. Nanoparticle measurement of EVs isolated from conditioned medium of Amprenavir- and DMSO-treated donor cells. Amprenavir-treated cells released comparable amounts of particles as DMSO-treated cells.
c. Donor clone N2a NM-HA^{agg} s2E was incubated with anti-MuLV Env antibody mAb83A25 for 1 h. Subsequently, donor cells were cocultured with recipient N2a NM-GFP^{sol} cells for 16 h (n=6).
d, e. Reduction of MuERV env (d) or gag (e) transcripts following siRNA treatment assessed by qRT-PCR. Shown are fold changes relative to non-silencing control (n=3, ****, p<0.0001; one-way ANNOVA).
f-h. Donor clone N2a NM-HA^{agg} s2E one passage post defrosting was treated with DNA methyltransferase inhibitors 5-azacytidine (Aza), 5-aza-2′-deoxycytidine (Dec) or DMSO for 2 d and cells were subsequently incubated in the absence of the drug for 5 d. Increased expression of MuERV env (f), gag (h) and pan-env (g) transcripts in donor cells 5 d post treatment were revealed by qRT-PCR (n=3, ****, p<0.0001; one-way ANOVA) i-k. Donor cells of high passage number (P21) were treated with methyl group donors L-methionine, Betaine, Choline chloride or medium control for 6 d. MuERV env (i), gag (j) and pan-env (k) transcripts decreased after treatment with methyl group donors (n=3, ****, p<0.0001; one-way ANOVA).

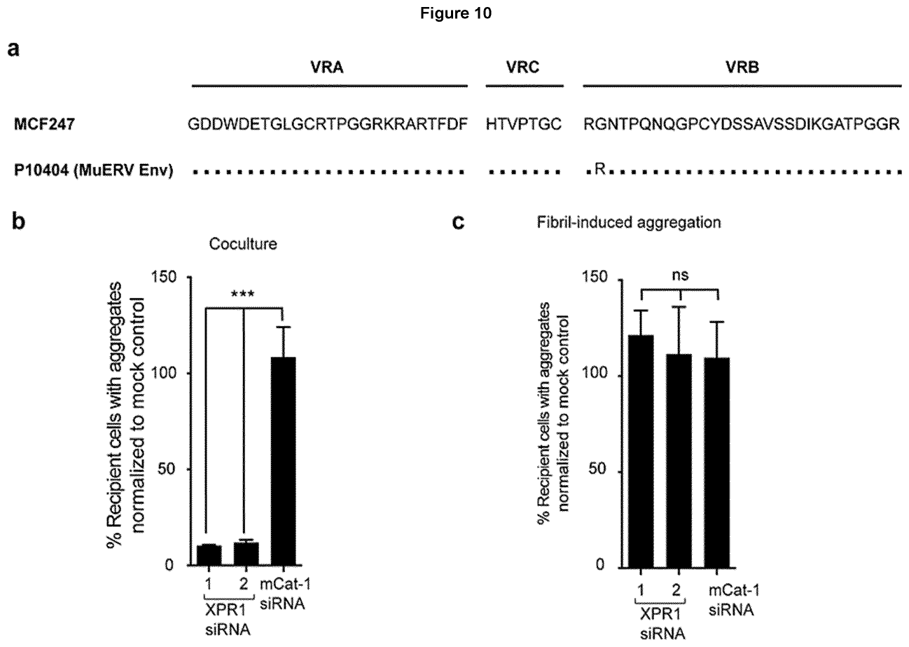

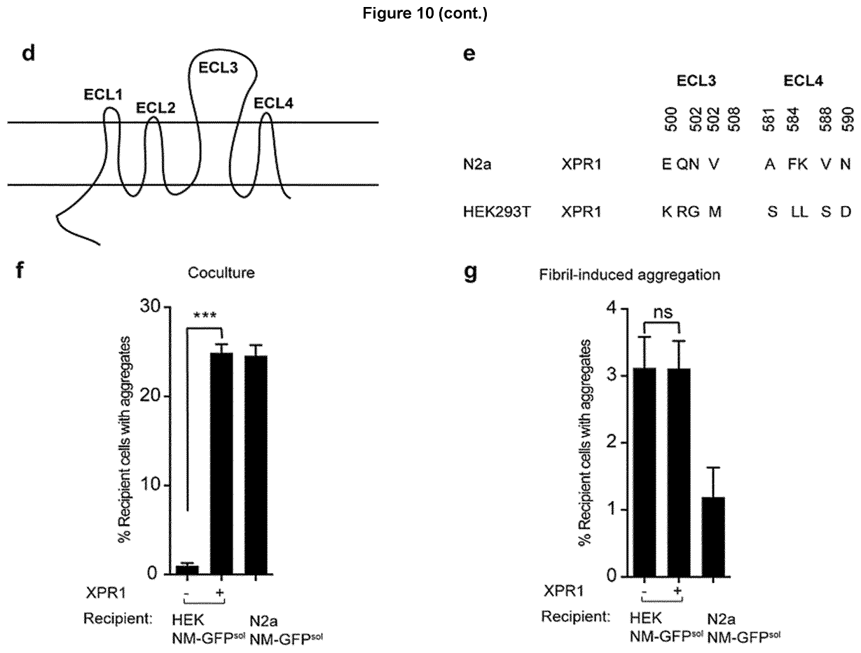

FIG. 10: Expression of the murine XPR1 receptor is required for efficient NM-GFP aggregate induction in recipient cells.
a. Alignment of MuERV Env polyprotein P10404 overexpressed in donor cell clone N2a NM-HA^{agg} s2E (P17) and the Env protein of a typical polytropic MuLV, MCF 247 (Yan et al., Retrovirology (2009), 6:87). Shown are the variable regions VRA, VRC and VRB in the surface domain SU that determine receptor usage by different X/P-MuLV subtypes. Identical amino acid residues are indicated by dots.
b, c. Murine XPR1 expression by recipient cells is required for efficient NM-GFP aggregate induction during coculture of donor and recipients, but not in fibril-induced NM-GFP aggregation. Recipient N2a NM-GFP^{sol} cells were transfected with two siRNAs against XPR1, one mCat-1 siRNA or a non-silencing siRNA control (mock). Two days later, these recipient cells were subsequently cocultured with untreated donor N2a s2E cells (P21), or exposed to 5 μM NM fibrils (monomer equivalent). Aggregate induction was monitored after 16 h. The number of recipient cells with aggregates transfected with non-silencing siRNA was set to 100%. Recipient cells transfected with siRNA against receptors that contained NM-GFP^{agg} were normalized to non-silencing siRNA control cells with NM-GFP^{agg}.
d. Transmembrane structure of XPR1. The receptor contains 4 extracellular loops (ECL 1-4) (Kozak et al., Retrovirology (2010), 7:101).
e. Polymorphic variants of xenotropic and polytropic (X/P-) MuLV receptor XPR1 in mouse N2a and human HEK cells. Shown are mismatches in the surface-exposed loops ECL 3 and 4. ECL3 and 4 are required for binding of X/P-MuLV (Kozak et al., Retrovirology (2010), 7:101).
f. Ectopic expression of the HA epitope-tagged XPR1 receptor variant of N2a cells in HEK NM-GFP^{sol} cells drastically increases NM-GFP aggregate induction when cells are cocultured with donor clone N2a NM-HA^{agg} s2E. Untransfected recipient N2a NM-GFP^{sol} served as positive controls. NM-GFP aggregate induction was measured 16 h post coculture.
g. Recipient HEK NM-GFP^{sol} cells with or without XRP1-HA or untransfected N2a NM-GFP^{sol} cells were exposed to in vitro formed NM fibrils (5 μM monomer equivalent). NM-GFP aggregate induction was measured 16 h post fibril addition (n=6, ***, p<0.001; one-way ANOVA).

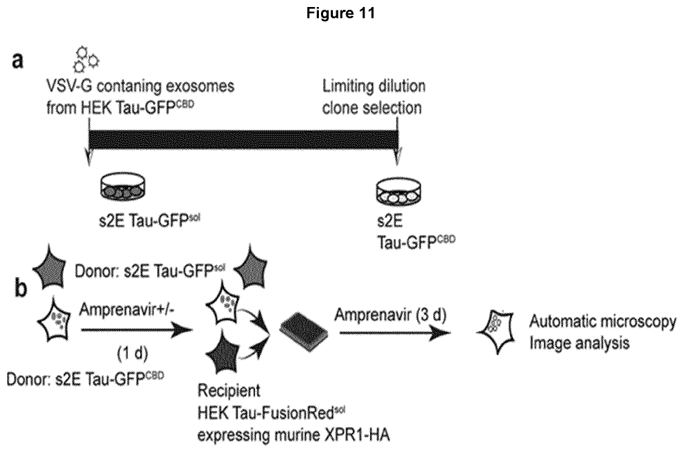

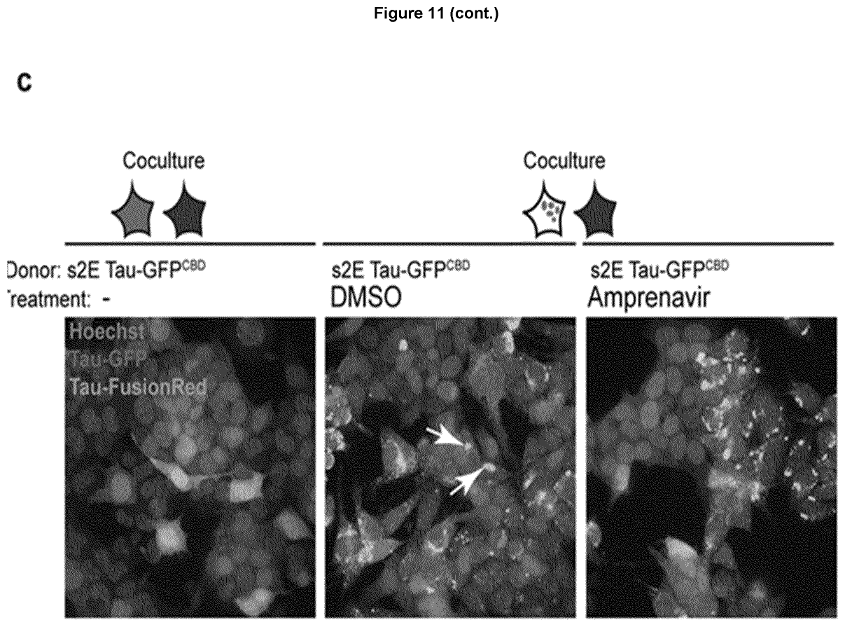

FIG. 11: Endogenous ERV increases Tau aggregate induction in recipient cells.
a. An N2a s2E cell clone (derived from cells transduced at P21) expressing soluble Tau-GFP (s2E Tau-GFP^{sol}) was exposed to VSV-G pseudotyped EVs isolated from conditioned medium of VSV-G transfected HEK Tau-GFP^{CBD} cells to induce Tau-GFP aggregates. Limiting dilution cloning was performed to isolate single cell clones that stably propagate Tau-GFP aggregates (s2E) Tau-GFP^{CBD}.
b. Experimental setup of Amprenavir treatment. N2a s2E Tau-GFP^{CBD} donor cells were exposed to 10 μM Amprenavir or DMSO for 1 d. Subsequently, donor cells were cocultured with recipient HEK Tau-FusionRed^{sol} XPR1-HA cells for 3 d in the presence of 10 μM Amprenavir or DMSO.
c. Tau aggregate induction in recipient HEK Tau-FusionRed^{sol} XPR1-HA cells following coculture with N2a s2E Tau-GFP^{CBD} was monitored via automated confocal microscopy. Coculture with control N2a s2E cells expressing soluble Tau (N2a s2E Tau-GFP^{sol}) did not lead to Tau aggregate formation in recipient cells. Amprenavir treatment of donor cells significantly decreased Tau-FusionRed aggregate induction in recipients. Arrows indicate induced Tau-FusionRed aggregates.

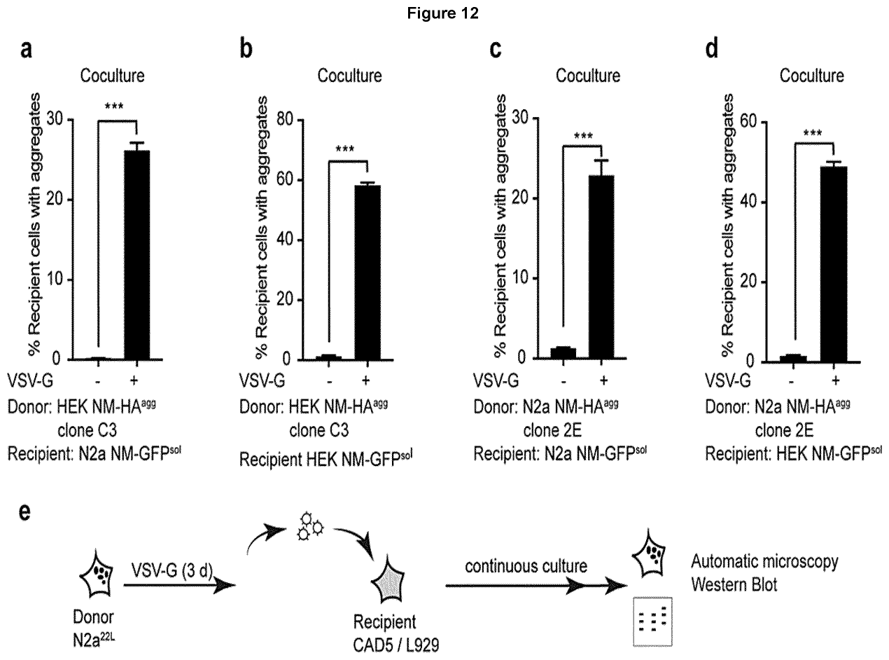

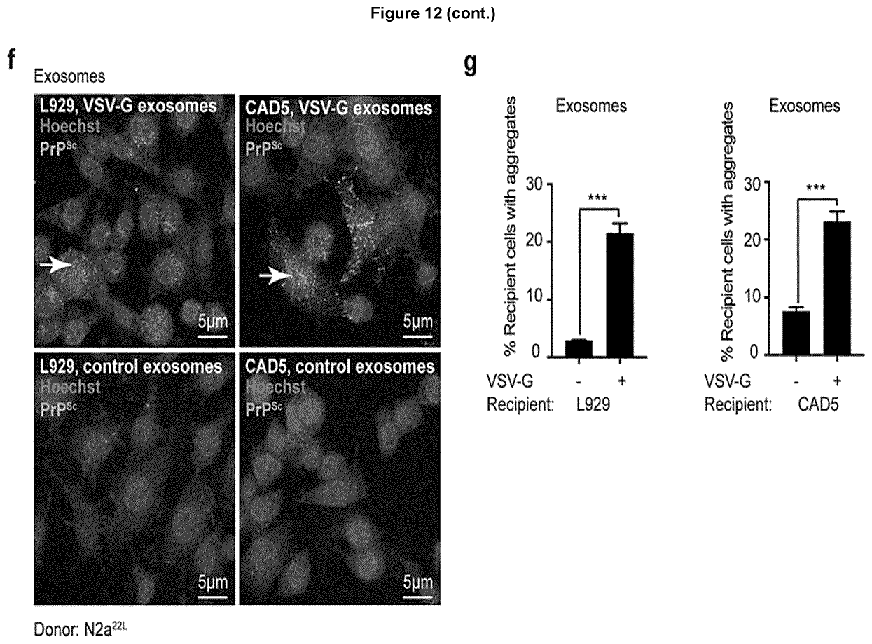

FIG. 12: Viral glycoproteins contribute to intercellular protein aggregate spreading.
a-d. Ectopic expression of VSV-G in donor cells. Donor clones HEK NM-HA^{agg} C3 and N2a NM-HA^{agg} 2E (precursor of clone s2E exhibiting low intercellular aggregate induction efficiency) were transiently transfected with a plasmid coding for VSV-G. As a control, cells were transfected with empty vector. Overexpression of VSV-G in donor clone HEK NM-HA^{agg} C3 increases the aggregate induction rate in cocultured N2a NM-GFP^{sol} (a) or HEK NM-GFP^{sol} recipient cells (b). Overexpression of VSV-G in donor clone N2a NM-HA^{agg} 2E increases the aggregate induction rate in cocultured recipient N2a NM-GFP^{sol} (c) or HEK NM-GFP^{sol} recipient cells (d) (n=6, ***, p<0.001; unpaired student t-test).
e. Workflow of the experiment. N2a cells persistently infected with TSE strain 22L (N2a^{22L}) cells were transfected with a VSV-G coding plasmid or empty vector control. Conditioned medium was harvested and CAD5 and L929 cells were exposed to isolated EVs derived from VSV-G expressing or control N2a^{22L} for 3 d. Subsequently, recipient cells were passaged 8 times to dilute out inoculum before accumulation of prions was monitored by immunofluorescence staining of guandidinium hydrochlorid- (GdnHCL-) unfolded PrP^{Sc} by confocal microscopy or proteinase K resistant PrP^{Sc} by Western blot.
f. Accumulation of PrP^{Sc} in recipient L929 and CAD5 cells was monitored via automated confocal microscopy following GdnHCl treatment and staining with anti-PrP mAb 4H11 (Ertmer et al., JBC (2004), 279:41918-41927). Arrowheads indicate PrP^{Sc} signal.
g. Percentage of recipient cells containing positive PrP^{Sc} puncta (n=6, p<0.001; unpaired student t test).

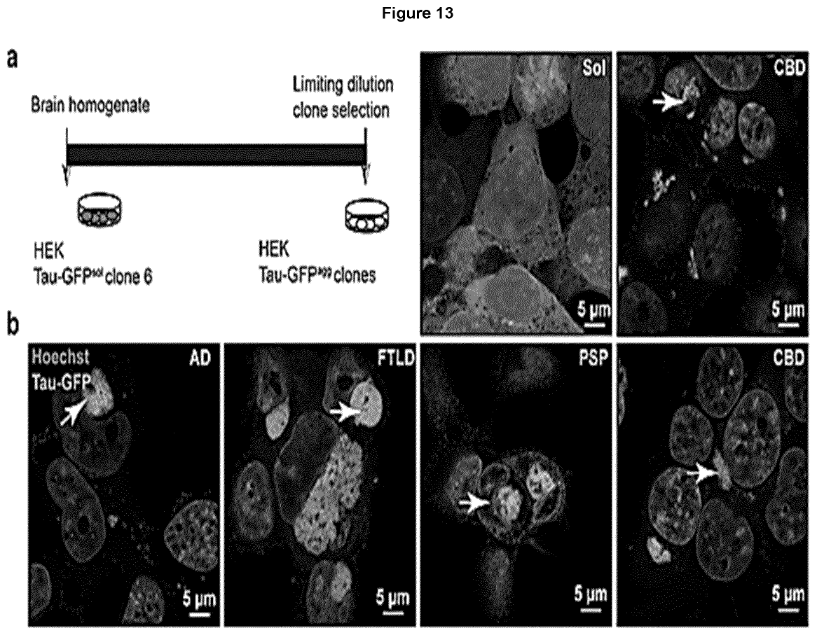

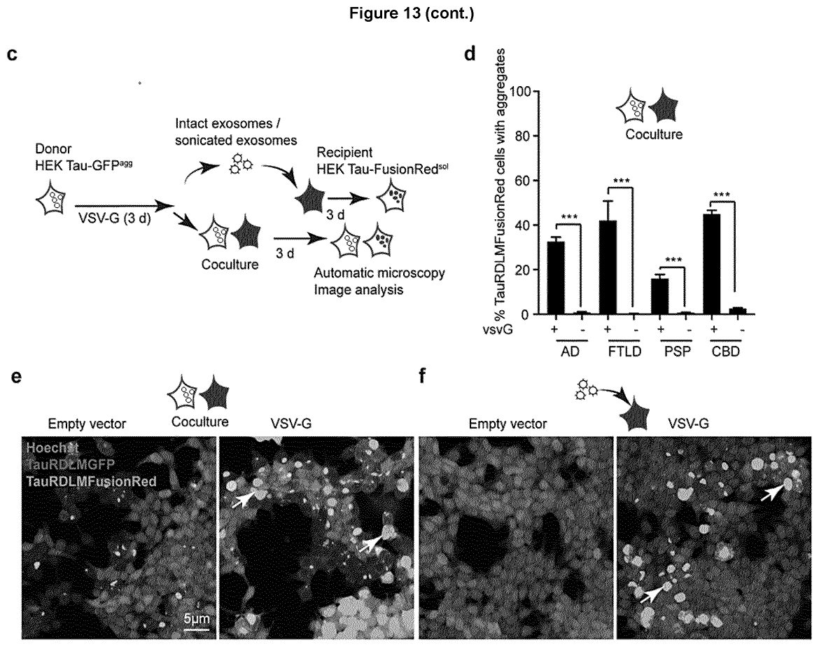

FIG. 13: Overexpression of VSV-G in HEK donor cells increases Tau aggregate induction in recipient cells.
a. A HEK cell clone stably expressing soluble Tau-GFP (HEK Tau-GFP^{sol}) was exposed to 1% brain homogenates from different tauopathy patients. Limiting dilution cloning was performed to isolate single cell clones that stably propagate Tau-GFP aggregates (HEK Tau-GFP^{AD}, Tau-GFP^{FTLD}, Tau-GFP^{PSP}, Tau-GFP^{CBD}).
b. Immunofluorescence staining of HEK Tau-GFP^{sol}, HEK Tau-GFP^{AD}, Tau-GFP^{FTLD}, Tau-GFP^{PSP} and Tau-GFP^{CBD} cells. Nuclei were stained with Hoechst. Representative, super resolution images were obtained using the LSM800 confocal microscope (Zeiss) with applied Airyscan detection. Arrows indicate Tau-GFP aggregates in different cell clones.
c. Workflow of the experiment. Different HEK Tau-GFP^{agg} clones were transfected with a plasmid coding for VSV-G or the empty vector as control. EVs were isolated from conditioned medium of donor cell populations. Recipient HEK cells stably expressing soluble Tau-FusionRed (HEK Tau-FusionRed^{sol}) were either cocultured with transfected donor cells or exposed to donor-derived EVs for 3 d.
d. Tau-FusionRed aggregation in recipient cells was monitored by automated confocal microscopy. Percentage of recipient cells with induced Tau-FusionRed aggregates following coculture with donors (n=6, ***, p<0.001; one-way ANOVA).
e, f. Tau aggregate induction in recipient HEK Tau-FusionRed^{sol} cells following coculture (e) or EV addition (f) was monitored by automated confocal microscopy. Arrows indicate induced Tau-FusionRed aggregates.

FIG. 14: Human endogenous retroviruses contribute to the spreading of protein aggregates.
a. Experimental workflow. A human breast cancer cell line MCF-7 (4) was engineered to stably express Tau-GFP and induced to propagate Tau aggregates by exposure to Alzheimer's disease brain homogenate (AD). A single cell clone was subsequently isolated and used in the experiment (MCF-7 Tau-GFPAD). This donor clone was treated with 2 μM 5-Aza-2-deoxycytidine (Aza) for 4 d to de-repress HERV expression. Pretreated donor cells were cocultured with HEK-Tau-FRsol cells.
b. Quantitative real-time PCR demonstrating increased HERV-W Env (Syncytin-1) expression in MCF-7 cells treated with Aza. Statistics: Unpaired Student's t-test (n=3).
c. Quantitative analysis of recipient cells with induced Tau-FR aggregates following coculture with DMSO-treated or Aza-treated donor cells. Statistics: Unpaired Student's t-test (n=6).
d. Syncytin-1 was downregulated in donor MCF-7 Tau-GFPAD cells by lentiviral shRNA transduction. Recipients were subsequently cocultured with transduced donors that had been passaged at least 5 times post transduction.
e. shRNA-mediated downregulation of Syncytin-1 mRNA in MCF-7 Tau-GFPAD cells assessed by quantitative real-time PCR. Statistics: Unpaired Student's t-test (n=3).
f. Quantitative analysis of recipient cells with induced Tau-FR aggregates following coculture with donors with decreased HERV-W Syncytin-1 expression. Statistics: Unpaired Student's t-test (n=6).

FIG. 15: Lopinavir treatment reduces intercellular protein aggregate spreading.
a. Melanoma cell line A375 (Oricchio E, Sciamanna I, Beraldi R, Tolstonog G V, Schumann G G, Spadafora C. 2007. Distinct roles for LINE-1 and HERV-K retroelements in cell proliferation, differentiation and tumor progression. Oncogene 26:4226-4233) was engineered to stably express Tau-GFP and subsequently exposed to AD brain homogenate to induce Tau aggregation. A clone stably propagating Tau-GFPagg was isolated and used for experiments. Donor A375 Tau-GFPAD cells were treated with 10 μM inhibitor Lopinavir to repress HERV-K protease for 3 d before EV were harvested. Donors or donor EV were then cultured with recipient HEK Tau-FRsol cells in the presence of 10 μM Lopinavir or DMSO for 3 d.
b. Quantitative analysis of recipient cells with Tau-FRagg upon coculture with donors. Statistical analysis was performed with unpaired Student's t-test (n=6).
c. Quantitative analysis of recipient cells with induced Tau-FRagg following exposure to donor-derived EV. Statistical analysis was performed with unpaired Student's t-test (n=3).

FIG. 16: HERV-W Env interactions with its receptors increase prion and Tau aggregate induction.
a. Experimental workflow. Donor HEK cells stably propagating aggregated NM-HA or Tau-GFPAD were transfected with plasmid coding for wildtype (WT) or mutated (MT) Myc epitope-tagged Syncytin-1 (Syn-Myc) and subsequently cocultured with recipient HEK cells expressing NM-GFPsol or Tau-FRsol, respectively.
b. Western blot analysis of donor clones transfected with plasmid coding for wildtype (WT) or mutant (MT) Syn-Myc.
c. Quantitative analysis of the percentage of recipient cells with induced aggregates upon coculture. Statistics were performed with one-way ANOVA (n=6).
d. Recipient cells were transfected with empty vector or vectors coding for receptors ASCT1/2 (genes: SLC1A4/5). Cells were cocultured with donor cells expressing Syn-Myc.
e. Quantitative analysis of the percentage of recipient cells with induced aggregates. Syn-Myc expressing donors were cocultured with recipient cells overexpressing the receptors (genes: SCL1A4/5) or transfected with pcDNA3.1(−) control. Statistics: Unpaired Student's t-test (n=6).
f. Expression of receptors was silenced by siRNA in recipient cells. Recipients were subsequently cocultured with donor cells expressing Syn-Myc.
g. Knock-down of SCL1A4 and SCL1A5 mRNAs by specific siRNAs. Statistics: Unpaired Student's t-test (n=3).
h. Knock-down of SCL1A4/5 in recipients decreases the percentage of recipient cells with induced aggregates. Statistics: Unpaired Student's t-test (n=6)
i. Donor HEK cells propagating NM-HAagg or Tau-GFPAD were transfected with empty plasmid or plasmid coding for Syn-Myc. EV were harvested 3 d later and added to recipient HEK NM-GFPsol or Tau-FRsol overexpressing SCL1A4/5, respectively.
j. Western blot demonstrating the presence of Syn-Myc in EV fraction of donor cells. Flotillin-1 and HSP70/72 served as EV markers.
k. Quantitative analysis of recipient cells with induced aggregates following exposure to EV from donor cells. Statistics: Unpaired Student's t-test (n=3).

The present invention is further illustrated by the following examples. Yet, the examples and specific embodiments described therein must not be construed as limiting the invention to such specific embodiments.

### EXAMPLES

### Example 1

Upregulation of Endogenous Retrovirus Increases Intercellular Protein Aggregate Induction

The prion domain NM of the Saccharomyces cerevisiae prion protein Sup35 stably expressed in the cytosol of mouse neuroblastoma N2a cells was induced to aggregate by exposure to amyloid fibrils of recombinant NM protein (Krammer et al., PNAS (2009), 106: 462-467). To study cellular mechanisms of protein aggregate spreading, subclone N2a s2E was used, selected by two rounds of limiting dilution cloning. This clone was selected due to its ability to potently induce NM aggregation in cells expressing soluble NM upon coculture and EV addition (Liu et al., mBio (2016), 7:00915-00916). Donor clone s2E was cocultured with recipient cell line N2a expressing soluble NM-GFP (NM-GFP^{sol}) and the percentage of recipient cells with induced NM-GFP aggregates was subsequently determined by automated microscopy (FIG. 1a). Surprisingly, it was found that aggregate induction in cocultured recipient cells was strongly increased when donor cells had been in culture over prolonged periods of time (FIG. 1b, left graph). EVs were isolated from conditioned medium of donor cell clone s2E and added to N2a NM-GFP^{sol} cells or primary cortical neurons ectopically expressing NM-GFP^{sol} (FIG. 1b, middle and right graph). Increased passage number also strongly increased EV-mediated NM aggregate induction in both N2a cells and primary cortical neurons (FIG. 1b). This effect was not due to increased EV secretion, as exosomal particle numbers did not change significantly over prolonged culture (FIG. 6a). Proteomic analyses of total cell lysates and exosomal fractions isolated from s2E donor cells passaged 7 or 16 times or passaged 6- or 15-times post cryoconservation, respectively, revealed a significant increase of endogenous retrovirus (ERV) gene products upon higher passage number (FIG. 6b, c). The presence of ERV proteins Env and Gag was confirmed by Western blot analyses (FIG. 1c) and the presence of env and gag mRNA qRT-PCR (FIG. 6d).

It was determined whether the highly efficient aggregate induction by coculture or by EVs from donor cells of later passage was associated with active endogenous retroviral particles present in the exosomal fraction. To this end, the reverse transcriptase (RT) activity of released particles from donor cell clone s2E (P16) was compared with N2a NM-HA^{agg} cell clones 1C and 3B that exhibit low aggregate induction rates in recipient cells (Hofmann et al., PNAS (2013), 10: 5951-5956; Liu et al., MBio (2016), 7). Only N2a NM-HA^{agg} clone s2E released particles with increasing RT activity upon prolonged culture (FIG. 6e). Infectivity of viral particles was tested on Hela cells and murine melan-a cells, a cell line permissive for endogenous retrovirus (Li et al., Int J Cancer (1998), 76: 430-436). Interestingly, viral particles released from donors were infectious to melan-a cells. Hela cells appeared refractory to infection, suggesting that functional receptors for the endogenous virus were absent in these cells (FIG. 60.

It was determined if endogenous retroviral particles or vesicles released by the donor cells contained infectious NM seeds. To separate EVs from viral particles, an Optiprep velocity gradient previously used to separate HIV-1 virions from non-viral extracellular vesicles was employed (Dettenhofer et al., J Virol (1999), 73: 1460-1467). Western blot analyses revealed the presence of NM-HA predominately in fractions that contained exosomal marker Alix (FIG. 1d). Viral proteins Gag and Env were distributed throughout the gradient, with highest levels found in Alix-positive fractions (fractions 2-6) that also contained highest levels of nanoparticles (FIG. 1e). However, electron microscopy demonstrated that fractions 9 and 10 contained membranous 80-100 nm spherical particles with an electron-dense core, characteristic of γ-retroviral particles (FIG. 10, while vesicles in fractions 2 and 3 exhibited a cup-shaped morphology, characteristic of EVs (FIG. 10. RT (Mn²⁺-dependent) activity was associated with viral fractions 8-11 containing only few EVs (FIG. 1e, g). Highest aggregate induction efficiency was associated with exosomal fractions, while the major viral particle fraction 10 failed to induce NM-GFP aggregation in recipient cells (FIG. 1h). This showed that increased NM aggregate seeding activity is associated with EVs isolated from conditioned medium of N2a s2E (P21) cells expressing endogenous retrovirus genes.

### Example 2

ERV Gene Products are Required for Intercellular Aggregate Induction Via EVs

Experiment 1 showed that upregulated ERV Env and Gag proteins in donor cells are associated with EVs and facilitate efficient EV-mediated aggregate transmission to recipient cells. To further investigate if EV-mediated NM aggregate induction depends on the fusogenic activity of Env, anti-HIV-1 drugs were screened for their effects on NM aggregate induction in coculture, as well on EV- and fibril-mediated NM aggregate induction in recipient cells (FIG. 7a). Strikingly, treatment of cocultures with HIV-1 protease inhibitors (particularly atazanavir and amprenavir) reduced the percentage of recipient cells with NM-GFP aggregates (FIG. 7b,c). By contrast, reverse transcriptase and integrase inhibitors as well as Hepatitis C virus (HCV) protease inhibitors had no effect on aggregate induction during coculture (FIG. 7d-f). Moreover, none of the compounds showed any effect on fibril- and EV-mediated aggregate induction in recipient cells (FIG. 8a-e). To test whether Amprenavir inhibited Env protein maturation in the donor cells and thus had no effect when only recipients were treated, donor cells were pre-treated with 10 μM Amprenavir for 3 days. Donors were subsequently cocultured with recipient cells in the absence of Amprenavir. Additionally, EVs isolated from Amprenavir-treated donors were tested for their aggregate induction efficiency in recipient cells (FIG. 9a). Western blot analyses of cell lysates and exosomal fractions from Amprenavir- or DMSO-treated donor cells demonstrated that Amprenavir efficiently inhibited Env protein maturation to TM p15E (FIG. 2a). Amprenavir treatment of donor cells significantly inhibited intercellular aggregate induction during coculture (FIG. 2b). Strikingly, Amprenavir treatment of donor cells also basically abolished EV-mediated aggregate induction in recipient cells (FIG. 2c), without affecting secreted particle numbers (FIG. 9b). These results demonstrate that maturation of ERV encoded gene products in donor cells or donor-derived EVs is essential for efficient aggregate induction in recipient cells.

Neutralization experiments with antibodies targeting Env protein revealed a dose-dependent reduction of NM-GFP aggregate positive recipient cells cocultured with donor cells (FIG. 9c) or exposed to EVs isolated from donor cells (FIG. 2d), confirming that Env proteins play a prominent role in intercellular aggregate transmission and induction. To silence MuERVs, s2E cells were transfected with three siRNAs targeting the specific overexpressed MuERVs Env P10404 or Gag A0A068F126 prior to coculture with recipient cells. Due to multiple integrations of ERVs, MuERV mRNA and proteins were only slightly reduced (FIG. 2e, g, FIG. 9d, e). Still, all siRNAs significantly decreased NM aggregate induction in N2a NM-GFP cells (FIG. 2f, h). These data suggest that both env- and gag-encoded components contribute to intercellular protein aggregate induction.

It was evaluated whether treatment with DNA methyl transferase inhibitors 5-Azacytidine (Aza) and Decitabine (Dec), capable of erasing epigenetic marks and thereby inducing ERV expression (Chiappinelli et al., Cell (2015), 162:974-986; Ramos et al., Epigenetics Chromatin (2015), 8:11) would result in increased intercellular aggregate induction efficiency. Clone s2E with low MuERVs expression (P1) was chosen for the experiment. Indeed, treatment of the cell clone s2E for three days with the epigenetic drugs and subsequent culture in the absence of the drugs for 5 days resulted in increased expression of total env and gag mRNA (FIG. 94f-h) and MuERV proteins (FIG. 2i). Importantly, both drugs also significantly increased NM aggregate induction in recipient cells when cocultured with pretreated donors (FIG. 2j). In a reverse experiment, we aimed to increase DNA methylation by 1-methionine, betaine or choline chloride treatment of donor cells s2E (P21, high MuERV expression) to reduce MuERV expression. Pretreatment of donors decreased total env and gag mRNA (FIG. 9i-k) and MuERV proteins (FIG. 2k). Further, treatment also decreased NM-GFP aggregate induction when donors were subsequently cocultured with recipient cells (FIG. 2l). We conclude that epigenetic regulation of ERV expression affects intercellular aggregate transmission.

Alignment analysis showed substantial similarity of P10404 with MCF247, a polytropic MuLV (FIG. 10a). Silencing of XPR1 but not mCat-1 (FIG. 2m) in recipient cells strongly reduced NM aggregate induction during coculture (FIG. 10b) and when recipient cells were exposed to EVs (FIG. 2n). By contrast, silencing of both receptors in recipient cells had no effect on NM aggregate induction by recombinant NM fibrils, demonstrating that NM aggregate uptake was mediated by EV-receptor contact and not direct receptor-independent internalization of released free (non-EV) NM seeds (FIG. 10c).

XPR1 is a multiple-membrane spanning receptor with eight putative transmembrane domains and four extracellular loops (ECL) (Battini et al., PNAS (1999), 96: 1385-1390). Polymorphisms in ECL 3 and 4 affect the entry of certain X/P-MuLV subtypes. Analysis of XPR1 of N2a cells demonstrated that its Env recognition domain differed at 9 residues within ECL 3 and 4 from XPR1 expressed by HEK cells (FIG. 10d, e). It was tested if direct cell contact or EVs derived from N2a donor clone s2E can also induce NM-GFP aggregation in human HEK cells. HEK cells were refractory to intercellular aggregate induction via coculture with N2a clone s2E and exposure to s2E-derived EVs, consistent with the idea that they express a non-permissive XPR1 receptor. However, expression of the N2a polymorphic XPR1 variant in HEK cells (FIG. 2o) conferred susceptibility via coculture (FIG. 10f) or EVs derived from N2a donor clone s2E (FIG. 2p). Expression of the murine polymorphic XPR1 variant had no effect on aggregate induction by recombinant NM fibrils (FIG. 10g). Thus, it was shown that efficient NM aggregate induction via coculture or EVs depends on specific Env/receptor interactions.

To examine this effect on Tau aggregate spreading, an N2a s2E cell clone stably propagating aggregated Tau-GFP^{CBD} was produced (FIG. 11a). To this end, N2a clone s2E (P21) was transduced with lentivirus coding for Tau-GFP. Cells were subsequently exposed to VSV-G pseudotyped EVs from HEK Tau-GFP^{CBD} cells (see Example 3) and individual clones were isolated that exhibited stable Tau-GFP aggregation. Cells and EVs were analyzed for the presence of aggregated Tau-GFP. Pronase-resistant Tau-GFP was found in both cell lysate and EVs (FIG. 2q). To inhibit MuERV gene product maturation, donor cells were treated with Amprenavir for three days. Donor cells and donor cell-derived EVs were subsequently exposed for three days to recipient HEK cells expressing soluble Tau-FusionRed and the murine XPR1 receptor variant required for MuERV Env interaction. Due to this long incubation time, the coculture and EV induction assays were performed in the presence of Amprenavir (FIG. 11b). Tau aggregate induction in cocultured recipient cells was low in the absence of the MuERV receptor variant XPR1, but was strongly enhanced when cells ectopically expressed murine XPR1. Importantly, inhibition of MuERV protease decreased Tau-FusionRed aggregate induction in Amprenavir-treated cocultures. Likewise, Amprenavir-treatment of EVs also decreased Tau-FusionRed aggregate induction in murine XPR1 expressing recipient cells (FIG. 2r) (FIG. 11c). Neutralization assays were performed using Env-specific antibodies to assess if Tau aggregate induction was dependent on the interaction of Env with its cognate receptor. Donor cells or EVs from donor cells were preincubated with antibodies for 1 h prior to adding them to recipient cells. As observed for the induction of NM aggregates, antibodies led to a dose-dependent reduction of the percentage of recipient cells with induced Tau aggregates in our coculture assay. Similar effects were observed when recipient cells were exposed to EVs preincubated with antibody (FIG. 2s), We conclude that upregulation of MuERV also contributes to the intercellular spreading of Tau aggregates.

### Example 3

Fusogenic Viral Glycoproteins Drastically Increase Intercellular Transmission of Proteinaceous Seeds

The foregoing experiments demonstrated that upregulation of endogenous retroviruses drastically increased intercellular aggregate transmission via receptor-ligand interactions. It was tested whether the expression of unrelated viral glycoproteins that target specific membrane proteins on recipient cells might also be able to increase intercellular aggregate transmission and induction. The vesicular stomatitis virus glycoprotein VSV-G is routinely used to pseudotype viral particles for efficient uptake by a broad spectrum of target cells expressing the LDL receptor. Recently, VSV-G has been successfully used to pseudotype EVs for enhanced protein delivery to recipient cells (Meyer et al., Int J Nanonmed (2017), 12: 3153-3170). It was tested if ectopic VSV-G expression also increased intercellular spreading of proteinaceous seeds. The N2a NM-HA^{agg} clone 2E (precursor clone of s2E) and HEK NM-HA^{agg} clone C3, two cell lines that are characterized by poor NM aggregate induction rates when cocultured with recipient cells, were transfected with plasmids coding for VSV-G. The presence of VSV-G on EVs isolated from both donor cell clones (FIG. 3a) strongly increased intercellular aggregate induction when EVs were added to HEK cells expressing NM-GFP^{sol} (FIG. 3b). Sonication of EV fractions abolished aggregate induction, arguing that intact EVs were required (FIG. 3b). Increased aggregate induction was also observed when VSV-G expressing donor cell clones were cocultured with either N2a or HEK NM-GFP^{sol} recipient cells (FIG. 12a-d). It was thus shown that viral fusogenic proteins of different origin expressed by donor cells can strongly increase intercellular protein aggregate transmission.

It was then tested if viral glycoproteins could also promote spreading of pathogenic protein aggregates between cells. Thus, the effect of VSV-G expression on the intercellular spreading of transmissible spongiform encephalopathy (TSE) agents was evaluated. TSE agents, the so far only bona fide mammalian prions, are composed of misfolded cellular prion protein PrP. The conversion of cellular (PrP^{C}), a protein tethered to the cell membrane by a glycosylphosphatidyl-anchor, into its infectious aggregated isoform (PrP^{Sc}), occurs on the cell surface or along the endocytic pathway. It has previously been shown that N2a cells release prion infectivity associated with EVs. N2a cells persistently infected with TSE strain 22L (N2a^{22L}) were transiently transfected with control plasmid or a plasmid coding for VSV-G. EVs were isolated from medium of transfected cells containing VSV-G (FIG. 3c). Murine fibroblast cell line L929 (Wolf et al., J Virol (2015), 89: 9853-9864) and CAD5 cells (Mahal et al., PNAS (2007), 104: 20908-20913), two cell lines highly permissive to TSE strain 22L, were then exposed to EVs from VSV-G transfected N2a^{22L} donor cells for three days. Treated recipient L929 and CAD5 cells were subsequently cultured in the absence of EVs for more than 8 passages and tested for the formation of PrP^{Sc} by Western blot and confocal microscopy (FIG. 12e). VSV-G expression drastically increased the number of cells containing PrP^{Sc} aggregates, as revealed by confocal microscopy (FIG. 12f, g) and also strongly increased total PrP^{Sc}, as determined by proteinase K treatment of cell lysates followed by Western blot analysis (FIG. 3d).

We further tested if VSV-G expression also increased the intercellular transmission of Tau aggregates and subsequent induction of Tau aggregation in a reporter cell line. To this end, we established a Tau cell model that had been described previously by Diamond and coworkers (Sanders et al., Neuron (2014), 82:1271-1288). HEK cells were engineered to stably express the aggregation competent Tau core spanning amino acid residues 244-372 with two point mutations P301L/V337M fused to GFP (hereafter termed Tau-GFP). Cells were exposed to brain homogenates from patients who had suffered from Alzheimer's disease (AD), cortical basal degeneration (CBD), progressive supranuclear palsy (PSP) or frontotemporal lobar degeneration (FTLD). Upon limiting dilution cloning, cell clones HEK Tau-GFP^{AD}, Tau-GFP^{FTLD} Tau-GFP^{PSP} and Tau-GFP^{CBD} stably producing Tau aggregates were established (FIG. 13a, b). Cell lysates and exosomal fractions of individual Tau cell lines were subjected to pronase treatment to reveal differential sensitivities to proteolysis. All different tauopathy patient seeds induced Tau-GFP aggregation, revealed by the resistance to pronase treatment. Interestingly, clones were associated with different pronase-resistant patterns, in line with different pronase-resistant Tau aggregate patterns induced by different tauopathy seeds (FIG. 3e, f). Protease-resistant Tau-GFP was also found in EV fractions isolated from conditioned medium of HEK Tau-GFP^{AD}, Tau-GFP^{FTLD} Tau-GFP^{PSP} and Tau-GFP^{CBD} clones (FIG. 3g, h). To pseudotype EVs, VSV-G was transiently expressed in individual clones, and cells were subsequently cocultured with recipient HEK cells expressing the soluble Tau aggregation domain fused to FusionRed (hereafter termed Tau-FusionRed) for three days (FIG. 13c). Alternatively, recipient cells were exposed to EVs isolated from VSV-G expressing donor clones for three days. Expression of VSV-G by donor cells drastically increased induction of Tau-FusionRed aggregates in recipient cells via coculture (FIG. 13d, e). Likewise, VSV-G pseudotyped EVs (FIG. 3i) also drastically increased Tau aggregate induction in recipients (FIG. 3j, FIG. 130. Furthermore, VSV-G pseudotyped EVs isolated from four donor clones induced detectable Tau-GFP aggregates (FIG. 3k) in recipient HEK cells expressing soluble Tau-GFP cells. It was thus shown that viral fusogenic proteins can pseudotype EVs and mediate efficient intercellular aggregate transmission and induction of pathogenic proteins.

### Example 4

Human Endogenous Retrovirus Proteins Contribute to Intercellular Protein Aggregate Transmission

To examine the effect of endogenous HERVs on protein aggregate transmission, T47D human breast tumor cells which exhibit highly increased HERV-K expression upon stimulation with female steroid hormones were used (Ono et al., J Virol (1987), 61: 2059-2062). It was first tested if HERV-K proteins contribute to the intercellular transmission of the model prion NM described above. To this end, a T47D cell clone stably expressing soluble NM-GFP (T47D NM-GFP^{sol}) was exposed to in vitro formed NM fibrils for one day. The resulting T47D NM-GFP^{agg} bulk cell population was cocultured with recipient HEK NM-mCherry^{sol} cells in the presence or absence of Amprenavir, shown to also be effective against HERV-K (Tyagi et al., Retrovirology (2017), 14:21) (FIG. 4a). While the receptor for HERV-K is unknown, HEK cells efficiently bind and internalize HERV-K Env pseudotyped HIV viruses (Lee et al., PLoS Pathog (2007), 3: e10; Kramer et al., Virology (2016), 487: 121-128). Importantly, Amprenavir treatment of cocultures reduced the number of recipient cells with induced NM-mCherry aggregates compared to the DMSO control, suggesting that HERV protein processing was required for efficient intercellular aggregate transmission and subsequent induction of new aggregates in recipient cells (FIG. 4b).

To examine the effect of HERV-K proteins on Tau aggregate spreading, a T47D donor cell line stably expressing Tau-GFP^{sol} was generated. As exposure of T47D cells to brain homogenates resulted in poor Tau-GFP aggregation (less than 0.5% of recipient cells), VSV-G pseudotyped EVs derived from HEK Tau-GFP^{AD} and Tau-GFP^{FTLD} cells (see FIG. 3k) were used to induce aggregation of Tau-GFP in T47D cells. T47D bulk populations with Tau-GFP^{AD} or Tau-GFP^{FTLD} were pretreated for one day with 10 μM Amprenavir or DMSO, and cells were subsequently cocultured with recipient HEK Tau-FusionRed^{sol} cells in the presence or absence of Amprenavir. A significant decrease in Tau aggregate induction in recipient cells was observed when cells were treated with the viral protease inhibitor (FIG. 4c, d). These results showed that expression of mature endogenous HERV proteins contributes to intercellular protein aggregate transmission. Importantly, it was shown that intercellular spreading of both non-pathogenic and pathogenic protein aggregates in HERV expressing cells can be reduced by drugs that prevent HERV protein maturation.

Elevated Transcripts of Distinct HERV Families in Postmortem Brains from Different Tauopathy Patients

The foregoing experiments indicated that murine and human ERV proteins expressed by donor cells facilitate efficient cell-to-cell and EV-mediated spreading of proteopathic seeds from donor to recipient cells. To test if HERV env expression is upregulated in tauopathies, quantitative real-time PCR was performed using predesigned primer sets against env sequences of nine HERV family members (de Parseval et al., J Virol (2003), 77:10414-10422; Strissel et al., Oncotarget (2012), 3:1204-1219). These primer sets locate in the coding elements that detect the expression of the coding copies of the env genes. It was found that transcripts of distinct HERVs were elevated in postmortem brain samples from individuals suffering from different tauopathies (FIG. 5). HERV-W env expression was highly upregulated in all three postmortem AD brains. By contrast, HERV-FRD, HERV-H and HERV-R(b) env transcripts were increased in all CBD brains tested. HERV-K and HERV-F(c)1 env expression was upregulated in three PSP patient samples.

Methods

Human Brain Samples

Frozen brain tissue samples from neuropathologically confirmed cases of AD, CBD, PSP and controls were provided by Brain Bank Tubingen.

Ethics Statement

For all the patient sample experiments, the ethical approval has been obtained from ‘Medizinische Fakultät Ethik-Kommission, Rheinische Friedrich-Wilhelms-Universität, Project no. 236/18(2018)’.

Molecular Cloning

For the expression of lentiviral constructs Tau-GFP and Tau-FusionRed, the four repeat domain 4RN1 of human Tau (amino acid residues 244 to 372) containing the mutations P301L and V337M was fused aminoterminally to GFP or FusionRed (Evrogen) with an 18-amino acid flexible linker (EFCSRRYRGPGIHRSPTA), as described previously (Woerman et al., PNAS (2016), 113:E8187-E8196). Coding regions were cloned into the lentiviral vector pRRL.sin.PPT.hCMV.Wpre via BamHI and SalI (Hofmann et al., PNAS (2013), 10: 5951-5956). Murine and human receptor XPR1 were amplified from cDNA of N2a or HEK cells, respectively. The coding region of murine XPR1 tagged aminoterminally with a hemagglutinin epitope (HA) was cloned into a PiggyBac expression vector PB510B-1 (System Biosciences) using XbaI and NotI restriction sites.

Cell Lines

N2a, Hela, L929, CAD5 and HEK293T cells are from ATCC and were cultured in Opti-MEM (Gibco) supplemented with glutamine, 10% (v/v) fetal bovine serum (FCS) (PAN-Biotech GmbH) and antibiotics. Melan-a cells are from Wellcome Trust Functional Genomics Cell Bank and were cultured in RPMI 1640 (Gibco) with 2 mM glutamine, 10% FCS, antibiotics and 200 nM 12-0-tetradecanoyl phorbol acetate PMA and incubated at 37° C. and 10% CO₂. T47D cells were cultured in DMEM (Gibco) supplemented with 2 mM Glutamine and 10 (v/v) FCS. Cells were incubated at 37° C. and 5% CO₂. The total numbers of viable cells and the viability of cells were determined using the Vi-VELL™XR Cell Viability Analyzer (Beckman Coulter).

Isolation of Cortical Neurons

Preparation of cortical neurons was performed using postnatal day 13 SWISS pups as described previously (Hofmann et al., PNAS (2013), 10: 5951-5956). Neurons were transduced with lentivirus 2 days post preparation on 96 well plates or Sarstedt 8 slice chambers. After 2 days, EVs were added and neurons were incubated for 2 days. Subsequently, neurons were fixed for microscopy and imaging analysis.

Production and Transduction with Lentiviral Particles

HEK293T cells were cotransfected with plasmids pRSV-Rev, pMD2.VSV-G, pMDI.g/pRRE (all plasmids were published in Dull T, Zufferey R, Kelly M, Mandel R J, Nguyen M, Trono D, Naldini L A third-generation lentivirus vector with a conditional packaging system. J Virol. 1998 November 72(11):8463-71), and pRRI.sin.PPT.hCMV.Wpre (plasmid published in Follenzi, A. and L. Naldini (2002) HIV-based vectors. Preparation and use. Methods in molecular medicine 69: 259-274) containing Tau-GFP/FusionRed. Supernatants were harvested 30 and 54 h later and concentrated using PEG according to published protocols (Follenzi et al., Methods Mol Med (2002), 69:259-274). Cell lines and primary neurons were transduced with lentivirus, and stable cell clones expressing Tau-GFP/-FusionRed were produced by limiting dilution cloning (Krammer et al., PNAS (2009), 106:462-467).

EV Isolation

To prepare EV-depleted medium, FCS was ultracentrifuged at 100,000×g for 20 h at 4° C. Medium supplemented with the EV-depleted FCS and antibiotics was subsequently filtered through 0.22 μM and a 0.1 μM filter-sterilization devices (Millipore). For EV isolation, 2-4×10⁶ cells were seeded in T175 flasks in 35 ml EV-depleted medium to reach confluence after 3 days. Cells and cell debris were pelleted by differential centrifugation (300×g, 10 min; 2,000×g, 20 min; 16,000×g, 30 min, 4° C.). The remaining supernatant (conditioned medium) was subjected to ultracentrifugation at 100,000×g for 1 h at 4° C. using rotors Ti45 or SW32Ti (Beckman Coulter). The pellet was rinsed in PBS and spun again using rotor SW55Ti at 100,000×g for 1 h at 4° C.

Aggregate Induction Assay

Recipient cells were cultured on CellCarrier-96 plates or 384 black microplates (PerkinElmer) at appropriate cell numbers for 1 h, and then treated with 5-10 μl of prepared samples (isolated EVs or recombinant NM fibrils). For aggregate induction by coculture, recipient and donor cells were mixed at different ratios based on the population doubling time of donor and recipient cells, and a total of 10⁴ cells/per well was plated. After additional incubation for 16 h or 72 h (NM or Tau, respectively), cells were fixed in 4% paraformaldehyde and nuclei were counterstained with 4 μM Hoechst for 15 min. Cells were imaged with the automated confocal microscope CellVoyager CV6000 (Yokogawa Inc.) using a 20× or 40× objective. Maximum intensity projections were generated from Z-stacks. Images from 16 fields per well were taken. On average, a total of 3-4×10⁴ cells per well and at least 3 wells per treatment were analyzed.

Sample Preparation for Mass Spectrometry

Cell pellets from five s2E cell culture replicates, and six replicates of EV pellets harvested from conditioned medium of s2E cells at passages 7 and 16 were collected for a quantitative proteomics analysis. Cell pellets were lysed in 150 μL SDT buffer (4% SDS (w/v), 100 mM Tris/HCl pH 7.6, 0.1 M DTT) by homogenization with a dounce tissue grinder and heated for 3 min at 95° C. Samples were sonicated 5 times for 30 s with intermediate cooling using a vialtweeter sonifier (amplitude 100%, duty cycle 50%; Hielscher, Germany). EV pellets were lysed in 100 μL STET lysis buffer (150 mM NaCl, 50 mM TrisHCl pH 7.5, 2 mM EDTA, 1% Triton X-100) on ice for 30 min with intermediate vortexing. Cell debris was removed by centrifugation at 16,000×g for 5 min. The protein concentration was determined using the colorimetric 660 nm assay (Thermo Fisher Scientific). For cell lysates, the assay solution was supplemented with the ionic detergent compatibility reagent (Thermo Fisher Scientific). A protein amount of 30 μg per sample for cell lysates and 10 μg for EV lysates was subjected to proteolytic digestion using the filter aided sample preparation (FASP) protocol (Wisniewski et al., Nat Methods (2009), 6:359-362) with 30 kDa Vivacon spin filters (Sartorius, Germany). Proteolytic peptides were desalted by stop and go extraction (STAGE) with C18 tips (Rappsilber et al., Anal Chem (2003), 75:663-670). The purified peptides were dried by vacuum centrifugation. Peptides from cell lysates and EV samples were dissolved in 40 or 20 μL of 0.1% formic acid, respectively.

LC-MS/MS Analyses

Samples were analyzed by LC-MS/MS for relative label free protein quantification. A peptide amount of approximately 1 μg per sample was separated on a nanoLC system (EASY-nLC 1000, Proxeon—part of Thermo Fisher Scientific) using in-house packed C18 columns (50 cm or 30 cm×75 μm ID, ReproSil-Pur 120 C18-AQ, 1.9 μm, Dr. Maisch GmbH, Germany) with a binary gradient of water (A) and acetonitrile (B) containing 0.1% formic acid at 50° C. column temperature and a flow rate of 250 nl/min. Peptides from cell lysates were separated on a 50 cm column using a gradient of 250 min length, whereas a 183 min gradient on a 30 cm column was used for peptides from EV samples (250 min. gradient: 0 min., 2% B; 5 min., 5% B; 185 min., 25% B; 230 min., 35% B; 250 min., 60% B; 183 min. gradient: 0 min., 2 B; 3:30 min., 5% B; 137:30 min., 25% B; 168:30 min., 35% B; 182:30 min., 60% B). The nanoLC was coupled online via a nanospray flex ion source (Proxeon—part of Thermo Fisher Scientific) equipped with a PRSO-V2 column oven (Sonation, Germany) to a Q-Exactive mass spectrometer (Thermo Fisher Scientific). Full MS spectra were acquired at a resolution of 70,000. The top 10 peptide ions were chosen for Higher-energy C-trap Dissociation (HCD) with a normalized collision energy of 25%. Fragment ion spectra were acquired at a resolution of 17,500. A dynamic exclusion of 120 s was used for peptide fragmentation.

Data Analysis and Label Free Quantification

The raw data was analyzed by the software Maxquant (maxquant.org, Max-Planck Institute Munich) version and 1.5.5.1 (Cox et al., Mol Cell Proteomics (2014), 13:2513-2526). The MS data was searched against a fasta database of Mus musculus from UniProt including also non-reviewed entries supplemented with databases of lentiviruses and murine leukemia viruses (download: Dec. 9, 2017, 52041+712+43 entries). Trypsin was defined as protease. Two missed cleavages were allowed for the database search. The option first search was used to recalibrate the peptide masses within a window of 20 ppm. For the main search, peptide and peptide fragment mass tolerances were set to 4.5 and 20 ppm, respectively. Carbamidomethylation of cysteine was defined as static modification. Acetylation of the protein N-term as well as oxidation of methionine were set as variable modifications. The false discovery rate for both peptides and proteins was adjusted to less than 1%. Label free quantification (LFQ) of proteins required at least two ratio counts of razor peptides. Only unique and razor peptides were used for quantification.

The LFQ values were log₂ transformed and a two sided Student's t-test was used to evaluate statistically significant changed abundance of proteins between cell lysates from passages 16 and 7 as well as EV lysates from passages 15 and 6. A p-value less than 5% was set as significance threshold. Additionally, a permutation based false discovery rate estimation was used to account for multiple hypotheses (Tusher et al., PNAS (2001), 98:5116-5121).

OptiPrep Density Gradient

For separating EVs and virus, the discontinuous iodixanol gradient in 1.2% increments ranging from 6 to 18% were prepared as previously described (Dettenhofer et al., J Virol (1999), 73:1460-1467). The 100,000×g pellet from 1050 ml culture supernatant (30 T175 flasks) was resuspended in 1 ml PBS and overlaid onto the gradient. The gradient was subjected to high-speed centrifugation at 100,000×g for 2 h at 4° C. using a SW41Ti rotor (Beckman Coulter). 12 fractions of 1 ml each were collected from the top of the gradient, diluted with PBS in 5 ml, and centrifuged at 100,000×g for 1 h at 4° C. The pelleted fractions were resuspended in 100 μl PBS, and then used for further experiments. The reverse transcriptase activity of the viruses was measured by using a colorimetric reverse transcriptase assay (Roche).

Determination of Extracellular Vesicles Size and Number

ZetaView PMX 110-SZ-488 Nano Particle Tracking Analyzer (Particle Metrix GmbH) was used to determine the size and number of isolated extracellular vesicles. The instrument captures the movement of extracellular particles by utilizing a laser scattering microscope combined with a video camera. For each measurement, the video data is calculated by the instrument, resulting in a velocity and size distribution of the particles. For nanoparticle tracking analysis, the Brownian motion of the vesicles from each sample was followed at 22° C. with properly adjusted equal shutter and gain. At least six individual measurements of 11 subvolumes (positions) within the measurement cell and around 2200 traced particles in each measurement were detected for each sample.

Electron Microscopy (EM)

EM imaging of extracellular vesicle preparations was performed as previously described (Thery et al., Curr Protoc Cell Biol (2006), Chapter3:Unit3 22). Briefly, the 100,000×g pellets from conditioned medium were fixed in 2% paraformaldehyde, loaded on glow discharged Formvar/carbon-coated EM grids (Plano GmbH), contrasted in uranyl-oxalat (pH 7) for 5 min and embedded in uranyl-methylcellulose for 5 min. Samples were examined using a JEOL JEM-2200FS transmission electron microscope at 200 kV (JEOL).

Infectivity Assay

The infectivity assay was performed as previously described (Pothlichet et al., Int J Cancer (2006), 119:815-822). Briefly, melan-a cells were exposed to conditioned medium from different cell clones at either low or high passsages in the presence of 4 μg polybrene/ml for 24 h. The medium was then replaced with normal culture medium. After five days, cells were lysed for western blot analysis of retroviral Env and Gag proteins.

Drug Treatments

The treatment of cells with Amprenavir (10 μM; Santa Cruz) and DMSO was performed for 72 h in EV-depleted medium in T175 flasks. Afterwards, the total numbers of viable cells and the viability upon drug treatment were determined using the Vi-VELL™ XR Cell Viability Analyzer (Beckman Coulter). EVs were isolated from the conditioned medium via ultracentrifugation and processed for the aggregate induction assay as described above. NM aggregate induction by coculture of donor and recipient cells or by exposure of recipient cells to donor-derived EVs was performed in the absence of the drugs. For coculture and EV treatment of recipient Tau-FusonRed cells, donor s2E P21 or T47D cells with Tau-GFP aggregates were pretreated as above. Isolated EVs or pretreated donor cells were then incubated with recipient cells in the presence of compounds for 72 h.

To inhibit methyltransferases, s2E P1 donor cells were treated for three days with methyltransferase inhibitors 5-Azacytidine (Aza) 200 nM, Decitabine (Dec) 100 nM or DMSO as solvent control. Subsequently, the cells were cultured in the absence of the drugs for 5 days. Pre-treated donor cells were subsequently cocultured with recipient cells as described above to monitor aggregate induction efficiency in recipient N2a NM-GFP^{sol} cells. Cell lysates of donor cells were also analyzed for MuERV Env and Gag expression levels by western blot. To increase DNA methylation, the s2E donor clone (P21) was treated with methyl group donors L-methionine (L-M) 80 mM, Betaine (B) 80 mM, Choline chloride (CC) 20 mM or medium control for 6 days. MuERV Env and Gag protein levels were analyzed by western blot. Subsequently, cells were cocultured with recipient cells for 16 h. The percentage of aggregate containing recipient cells was compared to the percentage of aggregate bearing recipients cocultured with solvent-treated donors.

Neutralization Assay

To block MuLVs Env on the surface of the donor cell clones s2E and s2E Tau-GFP^{CBD} and on secreted EVs, mAb83A25, reactive against a broad range MuLVs (Evans et al., J Virol (1990), 64:6176-6183) was incubated with either EVs or donor cells in serial dilutions for 1 h at 37° C. with rotation at 20 rpm. Donor cells were subsequently mixed with recipient cells for 16 h. Alternatively, antibody-treated and untreated EVs were added to recipient cells for 16 h (NM) or 3 days (Tau) incubation time.

Transfection of siRNAs and Plasmids

To transiently knock-down the upregulated specific MuLV Env and Gag genes in s2E clones, custom-designed Silencer select siRNAs (Thermo Fisher Scientific) against AA037244.2 (env) and AID54952 (gag) were used. Pre-designed siRNAs against murine XPR1 and mCat-1 genes were used to knock-down genes coding for putative receptors. For transfection, 2-4×10⁵ cells/well were seeded on 6 well plates. The next day, 30 nM siRNA or plasmid DNA was transfected using Lipofectamine RNAiMAX or Lipofectamine2000 transfection reagent, respectively, according to the manufacturer's instructions (Thermo Fisher Scientific). After 2 days, transfected cells were harvested for aggregate induction assays and qRT-PCR, western blot analysis.

PK Treatment for Detection of PrP^{Sc}

Cells from one well of 6 well plate were lysed in 1 ml lysis buffer. 900 μl of lysates were digested with 20 μg/ml proteinase K (PK) at 37° C. for 30 min for PrP^{Sc} detection. Proteolysis was terminated by adding 0.5 mM Pefabloc. To make the pellet visible, 10 μl blue dextran was added to each sample and the samples were centrifuged at 20,817×g for 1 h. Proteins in 100 μl untreated lysates were precipitated with 4× methanol overnight at −20° C. and pelleted at 2,120×g for 25 min at 4° C. Untreated samples were analysed with the PK-treated pellets for total PrP and PrPSc by western blot using monoclonal anti-PrP antibody 4H11.

Sedimentation Tau Polymers

The sedimentation assay was performed as described previously (Sanders et al., Neuron (2014), 82:1271-1288). Briefly, cell pellets were lysed in lysis buffer (150 mM NaCl (w/v), 50 mM (v/v) Tris-HCl, pH7.5, 1% (v/v) NP-40, protease inhibitor) on ice for 30 min. Cleared cell lysates were separated from cell debris by centrifugation at 2650×g for 2 min at 4° C. Cleared cell lysates adjusted to 100 μg total protein were subjected to centrifugation at 100,000×g for 1 h, 4° C. Pellets were washed with 1.5 ml PBS and insoluble material was pelleted again at 100,000×g for 30 min. Proteins in the supernatant fractions were precipitated with 4× methanol overnight at −20° C. and pelleted at 2,120×g for 25 min at 4° C. (soluble fraction). The pellet (insoluble fraction) and ⅓ of the soluble fraction dissolved in RIPA buffer with 4% SDS were loaded for western blot analysis.

Pronase digestion of Tau

The resistance of Tau aggregates to pronase treatment was probed as described previously (Sanders et al., Neuron (2014), 82:1271-1288). Briefly, 18 μl cleared cell lysates or brain homogenates (containing a total protein concentration of 20-100 μg dependening on Tau aggregates content) were incubated with 2 μl 1 mg/ml pronase (Roche) at 37° C. for 1 h. Afterwards, samples were boiled in 4× sample buffer with 1% SDS final. Pronase-resistant Tau bands were detected by western blot as described below with rabbit anti-Tau ab64193 (Abcam).

Preparation of Brain Homogenates

Frozen human brain samples were homogenized in complete OptiMEM culture medium (for cell culture), QIAzol lysis reagent (for RNA isolation) or lysis buffer (PBS with 1% Triton-X and protease, phosphotase inhibitors for protein analysis) using the Precellys® 24 (Bertin Instruments) with 1.4 mm ceramic beads at 4° C. for 4 cycles 5500 rpm 20 sec. For 10 brain homogenates for aggregate induction in cell cultures, crude homogenates were cleared of cell debris at 872×g for 5 min at 4° C. Supernatants were sonicated at 50% power for 6 min and stored at −80° C. RNA was isolated using the Qiagen RNeasy Lipid Tissue Mini Kit combined with genomic DNA digestion as described in the manufacturer's instruction. For protein analysis, brain homogenates were cleared of cell debris at 15000×g for 15 min, 4° C.

Tau Aggregate Induction Using Patient-Derived Brain Homogenate

To test the Tau aggregate induction by brain homogenates from different tauopathy patients, HEK Tau-GFP^{sol} cells were plated on a CellCarrier-96 black microplate (PerkinElmer) at 2000 cells/well in 50 μl complete medium. The next day, 6 μl 10% brain homogenate and 0.2 μl lipofectamine2000 were diluted into OptiMEM without antibiotics (final 60 μl) for 20 min at RT. Brain homogenate-liposome mixtures were added to recipient cells for 5 h and 50 μl complete medium were added to cells afterwards. The induced cells were fixed 3 days later in 4% paraformaldehyde. Nuclei were counterstained with Hoechst. Cells were imaged using the automated confocal microscope CellVoyager CV6000 (Yokogawa Inc.) and a 40× objective.

qRT-PCR

Total RNA from cell pellets or brain samples was isolated using the RNeasy Mini Kit or RNeasy Lipid Tissue Mini Kit (Qiagen). RNA concentration and quality were determined using the Agilent 2100 Bioanalyzer System. For a 20 μl reaction, 1 μg RNAs were reversely transcribed to cDNA using the iScript™ cDNA Synthesis Kit (Bio-Rad). For a 20 μl qRT-PCR reaction, 2 μl of synthesized cDNA was used as template. For qRT-PCR of murine env AA037244.2 and gag AID54952, custom designed TaqMan probes were used (Thermo Fisher Scientific). Pre-designed TaqMan probes by the company for murine pan-env, xpr1, mcat-1 and gapdh as housekeeping control and TaqMan™ Gene Expression Master Mix (Thermo Fisher Scientific) were used. qRT-PCR using TaqMan probes was performed as described in the manufacturer's instruction. For qRT-PCR analyses of HERV family members, primers were designed using the corresponding cDNA sequences (cf. SEQ ID NOs. 73-92). PowerUP SYBR™ Green Master Mix (Thermo Fisher Scientific) was mixed with different cDNAs and corresponding primers as indicated in the instruction. The fast cycling mode was used for all primers.

Western Blotting

For Western blot analysis, protein concentrations were measured using the Quick Start™ Bradford Protein assay (Bio-Rad). Proteins were separated on NuPAGE®Novex® 4-12 Bis-Tris Protein Gels (Life Technologies) followed by transfer onto a PVDF membrane (GE Healthcare). Western blot analysis was performed using rat hybridoma anti-MuERV Env mAb83A25; anti-xenotropic MuLV virus antibody ABIN457298 for detecting both Env and Gag (antibodies-online); mouse anti-MuERV Gag ab100970 (Abcam); mouse anti-Alix (1:1000; BD Bioscience); rat anti-HA 3F10 (1:1000; Roche); mouse anti-GAPDH 6C5 (1:5000; Abcam); mouse anti-Hsc/Hsp70 N27F3-4 (1:1000; ENZO); mouse anti-VSV-G A5977 (Sigma); rabbit anti-Tau ab64193 (Abcam); mouse anti-HERV K Env HERM-1811-5 (Amsbio); mouse anti-HERV K Gag HERM-1841-5 (Amsbio). The membrane was incubated with Pierce™ ECL Western Blotting Substrate (Thermo Fisher Scientific) according to the manufacturer's recommendations.

Image Analysis

The image analysis was performed using the CellVoyager Analysis support software. An image analysis routine was developed for single cell segmentation and aggregate identification (Yokogawa Inc.). The total number of cells was determined based on the Hoechst signal, and recipient cells were detected by their GFP/FusionRed signal. Green aggregates were identified via morphology and intensity characteristics. The percentage of recipient cells with aggregated NM-GFP or Tau-FusionRed/Tau-GFP was calculated as the number of aggregate-positive cells to total recipient cells set to 100%. False positive recipient cells were detected due to the heterogeneity of Tau-GFP/-FusionRed expression of individual cells. The mean percentage of false positives determined in control recipient cells was subtracted from all samples. Of note, negative values were sometimes obtained when no induction was observed. For data presentation, the minimum of the Y Axis was set to 0.

Immunofluorescence Staining and Confocal Microscopy Analysis of Prion-Infected Cells

Cells were fixed in 4% paraformaldehyde for 20 min at 37° C. and permeabilized in 0.1 Triton X-100 for 10 min at RT. For PrP^{Sc} staining, proteins were denatured in 6 M guanidine hydrochloride for 10 min at RT to reduce PrP^{C} staining and increase detection of PrP^{Sc} (Taraboulos et al., J Cell Biol (1990), 110:2117-2132). Cells were rinsed with PBS, blocked in 0.2% gelatine for 1 h and incubated for 2 h with anti-PrP 4H11 antibody hybridoma solution diluted 1:10 in blocking solution (Ertmer et al., JBC (2004), 279:41918-41927). After three washing steps in PBS, cells were incubated for 1 h with Alexa Fluor 488-conjugated anti-Mouse IgG secondary antibody diluted 1:800 in blocking solution (Thermo Fisher Scientific) and nuclei were counterstained for 15 min with 4 μg/ml Hoechst 33342 (Molecular Probes). 96 well plate was scanned with CellVoyager CV6000 (Yokogawa Inc.). Confocal laser scanning microscopy was performed on a Zeiss LSM 800 laser-scanning microscope with Airyscan (Carl Zeiss).

Statistical Analysis

All analyses were performed using the Prism 6.0 (GraphPad Software v.7.0c). Statistical inter-group comparisons were performed using the one-way ANOVA with a Bonferroni post-test or Student's t test. p values smaller than 0.05 were considered significant. All experiments were performed in triplicates or sextuplicates and repeated at least three times. Error bars represent the standard deviation (SD).

### Example 5

Downregulation of HERV-W Env Syncytin-1 Reduces Intercellular Aggregate Spreading

Human endogenous retroviruses (HERV) are usually silenced but become de-repressed during aging and in several human malignancies, including cancer, inflammatory diseases and neurodegeneration. To assess if HERV expression could affect intercellular spreading of protein aggregation, the inventors first made use of two cancer cell lines known to overexpress HERV. Human breast cell line MCF-7 was engineered to stably express Tau-GFP and exposed to AD brain homogenate to isolate clones propagating Tau-GFPAD. Cells were incubated with or without 5-Aza-2-deoxycytidine (Aza) for HERV de-repression and subsequently cocultured with recipient HEK Tau-FRsol cells (FIG. 14a). Aza treatment enhanced Syncytin-1 mRNA levels (FIG. 14b) and also increased recipient cells with aggregates in cocultures (FIG. 14c). Further, shRNA-mediated downregulation of Syncytin-1 expression in donor cells (FIG. 14d, e) significantly decreased induction of Tau-FR aggregates in cocultured recipients, suggesting that Syncytin-1 was involved in Tau aggregate spreading (FIG. 140.

HIV Protease Inhibitor Known to Inhibit HERV-K Maturation Reduces Intercellular Aggregate Spreading

The inventors further genetically engineered human A375 melanoma cells to express Tau-GFP and exposed them to AD brain homogenate to isolate a clone propagating Tau-GFPAD. Cells were treated with 10 μM Lopinavir, an HIV protease inhibitor shown to inhibit HERV-K protease required for HERV protein maturation. Upon coculture, the inventors observed a significant reduction of recipient cells with aggregates (FIG. 15a, b). Similarly, EV derived from Lopinavir-treated donors exhibited reduced Tau aggregate inducing activity in recipients (FIG. 15c). Combined, these data suggest that HERV could contribute to intercellular aggregate induction. The fact that HIV protease inhibitor Lopinavir also affected protein aggregate induction further suggests that HERV protease inhibitors represent promising drugs to inhibit proteopathic seed spreading.

HERV Env/Receptor Interactions Contribute to the Spreading of Proteopathic Seeds

To assess if HERV Env can mediate contact between donor and recipient membranes and thereby contribute to proteopathic seed spreading, the inventors overexpressed HERV-W Syncytin-1 in two HEK cell models propagating either aggregated NM-HA (HEK NM-HAagg) or aggregated Tau-GFP (HEK Tau-GFPAD) (FIG. 16a). Similar to HEK NM-HAagg cells, HEK Tau-GFPAD cells represent poor donor cells. Donors were subsequently cocultured with recipient cells expressing the respective soluble proteins. Importantly, overexpression of Myc epitope-tagged Syncytin-1 (Syn-Myc) was sufficient to increase intercellular aggregate induction in both cell culture systems (FIG. 16b, c). Expression of a Syn-Myc mutant incapable of mediating membrane fusion did not result in aggregate induction (FIG. 16b, c). The inventors reasoned that overexpression of Syncytin-1 specific receptors in recipient cells could increase aggregate induction (FIG. 16d). Indeed, ectopic expression of receptors ASCT1 (gene name SLC1A4) and ASCT2 (gene name SLC1A5) neutral amino acid transporters in recipients increased aggregate induction when recipients were cocultured with Syn-Myc expressing donor cells (FIG. 16e). By contrast, siRNA-mediated downregulation of receptor mRNAs in recipients reduced induction of protein aggregates upon coculture with Syn-Myc expressing donors (FIG. 16f-h). Next, the inventors tested if Syncytin-1 also affected aggregate induction by EV. To this end, EV from donor cell lines transiently expressing Syn-Myc were isolated and incubated with recipient cell lines overexpressing the Syncytin-1 specific receptors ASCT1/2 (genes: SLC1A4/SLC1A5). Induction of protein aggregates in recipient cells was monitored 3 d later (FIG. 16i). Western blot analysis revealed that Syn-Myc was associated with EV fractions harboring EV markers Flotillin-1, Hsp70/72 as well as NM-HA or Tau-GFP (FIG. 16j). When added to recipient cells, Syn-Myc containing EV resulted in a significant increase in protein aggregate induction in recipient cells (FIG. 16k). In conclusion, HERV Env/receptor interactions can contribute to the intercellular spreading of proteopathic seeds.

Materials and Methods

Molecular Cloning

To generate the expression vector coding for SLC1A4 or SLC1A5, the corresponding cDNA for SLC1A4 (cataloge nr. #EX-A3396-Lv213; GeneCopoeia) or SLC1A5 (cataloge nr. #EX-Z2810-Lv213; GeneCopoeia) was cloned into cataloge nr. #PB510B-1 vector (SBI) under the CMV promoter. To generate the phCMV-Syncytin-1-100UTR plasmid, Syncytin-1 cDNA tagged with a Myc epitope sequence (cataloge nr. #EX-T0264-Lv213; GeneCopoeia) was cloned into phCMV-EcoENV (Addgene #15802) using EcoRI and XhoI to replace EcoENV. The 100 bp sequence from 3′-UTR of Syncytin-1 shown to enhance gene expression was amplified using primers (SEQ ID NO: 95 forward: 5′-CCGCTCGAGAGCGGTCGTCGGCCAAC-3′/ SEQ ID NO: 96 reverse: 5′-GAAGATCTCCTTCCCAGCTAGGCTTAGGG-3′) and genomic DNA from MCF-7 cells as template. The sequence was cloned into phCMV-Syncytin-1 using XhoI and BglII restriction sites. The three point mutations R314A, N315A and K316A, shown to destroy fusogenic activity, were introduced using the Q5 site-directed mutagenesis Kit (NEB).

Cell Lines

MCF-7 (ATCC HTB-22) cells were cultured in MEM (Gibco) with 10% FCS, P/S, 10 nM estrogen and 0.01 mg/ml human recombinant insulin. A375 (ATCC CRL-1619) cells were cultured in DMEM (Gibco) with 10% FCS, P/S.

Brain Homogenate Preparation and Clarification

Frozen human brain samples were homogenized in lysis buffer (for protein analysis) via Precellys® 24 (Bertin Instruments) with 1.4 mm ceramic beads at 4° C. for 4 cycles 5500 rpm 20 s. To prepare 10% (w/v) clear brain homogenate for aggregate induction, crude homogenates were centrifuged at 872×g for 5 min at 4° C., and then the supernatants were sonicated with 50% power for 6 min. These homogenates were frozen at −80° C. until use. For protein analysis, cleared supernatants were prepared by centrifugation of the crude homogenates at 15,000×g for 15 min.

Tau Aggregate Induction by Brain Homogenate and Liposomes

To induce Tau aggregation in MCF7/A375 Tau-GFPsol cells with brain homogenates from AD patients, cells were plated on 6-well plates at 1×10⁶ cells/well in 2 ml complete medium one day before. Next day, 200 μl 10% brain homogenates and 4 μl lipofectamine2000 were incubated for 20 min and added to recipient cells to have final 1% brain homogenates on cells. After 3 days, cells were split and further expanded for limited dilution clone selection as previously described.

Production and Transduction with Lentiviral Particles

HEK293T cells were cotransfected with plasmids pRSV-Rev, pMD2.VSV-G, pMDI.g/pRRE (all plasmids were published in Dull T, Zufferey R, Kelly M, Mandel R J, Nguyen M, Trono D, Naldini L A third-generation lentivirus vector with a conditional packaging system. J Virol. 1998 November 72(11):8463-71), and pRRI.sin.PPT.hCMV.Wpre (plasmid published in Follenzi, A. and L. Naldini (2002) HIV-based vectors. Preparation and use. Methods in molecular medicine 69: 259-274) containing Tau-GFP for fluorescence tagged Tau expression or pSIH-shRNA-Syn GGCCCTCCCTTATCATATT (SEQ ID NO: 97) with the CTTCCTGTCAGA (SEQ ID NO: 98) loop sequence to silence Syncytin-1 expression, pSIH-puro-control (Addgene #26597) was used to produce control shRNA lentivirus. Supernatants were harvested and concentrated with PEG according to published protocols. MCF-7 and A375 cell lines were transduced with Tau-GFP lentivirus to produce MCF-7 and A375 Tau-GFPsol cells. MCF-7 Tau-GFPAD clones were transduced with shRNA-Syn or control lentiviruses, and selected with 2 μg/ml puromycin for 2 weeks.

Drug Treatment

To inhibit methyltransferase, MCF-7 Tau-GFPAD cells were treated with 2 μM Aza or DMSO for 4 d. Thereafter, the pretreated donor cells were cocultured with recipient HEK Tau-FRsol in the absence of the drugs for 3 d. The treatment of A375 melanoma cells with Lopinavir (10 μM; Selleckchem) and DMSO was performed for 72 h in EV-depleted medium in T175 flasks. Afterwards, the total numbers of viable cells and the viability upon drug treatments were determined using the Vi-VELLTMXR Cell Viability Analyzer (Beckman Coulter). EV were isolated from the conditioned medium via ultracentrifugation and processed for the assays as described above.

Transfection with siRNAs or Plasmids

To transiently knock-down specific genes, custom-designed Silencer select siRNAs from Thermo Fisher were used. Pre-designed siRNAs were used to knock-down genes. For transfection, cells were pre-seeded on 6 well plate one d before at 2×10⁵ cells/well. The next day, either a final 60 nM (1:1 SLC1A4 (#s12914)/SLC1A5 (#s12918)) siRNAs (Lifetechnologies) was mixed with 1:20 diluted Lipofectamine RNAiMAX for siRNAs or 2 μl plasmid was mixed with 4 μl TransIT-2020 (Mirusbio) diluted in Opti-MEM for 30 min before addition to cells. After 1-3 d, transfected cells were harvested for aggregate induction assays, qRT-PCR or Western blot analysis.

qRT-PCR

Total RNAs from cell pellets were isolated using the RNeasy Mini Kit or RNeasy Lipid Tissue Mini Kit (Qiagen). RNA concentration and quality were determined with Agilent 2100 Bioanalyzer System. RNAs were reversely transcribed to cDNA using the iScript™ cDNA Synthesis Kit (Bio-Rad). For mRNA analysis, pre-designed TaqMan assays for human SLC1A4 (Hs00983079_m1), SLC1A5 (Hs01056542_m1), GAPDH (Hs02786624_g1) or ACTB (Hs01060665_g1) as housekeeping control were utilized with TaqMan™ Gene Expression Master Mix (Thermo Fisher).

Western Blotting

For Western blot analysis, protein concentrations were measured by Quick Start™ Bradford Protein assay (Bio-Rad) and proteins were separated on NuPAGE®Novex® 4-12% Bis-Tris Protein Gels (Life Technologies) followed by transfer onto a PVDF membrane (GE Healthcare) in a wet blotting chamber. Western blot analysis was performed using rabbit anti-Flotillin-1 ab133497 (Abcam); rat anti-HA 3F10 (1:1000; Roche); mouse anti-GAPDH 6C5 (1:5000; Abcam); mouse anti-Hsp70/72 N27F3-4 (1:1000; ENZO); rabbit anti-Tau ab64193 (Abcam); rat anti-c-myc-HRP 130-092-113 (Miltenyi Biotec). The membrane was incubated with Pierce™ ECL Western Blotting Substrate (Thermo Fisher Scientific) according to the manufacturer's recommendations.

Image Analysis

The image analysis was performed using the CellVoyager Analysis support software. An image analysis routine was developed for single cell segmentation and aggregate identification (Yokogawa Inc.) The total number of cells was determined based on the Hoechst signal, and recipient cells were detected by their GFP/-FR signal. Green aggregates were identified via morphology and intensity characteristics. The percentage of recipient cells with aggregated NM-GFP or Tau-FR/Tau-GFP was calculated as the number of aggregate-positive cells per total recipient cells set to 100%. False positive induced recipient cells were detected due to the heterogeneity in GFP/FR expression of individual cells. The mean percentage of false positives determined in control recipient cells was subtracted from all samples. Of note, negative values were sometimes obtained when no induction was observed. For data presentation, the minimum range of Y Axis was set to 0.

Statistical Analysis

All analyses were performed using the Prism 6.0 (GraphPad Software v.7.0c). Statistical inter-group comparisons were performed using the one-way ANOVA with a Bonferroni post-test or unpaired Student's t test. p values smaller than 0.03 (*), 0.002 (**) and 0.0002 (***) were considered significant. All experiments were performed in triplicates or sextuplicates and repeated at least two times. Error bars represent the standard deviation (SD).

### REFERENCES

Voisset C, Weiss R A, Griffiths D J. 2008. Human RNA “rumor” viruses: the search for novel human retroviruses in chronic disease. Microbiol Mol Biol Rev 72:157-196, table of contents.
Grandi N, Tramontano E. 2018. HERV Envelope Proteins: Physiological Role and Pathogenic Potential in Cancer and Autoimmunity. Front Microbiol 9:462.
Christensen T. 2010. HERVs in neuropathogenesis. J Neuroimmune Pharmacol 5:326-335.
Yi J M, Kim H M, Kim H S. 2004. Expression of the human endogenous retrovirus HERV-W family in various human tissues and cancer cells. J Gen Virol 85:1203-1210.
Oricchio E, Sciamanna I, Beraldi R, Tolstonog G V, Schumann G G, Spadafora C. 2007. Distinct roles for LINE-1 and HERV-K retroelements in cell proliferation, differentiation and tumor progression. Oncogene 26:4226-4233.
Chiappinelli K B, Strissel P L, Desrichard A, Li H, Henke C, Akman B, Hein A, Rote N S, Cope L M, Snyder A, Makarov V, Budhu S, Slamon D J, Wolchok J D, Pardoll D M, Beckmann M W, Zahnow C A, Merghoub T, Chan T A, Baylin S B, Strick R. 2015. Inhibiting DNA Methylation Causes an Interferon Response in Cancer via dsRNA Including Endogenous Retroviruses. Cell 162:974-986.
Tyagi R, Li W, Parades D, Bianchet M A, Nath A. 2017. Inhibition of human endogenous retrovirus-K by antiretroviral drugs. Retrovirology 14:21.
Cheynet V, Ruggieri A, Oriol G, Blond J L, Boson B, Vachot L, Verrier B, Cosset F L, Mallet F. 2005. Synthesis, assembly, and processing of the Env ERVWE1/syncytin human endogenous retroviral envelope. J Virol 79:5585-5593.
Kitao K, Tanikaga T, Miyazawa T. 2019. Identification of a post-transcriptional regulatory element in the human endogenous retroviral syncytin-1. J Gen Virol 100:662-668.
Follenzi A, Naldini L. 2002. HIV-based vectors. Preparation and use. Methods Mol Med 69:259-274.

1. Inhibitor of HERV proteins comprising HERV Env and/or Gag, or fragments thereof, for use in treating a tauopathy, or Parkinson's disease.

2. Inhibitor of claim 1, wherein said inhibitor inhibits maturation or expression of said HERV Env and/or Gag proteins, and/or binding of said HERV Env protein to a receptor.

3. Inhibitor of claim 1 or 2, wherein said inhibitor inhibits maturation of said HERV Env and/or Gag proteins, and wherein said inhibitor is a HERV protease inhibitor.

4. Inhibitor of claim 1 or 2, wherein said inhibitor inhibits expression of said HERV Env and/or Gag proteins, and wherein said inhibitor is a nucleic acid molecule hybridizing to at least a portion of the nucleic acid sequence encoding said HERV Env and/or Gag proteins, respectively.

5. Inhibitor of claim 1 or 2, wherein said inhibitor inhibits binding of said HERV Env protein to a receptor.

6. Inhibitor of claim 5, which is an anti-HERV Env protein-antibody.

7. Inhibitor of a receptor binding a HERV Env protein for use in treating tauopathy, or Parkinson's disease.

8. Inhibitor of claim 7, wherein said inhibitor inhibits maturation or expression of said receptor, and/or binding of HERV Env protein to said receptor.

9. Inhibitor of claim 7 or 8, which is a nucleic acid molecule complementary to at least a portion of the nucleic acid sequence encoding said receptor.

10. Inhibitor of claim 7 or 8, which is an antibody binding to said receptor or a fragment thereof.

11. Inhibitor of any one of claims 7 to 10, wherein said receptor is selected from the group consisting of SLC1A4 and SLC1A5.

12. Molecule binding to HERV Env protein or a fragment thereof, or to a nucleic acid molecule encoding said HERV Env protein or a fragment thereof, for use in diagnosing a tauopathy, or Parkinson's disease.

13. Molecule of claim 12, which is an anti-HERV Env protein-antibody.

14. Molecule of claim 12, which is a nucleic acid molecule binding to the nucleic acid molecule encoding HERV Env protein or a fragment thereof.

15. Inhibitor of any one of claims 1 to 11 or molecule of any one of claims 12 to 14, wherein said tauopathy is selected from the group consisting of Alzheimer's Disease (AD), Argyrophilic Grain Disease (AGD), Cortical Basal Degeneration (CBD), Progressive Supranuclear Palsy (PSP), Pick's Disease (PiD), and Frontotemporal Dementia with Parkinsonism related to chromosome 17 (FTDP-17).


SEQUENCE LISTING (139 sequences)

SEQ ID NO: 1 - DNA, 1608 bp, Homo sapiens
atgacagagaaattccttttcctttatctttccctccttcccatgcccctactctcacaggcacagtggaatgaaaattcccttgtcagtttttccaaaataattgcttcgggaaaccatctaagcaactgttggatctgccacaacttcatcaccaggtcctcatcttaccaatatattttggtaagaaatttttctttaaacctaacatttggttcaggaatccctgaaggccaacataaatctgttccgctccaggtttcgcttgctaactcagcgcaccaagtcccctgcctggatctcactccacctttcaatcaaagctctaaaacttctttctatttctacaactgctcttctctaaaccaaacctgttgtccatgccctgaaggacactgtgacaggaagaacacctctgaggagggattccccagtcccaccatccatcccatgagcttctccccagcaggctgccaccctaacttgactcactggtgtccagctaaacaaatgaacgattatcgagacaagtcaccccaaaaccgctgtgcagcttgggaaggaaaagagctaatcacatggagggttctatattcgcttcccaaggcacacactgtccccacatggccaaaatctactgttcccctgggagggcctctatcccctgcatgcaatcaaactattccagcagggtggaaatcgcagttacacaagtggttcgacagccacatcccccggtgggcctgtacccctcctggctatgtatttttatgtgggccacaaaaaaataaactgccctttgatggaagtcctaagataacctattcaaccccccctgtggcaaacctctacacttgcattaataacatccaacatacgggagaatgtgctgtgggacttttgggaccacgggggataggtgtgaccatttataacaccacccaacccagacagaaaagagctctgggtctaatactggcagggatgggtgcggccataggaatgatcgccccatggggagggttcacttatcatgatgtcaccctcagaaatctctccagacaaatagacaacatagctaagagtaccagagatagcatctctaaactcaaggcctccatagattctctagcaaatgtagtcatggacaacagattggccttagattacctcttagcagagcagggtggagtctgtgcagtgatcaataaatcctgttgcgtttatgtcaataacagtggggcgatagaggaggatataaaaaagatctatgatgaggctacgtggctccatgactttggaaaaggaggtgcttcagcaagggccatctgggaggctgtgaagtctgccctcccctccctcaactggtttgtccctttactgggaccagcaacagttatactcttacttttcctctttggcccttgtttctttaatttactgattaagtgtgtctcttctaggataaagcaatttcacatgaagtccccccaaatggaaagatatcagctatctgtcattggaggccccagcacctataagcacatctcccccttggatgccagtgggcaaagattccgggaaactatggaggaattttctctctga

SEQ ID NO: 2 - DNA, 2100 bp, artificial
  HERV K Env consensus sequence from 11 genes
atgaacccatcggagatgcaaagaaaagcacctccgcggagacggagacaccgcaatcgagcaccgttgactcacaagatgaacaaaatggtgacgtcagaagaacagatgaagttgccatccaccaagaaggcagagccgccgacttgggcacaactaaagaagctgacgcagttagctacaaaatatctagagaacacaaaggtgacacaaaccccagagagtatgctgcttgcagccttgatgattgtatcaatggtggtaagtctccctatgcctgcaggagcagctgcagctaactatacctactgggcctatgtgcctttcccgcccttaattcgggcagtcacatggatggataatcctatagaagtatatgttaatgatagtgtatgggtacctggccccatagatgatcgctgccctgccaaacctgaggaagaagggatgatgataaatatttccattgggtatcgttatcctcctatttgcctagggagagcaccaggatgtttaatgcctgcagtccaaaattggttggtagaagtacctactgtcagtcccatcagtagattcacttatcacatggtaagcgggatgtcactcaggccacgggtaaattatttacaagacttttcttatcaaagatcattaaaatttagacctaaagggaaaccttgccccaaggaaattcccaaagaatcaaaaaatacagaagttttagtttgggaagaatgtgtggccaatagtgcggtgatattacaaaacaatgaatttggaactattatagattgggcacctcgaggtcaattctaccacaattgctcaggacaaactcagtcgtgtccaagtgcacaagtgagtccagctgttgatagcgacttaacagaaagtttagacaaacataagcataaaaaattgcagtctttctacccttgggaatggggagaaaaaggaatctctaccccaagaccaaaaatagtaagtcctgtttctggtcctgaacatccagaattatggaggcttactgtggcctcacaccacattagaatttggtctggaaatcaaactttagaaacaagagatcgtaagccattttatactgtcgacctaaattccagtctaacagttcctttacaaagttgcgtaaagcccccttatatgctagttgtaggaaatatagttattaaaccagactcccagactataacctgtgaaaattgtagattgcttacttgcattgattcaacttttaattggcaacaccgtattctgctggtgagagcaagagagggcgtgtggatccctgtgtccatggaccgaccgtgggaggcctcaccatccgtccatattttgactgaagtattaaaaggtgttttaaatagatccaaaagattcatttttactttaattgcagtgattatgggattaattgcagtcacagctacggctgctgtagcaggagttgcattgcactcttctgttcagtcagtaaactttgttaatgattggcaaaaaaattctacaagattgtggaattcacaatctagtattgatcaaaaattggcaaatcaaattaatgatcttagacaaactgtcatttggatgggagacagactcatgagcttagaacatcgtttccagttacaatgtgactggaatacgtcagatttttgtattacaccccaaatttataatgagtctgagcatcactgggacatggttagacgccatctacagggaagagaagataatctcactttagacatttccaaattaaaagaacaaattttcgaagcatcaaaagcccatttaaatttggtgccaggaactgaggcaattgcaggagttgctgatggcctcgcaaatcttaaccctgtcacttgggttaagaccattggaagtactacgattataaatctcatattaatccttgtgtgcctgttttgtctgttgttagtctgcaggtgtacccaacagctccgaagagacagcgaccatcgagaacgggccatgatgacgatggcggttttgtcgaaaagaaaagggggaaatgtggggaaaagcaagagagatcagattgttactgtgtctgtgtag

SEQ ID NO: 3 - DNA, 1881 bp, Homo sapiens
atgggtcccgaagcctgggtcaggccccttaaaactgcgcctaagccgggtgaagccattagattaattctttttatttacctctcttgtttctttttgcctgttatgtcctctgagccttcctactcctttctcctcacctctttcacaacaggacgtgtattcgcaaacactacttggagggccggtacctccaaggaagtctcctttgcagttgatttatgtgtactgttcccagagccagctcgtacccatgaagagcaacataatttgccggtcataggagcaggaagtgtcgaccttgcagcaggatttggacactctgggagccaaactggatgtggaagctccaaaggtgcagaaaaagggctccaaaatgttgacttttacctctgtcctggaaatcaccctgacgctagctgtagagatacttaccagtttttctgccctgattggacatgtgtaactttagccacctactctgggggatcaactagatcttcaactctttccataagtcgtgttcctcatcctaaattatgtactagaaaaaattgtaatcctcttactataactgtccatgaccctaatgcagctcaatggtattatggcatgtcatggggattaagactttatatcccaggatttgatgttgggactatgttcaccatccaaaagaaaatcttggtctcatggagctcccccaagccaatcgggcctttaactgatctaggtgaccctatattccagaaacaccctgacaaagttgatttaactgttcctctgccattcttagttcctagaccccagctacaacaacaacatcttcaacccagcctaatgtctatactaggtggagtacaccatctccttaacctcacccagcctaaactagcccaagattgttggctatgtttaaaagcaaaacccccttattatgtaggattaggagtagaagccacacttaaacgtggccctctatcttgtcatacacgaccccgtgctctcacaataggagatgtgtctggaaatgcttcctgtctgattagtaccgggtataacttatctgcttctccttttcaggctacttgtaatcagtccctgcttacttccataagcacctcagtctcttaccaagcacccaacaatacctggttggcctgcacctcaggtctcactcgctgcattaatggaactgaaccaggacctctcctgtgcgtgttagttcatgtacttccccaggtatatgtgtacagtggaccagaaggacgacaactcatcgctccccctgagttacatcccaggttgcaccaagctgtcccacttctggttcccctattggctggtcttagcatagctggatcagcagccattggtacggctgccctggttcaaggagaaactggactaatatccctgtctcaacaggtggatgctgattttagtaacctccagtctgccatagatatactacattcccaggtagagtctctggctgaagtagttcttcaaaactgccgatgcttagatctgctattcctctctcaaggaggtttatgtgcagctctaggagaaagttgttgcttctatgccaatcaatctggagtcataaaaggtacagtaaaaaaagttcgagaaaatctagataggcaccaacaagaacgagaaaataacatcccctggtatcaaagcatgtttaactggaacccatggctaactactttaatcactgggttagctggacctctcctcatcctactattaagtttaatttttgggccttgtatattaaattcgtttcttaattttataaaacaacgcatagcttctgtcaaacttacgtatcttaagactcaatatgacacccttgttaataactga

SEQ ID NO: 4 - DNA, 1617 bp, Homosapiens
atggccctcccttatcatatttttctctttactgttcttttaccctctttcactctcactgcaccccctccatgccgctgtatgaccagtagctccccttaccaagagtttctatggagaatgcagcgtcccggaaatattgatgccccatcgtataggagtctttctaagggaacccccaccttcactgcccacacccatatgccccgcaactgctatcactctgccactctttgcatgcatgcaaatactcattattggacaggaaaaatgattaatcctagttgtcctggaggacttggagtcactgtctgttggacttacttcacccaaactggtatgtctgatgggggtggagttcaagatcaggcaagagaaaaacatgtaaaagaagtaatctcccaactcacccgggtacatggcacctctagcccctacaaaggactagatctctcaaaactacatgaaaccctccgtacccatactcgcctggtaagcctatttaataccaccctcactgggctccatgaggtctcggcccaaaaccctactaactgttggatatgcctccccctgaacttcaggccatatgtttcaatccctgtacctgaacaatggaacaacttcagcacagaaataaacaccacttccgttttagtaggacctcttgtttccaatctggaaataacccatacctcaaacctcacctgtgtaaaatttagcaatactacatacacaaccaactcccaatgcatcaggtgggtaactcctcccacacaaatagtctgcctaccctcaggaatattttttgtctgtggtacctcagcctatcgttgtttgaatggctcttcagaatctatgtgcttcctctcattcttagtgccccctatgaccatctacactgaacaagatttatacagttatgtcatatctaagccccgcaacaaaagagtacccattcttccttttgttataggagcaggagtgctaggtgcactaggtactggcattggcggtatcacaacctctactcagttctactacaaactatctcaagaactaaatggggacatggaacgggtcgccgactccctggtcaccttgcaagatcaacttaactccctagcagcagtagtccttcaaaatcgaagagctttagacttgctaaccgctgaaagagggggaacctgtttatttttaggggaagaatgctgttattatgttaatcaatccggaatcgtcactgagaaagttaaagaaattcgagatcgaatacaacgtagagcagaggagcttcgaaacactggaccctggggcctcctcagccaatggatgccctggattctccccttcttaggacctctagcagctataatattgctactcctctttggaccctgtatctttaacctccttgttaactttgtctcttccagaatcgaagctgtaaaactacaaatggagcccaagatgcagtccaagactaagatctaccgcagacccctggaccggcctgctagcccacgatctgatgttaatgacatcaaaggcacccctcctgaggaaatctcagctgcacaacctctactacgccccaattcagcaggaagcagttag

SEQ ID NO: 5 - DNA, 1617 bp, Homo sapiens
atgggcctgctcctgctggttctcattctcacgccttcactagcagcctaccgccatcctgatttcccgttattggaaaaagctcagcaactgctccaaagtacaggatccccttactccaccaattgctggttatgtactagctcttccactgaaacaccagggacagcttatccagcctcgcccagagaatggacaagcatagaggcggaattacatatttcctatcgatgggaccctaatctgaaaggactgatgaggcctgcaaatagtcttctttcaacagtaaagcaagatttccctgatatccgccagaaacctcccattttcggacccatctttactaatatcaacctaatgggaatagcccctatttgtgttatggccaaaaggaaaaatggaacaaatgtaggcactcttccaagtacagtctgtaatgttactttcactgtagattctaaccaacagacttaccaaacatacacccacaaccaattccgccatcaaccaagattccccaaacctccaaatattacttttcctcagggaactttgctagataaatccagccggttttgccagggacgcccaagctcatgcagtactcgaaacttctggttccggcctgctgattataaccaatgtctgcaaatttccaacctcagctctacagcggaatgggttctattggaccaaactcgaaattctcttttttgggaaaataaaaccaagggagctaaccagagccaaacaccctgcgtccaagtcttagcaggcatgactatagccaccagctacctgggcatatcagcagtctcagaattttttggaacctccctcacccccttatttcatttccatatctctacatgccttaaaactcaaggagccttttatatttgtggccagtcgattcaccaatgcctccccagtaactggactggaacttgtaccataggctatgtaaccccagacatcttcatagcccctggcaatctctctcttccaataccaatctatgggaattccccgttgcccagggtgaggagggcaatccatttcattccccttctcgcgggactcggcattctagctggtacgggaaccggaattgctggaatcacaaaagcttccctcacctatagccagctctcaaaggaaatagccaacaacattgacaccatggctaaagccttaacgaccatgcaagaacaaatcgactctttagcagccgtagtccttcaaaatcgtcgaggactagacatgttaacggcagcacagggaggaatttgtttggccttagatgaaaaatgttgcttttgggtaaatcaatcaggaaaagtacaagacaacatcagacaactcctaaatcaagcctccagtttacgggaacgagccactcagggttggttaaattgggaaggaacttggaaatggttctcttgggttcttccccttacaggcccacttgttagtctcctacttttgctcctttttggtccatgtctcctaaatctaataacccaatttgtctcctctcgccttcaggccataaagctccagacgaatctcagtgcaggacgccatcctcgcaatattcaagagtcacccttctaa

SEQ ID NO: 6 - DNA, 1695 bp, Homosapiens
atgactaaaaccctgttgtatcacacttattatgagtgtgctgggacctgcctaggaacttgtactcacaaccagacaacctactcagtctgtgacccaggaaggggccagccttatgtgtgttatgaccctaagtcttcacctgggatctggtttgaaattcatgtcgggtcaaaggaaggggatcttctaaaccaaaccaaggtatttccctctggcaaggatgtcgtatccttatactttgatgtttgccagatagtatccatgggctcactctttcccgtaatcttcagttccatggagtactatagtagctgccataaaaataggtatgcacaccctgcttgttccaccgattccccagtaacaacttgctgggactgcacaacgtggtccactaaccaacaatcactagggccaattatgcttaccaaaataccattagaaccagattgtaaaacaagcacttgcaattctgtaaatcttaccatcttagagccagatcagcccatatggacaacaggtttaaagacaccgctaggggcacgagtcagcgatgaagaaattggcccaggagcctatgtctatctatatatcataaagaaaactcggacccgctcaacccaacagttgcgagtttttgagtcattctatgagcatgttaaccagaaattgcctgagccccctcccttggccagtaatttattcgcccaactggctgaaaacatagccagcagcctgcacgttgcttcatgttatgtctgtgggggaatgaacatgggagaccaatggccatgggaagcaagggaactaatgccccaagataatttcacactaaccgcctcttccctcgaacctgcaccatcaagtcagagcatctggttcttaaaaacctccattattggaaaattctgtattgctcgctggggaaaggcctttacagacccagtaggagagttaacttgcctaggacaacaatattacaacgagacactaggaaagactttatggaggggcaaaagcaataattctgaatcaccacacccaagcccattctctcgtttcccatctttaaaccattcttggtaccaacttgaagctccaaatacctggcaggcaccctctggcctctactggatctgtgggccacaagcatatcgacaactgccagctaaatggtcaggggcctgtgtactggggacaattaggccgtccttcttcctaatgcccctaaaacagggagaagccttaggataccccatctatgatgaaactaaaaggaaaagcaaaagaggcataactataggagattggaaggacagtgaatggcctcctgaaagaataattcaatattatggcccagccacctgggcagaagatggaatgtggggataccgcaccccagtttacatgcttaaccgcattataagattgcaggcagtactagaaatcattaccaatgaaactgcaggggccttgaatctgcttgcccagcaagccacaaaaatgagaaatgtcatttatcaaaatagactggccttagactacctcctagcccaggaagagggagtatgcggaaagttcagccttactaactgctgcctggaacttgatgacgaaggaaaggttatcaaagaaataactgctaaaatccaaaagttagctcacatcccagttcagacttggaaaggatag

SEQ ID NO: 7 - DNA, 1545 bp, Homo sapiens
atggatccactacacacgattgaaaaagttcctgcaagaagaaacatccacgacagaggacaccaaggccaccgaatgggagatggaacccctggaaggcctaagatttctgttcaacaaatgacaagattttcccttataatatttttcctttctgctccttttgttgttaatgcctctacctctaacgttttcctacaatgggcacacagttatgcagatggcttacaacaaggagacccttgctgggtctgtggttcgttacccgtcactaacaccatggagctaccttggtgggtctccccgctacaagggaaagactgggttttttttcaaagctttataggggatcttaaacaatggacaggggcacagatgactggggtaactagaaaaaacatttcagaatggcctataaataaaactttaaatgagccagggcatgataaaccattctcagtaaatgagacaagggataaagtaatagcctttgccatccccttgttggataccaaggtgtttgtccagacttccagacctcagaacactcaatatagaaatgggtttctccagatatgggacgggttcatttggctgacagccactaagggacacttaagccagatagctcccttatgctgggagcaaagaaatcactcccttgataactggccaaacacaactcgtgttatgggatggattccacctggacagtgccgacatactatactgttacaacagagggacctatttgccacagactggtctcagcaacctggcttgaattggtatgctcccaacggaacccagtggctctgcagcccaaacttatggccttggcttccctcaggttggttaggatgctgcactctaggtattccctgggcacaaggacgctgggtaaaaaccatggaagtctatccttatcttccacatgtggttaaccaagggactagggccattgttcacaggaatgatcacctacccacaatctttatgccctcagtaggtttaggaactgtaatacagcacatagaggctctagccaattttacccaacgggccctaaatgacagcctccaaagtatttctctcatgaatgctgaagtgtattatatgcacgaggacatcttacaaaaccgaatggccctagatattttaactgcggctgaaggaggaacctgtgccctcatcaaaactgaatgttgtgtgtatattcccaataactctagaaacatttccttggccttagaggatacatgtcggcaaatccaagtcatctccagctctgcactgtcactccatgactggatagcatctcagtttagtggaagaccttcctggtggcagaaaatcctcattgtccttgccaccctctggagcgtaggcatagcactgtgttgtggactgtatttttgtcgcatgttttcccaacacattccccaaactcattcgattatatttcaacaggaacttcccttgagccccccaagtcaggagcattaccagagccaaagagacatcttccactctaacgccccctga

SEQ ID NO: 8 - DNA, 1584 bp, Homo saoiens
atgaattctccatgtgacaggctccaacaatttattcaggttcttctcgaggaaagctggtcattccctagttttgctaacacccttcactggcctgaaaatctgttgtcctatatagacgaactggtatggcaaggctccctccagaactttcaccaacatgaagttcgctttgacaagccccctctcagactccctctcactgggttttcttccctcactgagaattggagttccagacaggcagtgtcctctagactagtagccacggcagcatccccgccagcagggtgtcaggcacccatagctttcctaggtctaaaattctcttccctaggcccggctagaaaaaaccctgcactttgcttcctctatgatcaaagtaactccaaatgcaataccagctgggtcaaagaaaatgtaggctgtccgtggcactggtgcaatatccatgaggcattaattcgtactgaaaaaggatctgacccaatgttctatgtcaatacctccactggaggacgggacggctttaacggatttaacctccaaatttctgacccttgggacccccgctgggcctccggtgtagatggaggactatatgagcacaaaacttttatgtatccagtagctaagatccgcattgccaggacccttaaaaccactgtcacagggttatccgacttagcctcctcaatccagtcagccgagaaagagcttaccagccagcttcaaccggcagctgaccaggccaagtcctcccgcttctcgtggttaactttaatctcagaaggtgcacaattgctccaatccacaggggtacaaaacctctcccactgcttcctctgtgcagccctcagaagacctccattagtagcagttcctctccctaccccctttaattatactataaattcatcaacccctataccaccggtcccaaaaggacaggtcccactattctcagaccctataagacataagttcccattctgttactctaccccaaatgcctcttggtgtaaccagactaggatgcttaccagcaccccggcaccgcccagaggctacttctggtgtaactccacgctaactaaagttcttaactcaactggtaatcacaccttgtgcttacccatctctctcatccctggcctgaccctatatagtcaggatgaacttagccatctgctagcctggaccgagccaaggccacaaaataaaagcaaatgggctattttcttacccttagtactaggcatctccttagcctcctccttagtggcatcagggctaggaaaaggagccctcacccactcaatccaaacatctcaagatctgtctactcacctgcagttggccattgaggcatcggccgagtccctggactccctacagcgacagatcactacagtagcacaggtcgcagcacagaacaggcaggccttagacttgcttatggcagaaaaaggaaggacatgtttattcttacaggaagaatgctgctattatctcaatgagtcaggggtagttgagaacagtttacaaactttaaaaaaaaaaaaaagctccaagaggagttaa

SEQ ID NO: 9 - DNA, 1974 bp, Homo sapiens
atggccagaccttccccactatgcctcctactcctcctgaccctcctaacccccatagtgcccagtaactccctcctaactgaacccccgttccgatggaggttctacctgcatgagacttggacccaaggcaaccggctctccactgtcacactggcaacggtggactgccaacctcacggttgtcaggcccaagtaacttttaacttcacttcctttaaaagtgttctgcggggctggtccaatcccaccatctgctttgtctatgatcaaacacacagcaactgccgcgactattgggtggacacaaacggaggatgcccctatgcctattgtcgtatgcatgtgacccagctccataccgccaagaaactccaacacacctatcgcctgacatctgatggaaggacaacttacttcctgaccatcccagacccatgggattctcggtgggtcagtggagtcactggtcgactgtaccggtggcccaccgactcctacccagttggcaaactccggatattcctgacttatatacgagttatcccccaggttttgtctaatttaaaggaccaagcagacaacattaagcatcaggaagaggtcatcaatactttggtgcagtcccatccgaaggctgacatggtcacctatgatgacaaggctgaggcaggaccgttttcatggataaccctagtccgccacggggctcgccttgttaatatggcaggcctagttaatctctcccactgtttcctttgcaccgccctcagccaaccaccactagtagctgtacccctaccccaggcttttaacacctctggtaaccacactgcccacccttccggcgtcttctctgagcaggtccctcttttccgagaccccctccagccccagttccccttctgctacaccactcctaactcatcctggtgcaaccagacctattctggctccctatctaacctctctgcaccggcaggtggctacttctggtgtaacttcacccttacaaaacatcttaatatttcctctaacaataccctttctagaaacttatgcctccccatctctctggtgcctcgactcactctgtacagcgaggctgaactctcttcccttgtcaacccgcctatgcgtcagaagcgggccgttttcccaccgctggtaataggtgtctccttgacctcctcacttgttgcctccgggctgggcacaggtgctattgtacatttcataagctcttcccaagatctctctattaagctccagatggccatcgaagcctcagccgaatccttagcctctctacagagacagattacgtctgtggccaaggtggccatgcagaaccggagagccctagatctcctcacagccgacaaaggcggaacctgcatgtttctcggggaagagtgctgttattacatcaatgaatcaggcttagtagaaaccagcctcctcacccttgataaaatccgggacggtctccatcgaccctcctcaactcccaactatggaggagggtggtggcaatcccctttaaccacttggattatccctttcataagccccatcctaatcatttgccttttacttctcatagccccctgtgtcctcaagttcatcaaaaaccgcatcagcgaagtctcccgggtgacggtcaaccaaatgttactacacccttactcccgtcttccgacctccgaagaccactatgacgacgccctcactcagcaggaagcagccagatgattacgtcgcccctttttcttacagtatgaggtcggaatgttaggcaggtcacccaagatggctgttcccccaggacccaagatggcggcacgaaccccttcttccccgccccccccaccgcttggagtctcccaccagattttcccgcccgacgggcactttccgatgacagcagccccgagaagtcgaaacctatcccagaaaaccgaaacttactaa

SEQ ID NO: 10 - DNA, 2049 bp, Homo sapiens
atgaggaagctcatcgtgggattcatttttcttacattttggacttacacagtaagggcttcaactgaccttactcaaactggggactgttcccagagtattcatcaggtcaccgaggtaggacagcaaattaaaacaaactttctgttctatagttattatgaatgtatgggaacattaaaagaaacttgtttgtataatgccactcagtacaaggtatgtagcccaggaaatgaccgacccgatgtgtgttataacccatctgagccccctgcaaccaccgtttttgaaataagattaagaactggccttttcctaggtgacacaagtaaaataataactagaacagtagaaaaaggaatccccaaacaaataactttaagatttgatgctcgtgcagccattaatagtaacaagttaggaacacgatgtggttctcttaactgggaaaggagctacacagtacaaaataaatatgtttgtcatgagtcaggggtttgtgaaaattgtgccttttggccatgtgttatttgggctacttggaaaaagaacaaaaaggaccctgttcatcttcagaagggggaagccaacccctcctgtgctgccggtcactgtaacccactagaactaataattaccaatcccctagatcctccttggaaaaagggagaacgtgtaaccctggggatcgatgggacagggttaaacccccaagttgccattttagttagaggggaagtccacaagcgctctcccaaaccagtgtttcaaaccttttatgaggagctgaatctgccagcaccagaacttccgaaaaagacaaaaagtttgtttctccaattagcaggaaatgtagctcattcacttaatgttacttcctgttatgtatgcaggggaaccactatcggagaccgatggccttgggaagcccgagagttggtgcctactgatccagctcctgatataattccagttcagaaggcccaagctagcaacttctgggtcctaaaaacctcaattattggacaatactgtatagctagagaagggaaagaatttatcgtccctgtaggaaagcttaattgtataggacagaagttgtacaacagcacaacaaagacaattacttggtggggcctaaaccacactgaaaagaacccatttagtaaattttctaaattaaaaactgcctgggctcatccagaatctcatcaggactggacggctcccaccggactataccggatatgtgggcacacagcctacattcagttacctaataaatgggcaggcagttgtgttattggcactattaagctgtcctttttcttattacccataaaaacgggtgagctcctaggtttccgtgtctatacctcccgagaaaagagaggcatagttataggaaactggaaagataatgagtggccccctgaaaggatcatacaatattatgggcctgccacatgggtacaagacggctcatggggataccaaacccccatctacatgctcaatcagatcatacggttgcagaccgtcttagaaataattactaatgaaactggcagagctttgactgttttagctcggcaggaaacccaaatgaggaatgctatctatcagaatagactggccttggactacttgctggcagctgaaggaggagtttgtggaaaatttaacttaaccaattgctgcctacaaatagatgatcaaggacaggtgattgaaaacatagtcagggacatgacaaagttggcacacacgcctatacaggtttggcacaagtttgatcctgagtctttatttggaaaatggtttccagctataggaggatttaaaaccctcattgtaggtgtattgctagtaataagaacttgcttgctgctcccctgtgtattacccttgctttttcaaatgataaaaggtattgttgctactttggttcatcagaaaacttcagcacacgtgaattatatgaatcactatcgctctatctcgcaaagagactcaaaaagtgaggatgagagtgagaactcccactaa

SEQ ID NO: 11 - DNA, 1998 bp, Homo sapiens
atggacccatcacacccgagtcaagaaagcaccgccccctccagcgtcatgggccatagtcccaggggaaaatcctaccaaactaaagctaaggaaagtttaattctctttcatctattctgttactccttcttctttccttgtgctcttgctagccacctcattattaacgtaactagatcagactcaccccagaccattaccttcgatgcttgtttagttataccttgtggggatctccaaagccagagacagcttgcagcagcagagaaatatctctgcccctccgaagcagatgcttctaccttgtttagctttccgttttgtcatacttgggaatatgtcgtttggaccactcaacgtcaagattgggtcccctcacaggatttcccgctagcggttctaaagccctatatccattttactaaaggaattgcccctcccaattgtcgatataaccaatgtaacccggtgcaaatttccatcaccatcccaactctccaagattcctcccccaccctaaaccgtttctatggtatgggagcagatgtaagagggaaagaccccataggattcttcgagttgcacctcagtacatctccatccctcatatctccacgtctatcctcttctacacctgctaaccagaccattgtctcttcatctaatgacaaaagcaaagtagctattgtagaggttaaaaatttaaaacaaacactgacaattgaaacaggatacaaagaaacaaatgcctggatggaatggattgaatattccgttcgcagtctaaacaaaagcgactgttacgcttgtgcgcaaggtaggccagaggcccaagtcgtcccctttccacttggatggtcttctgaccaaccgggcatgggctgcatggtggctcttttccaacaccctacagcctgggatagtgagttctgtcgaactctctctgtgctattccctgaaactcaacaccttgagggtgagcccccgagggccatccagcctccatctccagatgccaagtttacttcctgtctctcacgacagggaaaaaatttggagtttcttggggacctaaagggatgcagtgagcttaagtctttccaagagcttaccaatcagtctgcacttgttcatgcccgagctgatgtatggtggtattgtggtggtcacctgctagacactctgccaagtaactggagtggtacttgtgctctaattcaattggccatccctttcaccctggcatttcaacaaccagaaaaaaagaaaccacaacgccgtaaaacaagagaggcccctcaaggatctttcgactctcatgtctatgtagatgaaattggagtcccacagggggtacctgatagattcaaagcccgagacccagtagctgcaggatttgaatcattatttccaatggtagctattaataaaaatgtagcttggataaattacatctattataaccagcagcgatttattaattacactagggatgctatccaaggaatagctgaacaattagggcctactagccaaatggcttgggaaaacagaatggccttagatatgatattagcagaaaaaggtggagtttgtgttatgataggaacccaatgctgcacctacattcccaacaatacagctcctgatggaacaattacaaaagctttacaaggtcttacctccttatcagatgaattagccacaaattctgggataactgaccctttcacaggatggttagggcaatggtttggtaaatggaaaggactcatggcctctattgttacctctctcgcaatcgcaatagctgtgcttattcttgttggatgctgcatcatgccctgcattcgtggactagtccaaagacttatagagacagctagtaacaaaaccttccctagttcttcccaatcctatagtaacaaattcttccccgtgaacgaacacgaaatccgaatcatattagataggtttaaagcagaacatgtataa

SEQ ID NO: 12 - DNA, 1608 bp, Homo sapiens; identical to SEQ ID NO: 1

SEQ ID NO: 13 - DNA, 1692 bp, Homo sapiens
atgggctccctttcaaactatgccctgcttcaactaacccttactgcttttttgacaattctagtacaacctcagcacctgcttgctccagttttccggacactatctatcttgactaatcagtctaattgctggttatgtgaacatctagataatgcagaacaacccgaactagtttttgttcctgccagtgcaagcacctggtggacctattctggacaatggatgtatgaaagggtgtggtatccacaagcagaagtacagaatcactctacttcctcctatcgtaaagtgacttggcactgggaagcctccatggaagctcaaggtctatcctttgctcaagtaaggttattggagggaaatttttctctttgcgtagaaaataaaaatggcagtggacccttcctaggtaatatacctaaacaatactgtaatcaaatactatggtttgattctacagatggcaccttcatgccctctatagatgttacaaatgaatccaggaacgatgatgatgatacaagtgtttgcctaggcactagacaatgttcctggtttgcaggttgcacaaaccggacctggaacagctcagctgttcccttgattggtctgcccaatacccaagactacaaatgggtagatcgaaattctggattgacctggtcaggtaatgacacctgtctctatagctgccaaaaccaaaccaaaggccttctgtaccagctatttcgcaacctattttgctcttatggcctgacagaggcacatgggaaatggagatgtgcagatgccagcataactaatgacaaaggtcatgatggacaccggacccccacctggtggctcacaggttccaatctgaccttgtctgtgaacaactctggcctcttttttttgtgcggcaatggggtgtacaaagggtttccacctaaatggtctgggcgatgtggacttgggtatcttgtaccttccctcaccagatacctcaccttaaatgctagccaaattacaaacctgagatccttcattcataaagtaacaccgcatagatgcacccaaggagacacagacaatccacctctgtattgcaaccccaaggacaattcaacaataagggccctttttccaagtttgggaacttatgatttagaaaaggcaattctaaacatttccaaagcaatggaacaggaattcagtgccactaagcagaccttggaagcacaccaatcaaaagttagcagtttagcctctgcatcccgaaaggatcatgtcttggatataccgaccacccaacgacaaacggcttgtggaactgttggcaaacagtgttgcctctatataaattattcggaagaaataaagtctaatatacagcgtctccacgaagcatccgagaacctgaagaatgtaccgttacttgattggcaaggcatatttgcaaaagtgggagactggttcagatcatggggctatgtgcttttaattgttcttttctgcttattcatctttgttttaatctatgttcgtgtctttcgcaaatctcgcagatcccttaactcccaacctctgaacctagccttatctccacagcaatcagcacagctccttgtcagtgaaacttcatgtcaagtttcaaatagggcaatgaagggactaacaacccatcaatatgacacaagtctactttga

SEQ ID NO: 14 - DNA, 1599 bp, Homo sapiens
atgggcaaccttccaccctccattcctccttcttctcccttagcctgtgttctcaagaacttaaaacctcttcaactcacacctgacctaaaacctaaatgccttattttcttctgcaataccgcttggccccaatacaaacttgacagtggctctaaatggccagaaaatggcactttcaatttttccatcctacaagacctaaataatttttgtcgaaaaatgggcaaatggtctgaggtgcctgacgtccaggcatttttcacacatcgttccctccctagtctctgttcccaatgcaactcctcccaaatcttccttctttccctcccgcctgtcccctcagtcccaaccccaagcgtcgctgagtctttccaatcttccttttctacagacccatctgacctctcccctcctccccaggctgctcatcgccaggccgagctaggtcccaattcttcctcagcctccgctcctccaccctataatccttttatcacctcccctcctcacacccggtccggcttacagtttcgttccgtgactagccctcccccacctgcccagcaatttcctcttaaaaaggtggctggagctaaaggcatagtcaaggttaatgctcctttttctttatcagacctctcccaaatcagtcagcgtttaggctctttttcatcagaccccactaaatatatacaggaattccaacatctaactctgtcctacaatttaacctggagtgacttaaatgtcatcctgacttctaccctctccccagatgcacaggaaagagttttttctctagcccaatctcatgctgataaccgccggcttcatgagccagacctccaggaaggcattagagcagttccccgagaggatccccaatggaactaccaggcaaattccccaggtatagctaagcaagattacatggtttcctgcgtagttgaagggcttaaaaaagcagcttacaaagctattaattatgacaaacttaaagaaactacccaaggtaaagataaaaacccagcccagttcatggctcgtttggcagcaaccctcagacgctttacagccctagaccctaaaaggtcaaaaggccgtcttattctcaatatacattttattacccaatctgctcccgacattaaaaaactccaaaaattaaattctggccctcaaaccccacaacaggatttaattaacctcgccttcaaggtgtacaataatagaaaaaaagcagccagatggcaacgcatttctgagttgcaattccttgcctccactgtgagacaaaccccagccacatctccagcacacaagaacttccaaatgcctgaaccgcagcagccaggcattcctccagaacctcctcccccaggagcttgctacaagtgccagaaatctggccactgggccaaggaatgcccgcagcccaggattcctcctaagccgtgtcccatctgtgcaggaccccactggaaatcagactgttcaactcacctggcagccactcccagagcccctggaactctggcccaaggctctctgactgactccttcccagatctgctcggcttagcggctgaagactga

SEQ ID NO: 15 - DNA, 5638 bp, Homo sapiens
atggggcaaactaaaagtaaaattaaaagtaaatatgcctcttatctcagctttattaaaattcttttaaaaagagggggagttaaagtatctacaaaaaatctaatcaagctatttcaaataatagaacaattttgcccatggtttccagaacaaggaactttagatctaaaagattggaaaagaattggtaaggaactaaaacaagcaggtaggaagggtaatatcattccacttacagtatggaatgattgggccattattaaagcagctttagaaccatttcaaacagaagaagatagcgtttcagtttctgatgcccctggaagctgtataatagattgtaatgaaaacacaaggaaaaaatcccagaaagaaacggaaggtttacattgcgaatatgtagcagagccggtaatggctcagtcaacgcaaaatgttgactataatcaattacaggaggtgatatatcctgaaacgttaaaattagaaggaaaaggtccagaattagtggggccatcagagtctaaaccacgaggcacaagtcctcttccagcaggtcaggtgcccgtaacattacaacctcaaaagcaggttaaagaaaataagacccaaccgccagtagcctatcaatactggcctccggctgaacttcagtatcggccacccccagaaagtcagtatggatatccaggaatgcccccagcaccacagggcagggcgccataccctcagccgcccactaggagacttaatcctacggcaccacctagtagacagggtagtgaattacatgaaattattgataaatcaagaaaggaaggagatactgaggcatggcaattcccagtaacgttagaaccgatgccacctggagaaggagcccaagagggagagcctcccacagttgaggccagatacaagtctttttcgataaaaatgctaaaagatatgaaagagggagtaaaacagtatggacccaactccccttatatgaggacattattagattccattgctcatggacatagactcattccttatgattgggagattctggcaaaatcgtctctctcaccctctcaatttttacaatttaagacttggtggattgatggggtacaagaacaggtccgaagaaatagggctgccaatcctccagttaacatagatgcagatcaactattaggaataggtcaaaattggagtactattagtcaacaagcattaatgcaaaatgaggccattgagcaagttagagctatctgccttagagcctgggaaaaaatccaagacccaggaagtacctgcccctcatttaatacagtaagacaaggttcaaaagagccctatcctgattttgtggcaaggctccaagatgttgctcaaaagtcaattgccgatgaaaaagcccgtaaggtcatagtggagttgatggcatatgaaaacgccaatcctgagtgtcaatcagccattaagccattaaaaggaaaggttcctgcaggatcagatgtaatctcagaatatgtaaaagcctgtgatggaatcggaggagctatgcataaagctatgcttatggctcaagcaataacaggagttgttttaggaggacaagttagaacatttggaggaaaatgttataattgtggtcaaattggtcacttaaaaaagaattgcccagtcttaaacaaacagaatataactattcaagcaactacaacaggtagagagccacctgacttatgtccaagatgtaaaaaaggaaaacattgggctagtcaatgtcgttctaaatttgataaaaatgggcaaccattgtcgggaaacgagcaaaggggccagcctcaggccccacaacaaactggggcattcccaattcagccatttgttcctcagggttttcagggacaacaacccccactgtcccaagtgtttcagggaataagccagttaccacaatacaacaattgtcccccgccacaagcggcagtgcagcagtagatttatgtactatacaagcagtctctctgcttccaggggagcccccacaaaaaatccccacaggggtatatggccccctgcctgaggggactgtaggactaatcttgggaagatcaagtctaaatctaaaaggagttcaaattcatactagtgtggttgattcagactataaaggcgaaattcaattggttattagctcttcaattccttggagtgccagtccaggagacaggattgctcaattattactcctgccatatattaagggtggaaatagtgaaataaaaagaataggagggcttggaagcactgatccaacaggaaaggctgcatattgggcaagtcaggtctcagagaacagacctgtgtgtaaggccattattcaaggaaaacagtttgaagggttggtagacactggagcagatgtctctatcattgctttaaatcagtggccaaaaaattggcctaaacaaaaggctgttacaggacttgtcggcataggcacagcctcagaagtgtatcaaagtacggagattttacattgcttagggccagataatcaagaaagtactgttcagccaatgattacttcaattcctcttaatctgtggggtcgagatttattacaacaatggggtgcggaaatcaccatgcccgctccattatatagccccacgagtcaaaaaatcatgaccaagatgggatatataccaggaaagggactagggaaaaatgaagatggcattaaagttccagttgaggctaaaataaatcaagaaagagaaggaatagggtatcctttttaggggcggccactgtagagcctcctaaacccataccattaacttggaaaacagaaaaaccggtgtgggtaaatcagtggccgctaccaaaacaaaaactggaggctttacatttattagcaaatgaacagttagaaaagggtcatattgagccttcgttctcaccttggaattctcctgtgtttgtaattcagaagaaatcaggcaaatggcgtatgttaactgacttaagggctgtaaacgccgtaattcaacccatggggcctctccaacccgggttgccctctccggccatgatcccaaaagattggcctttaattataattgatctaaaggattgcttttttaccatccctctggcagagcaggattgtgaaaaatttgcctttactataccagccataaataataaagaaccagccaccaggtttcagtggaaagtgttacctcagggaatgcttaatagtccaactatttgtcagacttttgtaggtcgagctcttcaaccagttagagaaaagttttcagactgttatattattcattatattgatgatattttatgtgctgcagaaacgaaagataaattaattgactgttatacatttctgcaagcagaggttgccaatgctggactggcaatagcatctgataagatccaaacctctactccttttcattatttagggatgcagatagaaaatagaaaaattaagccacaaaaaatagaaataagaaaagacacattaaaaacactaaatgattttcaaaaattactaggagatattaattggattcggccaactctaggcattcctacttatgccatgtcaaatttgttctctatcttaagaggagactcagacttaaatagtaaaagaatgttaaccccagaggcaacaaaagaaattaaattagtggaagaaaaaattcagtcagcgcaaataaatagaatagatcccttagccccactccaacttttgatttttgccactgcacattctccaacaggcatcattattcaaaatactgatcttgtggagtggtcattccttcctcacagtacagttaagacttttacattgtacttggatcaaatagctacattaatcggtcagacaagattacgaataataaaattatgtggaaatgacccagacaaaatagttgtccctttaaccaaggaacaagttagacaagcctttatcaattctggtgcatggcagattggtcttgctaattttgtgggaattattgataatcattacccaaaaacaaagatcttccagttcttaaaattgactacttggattctacctaaaattaccagacgtgaacctttagaaaatgctctaacagtatttactgatggttccagcaatggaaaagcagcttacacagggccgaaagaacgagtaatcaaaactccatatcaatcggctcaaagagcagagttggttgcagtcattacagtgttacaagattttgaccaacctatcaatattatatcagattctgcatatgtagtacaggctacaagggatgttgagacagctctaattaaatatagcatggatgatcagttaaaccagctattcaatttattacaacaaactgtaagaaaaagaaatttcccattttatattactcatattcgagcacacactaatttaccagggcctttgactaaagcaaatgaacaagctgacttactggtatcatctgcactcataaaagcacaagaacttcatgctttgactcatgtaaatgcagcaggattaaaaaacaaatttgatgtcacatggaaacaggcaaaagatattgtacaacattgcacccagtgtcaagtcttacacctgcccactcaagaggcaggagttaatcccagaggtctgtgtcctaatgcattatggcaaatggatgtcacgcatgtaccttcatttggaagattatcatatgttcatgtaacagttgatacttattcacatttcatatgggcaacttgccaaacaggagaaagtacttcccatgttaaaaaacatttattgtcttgttttgctgtaatgggagttccagaaaaaatcaaaactgacaatggaccaggatattgtagtaaagctttccaaaaattcttaagtcagtggaaaatttcacatacaacaggaattccttataattcccaaggacaggccatagttgaaagaactaatagaacactcaaaactcaattagttaaacaaaaagaagggggagacagtaaggagtgtaccactcctcagatgcaacttaatctagcactctatactttaaattttttaaacatttatagaaatcagactactacttctgcagaacaacatcttactggtaaaaagaacagcccacatgaaggaaaactaatttggtggaaagataataaaaataagacatgggaaatagggaaggtgataacgtgggggagaggttttgcttgtgtttcaccaggagaaaatcagcttcctgtttggatacccactagacatttgaagttctacaatgaacccatcggagatgcaaagaaaagcacctccgcggagacggagacaccgcaatcgagcaccgttgactcacaagatgaacaaaatggtgacgtcagaagaacagatgaagttgccatccaccaagaaggcagagccgccgacttgggcacaactaaagaagctgacgcagttagctacaaaatatctagagaacacaaaggtgacacaaaccccagagagtatgctgcttgcagccttgatgattgtatcaatggtggtaagtctccctatgcctgcaggagcagctgcagctaa

SEQ ID NO: 16 - DNA, 5714 bp, Homo sapiens
gtcgacgagagatcccgagtacgtctacagtcagccttacggtaagcttgtgcgctcggaagaagctagggtgataatgggccagaccaagagcaagatcaagagcaagtacgccagctacctgagcttcatcaagatcctgctgaagaggggcggagtgaaggtgtccaccaagaacctgatcaagctgttccagatcatcgagcagttctgcccctggttccccgagcagggcaccctggacctgaaggactggaagcggatcggcaaagagctgaagcaggccggcaggaagggcaacatcatccccctgaccgtgtggaacgactgggccatcatcaaggccgccctggaacccttccagaccgaagaggacagcgtgagcgtgagcgacgcccctggcagctgcatcatcgactgcaacgagaaaacgcgtaagaagagccagaaagagaccgagagcctgcactgcgagtacgtggccgagcccgtgatggcccagagcacccagaacgtggactacaaccagctgcaggaagtgatctaccccgagaccctgaagctggaaggcaagggccccgaactggtgggccccagcgagagcaagcccaggggcaccagccctctgcctgccggccaggtgcccgtgaccctgcagccccagaaacaggtgaaagagaacaagacccagccccccgtggcctaccagtactggccccctgccgagctgcagtaccggcctccccccgagagccagtacggctaccccggcatgccccctgcccctcagggcagagccccctacccccagcctcccaccaggcggctgaaccccaccgcccctcccagcaggcagggcagcgagctgcacgagatcatcgacaagagcaggaaagagggcgacaccgaggcctggcagttccctgtgaccctggaacccatgcctcccggcgagggcgcccaggaaggcgagccccccaccgtggaggcccggtacaagagcttcagcatcaagatgctgaaggacatgaaagaaggcgtgaagcagtacggccccaacagcccctacatgcggaccctgctggacagcatcgcccacggccaccggctgatcccttacgactgggagatcctggccaagagcagcctgagccccagccagttcctgcagttcaagacctggtggatcgacggcgtgcaggaacaggtgcggcggaacagggccgccaacccccccgtgaacatcgacgccgaccagctgctgggcatcggccagaactggtccaccatcagccagcaggccctgatgcagaacgaggccatcgagcaggtgcgggccatctgcctgcgggcctgggagaagatccaggaccccggcagcacctgccccagcttcaacaccgtgagacagggcagcaaagagccctaccccgacttcgtggcccggctgcaggacgtggcccagaagagcatcgccgacgagaaggcccggaaggtgatcgtggagctgatggcctacgagaacgccaaccccgagtgccagagcgccatcaagcccctgaagggcaaggtgccagccggcagcgacgtgatcagcgagtatgtgaaggcctgcgacggcatcggcggagccatgcacaaggccatgctgatggcccaggccatcaccggcgtggtgctgggcggacaggtgcggaccttcggcggcaagtgctacaactgcggccagatcggccacctgaagaaaaactgccccgtgctgaacaagcagaacatcaccatccaggccaccaccaccggcagagagccccccgacctgtgcccccggtgcaagaagggcaagcactgggccagccagtgcagaagcaagttcgataaaaatgggcaaccattgtcgggaaacgagcaaaggggccagcctcaggccccacaacaaactggggcattcccaattcagccatttgttcctcagggttttcagggacaacaacccccactgtcccaagtgtttcagggaataagccagttaccacaatacaacaattgtcccccgccacaagcggcagtgcagcagtagatctgtgcacaatccaggccgtgagcctgctgcccggcgagcctccccagaagatccccaccggcgtgtacggccctctgcccgagggcaccgtgggcctgatcctgggccggtccagcctgaacctgaagggcgtgcagatccacactagtgtggtggacagcgactacaagggcgagatccagctggtgatcagcagcagcatcccctggtccgccagccctggcgaccggatcgcccagctgctgctgctgccctacatcaagggcggcaacagcgagatcaagcggatcggcggcctgggcagcaccgaccccacaggcaaggccgcctactgggcctcccaggtgtccgagaaccggcccgtgtgcaaggccatcatccagggcaagcagttcgagggcctggtggacacaggcgccgacgtgagcatcatcgccctgaaccagtggcccaagaactggcccaagcagaaagccgtgaccggcctggtgggcatcgggaccgccagcgaggtgtaccagagcacagagatcctgcactgtctgggccccgacaaccaggaaagcaccgtgcagcccatgatcaccagcatccccctgaatctgtggggcagggacctgctgcagcagtggggagccgagatcaccatgcctgcccccctgtacagccccacaagccagaaaatcatgaccaagatgggctacatccccggcaagggcctgggcaagaacgaggacggcatcaaagtgcccgtggaggctaaaataaatcaaaaaagagaaggaatagggtatcctttttaggggcggccactgtagagcctcctaaacccataccattaacttggaaaacagaaaaaccagtgtgggtaaatcagtggccgctacctaagcagaaactggaagccctgcacctgctggccaacgaacagctggaaaagggccacatcgagcccagcttcagcccctggaacagccccgtgttcgtgatccagaagaaaagcggcaagtggcggatgctgaccgacctgcgggccgtgaacgccgtgatccagcccatgggccccctgcagcctggcctgcccagccccgccatgatccccaaggactggcctctgatcatcatcgatctgaaggactgcttcttcaccatccccctggccgagcaggactgcgagaagttcgcctttaccatccccgccatcaacaacaaagagcccgccacccggttccagtggaaggtgctgccccagggcatgctgaacagccccaccatctgccagaccttcgtgggcagagctctgcagccagtgagagagaagtttagcgactgctacatcatccactacatcgacgacatcctgtgtgccgccgagaccaaggacaagctgatcgattgctacaccttcctgcaggccgaggtggccaatgccggcctggccatcgccagcgacaagatccagaccagcacccccttccactacctgggcatgcagatcgagaaccggaagatcaagcctcagaagatcgagatcaggaaggacaccctgaaaaccctgaacgacttccagaagctgctgggggacatcaactggatccggcccaccctgggcatccccacctacgccatgagcaacctgttcagcatcctgcggggcgacagcgacctgaacagcaagagaatgctgacccccgaggccaccaaagaaatcaaactggtggaggaaaagatccagagcgcccagatcaacaggatcgaccccctggcccctctgcagctgctgatcttcgccaccgcccacagccctaccggcatcatcatccagaacaccgatctggtggagtggagcttcctgccccacagcaccgtgaaaaccttcaccctgtacctggaccagatcgccaccctgatcggccagacccggctgcggatcatcaagctgtgcggcaacgaccccgacaagatcgtggtgcccctgaccaaagaacaggtgcgccaggccttcatcaacagcggcgcctggcagatcgggctggccaatttcgtgggcatcatcgataaccactaccccaagaccaagatcttccagttcctgaagctgaccacctggatcctgcccaagatcacacggcgggagcctctggaaaacgccctgaccgtgttcaccgacggcagcagcaacggcaaagccgcctataccggccccaaagaacgggtgatcaagaccccctaccagtccgcccagcgggccgaactggtggccgtgatcaccgtgctgcaggacttcgaccagcccatcaacatcatcagcgacagcgcctacgtggtgcaggccacccgggacgtggagaccgccctgatcaagtacagcatggacgaccagctgaaccagctgttcaatctgctgcagcagaccgtgcggaagcggaacttccccttctacatcacccacatccgggcccacaccaacctgccaggccctctgaccaaggccaatgagcaggccgacctgctggtgtccagcgccctgattaaggcccaggaactgcacgccctgacacacgtgaatgccgccggactgaagaacaagttcgacgtgacctggaagcaggccaaggacatcgtgcagcactgcacccagtgccaggtgctgcacctgcccacccaggaagccggcgtgaaccccaggggcctgtgccccaacgccctgtggcagatggacgtgacccacgtgcccagcttcggcagactgagctacgtgcacgtgaccgtggacacctacagccacttcatctgggccacctgccagaccggcgagagcaccagccacgtgaagaaacacctgctgtcctgcttcgccgtgatgggcgtgcccgagaagatcaagaccgacaacggccctggctactgcagcaaggccttccagaagttcctgagccagtggaagatctcccacaccacaggaattccttacaacagccagggccaggccatcgtggagcggaccaaccggacactgaaaacacagctggtgaagcagaaagaggggggcgactccaaagagtgcaccaccccccagatgcagctgaacctggccctgtacaccctgaactttctgaacatctaccggaaccagaccaccacctccgccgagcagcacctgaccggcaagaagaacagcccccacgagggcaagctgatttggtggaaggacaacaagaataagacctgggagatcggcaaggtgatcacctgggggagaggttttgcttgtgtttcaccaggagaaaatcagcttcctgtttggatacccactagacatttgaagttctacaatgaacccatcggagatgcaaagaaaagcacctccgcggagacggagacaccgcaatcgagcaccgttgactcacaagatgaacaaaatggtgacgtcagaagaacagatgaagttgccatccaccaagaaggcagagccgccgacttgggcacaactaaagaagctgacgcagttagctacaaaatatctagagaacacaaaggtgacacaaaccccagagagtatgctgcttgcagccttgatgattgtatcaatggtggtaagtctccctatgcctgcaggagcagctgcagctaa

SEQ ID NO: 17 - DNA, 9988 bp, Homo sapiens
tgcctaaccttgtttttactctaactcattactttgaattttgtcctgcttgtctctttaatcacctatccttgcttctcatgtaaataagactctctgtagctgggaaggccagacaaactccaactgaccccttaatttacaagacactaagggctcctttcccaacccccttttgtaaggagttggcctgggtaaacagatcctcagcatttcaaaggagtccaattaactgataaggtactaacaccaacaatgtatgaagttcccaggatttttctcaaagagataacaacataaaaccttgagttcatgtcttctatagactctatatctaattataatgaaagatttagaaccttgcacctggtacctttgctcttcttgtaaccatttgtcttttaagtggtttatctctctgtaaccattttgcttcttttgattcttgcatgtttttacttctgtagaattattgcatttgagtccccctccccttcctaaacctaggtataaaagttaatcaagccccttcctcggggccgagagaattttcagcattagccatctctttggccgccagcttaataaaggactcttaattcgtctcaaagtgtggcatattttttaactcgcttggatacaacataatggaggtcccagtgagaaattaactccactgggtgagagccggtctcactctgggctcccccggaaggacggctggcttggatggggggcgccacctgaggaaataatttttaaggtcccagaagagtaactgccttccggaggagagcggatcgaccaccgtgtcattgccctaaaattcaacatctgagtcctcagcttctgaccccggggtcaggtaagtcagatttgacttcgtttctggtaagaggggagtggccctgatgagggcgtccctcttttgactcagcctgttactctaggacactagtgggttgagccttggttttctggtaggtgcttttgtatcttggtttgggtaggaagtggtcctgacaaggaccctccactgacttagctcagggcccaggaggcgggagctaagccttggtttccggaagaccggactctcgatctctctctctctttctctcttgtctatcttacatccttctgttgttcaggtttcttggacaatctctgggaaagaaaaggaagaaaaaaagagaaaaaaattgttataaactctgtgtaaatggtgtgtgactgtgggaggacaaggggttgcatttgtcttccagtttgtagctctatggtgaaagctacagagttcaagtgggccctcacctgcggttccgtggcgacctcctaaggcttaaggcagcaccgggcatagctcgatctgagccaggggtttataccagcctgtcgatgctaagaggagcccaagtcccctctgggggatcagccaggcgggcatctgaatgacagcatcacgggaactccttcccttgtctgtctaataaagaaggtaaaaaagggaaaactgtcataattgtttacatgccctagggtcaattgtttgttttatgtttattgtttttttcagtgtctattgtcttgtttaatagttgtcaaggtgttatatgtcaagacatcgatattgcccaagacgtctaggtaaaaacttcttcaaggaccttagtgctgattttttgtcacaagaggttaaatttctcatcaatcatttaggctggccaccacagtcctgtcttttctgccagaaacaagtaaggtgttgttacggaaaagagtgtgaaaaacattcacctgatcggaatttctggcaccatgaaggttgcaggtatttagattgtcataccccacgtccaagtgattagacctcctctaaactaaaccagtggtggattcaaaacagccaccctgcagacctccttgctcacctcttttgtcattctgtaacttttcctgtgcccttaaataaaacactgtgtaaagaaacctacgaccgtaatgctttacttcgtttagactcctactctgttcccctgtggctacgctcccaccttaaaaatgatccgagtggtccttttccccctcatccctgccccctaccccacacatctcgttttccagtgtgacagcaagttcagcatctccaagacttggctctgctctcactccttaaacccttaaaagaaaaagctgaatttgaactatttgcctttgaatcgtggagacaccaaaaatatctaggatacaggtctagaggaagaagaagagggagaacgcctagatccaaccgacccaggagacctcaggctgacccctaatccccctccctcaatctaaaagctacagaaatgtagcaagtagtattagctgttgtagtttttctgttcttcctggtcatgttaattctgtttttccgacactccagccccccagggaaagaatttctctgcccgtgttggatcaacacttcctcccagccctataaaggttggagccctctccaaggtatgctgcagaatttttctctcggtttctcagaggattatggagtccgccttaaaaaaggcaagctccagacactctgcgaagtagaatggccaaagtttggagtcaggtggccccctgaagggtcattgaaccttacaattgttcaagctgtgtggcgggttgttactgaaactcccagccaccctgatcagtttccctacattgatcaatggctaagtttggtcaggagccctcctccatggctccgttcatgcgccattcataattctacctccaaggtcctcctgagccagaccgcgttttcgcctcgaccctcagccggttcagctccccctgtactgcctccctctgaagaagaggagagtctccctcacccagtcccaccgccttacaaccagcctgctcccttaaagtcatcccatgtctcctcgacgacgtcccctgtaggctcgccacccattgcctctcaatcgtgaccgtggcaggaagaagtagcccctctactaccactgagagaggcacaagtccctccaggtgatgagcgctcagcccccttcttagtttatgtccctttttctacttctgacttgtataattagaaaacccataatcctcccttctctgaaaagccccaggctttgacctcactgatggagtccgtactccggactcaccggcccacctaggatgactgccaacagctccttttaacccttttcacctctgaagagagggaacgtatccgaagagaggccagaaagtacttccttacatcagccggtagaccagaggaggaagctagagacctccttgaggaggtctttccctctacccggcctaattgggacccaaattcctcaagtggaaagagagctttagacgattttcaccggtatctcctcgcgggtattaaaggagccgctcagaaacccataaacttgtctaagacaactgaagttgtccaggggcctgatgagtcaccaggagcgtttttagaacgcctccaggaggcttatcggatttacaccccttttgacccggcagctcccgaaaatagccgtgctcttaatttggcatttgtggctcaggcagccccggatattaaaagaaaactccaaaaactggaaggatttgctggaatgaatatcagtcagcttttagaaatagcccaaaaagtttttgacaatcgagagtttgaaaaacaaaaacaagcaacacaggcagctgaaaaggccgctgataaagcattcaaaagacaaacaaaaatcttggtaacagctatccaagagggcagaaaggaaaggcccccattccagaaaaatggccaaggaacctcgggttcccaccagaaaagtaaaagaggtgaacaggcccctctaggaaaagaccaatgtgcttattgcaagcagactgggcactggaaaaaggagtgcccactactgccaaaagaaaaatcagaaaagaaaaaggtcctcaccctccccgcaacagaggagtctgatgattgatggggccagggctccctctctcttggcccccaggagcccatggtgactgctacagtggggggccagcctgtacgcttcctaatagacaccggggcagaacactcagtactgcagacacccttgggcagtgtctctaataaaaaagtggctgtacaagtaggggcaaactggagccagaaaagagtaacacactcatttcttcataagttacaggcctctatctccttctcagcccagcaggctcacctcacactaggaaatacaacgccccccactgcccaactcctgctaactacccctctgtcagaagaataccttctggtttcaccatcacaaccaccggagaataatactaatactctcctgttggacctacagacactctttccccgagtttgggccgagtcaaaccccccaggactggcaaagcaccatctgccagtagttgtagaactcctggccactgccctgccagtccaggtaaaacaatatcctatgagtcagcaggctagagaggggatcaatccccatattcagtgactgttacaagctggcatactcacaccatgtcagtccgcctggaatactccatttttgccggtccagaaacctggaacaaatgattaccagccagtacaggacttgagggaagttaacaagtggacagttactgtccatccaactgtccctaacccttatactttactcagcctgctcccaccagaacatacagtatacactgtccttgacttaaaggatgctttctttgctattcctctggcccccaaaagccaacctatctttgcttttgaatggacagatcctggctcaggagacaccacccaactgacttggactcagttacctcaccctttttggggaagccctccaacaagatcttataccattccgagccctaactgtactcttctccagtatgtagatgaccttttattagctactgaaactactgacagctgcctgcaacatactagggacctactttacctccttcaggaactcgggtatcgggtctcagccaagaaggcccagctttgtcttcccagagtgtcctacctggggtacgagataaacaaaggaaaaagggcactcaccagtgcccggaaggaagccatcctgcgaatccccactcccaccaccaagagacaggtacatgaattcctgggggccatgggatactgtcatctatggatattggggttcgcagagattgcaaagcccctgtatactgctacaggagggaatggcccactaatttggactgacacagaagaacaggcttttcaaaatctgaaaaaggcattaactgaagcccctgctttagccctcccaaatatctcaaagccatttcacctgtttgtccatgaaagccagggagttgctaaaggggtgcttactcagactttaagaccctggagacgcccagtggcctatttatctaagaggctggatcctgtggcctctggatggccaagttgtctgcgagccatagcggctacagcaagcctagtccaagaagctgataagttaactctaggccaaaatttaacccttacagctcctcatgccgtagagactttactacgaagtgcttctggcaaatggatgtcaaatgctcgcatcttgcagtatcagagtttactgttagatcagcctcgtttgactttctctcccacaaggtgtttaaatccagctacactacttcctgacccagactccactattcctgtccatgactgtcaagaactgttagaaactatcgaaactggccgacctgatcttcaagatgtgcccctagaaaaggcggatgccgccgtgttcacagacagtagcagcttcctcaagcaggaagtatgaaaagccagtgcagctgttaccacctcagcacaaaaggctgaattgatcgccctcactcaggctctccgatggggtaaggataaacgtattaacatttacactgacagcaggtacgcctttgctactgtgcatgtacatggagccatctaccaagagcgagggctactcacctcagcaggaaagactatcaaaaacaaagaagaaattttagccctgcttgaagctgtttggctccctcagcaggtggctgtaatccactgcaaaggacatcaaaaagaaaacacggccgttgcccgtggtaaccaaaaagctgattcagcagctcaggaagtggcacggtcttcagttacgcccataaacttgctgcccacagtctcctttccacagccagatctgcctgacaatcccgtatactcaacagaagaaaaaaaactggcttcagatctcagagccaataaaaatcaggaaggttggtggattcttcctgactctagaatcttcataccccgagctcttggagaaactttaatcagtcacctacattctaccacccatttaagaagagcaaagctacctcagctcctccggagccattttaagatcccccatcttcaaagcctaacagatcaagcagctctccagtgcacaacctgcgcccaggtaaatgccaaacaaggtcctaaacccagcccaggccaccgtctctgagaaaactcgccaggagaaaagtgggaaattgactttacagaagtaaaaccacaccgggctgagtacaaataccttctagtactagtagacaccttctccggatggactgaggcatttgctaccaaaaacgaaactgccaacacagtagttaagtttttactcaatgaaatcatccctcaacatgggctgcctgctgccatagggtctgataatggaccagccttcacctcgtccatagctcagtcagtcagtaaggcattaaacattcaatggaagctccattgtgcctatcgaccccagagctctgggcaggtagaacgcatgaaccacaccctaaaaaacactcttacaaaattaatcttagaaaccggtgaaaattgggtaagtctccttcctttagccctacttagagtaaggtgcaccccttaccaggctaggttctcaccttttgaaatcatgtataggagggcgccgcctatcttgcctaagctaagagatgcccatttagcagaaatatcacaagctaatttattacagtacctacagtctctccaacaggtacaagatatcatcctgccacttgttcgaggagcccatcccaatccaattcctgaccagacggggtcctgccattcgttccagccaggagacctagtgtttgttaaaaagttccagaaagaaggactcactcctgcttagaaaagacctcacaccgtcatcctcacgacgccaacggctctgaaagtggatggcattcctgcttggattcatcactcccgcatcaaaaaggccaacagagcccagctagaaacatgggtccccaagcctaggtcaggccccttaaaactgcacctaagtcaggtgaagccattagattaattctttttatttacctctcttgtttgtttttgcctgttatgtcctctgtgccttcctactcctttctcctcacctctttcacaacaggacgtgtatttgcaaacaccacttagaaggccagtacctccaaggaagtctcctttgcagttgatttatgtgtactgttcccaaagccagcccatacccacgaaaagcaacacaatctgccagtcccaggagcaggaagtgtcgaccttgcagcaggatttagacactccaggagccaaactagatgtggaagctccaaaggtgcagaaaaaggactccaaaatgttgacttttacctctgtcctggaaatcaccctgacgctagctgtcgagatacttatcagtttttctgccctgattggacatgtgtaactttagccacctactctggaagatcaaccagatcttcaactctttccataagtcgtgcttctcatcctaaattatgtactagaaaaaattgtaatcctcttactataactgtccatgaccctaattcagctcaatggtattatggcatgtcatggggattaagactttatatcccaggatttgatgttggaactatgttcaccatccaaaagaaaatcctggtctcatggagcccacccaagccaatcaggcctttaactgatctaggtgaccctatgttccagaaacaccctgacaaagttgatttaactgttcctccaccattcctagttcctaaaccccagctgcaaagacaacacctccaacccagcctgatgtccatactaggtggggtacatcacctccttaatctcacccagcctaaactagcccaagattgttggctatgtctaaaagcaaaacccccatattatgtaggattaggagtagaagccacacttaaaagtggccctttatcctgtcatgcacgaccccatgccctcacattaggagatgtgtctggaaatgcttcttgtctaattagtactggatataacttatctgcttctccctttcaggctacctgtaatcagtctctgcttacttccttaagcacctcagtctcctaccaggcacctaacaatacctggttggcctgcacttcaggtctcactcgctgcatcaatgggactgaaccaggacctctcctgtgtgtgttggttcatgtactcccccaggtctacgtgtacagtgggccagaaggacaacttctcattgctccccctgaattacatcccaggctacacagagctgccccactactggttcctcttttagccggtcttagcatagctggatcagcagccattggcacggctgccctggttcagggagaaactggactaatgtccctgtctcaacaagtagatgctgatttaagcaatctccagtcagccataaatatactacatacccaggtagagtctctagctgaagtagttcttcaaaaccgccgaggcttagatctgctatttctctcccaaggagggttatgcgcagctctaggagaaagttgttgcttctatgccaatcagtctggagtcataaaagatacactccaaaaagttcaagaaaatctagataggcgccaacaagaacaagaaaataacatcccctggtatcaaagcatgttcaactggaacccatggctaactactctaatcactgggttagccggacccctccccatcctactattaagtctaatttttgggccttgtatattaaattggtttcttaattttgtaaaacaacgcatagcttctgtcaaacttatgtatcttaaaactcaatataacccccttgttataactgaggaatcaacgatttgattcccctaaaacacaagtggggaaatgaaatgcctaaccttgtttttactctaactcattactttgaattttgtcctgcttgtctctttaatcacctatccttgcttctcatgtaaataagactctctgtagctgagaaggtcggaccaactccaattgaccccttaatttataagacactaagggctccttacccaacccccttttgtaaggagttggcctgggtaaacagatcctcagcatttcaaaagagcccaagtaactgacaaggtactaacaccaacaatgtatgaagttcccaggatttttctcaaagagatgacaacataaaacctggagttcatgtctggcatagaccctatatctaattataatgaaagatttagaaccttgcacctactaccattgctcttcttgtaaccatttgtcttttaagttgtttatttctctgtaaccagtttgcttcttttgattcttgcatgtttttacttctgtagaattattgcatttgagtccccctcctcttcctaaacctaggtataaaagttaatcaagccccttcctcggggccgagagaattttgagcattagccatctctttggcgcccagcttaataaaggactcttaattcatctcaaagtgtggtgtattttttaacttgcttgggtacaaca

SEQ ID NO: 18 - DNA, 10172 bp, Homo sapiens
actgagagacaggactagctggatttcctaggccgactaagaatccctaagcctagctgggaaggtgaccacgtccacctttaaacacggggcttgcaacttagctcacacctgaccaatcagagagctcactaaaatgctaattaggcaaagacaggaggtaaagaaatagccaatcatctattgcctgagagcacagcaggagggacaacaatcgggatataaacccaggcattcgagctggcaacagcagcccccctttgggtcccttccctttgtatgggagctgttttcatgctatttcactctattaaatcttgcaactgcactcttctggtccatgtttcttacggctcgagctgagcttttgctcaccgtccaccactgctgtttgccaccaccgcagacctgccgctgactcccatccctctggatcctgcagggtgtccgctgtgctcctgatccagcgaggcgcccattgccgctcccaattgggctaaaggcttgccattgttcctgcacggctaagtgcctgggtttgttctaattgagctgaacactagtcactgggttccatggttctcttctgtgacccacggcttctaatagaactataacacttaccacatggcccaagattccattccttggaatccgtgaggccaagaactccaggtcagagaatacgaggcttgccaccatcttggaagcggcctgctaccatcttggaagtggttcaccaccatcttgggagctctgtgagcaaggaccccccggtaacattttggcaaccacgaacggacatccaaagcggtgagtaatattggaccactttcacttgctattctgtcctatccttccttagaattggaggaaaataccgggcacctgtcggccagttaaaaacgattagcgtggccaccggacttaagactcaggtgtgaggctatctggggaagggctttctaacaacccccaacccttctgggttgggaacgttggtctgcctcgaagccagcttccactttcaattttcttggggaagccgagggccgactagaggcagaaagctgtcgtcccgaactcccggcagtagccggttgagatcatggcgcagccagaagtctctactcaacagtcgcccatgcatgcacccctacctttccttctgacccatacctcctgggtcccaaccacaactttcttcaaagtgtagccccaaaattctccttacctctgaatatacttcctctgatccctgcctcctaggtactaatggttcagacttccatttcctctagcaagttgtatctccaaagggatctaaggaagctctacgctgcgtccttaggcacctaggctatgaacccagggagtcttatccctggtgtccctcccaatttaggcatacagctctcgacatgggcagttatgtaggacccactccccaccacccttgccagggccccaagtttgtaaatggctaagagaaaagagagacagaggagagagagagaaatggaggagaaagagagagagacagagaggagagagagacagagagagagacagagagagagagagagacaaagaggagagagagagagtcaaagagagaaagaaagagaaagaaatagtaaaaaacagtgtgccctattcctttaaaagccagggtaaatttaaaacctataattgataattgaaggtcttctccatgaccctataacactccaataccaccttgtggtcagtgtaaacaagagcatagcccgaaaacactgagaccactgacaacccgtagcctccctatcaaaaatccttaacccagtaacccacagatggaccaaatgcattcaatcggtagcggcaactgctttgctaacagaaaaaagtagaaaagtaacttttagaggaaacctcattgtgagcacacctcaccagttcagaattattctaagtaaaaaaagcaaaaaggtagcttactaactcaaaaatcttaaagtatggggctattctgttagaaaaaggtaatgtaacaccaaccactgataattcccttaacccagcagatttcctaacaggggatttaaatcttaattaccatacaaaggtccgaccagacctaggaggaactcccttcaggacaggacgatagatggttcctcccaggtgattgaggaaaaaaaccacaatgggtattcagtaattgatacggagactcttgtggaagcagagttagaaaaattgcctaataactggtctcctcaaacgtgcgagctgtttgcactcagccaagccttaaagtacttacagaatcaaaagactatctcaatcctgactcaaaaggttacctacaccctctctgaaatgaatttgcataagaactgttgtttatgggaatgcatcttgatggggcagctgggttgttataaaatactcaggaacccagcccagctctaggactcacccctgagcacaaaggcaatgttgggcacgctggtaaaggaccactagaatccagcagcccagacccctttctttgtggtcaagaaaggcgggaaaacaggtgcaggactgctacatcggtaagcataactaatccgataaacagaggtccatgggtggttacgcaccctggaaaggaactcacccctgagcacaaaggcaatgttgggcacgctggtaaaggaccactagaatccagcagcccggacccctttctttgtggtcaagaaaggcaggaaaacaggtgcaggactgcaacatcagtgagcataactaatccgataagcagaggtccatgggtggtgacgcaccctggaaaggaataagcattaggaccatagaggacactccaggactaaagctcatcggaaaatgactaggggtgctggcatccctatgttcttttttcagatgggaaacgttccccccaaggcaaaaacgcccctaagatgtattctggagaattgggaccaatttgacccctcagacgctaagaaagaaatgacttatattcttctgcagtaccgcctggcgggaagtataaattataacaccatcttacagctagacctcttttgtagaaaagaaggcaaatggagtgaagtgccatatgtacaaactttcttttcattaagagacaactcgcaattatgtaaaaagtgtgatttatgccctacaggaagccctcagagtctacctccctaccccagcatccccccgactccttccccaactaataaggaccccccttcaacccaaatggtccaaaaggagatagacaaaggggtaaacaatgaaccaaagagtgccaatattccccgattatgccccctccaagcagtgggaggaggagaattcggcccagccagagtgcatgtacctttttctctctcagacttaaagcaaattaaaatagacctaggtaaattctcagataaccctgatggctatattgatgttttacaagggttaggacaatcctttgatctgacatggagagatataatgttactgctaaatcagacactaaccccaaatgagagaagtgccgccataactgcagcccgagagtttggcgatctctggtatctcagtcaggtcaatgataggatgacaacagaggaaagagaacgattccccacaggccagcaggcagttcccagtgtagaccctcactgggacacagaatcagaacatggagattggtgccgcagacatttgctaacttgcgtgctagaaggactaaggaaaactaggaagaagcctatgaattattcaatgatgtccactataacacagggaaaggaagaaaatcctactgcctttctggagagactaagggaggcattgaggaagcatacctctctgtcacctgactctattgaaggccaactaatcttaaaggataagtttatcactcagtcagctgcagacattagaaaaaaacttcaaaagtctgccttaggcccggagcaaaacttagaaaccctattgaacttggcaacctcggttttttataatagagatcaggaggagcaggcggaacgggacaaacgggattaaaaaaaaggccaccgctttagtcatggccctcaggcaagcggactttggaggctctggaaaagggaaaagctgggcaaatcgaatgcctaatagggcttgcttccagtgcggtctacaaggacactttaaaaaagattgtccaagtagaaataagccgccccctcgtccatgccccttatgtcaagggaatcactggaaggcccactgccccaggggatgaaggtcctctgagtcagaagccactaaccagatgatccagcagcaggactgagggtgcccggggcaagcgccagcccatgccatcaccctcacagagccccgggtatgcttgaccattgagggccaggaggttaactgtctcctggacactggcgcggccttctcagtcttactctcctgtcccggacaactgtcctccagatctgtcactatccgaggggtcctaggacagccagtcactagatacttctcccagccactaagttgtgactggggaactttactcttttcacatgcttttctaattatgcctgaaagccccactcccttgttagggagagacattctagcaaaagcaggggccattatacacctgaacataggagaaggaacacccgtttgttgtcccctgcttgaggaaggaattaatcctgaagtctgggcaacagaaggacaatatggacgagcaaagaatgcccatcctgttcaagttaaactaaaggattccgcctcctttccctaccaaaggcagtacccccttagacccgaggcccaacaaggactccaaaagattgttaaggacctaaaagcccaaggcctagtaaaaccatgcaatagcccctgcaatactccaattttaggagtacagaaacccaacggacagtggaggttagtgcaagatctcaggattatcaatgaggccgttgtccctctatacccagctgtacctaacccttatactctgctttcccaaataccagaggaagcagagtggtttacagtcctggaccttaaggatgcctttttctgcatccctgtacatcctgactctcaattcttgtttgcctttgaagatccttcgaacccaacgtctcaactcacctggactgttttaccccaagggttcagggatagcccccatctatttggccaggcattagcccaagacttgagccagttctcatacctggacactcttgtccttcggtacgtggatgatttacttttagccgcccgttcagaaaccttgtgccatcaagccacccaagcgctcttaaatttcctcgctacctgtggctacaaggtttccaaaccaaaggctcagctctgctcacagcaggttaaatacttagggctaaaattatccaaaggcaccagggccctcagtgaggaatgtatccagcctatactggcttatcctcatcccaaaaccctaaagcaactaagagggttccttggcataacaggcttctgccgaatatggattcccaggtacggcgaaatagccaggccattatatacactaattaaggaaactcagaaagccaatacccatttagtagatggacacctgaagcaggccctaacccaagccccagtgttaagcttgccaacggggcaagacttttctttatatgtcacagaaaaaacaggaatagctctaggagtccttacacaggtccgagggacgagcttgcaacccgtggcatacctgagtaaggaaattgatgtagtggcaaagggttggcctcattgtttacgggtagtggcggcagtagcagtcttagtatctgaagcagttaaaataatacagggaagagatcttactgtgtggacatctcatgatgtgaatggcatactcactgctaaaggagacttgtggctgtcagacaaccatttacttaaatatcaggctctattacttgaagggccagtgctgcgactgcgcacttgtgcaactcttaacccagccacatttcttccagacaatgaagaaaagatagaacataactgtcaacaagtaattgctcaaacctacgccactcgaggggaccttttagaggttcccttgactgatcccgacctcaacttgtatactgatggaagttcctttgtagaaaaaggacttcgaaaagcggggtatgcagtggtcagtgataatggaatacttgaaagtaatcccctcactccaggaactagtgctcagctggcagaactaatagccctcactcgggcactagaattaggagaaggaaaaagggtaaatatatatacagactctaagtatgcttacctagtcctccatgcccatgcagcaatatggagagaaagggaattcctaacttccgagggaacacctatcaaacatcaggaagccattaggagattattattggctgtacagaaacctaaagaggtggcagtcttacactgccggggtcatcagaaaggaaaggaaagggaaatagaagggaaccgccaagcggatattgaagccaaaagagccgcaaggcaggaccctccattagaaatgcttatagaaggacccctagtatggggtaatcccctccgggaaaccaagccccagtactcagcagaagaaatagaatggggaacctcacgaggacatagtttcctcccctcaggatggctagccaccgaagaaggaaaaatacttttgcctgcagctaaccaatggaaattacttaaaacccttcaccaaacctttcacttaggcattgatagcacccatcagatggccaaatcattatttactggaccaggccttttcaaaactatcaagcagatagtcagggcctgtgaagtgtgccaaagaaataatcccctgccttatcgccaagctccttcaggagaacaaagaacaggccattacccaggagaagactggcaactagattttacccacatgcccaaatctcagggatttcagtatctactagtctgggtagatactttcactggttgggcagaggccttcccttgtaggacagaaaaggcccaagaggtaataaaggcactaattcatgaaataattcccagattcggacttccccgaggcttacagagtgacaatggccctgctttcaaggctgcagtaacccagggagtatcccaggcgttaggcatacaatatcacttacactgcgcctggaggccacaatcctcaggaaaagtcgagaaaatgaatgaaacactcaaacgacatctaaaaaagctaacccaagaaacccacctcgcatggcctgctctgttgcctatagccttactaagaatccaaaactctccccaaaaagcaggacttagcccatacgaaatgctgtatggacagcccttcctaaccaatgaccttgtgcttgaccgagagatggccaacttagttgcagacatcacctccttagccaaatatcaacaagttcttaaaacattacaaggaacctgtccctgagaggagggaaaggaattattccaccctggtgacatggtattagtcaagtcccttccctctaattccccatccctagatacatcctgggaaggaccctacccagtcattttatctaccccaaccgcggttaaagtggctggagtggagtcttggatacatcacactcgagtcaaaccctggatactgccaaaggaacccgaaaatccaggagacaacgctagctattcctgtgaacctctagaggatctgcgcctgctcttcaagcgacaaccgtgaggaaagtaactaaaatcgtaaatccccatggccctcccttatcatatttttctctttactgttctcttaccccctttcactctcactgcaccccctccatgccgctgtactaccagtagctccccttaccaagagcttctatggagaatgcagcttcccagaaatattgatgccccatcgtataggagtttttctaaaggaaaccccactttcaccgcccacacccatatgccccacaactgctataatttgcatgcatgcaaatagttgtcctggaggacttggagccactgtctgttggacttacttcacccataccggtatgtctgatgggggtggagttcaagatcaggcaagagaaaaacacgtaaaggaagtaatctcccaactgacccgggtacatagcacccctagcccctacaaaggactagattctctcaaaactacatgaaaccctccgtacccatactcgcctggtaagcctatttaataccaccctcactgggctccatgaggtctcggcccaaaaccctactaactgttggatgtgcctccccctgcacttcaggccatacatttcaatccctgtacctgaacaatggaacaacttcagcacagaaataaacaccacttccgttttagtaggacctcttgtttccaatctggaaataacccatacctcaaaacctcacctgtgtaaaatttagcaatactatagacacaaccaactcccaatgcatcaggtgggtaactcctcccacacgaatagtctgcctaccctcaggaatattttttgtctgtggtacctcagcctatcgttgtttgaatggctcttcagaatctatgtgcttcctctcattcttagtgccccctatgaccatctacactgaacaagatttatacaatcatgttgtacctaagccccgcaacaaaagagtacccattcttccttttgttatcggagcaggagtgctaggtggactaggtactggcattggcggtatcacaacctctactcagttctactacaaactatctcaagaactaaatggtgacatggaacgggtcgccgactccctggtcaccttgcaagatcaacttaactccctagcagcagtagtccttcaaaatcgaagagctttagacttgctaaccgccgaaagagggggaacctgtttatttttaggggaagaatgctgttattatgttaatcaatccggaatcgtcaccgagaaagttaaagaaattcaagatcgaatacaacgtagagcagaggagcttcaaaacactggaccctggggcctcctcagccaatggatgccctggattctccccttcttaggacctctagcagctataatattgttactcctctttggaccctgtatctttaacctccttgttaagtttgtctcttccagaatcgaagctgtcaaatggagccccagatgcagtccatgactaagatctaccgcggacccctggaccggcctgctagcccatgctccgatgttaatgacatcgaaggcacccctcccgaggaaatctcaactgcacaacccctactatgccccaattcagcaggaagcagttagagcggtcatcggccaacctccccaacagcacttaggttttcctgttgagaggggggactgagagacaggactagctggatttcctaggctgactaagaatccctaagcctagctgggaaggtgaccacatccacctttaaacacggggcttgcaacttagctcacacctgaccaatcagagagctcactaaaatgctaattaggcaaagacaggaggtaaagaaatagccaatcatctattgcctgagagcacagcaggagggacaatgatcgggatataaacccaagtcttcgagccggcaacggcaaccccctttgggtcccctccctttgtatgggagctctgttttcatgctatttcactctattaaatcttgcaactgcactcttctggtccatgtttcttacggcttgagctgagctttcgctcgccatccaccactgctgtttgccgccaccgcagacccgccgctgactcccatccctctggatcatgcagggtgtccgctgtgctcctgatccagcgaggcacccattgccgctcccaatcgggctaaaggcttgccattgttcctgcatggctaagtgcctgggttcatcctaattgagctgaacactagtcactgggttccatggttctcttctgtgacccacagcttctaatagagctataacactcaccgcatggcccaaggttccattccttgaatccataaggccaagaaccccaggtcagagaacacgaggcttgccaccatcttgggagc

SEQ ID NO: 19 - DNA, 9607 bp, Homo sapiens
ggcaggaaatataaaaggaaaaacaagtaaagggaaaacaagtccttccctgatcagtctgactcactccaaagtcctgctggagctatgataacattatctgcaaggccaggcagggaccccaaaagaatgggctccaggagcagagatgagaaaaacaagttctccttatcagtttccgccttgaaattctttccccataccattattctttgttctgctctcacaactatttttgtaactatttctgcaagtttgcaaagatttcataagttcctgtttttctttctgtagcacggcaaggtcacaagacatgcttaagtaagatagggtcatgttgcaaatcctgttgtaaaacctgtcacggtatgattaactgtctttgttctgcttctgtaagactgctttcctgtctcacaggtttcatgccaaaaacctgacccgcccctgttggttgcatgtataaaagtcaagccctgtcattgttcagggctcagcctttggatgttcatcggctgggctggtggtcacctaaataaaatcctcctgttccaccaagtggtctctccagcctcctgattcccacaacattttggcgagccagccaggaggggagactacaggtttactgtctcctttggctgtgggactggagccccgggctgggggagacccgtgacccaaggcaccattgggagaccttcaacccggaggggagattggctctcccatgacccggcgcccctccctgacagtgcattggaacctaaggggctacaggacgattccaggacttcacgctacaggactgcagtaaggtttgggcccaaggcaggacccgtcccataaggatggaaggggagcctgatcacctcctggggtgtacctagtaatccgactcaggacatgagaggggctcgcaaagttggaagaaacctacaccccaactgacccaggacgtgagagtggctcactaagttggttaagaaaggaaactggaagtggggaggtgtgtgaataaatgtgaaagagatggttccaaaaggaaccaacatgctgagtgacgtgtgtggagccacaggtctcttagcatagactgtaccatctgagtgaagtgtggaaccgaccgagactagtggcaaacatcctttggggctacggcatatggcttagggaggtgccccacaacttatagattgtggtggtccgggttcaggactcatatgaaccctccattaaatctaagaagtgtctgaaatactcctgcaagggacacggtctaatcagtctgaaatgaaaggaagagtgagtgggttctgccataactgggaggaaatgggagggaagtcatcaaaacccaccccattagaatgtatgttaaagaactttaagaaaggttatacaggggattatgggatcaagttgaccccccagaggttgagaactctctgtgaaatagaatggccctcttttaatgttggatggccggccgaaggaactatagatagggaaacaattggccatgtatttaaggtggtgactggggtcagaggacagccagggcatccagaccaatttccttatattgactcatggctaaatatagttcaaactcaacctgcatggctgcagccctgcctggcagcttattgcaaaacgctcgtagctcgagccgagcctaaagtgaaagaaaaatcagcttcactgtcagctacagagacaaagggaaagccacaagaaaaaccagttttgcaggaaccaccagaggagatagaaatccctcctccctataccccaatctacccccctttaccaaggccggcccctgagcagccagactcagatggtgacacaccccaggctacaccccaaagggagaaatctgagcccccaccccaggaggtcaaggaggaaagtcaggatgatcaagcaggccgccttcgacctggccacaccagagtgtggcagatgcccctccaggaaacacggggacccatctattatgatgaacagggccaggttcaagggggggcaaccgacttttatttaccagcctttttcaaccactgatctcctactggaaacaccatactccctactccctcctacatggagaagccccaggccctcatagatctgatgcagtccatcttccagacacacaatccaacctggccagattgcaaacagctcctcctgacactgtttaacaccgaggagtgccgaagggtgacccaggcagccctccactggctagaagccaatgcaccagaaggcacacttaatgtccaggcatatgctcagggccagttcccagaagcagacccacactgggacccaaatgatgcaacccaattacagcacctgcagaggtaccgagaggcactcctgcaagggctaaaggaaggtgggaaaaaggcagtcaacatggggaaaatcttggaagtgctgcagggaactgatgaaagccctagccagttttatgagagactctgtgaggcattccagctttacactccgtttgaccctgaggctactgaaaatcagtgcatggtgaacacagcatttgtaggacaagcccagggggacatcaggcgaaagctgcaaaagctagagggtttcgcaggcatgaatgccacccagcttttagaagtggccaccaaggtgtatgttaaccgtgaccaggaggcaaagagaggctgatcagagactggaaaaaggccgatctgctagtggcagccctcacagaaagggaaactagcatcgcgagtgggtgtggacatagccatggacatggaggggttcaggccaggcagagacctgaaagtcaaccaagactatgtaggaatcaatgtgcacaatgcaaaaagaagggacactggaaaaatgaatgtccagagggcaatgaagaaaatgacagagactataaaactggaaaactgccagccaagggctattgtgtcccaagggagccagataccaacctgatcaggctggcagggactgaagaatatgaagactaggccagactgggctccttctccttaggcccccaggagcccatggtcacattagaagtagggggccaactaatggactttatggtagacactggggctgaacactcggtagtgacccagcccatagggccactatccaaaaaccatacaactattgttggggctacaggggtcccagagaagaggccattttgtcagccaaggaggtgtgtcataggaggacgagaagtccaacatgaattcctgtacctcccaaattgtccagttcccttgctgggaagagacctactccaaaaactgcaagcacagattgcttttgggccacaaggagatatgactttaaacctaactcacccaaaggccatggtgttaacccttaccatcccacaggctgaggaatggagactatacatgaagtacaggcaccaatgcagccttgtacgcaggaggaagagaaattattatttaggccaattgataaaattcctggagtatgggctgaagacaacccacctgggctggctgtaaatcaggcactggtagtagaattaaaaccaggagcaactccagttcaggttcgtcagtacccactttcccaagaggatatatggggcatttataaacatttaaagtggctctgtgaacatggaatcatagtccaatgccagtcaccctggaacactccacttttgctggtacaaaaaccattgccaggaccaggatctgatgagtatagaccggtgcaggacttgcatgctgtaaaccaagccacggtgaccatccatccagtagtaccaaacctgtatactttaatgggacttattccagcaagtgctacctggtttacagtcctggacttaaaggatgctttcttctgtctctacctggcaccaattagtcagcccatctttgcatttcaatgggacaattcagtcacaggcacaggggaacagctcacctggactagactcccacaaggattcaaaaactctcccacaatctttggagaagcactggcctcagacctcaaggcctacaccccaccaaatgacaactgcgccttgctgcaatacatagacgaccttcttttggcagccccaacccaagaggactgttaccaaggaacccaagacctcctccatctcttatggaaagcaggttataaagtatccaaaaagaaagcccaaatttgccatgaaaaggttaaatatttaggcttcatagtaagccaaggggaacgctggcttggcaatgaatgaaagcaggctgtttgtgcacttccaactccaaccacccggcgccaaataagagaattcttaggggcagcagggttctgccatatctggatcccaaatttctcacttatggccaagcccttatatgaagccacaaaggagggggaaaaggaacccctcctctgggaggctgaccaggagaaggcatttaaacaaatcaaagaagccttaactcaggccccagccttaggactgccagatataactaagcctttctttctatatgtccatgaatgaaagggaatggctataggggtcctgactcaagtcataggatcatggcatcgcccggtggcatacttatccaaacacttgactctgtggtgcggatggcctccttgccttaaagcactagctgccaccaccctattggcacaagaagctgacaaactgactctagggcaacaactgaccatccaggtaccacactcggttataactttaatggatcagagagggcaccattggttatcaaatccaagaatgactcagtaccaagggctcctatgtgaaaatccccacataactttagaaacagtaacacccttaacccagctaccctgctcccagtcaaaccgggaatcaccctccatgactgtggaaacagtagacgaggtattctcaagtcggggagaccttacagaccaacccctcaaggacccagatgttgaatacttcacagatggaagcagttttgtactggaaggggtccgctgggctgggtatgcagtggtaacattagactcagtggtggaggctcagcctctgcctactggaacatcagcccaaaaggcagagctaatagccctaacaagagctctcttgctggcaaaagacaaaaagatcaatgtttatactgattccaaatatgcttttgccacattgcatgttcatggagctatatataaagaaagaggactcttaactgctggaggcaaagaaataaagtacaaagaagaaattctacagctcttagatgctgtatgggccccaaagaaagtagctgttatgcactgcagggggcaccaaaaggcaggaacactagaggccaaaggaaacagaaaggcagacagggaggcaaaacgggcagcaatgactacaccgcattttaaaaaggaagccctagctatgcctctcctcccagagcctcccctcccagaggtcccaagttactctccaaatgagaaggcctggtttgcccaagaaactggaaaatatattgaaggaggatggtggaaattctctgatgggaggctagccattcctgaaatggtggcccccaaatttgtgaagcaattccatcaaggaactcatatgggaaaaacggcactagaaacgctactaggacaccatttctatgtgccacggctcactgccatcacccgagccgtttgtgaacaatgtctaacttgtgcccagaacaacccatgacaagggcccactcggcccccaggaattcaggaaataggagccacaccctgtgaaaacctgcttatggacttcactgagctgccccgagcagggggctatcggtacatgttagtacttgtctgcaccttttcaggatgggtcgaggctttccccaccagaacagagaaagcatgagaagtgactaaagtactgttaagagacattatccccagatttggactgcccctgactctagggtcagacaatggaccggcatttgtagctgaaatagttcaagaactaacacggctgttaaaaataaaatggaaattacacacagcctaccggccacagagctcaggaaaagtggagcacatgaactggacactcaaacagctactgaagaaatattgccaagaaactcatctgagatgggatcaggtcctgcccatggtcctcctccgagtcaggtgcacccccaccaaacaaactgggtattcgccctatgagatcttgttcggccagccacccccaatcataggtcaaattaagggtgatctccgtgaactaggggaattaactttaagaaggcaaatgcaggctttagggatagccatgcaaagtgtccatggctgggtacaggaaagaatgcccataagcctgacagacccagcacacccctttaaacctggggactctgtttgggttaagaaatggaatccaaccactctgggacccatatgggatgggccccatactgtaatcttgtccactcccactgctgttaaagttgcaggaattgtgccttggatccaccacagtagtcggctgaaaccagcagcccaggacaagtggaccagccaacaggacccagaccatccaacccagctgatcctacgatgggaccaagttgccagtgaagatgacaacagccctgctctggtcactccggaagctgaccagtctatacacggctgaagcttgaggagacaacagccctgctctagtcaccccagaagctgactagtctatgcacggccgaagcttgagtcatcatcagggaagtaaatgtggttagaaatcttaagcctagtaattttccttgtaatattaattactttactattaacctgtcactttgctcaacctcctccccccaggaaaaagccttttctgtccatgctgggtatgaacatgctactcattactttgttcttgctactccccttatccatgttaaaaggagaaccctgggagggatgcccccactgcacacacactacgtggtcggggagcaccataactagaaccctgttgtaccacacttattatgagtgtacagggacccacctaggaacttgtactcacaaccagacaacctactcagtctgtgacccaggaaatggccagccttatatatgttatgaccctaagtcctcacctgggacctggtttgaaattcatgtcgggtcaaaggaaggggatcttctaaaccaaaccaaggtatttccctctggcaaggatgtcgtatccttatactttgatgtttgccagataacatccatgggctcactctttcccgtaatcttcagttccatggagtactatagtagctgccataaaaataggtatgcacaccctgcttgttccaccgattccccagtaacaacttgctgggactgcacaacatggtccactaaccaacaatcactagggccaattatgcttaccaaaataccattaaaaccagattgtaaaacaagcacttgcaatcctgtaaatcttaccatcttagagccagatctgcccatatggactacaggtttaaaagcaccgctaggggcacgagtcagcggtgaagaaattggcccaggagcctatgtctatctatatattataaagaaaactcggacccgttcaacccaacaattccgagtttttgagtcattctatgagcatatcaaccagaagttgcctgaaccccctcccttggccagaaacctattcgcccaactggctgaaaacatagccagcagcctacacatctcctcatgctatgtctgtgggggaaccaacatgggagaccaatggccatgggaagcaagggagctaatgccccaagataacttcactctaactgtctcttcccccgaacctgcgttcacaagccagagcatctggttcttaaaaacctctattattggaagattctgcattgctcgctggggaaaagcctttacagacccagtaggagaattaacttgcctaggacaacaatattacaatgaaacactaggaaaaactttatggtggggcaaaaagaataattccaaatcaccccacccaagcccattctcccgtttcccttctttaaaccactcttggtaccaacttgaagctccaaatacctggcaggcaccctctggcctctactggatctgtgggccacaggcatattggcaactgccagctaaatggtcaggggcctgtgtactggggacaattaggccgtccttcttcctaatccctctaaagcaaggagaagccttaggataccccatctatgatgaaactaaaaggagaaacaaaaggggcataaccataggagattggaaggacaatgaatggccccctgaaagaataatccaatattatggcccagccacctgggcagaagatggaatgtggggataccgcacccctatttacatgctcaaccgcatcataaggttgcaggcagtacttgaaatcattactaatgaaactgcaagggccttaaatctgctggcccagcaagccacaaaaatgagaaatgccatttatcaaaatagactggccttagactacctcctagcccaggaaggaggagtatgtggaaagttcaatctaactaattgctgcctagaaattgatgacaatggaaaggtcatcaaagaaataactgcaaaaatccaaaaattagcccatgttccagtccagacttggaaaggatggtctccagattccctcttcggaggctggttttcatcccttggaggatttaaaaccttagtaggaatagttctggccatactaggaggttgcctaatactcccttgtctcttacccctccttgtcaaaaacatccaaacagccatagaggctcttgtaaacagacagactaccacacaactaatggccctaactaagtattaacccctgccaagaaaagagctacttcctcttgaagtaaatgaagatagtgatgctttctattaaactttacttataaaaagcatcaaaggggggaatgaagcaggaaatataaaaggaaaaacaagtaaagggaaaacaagtcctttcctgaccagtctgactcactccaaagtcctgctggagctatgataattatctgcaaggccaggcaggggctccgaaggagtgggctccaggagcagggatgagaaaaacaagttctccttatcagtttccctgtttgaaattctctccccataacattattctttgttctgctctcacaactatttttgtaactatttctgcaagtctgtaaagattttgtaagttcttgtttttctttctgtagcatggcaaggtcacaagacatgtttaagtaaggtaggctcatgttgcaaatcctgttgtaaaacctgtcacggtatgattaactgcctttgttctgcttctgtaagactgctttctcacctcgcaggttttgcgccaaaaacccgacttgcccctgcctgatgcatgtataaaagtcaagccctgtctttgttcagggctcagcctttggatgttaatccgctgggccagtggccacctaaataaaaccttcctgttgcacccagtgatctctccggcctcctgattcccacaaca

SEQ ID NO: 20 - DNA, 8843 bp, Homo sapiens
tatggtaggagaccaccacttctcctgttgtccttcccagtttctccccaacctccccttttccctagtttataagacaggagaaaagggagaaagcaaaaagttggaaagaaacagaagtaagataaatagctagacgaccttggcgccaccacctggccctggtggttaaaatgataataatattaacccctgaccaaaactactggtgttatctgtaaattccagacattatatgagaaagcactgtaaaactttttgtactgttagctgatgtaggtagcccccagtcacgttcctcacacttacttgatctgtgatgactctttcacgtagaccccttagagttgtaagcccttcaaagggctaggaatttctttttcggggagctcggctcttaagatgcgactctgccgatgctcctggccgaataaaaacctcttccttctttaatccagtgtctgaggagttttgtctgcgactcatcctgctacatttcttggttccctgcctgggaagcgaggtaattgatggacggagggtcgaggcagccccttaggtggcttaggcctgccctgtggagcatccctgtgggggactctggccagcttgagtgacgcagatcctgagaccactcccgggtaggcaattgccccggtggaacgcctcatcagagcagtgcgtggcaggcccctgtggaggatcaacgcagtggctgaacaccgggaaggaatgggcacttggagtccggacatttgaaacttggtaagactggtctttggaacttgcccactccatctgagtggaagcgtggcctgatcacccatggcgtgcctgtaccggcactttggtttttgtttttgacttgacttgaattgcttgatattttggttttggttttgacctggcttggacttctggatactcggattttggttttgattctggtttggtgtaaactgaaaaagtgtgtgtgtgccctttttacctgttctttgttctgtggtgtgtgtgtggtgtaagcttggtgttttgtcttgaggaaacacgggtcagacacaaagtaagcctactccgctaggaactatgttgaaaaatttcaagaaaggatttaatggagactatggagttactatgacaccaggaaaacttagaactttgtgtgaaatagattggccagcattagaagtgggttggccatcagaaggaagcctggacaggtcccttgtttctaaggtatggcacaaggtaactggtaagtcaggacacccagaccagtttccatacatagacacttggttacagctggttttagaccccccacagtggttaagaggacaggcagcagcagtactagtagcaaagggacagatagccaaggaaggatcccgctccacccgctgagggaaatcaactcctgaagttctgttcgacccaacatcagaagatccattgcaggagatggcaccagtgatcccagtggtgccctccccttaccagggagagaggctccccacttttgagtccacagtgcttgcgcctccacaagacaaacatatccctaggccacccagagtagacaagagaggaggtgaagcctcgggagaaacccctcccttggcagctcgtttaagacccaaaactgggatacaaatgcccctgagagagcagcggtatactgggatagatgaggatggacacatggtggagaggcgtgtttttgtgtaccagcccttcacctctgccgacctcctcaactggaaaaataataccccatcctatactgaaaagcctcaagctctaattgatttgctccaaactattatccagacccataaccccacttgggctgattgccaccagttgctcatgtacctctttaacacagatgaaaggcggagagtgctccaagcagcaactaagtggctagaggaacatgcaccagctgattaccaaaacccccaagagtatgtaaggacccagttaccaggaaccgacccccagtgggacccaaatgaaagagaggatatgcaaaggctaaaccgatacagggaagctctcttggaaggattaaagaggggagcccagaaggccacaaacgttaacaaggtctctgaggtcattcaaggaaaagaagaaagtccagcacaattctatgagagactgtgtgaggcctatcgtatgtatactccctttgatcccgatagccctgaaaatcagcgcatgattaacatggctttagttagtcaaagcgcagaagacattagaagaaaactgcagaaacaggctgggtttgcagggatgaacacatcacagttattagaaatagctaaccaggtgtttgtaaacagggatgcagtaagccgtaaggaaaaccacagagagaatgaacgtcaggcccagcgaaacgccgacctgttagctgcagcaatcagaggggtccccccaaagaggcaagggaaggggggccccgggaaagaaactcagcctggctgtcagagcttgcagcgtaatcagtgtgcttattgtaaagaaataggacattggaagaacaaatgccctcagctaaaaggaaaacaaggtgactcggagcaggaggccccagacaaggaggaaggggccctgctcaacctggcagaagggttattggactgagggggactgggctcaagggcccccaaagagcccatggtcaggatgacagttgggggtaaagacattgattttcttgtagatactggtgctgaacattcagtagtaaccaccccggtcgcccccttatccaaaaagactattgatataatcggagccacaggagtttcagcaaagcaagctttctgcttgccccggacttgtactgtaggaggacataaagtgattcatcagtttttgtacatgcctgactgtcccttgcccttgttgggaagggacttgcttagcaagctgagagccactatctcttttacaaagcatggctctttacagctaaagttacccggaacaggagtcattatgacccttacggtcccccgagaggaagaatggagacttttcttaactgagccaggccaagagataagaccagctctggctaagcggtggccaagagtgtgggcagaagacaaccctccagggttggcagtcaaccaagcccccgtactcatagaagttaagcctggggcccagccagttaggcaaaaacagtatccggtccccagagaagctcttgaaggtatccaggtccatctcaagcgcctaagaacctttggaattatagttccttgtcagtctccatggaacactcccctcctgcctgttcccaagccagggaccaaggactacaggccggtacaggatttgcgcttggtcaatcaagctacagtgactttacatccaacagtacctaacccgtacacattgttggggttgctgccagctgaggacagctggttcacctgcctggacctgaaagatgctttctttagcatcagattagcccctgagagccagaagctgtttgcctttcagtgggaagatccggagtcaggtgtcactactcagtacacttggacccggcttccccaagggttcaagaactcccccaccatcttcggggaggcattggctcgagacctccagaagtttcccaccagagacctaggctgcgtgttgctccagtacgttgatgaccttctgctgggacaccccacggcagtcgggtgcgccaaaggaacagatgccctactccggcacctggaggactgtgggtataaggtgtccaagaagaaagctcagatctgcagacagcaggtacgttacctgggatttactatccgacagggggagcgcagcctgggatcagaaagaaagcaggtcatttgcaatctaccggagcctaagaccagaaggcaggtgagagaattcttaggagctgtggggttttgcagactgtggatcccaaactttgcagtattagccaagcctttgtatgaggtcacaaaggggggggaacgggaaccttttgaatggggatcccaacaacagcaagcctttcatgagttaaaggaaaaacttatgtcagccccagccctggggctaccagatctgacaaagccttttacattgtatgtgtcagagagagaaaagatggcagttggagttttaacccaaactgtggggccctggccgaggccagtggcctacctctctaaacaactagacggggtttctaaaggatggcccccatgtttgagggccttggcagcaactgccctgctagtacaagaagcagataagctgactcttgggcaaaacctgaacataaaggccccccatgctgtggtgactttaatgaataccaaaggacatcattggctaacgaatgctagactaactaagtaccaaagcttgctctgtgaaaatccccgtataaccattgaagtttgtaacaccctgaaccccgccaccttgctcccagtatcagagagccctgtcgagcataactgtgtagaagtgttggactcagtttattctagcagacctgacctccgggaccagccttgggcatcagtagactgggagctatacgtggatgggagcagcttcatcaacccacaaggagagagatgtgcaggatatgcggtggtaactctggacactgttattgaagccaaatcgttgccccagggcacttcagcccagaaagctgaactcattgctttaattcgggccttagaactcagtgaaggtaagactgtaaacatttacactgactctcgatatgcctttttaacccttcaagtgcatggagcattatataaagaaaagggcctattgaactctgggggaaaggacataaaatatcaacaagaaatcttgcaattattagaagcagtatggaaaccccacaaggtggcagttatgcattgcagaggacaccagcgagcttccaccttggtgggtttagggaattcccgcgctgactcagaggctcgaaaagcagcatctacccccttccgggcatcagtcacagcccccctgctccctcaagcacctgatcttgtacctacttattctaaagaagaaaaggactttctccaggcagagggaggacaagtgatagaggaaggatggattcggttaccagatgggagagtagctgtgccacagctgctaggagccgcagttgtactggctgtgcatgaaaccacccatctaggccaggagtcacttgaaaagttgttaggctggtatttctacatctcacatttgtcagcccttgccaaaacagtgacgcagcggtgtgttacctgccgacagcataatgcgaggcaaggtccagctgttccacccggcatacaagcttatggagcagccccctttgaagatctccaggtggacttcacagagatgccaaaatgtggaggtaacaagtatttactagttcttgtgtgtacctactctgggtgggtggaggcttatccaacacgaactgagaaagctcgtgaagtaacccgtgtgcttcttcgagatcttattcctaggtttggactgcccttacggatcggctcagataatgggccggcatttgtggctgacttggtacagaagacagcaaaggtattggggatcacatggaaactacatgccgcctaccgacctcagagttccggaaaggtggagcgaatgaatcggactatcaaaaatagtttagggaaagtatgtcaggaaacaggattaaaatggatacaggctctccctatggtattgtttaaaattagatgtaccccttctaaaagaacaggatattccccttatgaaatattatatcataggccccctcctatattgcggggacttccaggcactccccgagagttaggtgaaattgagttacagcgacagctacaggctttaggaaaaattacacaaacaatctcagcctgggtaaatgagagatgccctgttagcttattctccccagttcaccctttctccccaggtgatcgagtgtggatcaaggactggaacgtagcctctttgtggccacggtggaaaggaccccagactgtcatcctgaccactcccaccgctgtgaaggtagaaggaatcccagcctggatccaccacagccatgtaaaacctgcagcgcctgaaacctgggaggcaagaccaagcccagacaacccctgcaaagtgaccctgaagaagacgacaagccctgctccagtcacacccggaagctgactggtccacgcacggccgaagcatgaggaagctcatcgtgggactcatttttcttaaattttggacttatacagtaagggcttcaactgaccttactcaaactggggactgttcccagtgtattcatcaggtcactgaggtaggacaacaagttaaaacaatctttctgttctatagttattatgaatgtataggaactttaaaaggaacttgtttgtataatgccactcagtacaaggtatgtagcccaggaaatgaccgacctgatgtgtgttataacccatctgagccccctgcaaccaccgtttttgaaataagattaagaactggccttttcctaggtgatacaagtaaaataataactagaacagaagaaaaaggaatccccaaacaaataactttaagatttgatgcttgtgcagccattaatagtaaaaagctaggaataggatgtggttctcttaactgggaaaggagctacagagtagaaaataaatatgtttgtcatgagtcaggggtttgtgaaaattgtgcctattggccatgtgttatttgggctacttggaaaaagaacaaaaaggacccggtttatcttcagaagggggaagccaacccctcctgtgctgccggtcactgtaacccactagaactaataattaccaatcccctagatccccattggaaaaagggagaacgtgtaaccctggggatcaatgggacagggttaaacccccaagttgccattttaattagaggggaggtccacaagtgctctcccaaaccagtatttcaaaccttttatgaggagctgaatctgccagcaccagaacttccgaaaaagacaaaaaatttgtttctccaattagcagaaaatgtagctcattcccttaatgttacttcttgttatgtatgcgggggaaccactatcggagaccgatggccttgggaagcccgagagttggtgcctactgatccagctcctgatataattccagttcagaaggcccaagctagcaacttctgggtcctaaaaacctcaattattggacaatactgcatagctagagaagggaaagactttatcatccctgtaggaaagcttaattgtataggacagaagttgtataacagcacaacaaagacaattacttggtggggcctaaaccacactgaaaagaatccatttagtaaattttctaaattaaaaactgcttgggctcatccagaatctcatcaggactggacagctcccgctggactatactggatatgtgggcacagagcctacattcggttacctaataaatgggcaggcagttgtgttattggcactattaagccatcctttttcttattacccataaaaacaggtgagctcctaggtttccctgtctatgcctcccgagaaaagagaagcatagctataggaaactggaaagataatgagtggccccctgaaagaatcatacagtattatgggcctgccacatgggcacaagacggctcatggggataccgaacccccatctacatgctcaaccggatcatacggttacaggctgtcttagaaataattactaatgaaactggcagagctttgactgttttagcttggcaagaaacccaaatgaggaatgctatctatcagaatagactggccttagactacttgctagcagctgaaggaggagtttgtggaaaatttaacttaaccaattgctgtctacaaatagatgatcaaggacaagtggttgaaaacatagtcagagacatgacaaagctggcacatgtgcctgtacaggtttggcacgagtttgatcctgagtctttatttggaaaatggtttccagctataggaggatttaaaaccctcattgtaggtgtattaatagtaataggaacctgcttgctgctcccctgtttgctacccttgctttttcaaatgataaaaagctttgttgctactttagttcatcaaaatacttcagcacaagtgtattatatgaatcactatcgctctatctcacaaaaagactcagaaagtgaggatgagagtgagaactcccactaataaaaagtgaaaattctcaaagggggggaatatggtacgagaccaccacttctcctgttgtccttcccagtttctccccaacctccccttttccctagtttataagacaggagaaaagggagaaagcaaaaagttggaaagaaacagaagaaagataaatagctagacgaccttggcgccaccacctggccctggtggttaaaatgataataatattaacccctgaccaaaactactggtgttatctgtaaattccagacattgtgtgagaaagcactgtaaaactttttgtagtgttagctgatgtaggtagcccacagtcacattcctcacacttacttgatctattatgactctttcacgtagaccccttagagttgtaagcccttcaaagggctaggaatttctttttcggggagctcggctcttaagatgcgagtctgctgatgctcccggccgaataaaaaacctcttccttctttaatccggtgtctcaggagttttgtctgcgactcgtcctgctaca

SEQ ID NO: 21 - DNA, 2482 bp, Homo sapiens
acagactccttcttttcaggagcccactcggttctgtcgttcagggtgtcgcttctttctgagctcctgtctctttaaatgaagggcatttaccctttaactctttctgcgcatctcttgactgaatccaagaagaccaagaaccagaggacttccacacccctctgggtaacatctggtgttttcacctggttacctggttacctcgcctggttatccagctatcaacatttctggcatctccagttggatacatcagtctcaagtgaaactgtgggaaggtcccgaagaaccacagaagaacaacagaattcagcacctgagcattcttgtgagccactggagaacttaaaattccacttcaagtgaaaagataagtaaagccttctctctgttcacccaaaactaaagtcaatctcagtacggggaatcttggttgcggtggcattggttcttctccttattttgacccaactggcatgccacctgaagtcccgataacagcctgatttctcactaaacactccatcgaaccacttcattatttgtctctcctattttcagcactttccttttgctttcacaaagcttaagggagaatcagctatgacagagaaattccttttcctttatctttccctccttcccatgcccctactctcacaggcacagtggaatgaaaattcccttgtcagtttttccaaaataattgcttcgggaaaccatctaagcaactgttggatctgccacaacttcatcaccaggtcctcatcttaccaatatattttggtaagaaatttttctttaaacctaacatttggttcaggaatccctgaaggccaacataaatctgttccgctccaggtttcgcttgctaactcagcgcaccaagtcccctgcctggatctcactccacctttcaatcaaagctctaaaacttctttctatttctacaactgctcttctctaaaccaaacctgttgtccatgccctgaaggacactgtgacaggaagaacacctctgaggagggattccccagtcccaccatccatcccatgagcttctccccagcaggctgccaccctaacttgactcactggtgtccagctaaacaaatgaacgattatcgagacaagtcaccccaaaaccgctgtgcagcttgggaaggaaaagagctaatcacatggagggttctatattcgcttcccaaggcacacactgtccccacatggccaaaatctactgttcccctgggagggcctctatcccctgcatgcaatcaaactattccagcagggtggaaatcgcagttacacaagtggttcgacagccacatcccccggtgggcctgtacccctcctggctatgtatttttatgtgggccacaaaaaaataaactgccctttgatggaagtcctaagataacctattcaaccccccctgtggcaaacctctacacttgcattaataacatccaacatacgggagaatgtgctgtgggacttttgggaccacgggggataggtgtgaccatttataacaccacccaacccagacagaaaagagctctgggtctaatactggcagggatgggtgcggccataggaatgatcgccccatggggagggttcacttatcatgatgtcaccctcagaaatctctccagacaaatagacaacatagctaagagtaccagagatagcatctctaaactcaaggcctccatagattctctagcaaatgtagtcatggacaacagattggccttagattacctcttagcagagcagggtggagtctgtgcagtgatcaataaatcctgttgcgtttatgtcaataacagtggggcgatagaggaggatataaaaaagatctatgatgaggctacgtggctccatgactttggaaaaggaggtgcttcagcaagggccatctgggaggctgtgaagtctgccctcccctccctcaactggtttgtccctttactgggaccagcaacagttatactcttacttttcctctttggcccttgtttctttaatttactgattaagtgtgtctcttctaggataaagcaatttcacatgaagtccccccaaatggaaagatatcagctatctgtcattggaggccccagcacctataagcacatctcccccttggatgccagtgggcaaagattccgggaaactatggaggaattttctctctgagacagagcaagagagggagaccctgatgacttcttcgccccatgtcagcaggaagtagttacagaagacccacgacgtccttacaaccagagcttttcagggtctccatctcttgaggagggaaatattagggtaggcaggtaggcaggcatgagcaggcaagagagcccttgggaaaggaatctttagaaacgcagcccactgatagcttccttggtgatgctgcccacagacagtcagcacttctctaataacccatcctagaaca

SEQ ID NO: 22 - protein, 577 aa, Homo sapiens
MGSTQSKIVQNTPLGCLLRNLPTLQLDQDLKRKRLIFFCTVAWPQYTLDNQSRWPPEGTLNFNILNDLTNFCQKQGKWSEIKYVQGFWDLHSPPDLCAPCSLVQVLLAKTSPKTSPDPDKDDLSPLSDPIDNLSSPPLQTAAHALPPPYASPPPSAPPSGHAPPTSSRVPPSPITPPPPPPPSSPPPPPLPSPAPPKSPVAAHTQARPALLAPLREVAGAEGIVRVHVPFSLVDLSKIERHLGSFSTNPTLFTKEFHYLCQVYDLTWHDIHIILTSTLSPEERERILIAARQHANQLHLTDPNVPVGTQAVPSTDPEWDYQVGQAGRRRRDIMVQCLLAGMQVASNKSVNFDKLKEIVQYPDENPAVFLNRLTDALVHYTRLDPASPAGATILATYFISQSATDIRKKLKKAEEGLQTPIQDLVKLAFKVFNSREEAAEVQQQARLKQKVQLQTQALAAAVQPAFPKSPGKKGRGTISRAPSGACFKCGNSGHWASRCPSQQQPSCLPYNCFKCGNPGHWAKQCPNPKPPMRPCPNCRQMGHWRSDCPGLRAAAVSPHGDPSPDGEGTFQLLQLDDD

SEQ ID NO: 23 - protein, 526 aa, Homo sapiens
MGGAQSKIDPKTPLGCLLANFEALGLSMDLKRKRLIFFCLVAWPQYKLDNQSRWPPEGTFDFQILQDLDNLCRRQGKWSEVPYVQAFWDLRSRPDLCAKCSLGQVLLAKASPSNKEPDSSPLSEPPEALALPPLPAALPPPYPGSSGPTPTAPPLPPTPPSSPANPPASALPPPSPVSAHTRSKTDLLCLLREVAGAEGVVRVHVPFSLTDLSKIEKRPGSFSANPTLYIKQFRYLCQAYDLTWRDLHIILTSTLSPEERERVQAVARQHADQIHLTDPAMPVGTLAVPAAEPDWDYQAGQTGRRRRDQMVQCLLASMQAASNKTVNFDKLREIIQGSDENPAVFLNCLTEALIQYTRLDPTSPAGATVLATHVISQSAGDIRKKLKKVEEGPQTPIQDLVKMAFRVYNSREETAEAQRQARLKQKVQFQTQALVAAPRLAGSGSQPKGGSGHRAPPGACFKCGNEGHWAWQCPYPKEPTRPCPNCHQMGHWKSECPSVGASTVPLRCENSETTGGAFQLLSMDDD

SEQ ID NO: 24 - DNA, 21 bp, artificial
  siRNA ID s523645
cgcuugguccaguuuguaatt

SEQ ID NO: 25 - DNA, 21 bp, artificial
  siRNA ID s523645 MuERV Env P10404
uuacaaacuggaccaagcggt

SEQ ID NO: 26 - DNA, 21 bp, artificial
  siRNA ID s523644 MuERV Env P10404
cgggcauacuguaccaacatt

SEQ ID NO: 27 - DNA, 21 bp, artificial
  siRNA ID s523644 MuERV Env P10404
uguugguacaguaugcccggg

SEQ ID NO: 28 - DNA, 21 bp, artificial
  siRNA ID s523643 MuERV Env P10404
ccaacuuaaugacaggacatt

SEQ ID NO: 29 - DNA, 21 bp, artificial
  siRNA ID s523643 MuERV Env P10404
uguccugucauuaaguuggta

SEQ ID NO: 30 - DNA, 21 bp, artificial
  siRNA ID s529374 MuERV Gag
ggaaccaccuaguucucuatt

SEQ ID NO: 31 - DNA, 21 bp, artificial
  siRNA ID s529374 MuERV Gag
uagagaacuaggugguuccta

SEQ ID NO: 32 - DNA, 21 bp, artificial
  siRNA ID s529085 MuERV Gag
ggucgaaaguuagagcggutt

SEQ ID NO: 33 - DNA, 21 bp, artificial
  siRNA ID s529085 MuERV Gag
accgcucuaacuuucgaccaa

SEQ ID NO: 34 - DNA, 21 bp, artificial
  siRNA ID s535486 MuERV Gag
aguccguagaugucaagaatt

SEQ ID NO: 35 - DNA, 21 bp, artificial
  siRNA ID s535486 MuERV Gag
uucuugacaucuacggacuga

SEQ ID NO: 36 - DNA, 21 bp, artificial
  siRNA ID 5406 XPR1
ggauaugcuguauucagcutt

SEQ ID NO: 37 - DNA, 21 bp, artificial
  siRNA ID 5406 XPR1
agcugaauacagcauaucctt

SEQ ID NO: 38 - DNA, 21 bp, artificial
  siRNA ID 5493 XPR1
gguauugauagaagacacatt

SEQ ID NO: 39 - DNA, 21 bp, artificial
  siRNA ID 5493 XPR1
ugugucuucuaucaauacctt

SEQ ID NO: 40 - DNA, 21 bp, artificial
  siRNA ID 60216 Slc7a1 (mCAT-1)
ggacuguuaacucuuggcgtt

SEQ ID NO: 41 - DNA, 21 bp, artificial
  siRNA ID 60216 Slc7a1 (mCAT-1)
cgccaagaguuaacagucctg

SEQ ID NO: 42 - DNA, 21 bp, artificial
  siRNA
gcaaacaacugcuggcuuuuu

SEQ ID NO: 43 - DNA, 20 bp, artificial
  siRNA
gcaaagacccaccggaauuu

SEQ ID NO: 44 - DNA, 21 bp, artificial
  siRNA
cggcaaagacccaccggaauu

SEQ ID NO: 45 - DNA, 19 bp, artificial
  shRNA
cctgaacatccagaattat

SEQ ID NO: 46 - DNA, 21 bp, artificial
  siRNA
aggagaaacuggacuaauauu

SEQ ID NO: 47 - DNA, 21 bp, artificial
  siRNA
gggugaagccauuagauuauu

SEQ ID NO: 48 - DNA, 21 bp, artificial
  siRNA
acgcauagcuucugucaaauu

SEQ ID NO: 49 - DNA, 21 bp, artificial
  shRNA
gcagcgtcccggaaatattga

SEQ ID NO: 50 - DNA, 21 bp, artificial
  siRNA
ggaaatctcagctgcacaacc

SEQ ID NO: 51 - DNA, 21 bp, artificial
  siRNA
ccauaaagcuccagacgaauu

SEQ ID NO: 52 - DNA, 21 bp, artificial
  siRNA
ggguaaaucaaucaggaaauu

SEQ ID NO: 53 - DNA, 21 bp, artificial
  siRNA
ggagcuaaccagagccaaauu

SEQ ID NO: 54 - DNA, 19 bp, artificial
  siRNA
aggcauaacuauaggagau

SEQ ID NO: 55 - DNA, 19 bp, artificial
  siRNA
gccaaacacaactcgtgtt

SEQ ID NO: 56 - DNA, 19 bp, artificial
  siRNA
gccgacatactatactgtt

SEQ ID NO: 57 - DNA, 19 bp, artificial
  siRNA
gcttccctcaggttggtta

SEQ ID NO: 58 - DNA, 19 bp, artificial
  siRNA
gcaataccagctgggtcaa

SEQ ID NO: 59 - DNA, 19 bp, artificial
  siRNA
gcactggtgcaatatccat

SEQ ID NO: 60 - DNA, 19 bp, artificial
  siRNA
ggtgtagatggaggactat

SEQ ID NO: 61 - DNA, 19 bp, artificial
  siRNA
ccgacccgatgtgtgttat

SEQ ID NO: 62 - DNA, 19 bp, artificial
  siRNA
gctcgtgcagccattaata

SEQ ID NO: 63 - DNA, 19 bp, artificial
  siRNA
gcaggaaatgtagctcatt

SEQ ID NO: 64 - DNA, 19 bp, artificial
  siRNA
gctagccacctcattatta

SEQ ID NO: 65 - DNA, 19 bp, artificial
  siRNA
gcttgcagcagcagagaaa

SEQ ID NO: 66 - DNA, 19 bp, artificial
  siRNA
ccagatgccaagtttactt

SEQ ID NO: 67 - DNA, 19 bp, artificial
  siRNA
tcaactggtttgtcccttt

SEQ ID NO: 68 - DNA, 19 bp, artificial
  siRNA
ggcccttgtttctttaatt

SEQ ID NO: 69 - DNA, 19 bp, artificial
  siRNA
gggcaaagattccgggaaa

SEQ ID NO: 70 - DNA, 19 bp, artificial
  siRNA
gcaccttcatgccctctat

SEQ ID NO: 71 - DNA, 19 bp, artificial
  siRNA
gcagatgccagcataacta

SEQ ID NO: 72 - DNA, 19 bp, artificial
  siRNA
ccagatacctcaccttaaa

SEQ ID NO: 73 - DNA, 20 bp, artificial
  primer
actcttccagccttccttcc

SEQ ID NO: 74 - DNA, 20 bp, artificial
  primer
caatgccagggtacatggtg

SEQ ID NO: 75 - DNA, 21 bp, artificial
  primer
tccccagacatttcacatcag

SEQ ID NO: 76 - DNA, 20 bp, artificial
  primer
gagcggccacaggaatagta

SEQ ID NO: 77 - DNA, 20 bp, artificial
  primer
tggccaatagtgcggtgata

SEQ ID NO: 78 - DNA, 20 bp, artificial
  primer
tcgctatcaacagctggact

SEQ ID NO: 79 - DNA, 18 bp, artificial
  primer
ccaggatttgatgttggg

SEQ ID NO: 80 - DNA, 24 bp, artificial
  primer
ggtgtttctggaatatagggtcac

SEQ ID NO: 81 - DNA, 19 bp, artificial
  primer
cccatcgtataggagtctt

SEQ ID NO: 82 - DNA, 20 bp, artificial
  primer
ccccatcagacataccagtt

SEQ ID NO: 83 - DNA, 20 bp, artificial
  primer
gcctgcaaatagtcttcttt

SEQ ID NO: 84 - DNA, 20 bp, artificial
  primer
ataggggctattcccattag

SEQ ID NO: 85 - DNA, 20 bp, artificial
  primer
ccatgggaagcaagggaact

SEQ ID NO: 86 - DNA, 20 bp, artificial
  primer
ctttccccagcgagcaatac

SEQ ID NO: 87 - DNA, 20 bp, artificial
  primer
ggacagtgccgacatactat

SEQ ID NO: 88 - DNA, 20 bp, artificial
  primer
tagagtgcagcatcctaacc

SEQ ID NO: 89 - DNA, 22 bp, artificial
  primer
atggaggactatatgagcacaa

SEQ ID NO: 90 - DNA, 22 bp, artificial
  primer
attaaagttaaccacgagaagc

SEQ ID NO: 91 - DNA, 24 bp, artificial
  primer
gctacaccactcctaactcatcct

SEQ ID NO: 92 - DNA, 24 bp, artificial
  primer
ttgtaagggtgaagttacaccaga

SEQ ID NO: 93 - protein, 61 aa, artificial
  Mus musculus modified
  MCF247 (1)..(61): MuERV  Env  polyprotein  P10404, variable regions VRA, VRC and VRB in the surface domain SU that determine receptor usage  by different X/P-MuLV subtypes
  MCF247 (1)..(61): Env  protein of  a  typical  polytropic MuLV, MCF  247 , variable regions VRA, VRC and VRB in the surface domain SU that determine receptor usage  by different X/P-MuLV subtypes
GDDWDETGLGCRTPGGRKRARTFDFHTVPTGCRGNTPQNQGPCYDSSAVSSDIKGATPGGR

SEQ ID NO: 94 - protein, 61 aa, artificial
  Mus musculus mutated
  P10404 (MuERV Env) (33)..(33): Xaa=R, MuERV  Env  polyprotein  P10404,variable regions VRA, VRC and VRB in the surface domain SU that determine receptor usage by different X/P-MuLV subtypes
  misc_feature (34)..(34): Xaa can be any naturally occurring amino acid
GDDWDETGLGCRTPGGRKRARTFDFHTVPTGCRXNTPQNQGPCYDSSAVSSDIKGATPGGR

SEQ ID NO: 95 - DNA, 26 bp, artificial
  primer Syncytin-1 f
ccgctcgagagcggtcgtcggccaac

SEQ ID NO: 96 - DNA, 29 bp, artificial
  primer Syncytin-1 r
gaagatctccttcccagctaggcttaggg

SEQ ID NO: 97 - DNA, 19 bp, artificial
  pSIH-shRNA-Syn
ggccctcccttatcatatt

SEQ ID NO: 98 - DNA, 12 bp, artificial
  loop sequence to silence Syncytin-1
cttcctgtcaga

SEQ ID NO: 99 - DNA, 1599 bp, artificial
  SLC1A4
atggagaagagcaacgagaccaacggctaccttgacagcgctcaggcggggcctgcggccgggcccggagctccggggaccgcggcgggacgcgcacggcgttgcgcgggcttcctgcggcgccaagcgctggtgctgctcaccgtgtccggggtgctggcgggcgcgggcctgggcgcggcgttgcgcgggctcagcctgagccgcacgcaggtcacctacctggccttccccggcgagatgctgctccgcatgctgcgcatgatcatcctgccgctggtggtctgcagcctggtgtcgggcgccgcctcgctcgatgccagctgcctcgggcgtctgggcggcatcgctgtcgcctactttggcctcaccacactgagtgcctcggcgctcgccgtggccttggcgttcatcatcaagccaggatccggtgcgcagacccttcagtccagcgacctggggctggaggactcggggcctcctcctgtccccaaagagacggtggactctttcctcgacctggccagaaacctgtttccctccaatcttgtggttgcagctttccgtacgtatgcaaccgattataaagtcgtgacccagaacagcagctctggaaatgtaacccatgaaaagatccccataggcactgagatagaagggatgaacattttaggattggtcctgtttgctctggtgttaggagtggccttaaagaaactaggctccgaaggagaagacctcatccgtttcttcaattccctcaacgaggcgacgatggtgctggtgtcctggattatgtggtacgtacctgtgggcatcatgttccttgttggaagcaagatcgtggaaatgaaagacatcatcgtgctggtgaccagcctggggaaatacatcttcgcatctatattgggccatgttattcatggaggaattgttctgccacttatttattttgttttcacacgaaaaaacccattcagattcctcctgggcctcctcgccccatttgcgacagcatttgctacctgctccagctcagcgacccttccctctatgatgaagtgcattgaagagaacaatggtgtggacaagaggatcagcaggtttattctccccatcggggccaccgtgaacatggacggagcagccatcttccagtgtgtggccgcggtgttcattgcgcaactcaacaacgtagagctcaacgcaggacagattttcaccattctagtgactgccacagcgtccagtgttggagcagcaggcgtgccagctggaggggtcctcaccattgccattatcctggaggccattgggctgcctactcatgacctgcctctgatcctggctgtggactggattgtggaccggaccaccacggtggtgaatgtggaaggggatgccctgggtgcaggcattctccaccacctgaatcagaaggcaacaaagaaaggcgagcaggaacttgctgaggtgaaagtggaagccatccccaactgcaagtctgaggaggagacatcgcccctggtgacacaccagaaccccgctggccccgtggccagtgccccagaactggaatccaaggagtcggttctgtga

SEQ ID NO: 100 - DNA, 1626 bp, Homo sapiens
atggtggccgatcctcctcgagactccaaggggctcgcagcggcggagcccaccgccaacgggggcctggcgctggcctccatcgaggaccaaggcgcggcagcaggcggctactgcggttcccgggaccaggtgcgccgctgccttcgagccaacctgcttgtgctgctgacagtggtggccgtggtggccggcgtggcgctgggactgggggtgtcgggggccgggggtgcgctggcgttgggcccggagcgcttgagcgccttcgtcttcccgggcgagctgctgctgcgtctgctgcggatgatcatcttgccgctggtggtgtgcagcttgatcggcggcgccgccagcctggaccccggcgcgctcggccgtctgggcgcctgggcgctgctctttttcctggtcaccacgctgctggcgtcggcgctcggagtgggcttggcgctggctctgcagccgggcgccgcctccgccgccatcaacgcctccgtgggagccgcgggcagtgccgaaaatgcccccagcaaggaggtgctcgattcgttcctggatcttgcgagaaatatcttcccttccaacctggtgtcagcagcctttcgctcatactctaccacctatgaagagaggaatatcaccggaaccagggtgaaggtgcccgtggggcaggaggtggaggggatgaacatcctgggcttggtagtgtttgccatcgtctttggtgtggcgctgcggaagctggggcctgaaggggagctgcttatccgcttcttcaactccttcaatgaggccaccatggttctggtctcctggatcatgtggtacgcccctgtgggcatcatgttcctggtggctggcaagatcgtggagatggaggatgtgggtttactctttgcccgccttggcaagtacattctgtgctgcctgctgggtcacgccatccatgggctcctggtactgcccctcatctacttcctcttcacccgcaaaaacccctaccgcttcctgtggggcatcgtgacgccgctggccactgcctttgggacctcttccagttccgccacgctgccgctgatgatgaagtgcgtggaggagaataatggcgtggccaagcacatcagccgtttcatcctgcccatcggcgccaccgtcaacatggacggtgccgcgctcttccagtgcgtggccgcagtgttcattgcacagctcagccagcagtccttggacttcgtaaagatcatcaccatcctggtcacggccacagcgtccagcgtgggggcagcgggcatccctgctggaggtgtcctcactctggccatcatcctcgaagcagtcaacctcccggtcgaccatatctccttgatcctggctgtggactggctagtcgaccggtcctgtaccgtcctcaatgtagaaggtgacgctctgggggcaggactcctccaaaattacgtggaccgtacggagtcgagaagcacagagcctgagttgatacaagtgaagagtgagctgcccctggatccgctgccactccccactgaggaaggaaaccccctcctcaaacactatcgggggcccgcaggggatgccacggtcgcctctgagaaggaatcagtcatgtag

SEQ ID NO: 101 - protein, 699 aa, Homo sapiens
MNPSEMQRKAPPRRRRHRNRAPLTHKMNKMVTSEEQMKLPSTKKAEPPTWAQLKKLTQLATKYLENTKVTQTPESMLLAALMIVSMVVSLPMPAGAAAANYTYWAYVPFPPLIRAVTWMDNPIEVYVNDSVWVPGPTDDRCPAKPEEEGMMINISIGYRYPPICLGRAPGCLMPAVQNWLVEVPTVSPISRFTYHMVSGMSLRPRVNYLQDFSYQRSLKFRPKGKPCPKEIPKESKNTEVLVWEECVANSAVILQNNEFGTIIDWAPRGQFYHNCSGQTQSCPSAQVSPAVDSDLTESLDKHKHKKLQSFYPWEWGEKGISTPRPKIISPVSGPEHPELWRLTVASHHIRIWSGNQTLETRDRKPFYTIDLNSSLTVPLQSCVKPPYMLVVGNIVIKPDSQTITCENCRLLTCIDSTFNWQHRILLVRAREGVWIPVSMDRPWEASPSVHILTEVLKGVLNRSKRFIFTLIAVIMGLIAVTATAAVAGVALHSSVQSVNFVNDWQKNSTRLWNSQSSIDQKLANQINDLRQTVIWMGDRLMSLEHRFQLQCDWNTSDFCITPQIYNESEHHWDMVRRHLQGREDNLTLDISKLKEQIFEASKAHLNLVPGTEAIAGVADGLANLNPVTWVKTIGSTTIINLILILVCLFCLLLVCRCTQQLRRDSDHRERAMMTMVVLSKRKGGNVGKSKRDQIVTVSV

SEQ ID NO: 102 - protein, 698 aa, Homo sapiens
MNPSEMQRKAPPRRRRHRNRAPLTHKMNKMVTSEEQMKLPSTKKAEPPTWAQLKKLTQLATKYLENTKVTQTPESMLLAALMIVSMVVSLPMPAGAAAANYTNWAYVPFPPLIRAVTWMDNPIEVYVNDSVWVPGPIDDRCPAKPEEEGMMINISIGYRYPICLGRAPGCLMPAVQNWLVEVPIVSPICRFTYHMVSGMSLRPRVNYLQDFSYQRSLKFRPKGKPCPKEIPKESKNTEVLVWEECVANSAVILQNNEFGTIIDWTPQGQFYHNCSGQTQSCPSAQVSPAVDSDLTESLDKHKHKKLQSFYPWEWGEKGISTPRPKIISPVSGPEHPELWRLTVASHHIRIWSGNQTLETRDRKPFYTVDLNSSLTLPLQSCVKPPYMLVVGNIVIKPDSQTITCENCRLLTCIDSTFNWQHRILLVRAREGVWIPVSMDRPWEASPSIHILTEVLKGVLNRSKRFIFTLIAVIMGLIAVTATAAVAGVALHSSVQSVNFVNDGQKNSTRLWNSQSSIDQKLANQINDLRQTVIWMGDRLMSLEHRFQLQCDWNTSDFCITPQIYNESEHHWDMVRRHLQGREDNLTLDISKLKEQIFEASKAHLNLVPGTEAIAGVADGLANLNPVTWVKTIGSTTIINLILILVCLFCLLLVCRCTQQLRRDSDHRERAMMTMAVLSKRKGGNVGKSKRDQIVTVSV

SEQ ID NO: 103 - protein, 438 aa, Homo sapiens
MVTPVTWMDNPIEVYVNDSVRVPGPTDDRCPIKPEEEGIMINISTGYRYPICLGRAPGCLIHAVQNWLVEVPTVSPNGRFTYHMVSGMSLRPRVNYLQDFSYQRSLKFRPKGKPCPKEIPKESKNTEVLVWEECVANSAVILQNNEFGTIIDWAPRGQFDHNCSGQTQLCPSAQVSPAVDSDLTESLDKHKHKKLQSLYPWEWGEKGISTPRPKIISPVSGPEHPELWRLIVASHHIRIWSGNQTSETRDRKPFYTIDLNSSLTVPLQSCVKPPYMLVVGNIVIKPDSQTITCENCRLFTCIDSTFNWQQRILLVRAREGVWIPVSMDRPWEASPSIHILTEVLKGILNRSKRFIFTLIAVIMGLIAVTATAAVAGVALHSSVQSVNFVNDWQKNSARLWNSQSSIDQKLANQINHLRQTHLDRRQTHELRTSFPVTV

SEQ ID NO: 104 - protein, 603 aa, Homo sapiens
YNEPIRDAKKRASTEMVTPVTWMDNPIEVYVNDSEWVPGPTDDRCPAKPEEEGTMLNISIGYRYPPICLGTAPGCLMPAVQNWLVEVPIVSPISRFTYHMVSGMSLRPRVNYLQDFPYQRSLKFRPKGKPCPKEIPKESKNTEILVWEECVANSAVILQNNEFGTIIDWAPRGQFYHNCSGQTQSCPSAQVSPAVDSDLTESLDKHKHKKLQSFYPWEWGEKGISTPRPKIISPVSGPEHPELWRLTVASHHIRIWSGNQTLETRDRKPFYTVDLNSSLTLPLQSCVKPPYMLVVGNIVIKPDSQTITCENCRLLTCIDSTFNWQHRILLVRAREGVWILVSMDRPWEASPSVHILTEVLKGVLNRSKRFIFTLIAVIMGLIAVTATGAVAGVALHSSVQSVNFVNDWQKNSTRLWNSQSSIDQKLANQINDLRQTVIWMGDRLMSLEHRFQLQCDWNTSDFCITPQIYNESEHHWDMVRHHLQGREDNLTLDISKLKEQIFEASKAHLNLVPGTEAIAGVADGLANLNPVTWVKTIGSTTIINLILILVCLFCLLLVCRCTQQLRRDSDHRERAMMTMTVLSKRKGGNVGKSKRDQIVTVSV

SEQ ID NO: 105 - protein, 699 aa, Homo sapiens
MNPSEMQRKAPPRRRRHRNRAPLTHKMNKMVTSEEQMKLPSTKKAEPPTWAQLKKLTQLATKYLENTKVTQTPESMLLAALMIVSMVVSLPMPAGAAAANYTYWAYVPFPPLIRAVTWMDNPTEVYVNDSVWVPGPIDDRCPAKPEEEGMMINISIGYHYPPICLGRAPGCLMPAVQNWLVEVPTVSPICRFTYHMVSGMSLRPRVNYLQDFSYQRSLKFRPKGKPCPKEIPKESKNTEVLVWEECVANSAVILQNNEFGTIIDWAPRGQFYHNCSGQTQSCPSAQVSPAVDSDLTESLDKHKHKKLQSFYPWEWGEKGISTPRPKIVSPVSGPEHPELWRLTVASHHIRIWSGNQTLETRDRKPFYTIDLNSSLTVPLQSCVKPPYMLVVGNIVIKPDSQTITCENCRLLTCIDSTFNWQHRILLVRAREGVWIPVSMDRPWEASPSVHILTEVLKGVLNRSKRFIFTLIAVIMGLIAVTATAAVAGVALHSSVQSVNFVNDWQKNSTRLWNSQSSIDQKLANQINDLRQTVIWMGDRLMSLEHRFQLQCDWNTSDFCITPQIYNESEHHWDMVRRHLQGREDNLTLDISKLKEQIFEASKAHLNLVPGTEAIAGVADGLANLNPVTWVKTIGSTTIINLILILVCLFCLLLVCRCTQQLRRDSDHRERAMMTMAVLSKRKGGNVGKSKRDQIVTVSV

SEQ ID NO: 106 - protein, 699 aa, Homo sapiens; identical to SEQ ID NO: 105

SEQ ID NO: 107 - protein, 699 aa, Homo sapiens; identical to SEQ ID NO: 105

SEQ ID NO: 108 - protein, 699 aa, Homo sapiens; identical to SEQ ID NO: 105

SEQ ID NO: 109 - protein, 124 aa, Homo sapiens
MDNPIEVYVNDSVWVPGPTDDRCPAKPEEERMMINISIGYRYPPICLGRAPGCLMPAVQNWLVEVPTVSPISRFTYHMVSGMSLRPQVNYLQDFSYQRSLKFRPKRKPCLKEIPKESKNTEVLV

SEQ ID NO: 110 - protein, 1360 aa, Homo sapiens
MGPLQPGLPSPAMIPKDWPLIIIDLKDCFFTIPLAEQDCEKFAFTIPAINNKEPATRFQWKVLPQGMLNSPTICQTFVGRALQPVREKFSDCYIIHYIDDILCAAETKDKLIDCYTFLQAEVANAGLAIASDKIQTSTPFHYLGMQIENRKIKPQKIEIRKDTLKTLNDFQKLLGDINWIRPTLGIPTYAMSNLFSILRGDSDLNSQRILTPEATKEIKLVEEKIQSAQINRIDPLAPLQLLIFATAHSPTGIIIQNTDLVEWSFLPHSTVKTFTLYLDQIATLIGQTRLRITKLCGNDPDKIVVPLTKEQVRQAFINSGAWQIGLANFVGLIDNHYPKTKIFQFLKLTTWILPKITRREPLENALTVFTDGSSNGKAAYTGPKERVIKTPYQSAQRDELVAVITVLQDFDQPINIISDSAYVVQATRDVETALIKYSMDDQLNQLFNLLQQTVRKRNFPFYITYIRAHTNLPGPLTKANEQADLLVSSALIKAQELHALTHVNAAGLKNKFDVTWKQAKDIVQHCTQCQVLHLPTQEAGVNPRGLCPNALWQMDVTHVPSFGRLSYVHVTVDTYSHFIWATCQTGESTSHVKKHLLSCFAVMGVPEKIKTDNGPGYCSKAFQKFLSQWKISHTTGIPYNSQGQAIVERTNRTLKTQLVKQKEGGDSKECTTPQMQLNLALYTLNFLNIYRNQTTTSAEQHLTGKKNSPHEGKLIWWKDNKNKTWEIGKVITWGRGFACVSPGENQLPVWLPTRHLKFYNEPIGDAKKRASTEMVTPVTWMDNPIEVYVNDSIWVPGPIDDRCPAKPEEEGMMINISIGYRYPPICLGRAPGCLMPAVQNWLVEVPTVSPISRFTYHMVSGMSLRPRVNYLQDFSYQRSLKFRPKGKPCPKEIPKESKNTEVLVWEECVANSAVILNNEFGTIIDWAPRGQFYHNCSGQTQSCPSAQVSPAVDSDLTESLDKHKHKKLQSFYPWEWGEKGISTPRPKIVSPVSGPEHPELWRLTVASHHIRIWSGNQTLETRDCKPFYTVDLNSSLTVPLQSCVKPPYMLVVGNIVIKPDSQTITCENCRLLTCIDSTFNWQHRILLVRAREGVWIPVSMDRPWEASPSVHILTEVLKGVLNRSKRFIFTLIAVIMGLIAVTATAAVAGVALHSSVQSVNFVNDWQKNSTRLWNSQSSIDQKLANQINDLRQTVIWMGDRLMSLEHRFQLQCDWNTSDFCITPQIYNESEHHWDMVRRHLQGREDNLTLDISKLKEQIFEASKAHLNLVPGTEAIAGVADGLANLNPVTWVKTIGSTSIINLILILVCLFCLLLVCRCTQQLRRDSDHRERAMMTMAVLSKRKGGNVGKSKRDQIVTVSV

SEQ ID NO: 111 - protein, 245 aa, Homo Sapiens
MVTPVTWMDNPIEVYVNDSVWVPGPTDDRCPAKPEEEGMMINISIVYRYPPICLGRAPGCLMPAVQNWLVEVPTVSPNSRFTYHMVSGMSLRPRVNYLQDFSYQRSLKFRPKGKPCPKEIPKESKNTEVLVWEECVANSAVILQNNEFGTIIDWAPRGQFYHNCSGQTQSCPSAQVSPAVDSDLTESLDKHKHKKLQSFYPWEWGEKGISTPRPEIISPVSGPEHPELWRLWPDTTLEFGLEIKL

SEQ ID NO: 112 - protein, 667 aa, Homo Sapiens
MGQTKSKTKSKYASYLSFIKILLKRGGVRVSTKNLIKLFQIIEQFCPWFPEQGTLDLKDWKRIGEELKQAGRKGNIIPLTVWNDWAIIKAALEPFQTKEDSVSVSDAPGSCVIDCNEKTGRKSQKETESLHCEYVTEPVMAQSTQNVDYNQLQGVIYPETLKLEGKGPELVGPSESKPRGPSPLPAGQVPVTLQPQTQVKENKTQPPVAYQYWPPAELQYLPPPESQYGYPGMPPALQGRAPYPQPPTVRLNPTASRSGQGGTLHAVIDEARKQGDLEAWRFLVILQLVQAGEETQVGAPARAETRCEPFTMKMLKDIKEGVKQYGSNSPYIRTLLDSIAHGNRLTPYDWESLAKSSLSSSQYLQFKTWWIDGVQEQVRKNQATKPTVNIDADQLLGTGPNWSTINQQSVMQNEAIEQVRAICLRAWGKIQDPGTAFPINSIRQGSKEPYPDFVARLQDAAQKSITDDNARKVIVELMAYENANPECQSAIKPLKGKVPAGVDVITEYVKACDGIGGAMHKAMLMAQAMRGLTLGGQVRTFGKKCYNCGQIGHLKRSCPVLNKQNIINQAITAKNKKPSGLCPKCGKGKHWANQCHSKFDKDGQPLSGNRKRGQPQAPQQTGAFPVQLFVPQGFQGQQPLQKIPPLQGVSQLQQSNSCPAPQQAAPQ

SEQ ID NO: 113 - protein, 699 aa, Homo Sapiens
MNPSEMQRKAPPRRRRHRNRAPLTHKMNKMVTSEEQMKLPSTKKAEPPTWAQLKKLTQLATKYLENTKVTQTPESMLLAALMIVSMVVSLPMPAGAAAANYTYWAYVPFPPLIRAVTWMDNPIEIYVNDSVWVPGPTDDCCPAKPEEEGMMINISIGYRYPPICLGRAPGCLMPAVQNWLVEVPTVSPISRFTYHMVSGMSLRPRVNYLQDFSYQRSLKFRPKGKPCPKEIPKESKNTEVLVWEECVANSAVILQNNEFGTLIDWAPRGQFYHNCSGQTQSCPSAQVSPAVDSDLTESLDKHKHKKLQSFYPWEWGEKGISTARPKIISPVSGPEHPELWRLTVASHHIRIWSGNQTLETRDRKPFYTIDLNSSLTVPLQSCVKPPYMLVVGNIVIKPDSQTITCENCRLLTCIDSTFNWQHRILLVRAREGVWIPVSMDRPWEASPSVHILTEVLKGVLNRSKRFIFTLIAVIMGLIAVTATAAVAGVALHSSVQSVNFVNDWQNNSTRLWNSQSSIDQKLANQINDLRQTVIWMGDRLMSLEHRFQLQCDWNTSDFCITPQIYNESEHHWDMVRCHLQGREDNLTLDISKLKEQIFEASKAHLNLVPGTEAIAGVADGLANLNTVTWVKTIGSTTIINLILILVCLFCLLLVYRCTQQLRRDSDHRERAMMTMVVLSKRKGGNVGKSKRDQIVTVSV

SEQ ID NO: 114 - protein, 580 aa, Homo Sapiens
MVTPVTWMDNPIEIYVNDSVWVPGPIDDRCPAKPEEEGMMINISIGYRYPPICLGRAPGCLMPAVQNWLVEVPTVSPISRFTYHMVSGMSLRPRVNYLQDFSYQRSLKFRPKGKPCPKEIPKESKNTEVLVWEECVANSAVILQNNEFGTIIDWAPRGQFYHNCSGQTQSCPSAQVSPAVDSDLTESLDKHKHKKLQSFYPWEWGEKGISTPRPKIVSPVSGPEHPELWRLTVASHHIRIWSGNQTLETRDCKPFYTIDLNSSLTVPLQSCVKPPYMLVVGNIVIKPDSQTITCENCRLLTCIDSTFNWQHRILLVRAREGVWIPVSMDRPWEASPSVHILTEVLKGVLNRSKRFIFTLIAVIMGLIAVTATAAVAGVALHSSVQSVNFVNDWQKNSTRLWNSQSSIDQKLANQINDLRQTVIWMGDRLMSLEHRFQLQCDWNTSDFCITPQIYNESEHHWDMVRRHLQGREDNLTLDISKLKEQIFEASKAHLNLVPGTEAIAGVADGLANLNPVTWVKTIGSTTIINLILILVCLFCLLLVCRCTQQLRRDSDHRERAMMTMAVLSKRKGGNVGKSKR

SEQ ID NO: 115 - protein, 514 aa, Homo Sapiens
MNPSEMQRKAPPRRRRHRNRAPLTHKMNKMVTSEEQMKLPSTKKAEPPTWAQLKKLTQLATKYLENTKVTQTPESMLLAALMIVSMVVSLPMPAGAAAANYTYWAYVPFPPLIRAVTWMDNPIEVYVNDSVWVPGPTDDHCPAKPEEEGMMINISIGYRYPPICLGRAPGCLMPAVQNWLVEVPTVSPISRFTYHMVSGMSLRPRVNYLQDFSYQRSFKFRPKGKPCPKEIPKESKNTEVLVWEECVANSAVILQNNEFGTIIDWAPRGQFYHNCSGQTQSCPSAQVSPAVDSDLTESLDKHKHKKLQSFYPWEWGEKGISTPRPKIISPVSGPEHPELWRLTVASHHIRIWSGNQTLETRDRKPFYTVDLNSSVTVPLQSCIKPPYMLVVGNIVIKPDSQTITCENCRLLTCIDSTFNWQHRILLVRAREGVWIPVSMDRPWETSPSIHTLTEVLKGVLNRSKRFIFTLIAVIMGLIAVTATAAVAGVALHSSVQSVNFVNDWQKNSTRLWNS

SEQ ID NO: 116 - protein, 699 aa, Homo Sapiens; identical to SEQ ID NO: 113

SEQ ID NO: 117 - protein, 699 aa, Homo Sapiens
MNPSEMQRKAPPRRRRHRNRAPLTHKMNKMVTSEEQMKLPSTKKAEPPTWAQLKKLTQLATKYLENTKVTQTPESMLLAALMIVSMVVSLPMPAGAAVANYTNWAYVPFPPLIRAVTWMDNPIEVYVNDSVWVPGPIDDRCPAKPEEEGMMINISIGYRYPPICLGRAPGCLMPAVQNWLVEVPTVSPISRFTYHMVSGMSLRPRVNYLQDFSYQRSLKFRPKGKPCPKEIPKESKNTEVLVWEECVANSAVILQNNEFGTIIDWAPRGQFYHNCSGQTQSCPSAQVSPAVDSDLTESLDKHKHKKLQSFYPWEWGEKRISTPRPKIVSPVSGPEHPELWRLTVASHHIRIWSGNQTLETRDRKPFYTVDLNSSLTLPLQSCVKPPYMLVVGNIVIKPDSQTITCENCRLLTCIDSTFNWQHRILLVRAREGVWIPVSMDRPWEASPSVHILTEVLKGVLNRSKRFIFTLIAVIMGLIAVTATAAVAGVALHSSVQSVNFVNDGQKNSTRLWNSQSSIDQKLANQINDLRQTVIWMGDRLMSLEHRFQLQCDWNTSDFCITPQIYNDSEHHWDMVRRHLQGREDNLTLDISKLKEQIFEASKAHLNLVPGTEAIAGVADGLANLNPVTWVKTIGSTTIINLILILVCLFCLLLVCRCTQQLRRDSDHRERAMMTMAVLSKRKGGNVGKSKRDQIVTVSV

SEQ ID NO: 118 - protein, 812 aa, Homo Sapiens
NKSRKRRNRVSFLGVTTVEPPKPIPLTWKTEKLVWVNQWPLPKQKLEALHLLANEQLEKGHIEPSFSPWNSPVFVIQKKSSKWRMLTDLRAVNAVIQPMGPLQPGLPSPAMIPKDWPLIIIDLKDCFFTIPLAEQDCEKFAFTIPAINNKEPATRFQWKVLPQGMLNSPTICQTFVGRALQPVRDKFSDCYIIHYFDDILCAAETKDKLIDCYTFLQAEVANAGLAIASDKIQTSTPFHYLGMQIENRKIKPQKIEIRKDTLKTLNDFQKLLGDINWIRPTLGIPTYAMSNLFSILRGDSDLNSKRMLTPEATKEIKLVEEKIQSAQINRIDPLAPLQLLIFATAHSPTGIIIQNTDLVEWSFLPHSTVKTFTLYLDQIATLIGPTRLRIIKLCGNDPDKIVVPLTKEQVRQAFINSGAWQIGLANFVGIIDNHYPKTKIFQFLKLTTWILPKITRREPLENALTVFTDGSSNGKVAYTGPKERVIKTPYQSAQRAELVAVITVLQDFDQPINIISDSAYVVQATRDVETALIKYSMDDQLNQLFNLLQQTVRKRNFPFYITHIRAHTNLPGPLTKANEQADLLVSSAFIKAQELHALTHVNAAGLKNKFDVTWKQAKDIVQHCTQCQVLDLPTQEAGVNPEVCVLMHYGKWMSHMYLHLGRLSYVHVTVDTYSHFMCATCQTGESTSHVKKHLLSCFAVMGVPEKIKTDNGPGYCSKAFQKFLSQWKISHTTGIPYNSQGQAIVERTNRTLKTQLVKQKEGGDSKECTTPQMQLNLALYTLNFLNIYRNQTTTSAEHLTGKKNSPHEGKLI

SEQ ID NO: 119 - protein, 482 aa, Homo Sapiens
MWTVPSFTNDSYQVYNVFSTNSFQLLTVKRTPHEAWRVPLTTKTNKTKGLPDCPKKPTNGPFIVTSILWDNCNAPKAVVLQTLAMGIVIDWAPKGHYWQDCSSKNTLCSEFIYSLDYIEHGWQSYTMRQRVSPYPFKWMDTGIAPPRPKIIHPFFTPEHPELWKLAAALSGIKIWNTTYQLLRTKTKTPTFNITLISEWVIPIRSCVKPPYMLLVGNIIMMPDAQTIECHNCKLFTCIDATFNPTTSILLVRAREGVWIPVSLHRPWESSPSIHIVNEVLKDILKRTKRFIFTLIAVLAGLLAVTATAATAGVAIRSSVQTAHYVEACQKNSSRLWNSQAQIDQKLANQINDLRQSVTWLGDRVMNLQHRMQLQCDWNTSDYCITPYAYNQDQHSWENVSRHLKAWDDNLTLDISQLKEQIFEASQAHLSTVPGSHIFEGITKQLPDFNPFKWLKPVRGSLLLLALLILVCLCCLLLVCRCL

SEQ ID NO: 120 - protein, 245 aa, Homo Sapiens; identical to SEQ ID NO: 111

SEQ ID NO: 121 - protein, 156 aa, Homo Sapiens
WASQVSENRPVCKAVIQGKQLEGLVDTGADVSIIALNQWPKNWPKQKTVTGLVGIVTASEVYQSTEILHCLGPHNQESTVQPMITSIPLNLWGRDLLQQWGAEITMTATLYSPMSQKIMTKMGYIPGKGLGKNEDGIKVPIEAKINHGREGTGYPF

SEQ ID NO: 122 - protein, 661 aa, Homo sapiens
MNPSEMQRKAPPRRRRHRNRAPLTHKMNKMVTSEEQMKLPSTKKAEPPTWAQLKKLTQLATKYLENTKVTQTPESMLLAALMIVSMVVSLPMPAGAAAANYTYWAYVPFPPLIRAVTWMDNPIEVYVNDSVWVPGPIDDRCPAKPEEEGMMINISIGYRYPPICLGTAPGCLMPAVQNWLVEVPIVSPISRFTYHMVSGMSLRPRVNYLQDFSYQRSLKFRPKGKPCPKEIPKESKNTEVLVWEECVANSAVILQNNEFGTIIDWAPRGQFYHNCSGQTQSCPSAQVSPAVDSDLTESLDKHKHKKLQSFYPWEWGEKGISTPRPKIVSPVSGPEHPELWRLTVASHHIRIWSGNQTLETRDRKPFYTVDLNSSLTVPLQSCVKPPYMLVVGNIVIKPDSQTITCENCRLLTCIDSTFNWQHRILLVRAREGVWIPVSMDRPWEASPSIHILTEVLKGVLNRSKRFIFTLIAVIMGLIAVTATGAVAGVALHSSVQSVNFVNDWQKNSTRLWNSQSSIDQKLANQINDLRQTVIWMGDRLMSLEHRFQLQCDWNTSDFCITPQIYNESEHHWDMVRRHLQGREDNLTLDISKLKEQIFKASKAHLNLVPGTEAIAGVADGLANLNPVTWVKTIGSTTIINLILILVCLFCLLLVCRCTQQL

SEQ ID NO: 123 - protein, 191 aa, Homo sapiens
MPGAIDDHCPAQPGEEGTAFNVTMGYKYPPLCLGHATRCIHLETQVWAAYLLERLATGKWGHLVSGLSLCPLRQMKRGVIGDTPYFQYKPVGKLCPKNFEGPSKTLIWGDCVNSHAVVLKNDSYALVIDWAPKGYLKNTCSSGGGEFLEATYFISYWEDEDHHPTLHRWFGSFFTLKWEDKDITLHPQGLV

SEQ ID NO: 124 - protein, 588 aa, Homo sapiens
MVTPVTWMDNPIEIYVNDSVWVPGPIDDRCPAKPEEEGMMINISIGYRYPPICLGRAPGCLMPAVQNWLVEVPTVSPISRFTYHMVSGMSLRPRVNYLQDFSYQRSLKFRPKGKPCPKEIPKESKNTEVLVWEECVANSAVILQNNEFGTIIDWAPRGQFYHNCSGQTQSCPSAQVSPAVDSDLTESLDKHKHKKLQSFYPWEWGEKRISTPRPKIVSPVSGPEHPELWRLTVASHHIRIWSGNQTLETRDCKPFYTIDLNSSLTVPLQSCVKPPYMLVVGNIVIKPDSQTITCENCRLLSCIDSTFNWQHRILLVRAREGVWIPVSMDRPWEASPSVHILTEVLKGVLNRSKRFIFTLIAVIMGLIAVTATAAVAGVALHSSVQSVNFVNDWQKNSTRLWNSQSSIDQKLANQINDLRQTVIWMGDRLMSLEHRFQLQCDWNTSDFCITPQIYNESEHHWDMVRRHLQGREDNLTLDISKLKEQIFEASKAHLNLVPGTEAIAGVADGLANLNPVTWVKTIGSTTIINLILILVCLFCLLLVCRCTQQLRRDSDHRERAMMTMAVLSKRKGGNVGKSKRDQIVTVSV

SEQ ID NO: 125 - protein, 588 aa, Homo sapiens
MVTPVTWMDNPIEVYVNDSEWVPGPTDDRCPAKPEEEGMMINISIGYRYPPICLGTAPGCLMPAVQNWLVEVPIVSPISRFTYHMVSGMSLRPRVNYLQDFPYQRSLKFRPKGKPCPKEIPKESKNTEVLVWEECVANSAVILQNNEFGTIIDWAPRGQFYHNCSGQTQSCPSAQVSPAVDSDLTESLDKHKHKKLQSFYPWEWGEKGISTPRPKIISPVSGPEHPELWRLTVASHHIRIWSGNQTLETRDRKPFYTVDLNSSLTLPLQSCVKPPYMLVVGNIVIKPDSQTITCENCRLLTCIDSTFNWQHRILLVRAREGVWILVSMDRPWEASPSVHILTEVLKGVLNRSKRFIFTLIAVIMGLIAVTATGAVAGVALHSSVQSVNFVNDWQKNSTRLWNSQSSIDQKLANQINDLRQTVIWMGDRLMSLEHRFQLQCDWNTSDFCITPQIYNESEHHWDMVRHHLQGREDNLTLDISKLKEQIFEASKAHLNLVPGTEAIAGVADGLANLNPVTWVKTIGSTTIINLILILVCLFCLLLVCRCTQQLRRDSDHRERAMMTMAVLSKRKGGNVGKSKRDQIVTVSV

SEQ ID NO: 126 - protein, 698 aa, Homo sapiens
MHPSEMQRKAPPRRRRHRNRAPLTHKMNKMVTSEQMKLPSTKKAEPPTWAQLKKLTQLATKYLENTKVTQTPESMLLAALMIVSMVVSLPMPAGAAAANYTNWAYVPFPPLIRAVTWMDNPIEVYVNDSVWVHGPIDDRCPAKPEEEGMMINISIGYHYPPICLGRAPGCLMPAVQNWLVEVPTVSPISRFTYNMVSGMSLRPRVNYLQDFSYQRSLKFRPKGKPCPKEIPKESKNTEVLVWEECVANSVVILQNNEFGTIIDWAPRGQFYHNCSGQTQSCPSAQVSPAVDSDLTESLDKHKHKKLQSFYPWEWGEKGISTPRPKIISPVSGPEHPELWRLTVASHHIRIWSGNQTLETRDRKPFYTVDLNSSLTVPLQSCVKPPYMLVVGNIVIKPDSQTITCENCRLLTCIDSTFNWQHRILLVRAREGVWIPVSMDRPWEASPSIHILTEVLKGVLNRSKRFIFTLIAVIMGLIAVTAMAAVAGVALHSFVQSVNFVNDWQKNSTRLWNSQSSIDQKLANQINDLRQTVIWMGDRLMSLEHRFQLQCDWNTSDFCITPQIYNESEHHWDMVRRHLQGREDNLTLDISKLKEQIFEASKAHLNLVPGTEAIAGVADGLANLNPVTWVKTIGSTTIINLILILVCLFCLLLVCRCTQQLRRDSDHRERAMMTMVVLSKRKGGNVGKSKRDQIVTVSV

SEQ ID NO: 127 - protein, 699 aa, Homo sapiens
MNPSEMQRKAPPRRRRHRNRAPLTHKMNKMVTSEEQMKLPSTKKAEPPTWAQLKKLTQLATKYLENTKVTQTPESMLLAALMIVSMVVSLPMPAGAAAANYTYWAYVPFPPLIRAVTWMDNPIEVYVNDSVWVPGPTDDHCPAKPEEEGMMINISIGYRYPPICLGRAPGCLMPAVQNWLVEVPTVSPISRFTYHMVSGMSLRPRVNYLQDFSYQRSFKFRPKGKPCPKEIPKESKNTEVLVWEECVANSAVILQNNEFGTIIDWAPRGQFYHNCSGQTQSCPSAQVSPAVDSDLTESLDKHKHKKLQSFYPWEWGEKGISTPRPKIISPVSGPEHPELWRLTVASHHIRIWSGNQTLETRDRKPFYTVDLNSSVTVPLQSCIKPPYMLVVGNIVIKPDSQTITCENCRLLTCIDSTFNWQHRILLVRAREGVWIPVSMDRPWETSPSIHTLTEVLKGVLNRSKRFIFTLIAVIMGLIAVTATAAVAGVALHSSVQSVNFVNDWQKNSTRLWNSQSSIDQKLANQINDLRQTVIWMGDRLMSLEHRFQLQCDWNTSDFSITPQIYNESEHHWDMVRRHLQGREDNLTLDISKLKEQIFEASKAHLNLVPGTEAIAGVADGLANLNPVTWVKTIGSTTIINLILILVCLFCLLLVCRCTQQLRRDSDHRERAMMTMAVLSKRKGGNVGKSKRDQIVTVSV

SEQ ID NO: 128 - protein, 560 aa, Homo sapiens
MVTPVTWMDNPIEVYVNDSVWVPGPTDDRCPAKPEEEGMMINISIGYHYPPICLGRAPGCLMPAVQNWLVEVPTVSPNSRFTYHMVSGMSLRPRVNCLQDFSYQRSLKFRPKGKTCPKEIPKGSKNTEVLVWEECVANSVVILQNNEFGTIIDWAPRGQFYHNCSGQTQSCPSAQVSPAVDSDLTESLDKHKHKKLQSFYLWEWEEKGISTPRPKIISPVSGPEHPELWRLTVASHHIRIWSGNQTLETRYRKPFYTIDLNSILTVPLQSCVKPPYMLVVGNIVIKPASQTITCENCRLFTCIDSTFNWQHRILLVRAREGMWIPVSTDRPWEASPSIHILTEILKGVLNRSKRFIFTLIAVIMGLIAVTATAAVAGVALHSSVQSVNFVNYWQKNSTRLWNSQSSIDQKLASQINDLRQTVIWMGDRLMTLEHHFQLQCDWNTSDFCITPQIYNESEHHWDMVRRHLQGREDNLTLDISKLKEQIFEASKAHLNLVPGTEAIAGVADGLANLNPVTWIKTIRSTMIINLILIVVCLFCLLLVCRCTQQLRRDSDIENGP

SEQ ID NO: 129 - protein, 538 aa, Homo sapiens
MALPYHIFLFTVLLPSFTLTAPPPCRCMTSSSPYQEFLWRMQRPGNIDAPSYRSLSKGTPTFTAHTHMPRNCYHSATLCMHANTHYWTGKMINPSCPGGLGVTVCWTYFTQTGMSDGGGVQDQAREKHVKEVISQLTRVHGTSSPYKGLDLSKLHETLRTHTRLVSLFNTTLTGLHEVSAQNPTNCWICLPLNFRPYVSIPVPEQWNNFSTEINTTSVLVGPLVSNLEITHTSNLTCVKFSNTTYTTNSQCIRWVTPPTQIVCLPSGIFFVCGTSAYRCLNGSSESMCFLSFLVPPMTIYTEQDLYSYVISKPRNKRVPILPFVIGAGVLGALGTGIGGITTSTQFYYKLSQELNGDMERVADSLVTLQDQLNSLAAVVLQNRRALDLLTAERGGTCLFLGEECCYYVNQSGIVTEKVKEIRDRIQRRAEELRNTGPWGLLSQWMPWILPFLGPLAAIILLLLFGPCIFNLLVNFVSSRIEAVKLQMEPKMQSKTKIYRRPLDRPASPRSDVNDIKGTPPEEISAAQPLLRPNSAGSS

SEQ ID NO: 130 - protein, 542 aa, Homo sapiens
MALPYHTFLFTVLLPPFALTAPPPCCCTTSSSPYQEFLWRTRLPGNIDAPSYRSLSKGNSTFTAHTHMPRNCYNSATLCMHANTHYWTGKMINPSCPGGLGATVCWTYFTHTSMSDGGGIQGQAREKQVKEAISQLTRGHSTPSPYKGLVLSKLHETLRTHTRLVSLFNTTLTRLHEVSAQNPTNCWMCLPLHFRPYISIPVPEQWNNFSTEINTTSVLVGPLVSNLEITHTSNLTCVKFSNTIDTTSSQCIRWVTPPTRIVCLPSGIFFVCGTSAYHCLNGSSESMCFLSFLVPPMTIYTEQDLYNHVVPKPHNKRVPILPFVIRAGVLGRLGTGIGSITTSTQFYYKLSQEINGDMEQVTDSLVTLQDQLNSLAAVVLQNRRALDLLTAKRGGTCLFLGEERCYYVNQSRIVTEKVKEIRDRIQCRAEELQNTERWGLLSQWMPWTLPFLGPLAAIIFLLLFGPCIFNFLVKFVSSRIEAVKLQIVLQMEPQMQSMTKIYRGPLDRPARLCSDVNDIEVTPPEEISTAQPLLHSNSVGSS

SEQ ID NO: 131 - protein, 410 aa, Homo sapiens
MALPYHIFLFTVVSPSFTLTAPPPCRCMTSSSPYQEFLWRMQRPGNIDAPSYRSLCKGTPTFTAHTHMPRNCYHSATLCMHANTHYWTGKMINPSCPGGLGVTVCWTYFTQTGMSDGGGVQDQAREKHVKEVISQLTRVHGTSSPYKGLDLSKLHETLRTHTRLVSLFNTTLTGLHEVSAQNPTNCWICLPLNFRPYVSIPVPEQWNNFSTEINTTSVLVGPLVSNLEITHTSNLTCVKFSNTTYTTNSQCIRWVTPPTQIVCLPSGIFFVCGTSAYRCLNGSSESMCFLSFLVPPMAIYTEQDLYSYVISKPRNKRVPILPFVIGAGVLGALGTGIGGITTSTQFYYKLSQELNGDMERVADSLVTLQDQLNSLAAVVLQNRRALDLLTAERGGTCLFLGEECCYYVNQ

SEQ ID NO: 132 - protein, 555 aa, Homo sapiens
MILAGRAPSNTSTLMKFYSLLLYSLLFSFPFLYHPLPLPSYLHHTINLTHSLPAASNPSLANNCWLCISLSSSAYIAVPTLQTDRATSPVSLHLRTSFNSPHLYPPEELIYFLDRSSKTSPDISHQPAAALLHIYLKNLSPYINSTPPIFGPLTTQTTIPVAAPLCISRQRPTGIPLGNISPSRCSFTLHLQSPTTHVTETIGVFQLHIIDKPSINTDKLKNVSSNYCLGRHLPYISLHPWLPSPCSSDSPPRPSSCLLTPSPQNNSERLLVDTQRFLIHHENRTSSSMQLAHQSPLQPLTAAALAGSLGVWVQDTPFSTPSHPFSLHLQFCLTQGLFFLCGSSTYMCLPANWTGTCTLVFLTPKIQFANGTKELPVPLMTLTPQKRVIPLIPLMVGLGLSASTIALSTGIAGISTSVTTFRSPSNDFSASITDISQTLSVLQAQVDSLAAVVLQNRRGLGLSILLNEECCFYLNQSGLVYENIKKLKDRAQKLANQASNYAESPWALSNWMSWVLPILSPLIPIFLLLLFGPCIFHLVSQFIQNRIQAITNHSI

SEQ ID NO: 133 - protein, 584 aa, Homo sapiens
MIFAGKAPSNTSTLMKFYSLLLYSLLFSFPFLCHPLPLPSYLHHTINLTHSLLAASNPSLVNNCWLCISLSSSAYTAVPAVQTDWATSPISLHLRTSFNSPHLYPPEELIYFLDRSSKTSPDISHQQAAALLRTYLKNLSPYINSTPPIFGPLTTQTTIPVAAPLCISWQRPTGIPLGNLSPSRCSFTLHLRSPTTNINETIGAFQLHITDKPSINTDKLKNISSNYCLGRHLPCISLHPWLSSPCSSDSPPRPSSCLLIPSPENNSERLLVDTRRFLIHHENRTFPSTQLPHQSPLQPLTAAALAGSLGVWVQDTPFSTPSHLFTLHLQFCLAQGLFFLCGSSTYMCLPANWTGTCTLVFLTPKIQFANGTEELPVPLMTPTQQKRVIPLIPLMVGLGLSASTVALGTGIAGISTSVMTFRSLSNDFSASITDISQTLSVLQAQVDSLAAVVLQNRRGLDLLTAEKGGLCIFLNEECCFYLNQSGLVYDNIKKLKDRAQKLANQASNYAEPPWALSNWMSWVLPIVSPLIPIFLLLLFGPCIFRLVSQFIQNRIQAITNHSIRQMFLLTSPQYHPLPQDLPSA

SEQ ID NO: 134 - protein, 563 aa, Homo sapiens
MIFAGRASSNTSTLMKFYSLLLYSLLFSFPILCHPLPLPSYLHHTINLTHSLLAVSNPSLAKNCWLCISLPSSAYPAVPALQTDWGTSPVSPHLRTSFNSPHLYPPEKLIYFLDRSSKTSPDISHQQAAALLCTYLKNLSPYINSTPPTFGPLTTQTTIPVAAPLCISRQRPTGIPLGNLSPSRCSFTLHLRSPTTHITETNGAFQLHITDKPSINTDKLKNVSSNYCLGRHLSCISLHPWLFSPCSSDSPPRPSSCLLIPSPKNNSESLLVDAQRFLIYHENRTSPSTQLPHQSPLQPLTAAPLGGSLRVWVQDTPFSTPSHLFTLHLQFCLVQSLFFLCGSSTYMCLPANWTGTCTLVFLTSKIQFANGTEELPVPLMTPTRQKRVIPLIPLMVGLGLSASTVALGTGIAGISTSVTTFRILSNDFSASITDISQTLSGLQAQVDSSAAVVLQNRQGLDLLTAEKGGLCIFLNEESYFYLNQSGLVYDNIKKLKDKAQNLANQASNYAEPPWPLSNWMSWVLPILSPLIPIFLLLFFRPCIFHLVSQFIQNHIQAITDHIS

SEQ ID NO: 135 - protein, 402 aa, Homo sapiens
MIFAGKAPSNTSTLMKFYSLLLYSLLFSFPFLCHPLPLPSYLHHTVNLTHSLLAASNPSLANNCWLCISLSSSAYTAVPVLQTDWATSPVSLHLRTSFNSPHLYPPEELIYFLDRSSKTSPDISHQQAAALLRTYLKNLSPYINSTPPIFGPLTTQTTIPVAAPLCISRQRPTGIPLGNLSPSRCSFTLHLRSPTTHITETIGAFQLHITDKPSINTDKLKNISSNYCLGRHVPCISLHPWLSSPCSSDSPPRPSSCLLIPSPENNSERLLVDTRRFLIHHENRTFPSTQLPHQSPLQPLTAAALAGSLGVWVQDTPFSTPSHLFTLHLQFCLAQALFFLCGSSTYMCLPANWTGTCTLVFLTRKIQFANGTEELPVPLMTPTQQKRVIPLIPLMVGLGLSA

SEQ ID NO: 136 - protein, 402 aa, Homo sapiens
MIFAGKAPSNTSTLMKFYSLLLYSLLFSFPFLCHPLPLPSYLHHTINLTHSLLAASNPSLVNNCWLCISLSSSAYTAVPTLQTDWATSPISLHLRTSCNSPHLYPPEELIYFLDRSSKTSPDISHQQAAALLRTYLKNLSPYINSTPPIFGPLTTQTTIPVAAPLCISWQRPTGIPLGNLSPSRCSFTLHLRSPTTNINETIGAFQLHITDKPSINTDKLKNISSNYCLGRHLPCISLHPWLSSPCSSDSPPRPSSCLLIPSPENNSERLLVDTRRFLIHHENRTFPSTQLPHQSPLQPLTAAALAGSLGVWVQDAPFSTPSHLFTLHLQFCLAQGLFFLCGSSTYMCLPANWTGTCTLVFLTPKIQFANGTEELPVPLMTPTQQKRVIPLIPLMVGLGLSA

SEQ ID NO: 137 - protein, 584 aa, Homo sapiens [sequence omitted]

SEQ ID NO: 138 - protein, 604 aa, Homo sapiens
MLGMNMLLITLFLLLPLSMLKGEPWEGCLHCTHTTWSGNIMTKTLLYHTYYECAGTCLGTCTHNQTTYSVCDPGRGQPYVCYDPKSSPGTWFEIHVGSKEGDLLNQTKVFPSGKDVVSLYFDVCQIVSMGSLFPVIFSSMEYYSSCHKNRYAHPACSTDSPVTTCWDCTTWSTNQQSLGPIMLTKIPLEPDCKTSTCNSVNLTILEPDQPIWTTGLKAPLGARVSGEEIGPGAYVYLYIIKKTRTRSTQQFRVFESFYEHVNQKLPEPPPLASNLFAQLAENIASSLHVASCYVCGGMNMGDQWPWEARELMPQDNFTLTASSLEPAPSSQSIWFLKTSIIGKFCIARWGKAFTDPVGELTCLGQQYYNETLGKTLWRGKSNNSESPHPSPFSRFPSLNHSWYQLEAPNTWQAPSGLYWICGPQAYRQLPAKWSGACVLGTIRPSFFLMPLKQGEALGYPIYDETKRKSKRGITIGDWKDNEWPPERIIQYYGPATWAEDGMWGYRTPVYMLNRIIRLQAVLEIITNETAGALNLLAQQATKMRNVIYQNRLALDYLLAQEEGVCGKFNLTNCCLELDDEGKVIKEITAKIQKLAHIPVQTWKG

SEQ ID NO: 139 - protein, 604 aa, Homo sapiens [sequence omitted]